(19) United States
(12) Patent Application Publication
(10) Pub. No.: US 2022/0344596 A1
(43) Pub. Date: Oct. 27, 2022

(54) LUMINESCENCE DEVICE AND COMPOUND INCLUDING NITROGEN FOR SAME

(71) Applicant: Samsung Display Co., Ltd., Yongin-si (KR)

(72) Inventor: Akinori YAMATANI, Yokohama (JP)

(21) Appl. No.: 17/687,710

(22) Filed: Mar. 7, 2022

(30) Foreign Application Priority Data
Apr. 5, 2021 (KR) 10-2021-0044251

(51) Int. Cl. H01L 51/00; C07D 209/86; C09K 11/06; C07D 405/10

(52) U.S. Cl. CPC H01L 51/0072; C07D 209/86; C09K 11/06; C07D 405/10; H01L 51/0073; H01L 51/5056

(57) ABSTRACT

A luminescence device, includes: a first electrode; a hole transport region disposed on the first electrode; an emission layer disposed on the hole transport region; an electron transport region disposed on the emission layer; and a second electrode disposed on the electron transport region, wherein the hole transport region includes a compound of Formula 1:

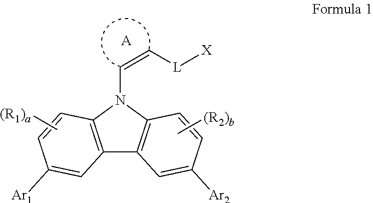

wherein in the Formula 1, the variables are described herein.

### CROSS-REFERENCE TO RELATED APPLICATIONS

This application claims priority from and the benefit of Korean Patent Application No. 10-2021-0044251, filed on Apr. 5, 2021, which is hereby incorporated by reference for all purposes as if fully set forth herein.

### BACKGROUND

### Field

Embodiments of the invention relate generally to display devices, and more particularly, a luminescence device and a nitrogen-containing compound for a luminescence device.

### Discussion of the Background

Recently, the use of an organic electroluminescence display as an image display is being actively developed. The organic electroluminescence display is different from a liquid crystal display and is so-called a self-luminescent display in which holes and electrons injected from a first electrode and a second electrode recombine in an emission layer so that a light-emitting material including an organic compound in the emission layer emits light to achieve display.

There is a demand for use of an organic electroluminescence device as a display, the decrease of its driving voltage, and the increase of emission efficiency and the lifespan of the organic electroluminescence device. Accordingly, there also is demand for materials for an organic electroluminescence device that can stably achieving these characteristics.

The above information disclosed in this Background section is only for understanding of the background of the inventive concepts, and, therefore, it may contain information that does not constitute prior art.

### SUMMARY

When luminescence devices include nitrogen-containing compounds made according to principles and illustrative implementations of the invention, the luminescence devices have high efficiency. Thus, nitrogen-containing compounds made according to the principles and illustrative implementations of the invention are capable of improving the efficiency and lifespan of luminescence devices that include such nitrogen-containing compounds in a hole transport region of the luminescence devices.

Additional features of the inventive concepts will be set forth in the description which follows, and in part will be apparent from the description, or may be learned by practice of the inventive concepts.

According to one aspect of the invention, a luminescence device includes: a first electrode; a hole transport region disposed on the first electrode; an emission layer disposed on the hole transport region; an electron transport region disposed on the emission layer; and a second electrode disposed on the electron transport region, wherein the hole transport region includes a compound of Formula 1:

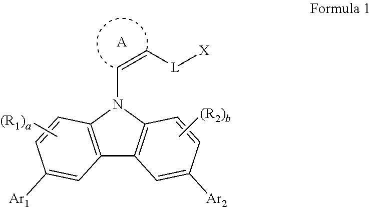

wherein in the Formula 1, the variables are described herein.

The hole transport region may include: a hole injection layer disposed on the first electrode; and a hole transport layer disposed on the hole injection layer, and the compound of the Formula 1 may be included in at least one of the hole injection layer and the hole transport layer.

The hole transport region may include: a hole transport layer disposed on the first electrode; an electron blocking layer disposed on the hole transport layer, and the electron blocking layer may include the compound of the Formula 1.

The group X may be a substituted or an unsubstituted heteroaryl group of 2 to 30 ring-forming carbon atoms, including any one of a furan ring, a thiophene ring, a pyrrole ring, or a silole ring.

The group X of the Formula 1 may be of Formula 3-1 or Formula 3-2, as described herein.

The ring B of the Formula 2 may be a substituted or an unsubstituted aryl ring of 6 to 18 ring-forming carbon atoms, or a substituted or an unsubstituted heteroaryl ring of 2 to 30 ring-forming carbon atoms, including any one of a furan ring, a thiophene ring, a pyrrole ring, or a silole ring.

The Formula 2 may be of Formula 4, as described herein.

The variable L of the Formula 1 may be a direct linkage or a substituted or an unsubstituted arylene group of 6 to 18 carbon atoms.

The Formula 1 may be of Formula 5, as described herein.

The variables Ar₁ and Ar₂ of the Formula 1 may be the same.

The Formula 5 may be of Formula 6-1, as described herein.

The Formula 5 may be of Formula 6-2, as described herein.

The compound of the Formula 1 may be at least one compound of Compound Group 1, as described herein.

According to another aspect of the invention, a compound is of Formula 1:

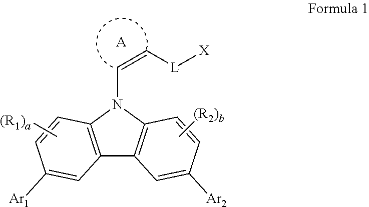

wherein in the Formula 1, the variables are described herein.

The Formula 1 may be of Formula 3-1 or Formula 3-2, as described herein.

The Formula 2 may be of Formula 4, as described herein.

The Formula 1 may be of Formula 5, as described herein.

The Formula 5 may be of Formula 6-1, as described herein.

The Formula 5 may be of Formula 6-2, as described herein.

The compound of the Formula 1 may be at least one compound of Compound Group 1, as described herein.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 is a plan view of an embodiment of a display apparatus constructed according to the principles of the invention.

FIG. 2 is a cross-sectional view taken along line I-I′ of FIG. 1.

FIG. 3 is a schematic cross-sectional view of an embodiment of a luminescence device constructed according to the principles of the invention.

FIG. 4 is a schematic cross-sectional view of another embodiment of a luminescence device constructed according to the principles of the invention.

FIG. 5 is a schematic cross-sectional view of a further embodiment of a luminescence device constructed according to the principles of the invention.

FIG. 6 is a schematic cross-sectional view of yet another embodiment of a luminescence device constructed according to the principles of the invention.

FIG. 7 is a cross-sectional view of another embodiment of a display apparatus including a luminescence device constructed according to the principles of the invention.

FIG. 8 is a cross-sectional view of a further embodiment of a display apparatus including a luminescence device constructed according to the principles of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, plates, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements, and repetitive explanations are omitted to avoid redundancy.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, numerals, integers, steps, operations, elements, parts, components, and/or groups thereof, but do not preclude the presence or addition of one or more other features, numerals, integers, steps, operations, elements, parts, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

### Definitions

As used herein, the term “atom” may mean an element or its corresponding radical bonded to one or more other atoms.

The terms “hydrogen” and “deuterium” refer to their respective atoms and corresponding radicals with the deuterium radical abbreviated “-D”, and the abbreviations “—F, —Cl, —Br, and —I” are radicals of, respectively, fluorine, chlorine, bromine, and iodine.

As used herein, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the exemplified substituents may be substituted or unsubstituted. For example, a biphenyl group may be interpreted as an aryl group or a phenyl group substituted with a phenyl group.

As used herein, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

As used herein, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

As used herein, the halogen atom may be a fluorine atom, a chlorine atom, a bromine atom, or an iodine atom.

As used herein, the alkyl group may be a linear, branched or cyclic type. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

As used herein, the hydrocarbon ring group means an optional functional group or substituent derived from an aliphatic hydrocarbon ring. The hydrocarbon ring group may be a saturated hydrocarbon ring group of 5 to 20 ring-forming carbon atoms.

As used herein, the aryl group means an optional functional group or substituent derived from an aromatic hydrocarbon ring. The aryl group may be a monocyclic aryl group or a polycyclic aryl group. The carbon number for forming the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

As used herein, the fluorenyl group may be substituted, and two substituents may be combined from each other to form a spiro structure. Examples of a substituted fluorenyl group are as follows. However, an embodiment is not limited thereto:

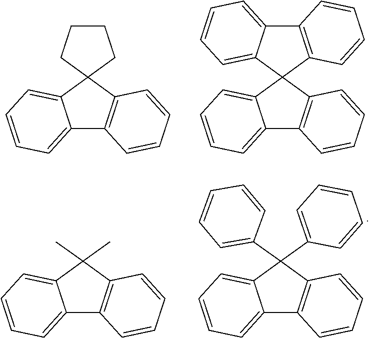

As used herein, the heterocyclic group may mean an optional functional group or substituent derived from a ring including one or more of B, O, N, P, Si and S as one or more heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

As used herein, the heterocyclic group may include one or more of B, O, N, P, Si and S as heteroatoms. In case where the heterocyclic group includes two or more heteroatoms, the two or more heteroatoms may be the same or different. The heterocyclic group may be a monocyclic heterocyclic group or a polycyclic heterocyclic group and has the concept including a heteroaryl group. The carbon number for forming rings of the heterocyclic group may be 2 to 30, 2 to 20, or 2 to 10.

As used herein, the aliphatic heterocyclic group may include one or more of B, O, N, P, Si and S as heteroatoms. The carbon number for forming rings of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

As used herein, the heteroaryl group may include one or more of B, O, N, P, Si and S as heteroatoms. If the heteroaryl group includes two or more heteroatoms, two or more heteroatoms may be the same or different. The heteroaryl group may be a monocyclic heterocyclic group or a polycyclic heterocyclic group. The carbon number for forming rings of the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofurane, phenanthroline, thiazole, isooxazole, oxazole, oxadiazole, thiadiazole, phenothiazine, dibenzosilole, dibenzofuran, etc., without limitation.

As used herein, the explanation on the aryl group may be applied to an arylene group except that the arylene group is a divalent group. The explanation on the heteroaryl group may be applied to a heteroarylene group except that the heteroarylene group is a divalent group.

As used herein, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

As used herein, a thio group may include an alkyl thio group and an aryl thio group. The thio group may mean the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

As used herein, the alkenyl group may be a linear chain or a branched chain. The carbon number is not specifically limited but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

As used herein, the carbon number of the amine group is not specifically limited, but may be 1 to 30. The amine group may include an alkyl amine group, an aryl amine group, or a heteroaryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

As used herein, the alkyl group in an alkylthio group, an alkylsulfoxy group, an alkylaryl group, an alkylamino group, an alkylboron group, an alkyl silyl group, and an alkyl amine group may be the same as the examples of the above-described alkyl group.

As used herein, the aryl group in an aryloxy group, an arylthio group, an arylsulfoxy group, an aryl amino group, an arylboron group, and an aryl silyl group may be the same as the examples of the above-described aryl group.

As used herein, a direct linkage may mean a single bond.

As used herein, “ [symbol] ” means a position to be connected.

Hereinafter, embodiments are explained referring to the drawings.

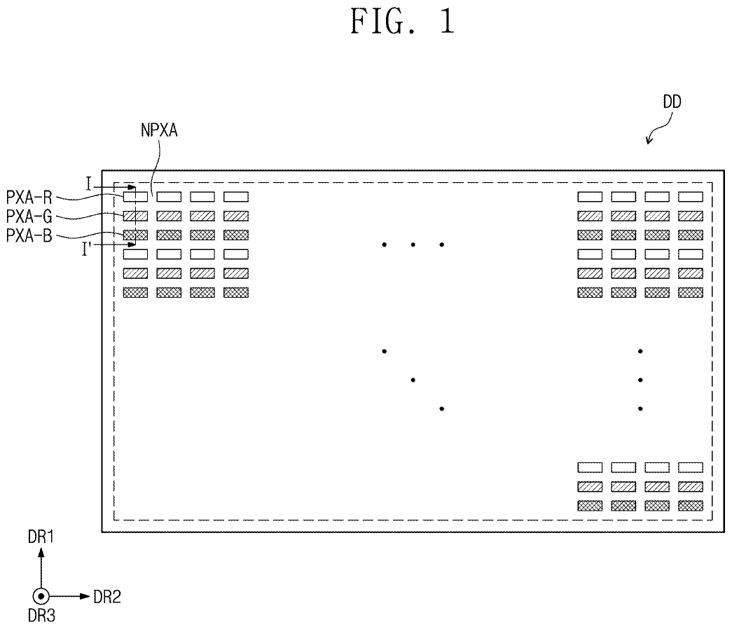

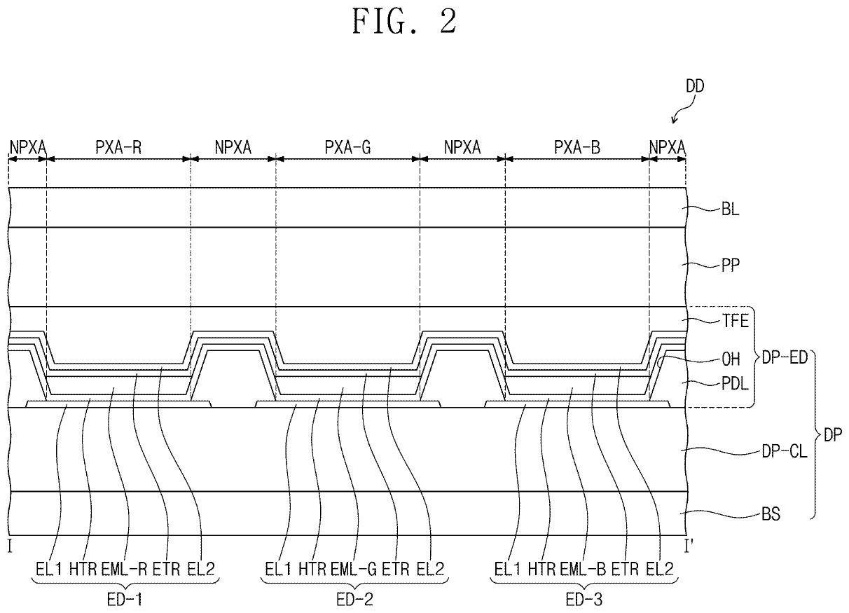

### FIGS. 1-2

FIG. 1 is a plan view of an embodiment of a display apparatus constructed according to the principles of the invention. FIG. 2 is a cross-sectional view taken along line I-I′ of FIG. 1.

The display apparatus DD may include a display panel DP and an optical layer PP disposed on the display panel DP. The display panel DP includes luminescence devices ED-1, ED-2 and ED-3. The display apparatus DD may include multiple luminescence devices ED-1, ED-2 and ED-3. The optical layer PP may be disposed on the display panel DP and control reflected light by external light at the display panel DP. The optical layer PP may include, for example, a polarization layer or a color filter layer. Unlike the view illustrated in FIG. 2, the optical layer PP may be omitted in the display apparatus DD.

On the optical layer PP, a base substrate BL may be disposed. The base substrate BL may be a member providing a base surface where the optical layer PP is disposed. The base substrate BL may be a glass substrate, a metal substrate, a plastic substrate, etc. However, embodiments of the invention are not limited thereto, and the base substrate BL may be an inorganic layer, an organic layer or a composite material layer. In addition, Unlike the drawings, the base substrate BL may be omitted in an embodiment.

The display apparatus DD may further include a plugging layer. The plugging layer may be disposed between a display device layer DP-ED and a base substrate BL. The plugging layer may be an organic layer. The plugging layer may include at least one of an acrylic resin, a silicon-based resin and an epoxy-based resin.

The display panel DP may include a base layer BS, a circuit layer DP-CL provided on the base layer BS and a display device layer DP-ED. The display device layer DP-ED may include a pixel definition layer PDL, the luminescence devices ED-1, ED-2 and ED-3 disposed in the pixel definition layer PDL, and an encapsulation layer TFE disposed on the luminescence devices ED-1, ED-2 and ED-3.

The base layer BS may be a member providing a base surface where the display device layer DP-ED is disposed. The base layer BS may be a glass substrate, a metal substrate, a plastic substrate, etc. However, the embodiments are not limited thereto, and the base layer BS may be an inorganic layer, an organic layer or a composite material layer.

In an embodiment, the circuit layer DP-CL is disposed on the base layer BS, and the circuit layer DP-CL may include multiple transistors. Each of the transistors may include a control electrode, an input electrode, and an output electrode. For example, the circuit layer DP-CL may include switching transistors and driving transistors for driving the luminescence devices ED-1, ED-2 and ED-3 of the display device layer DP-ED.

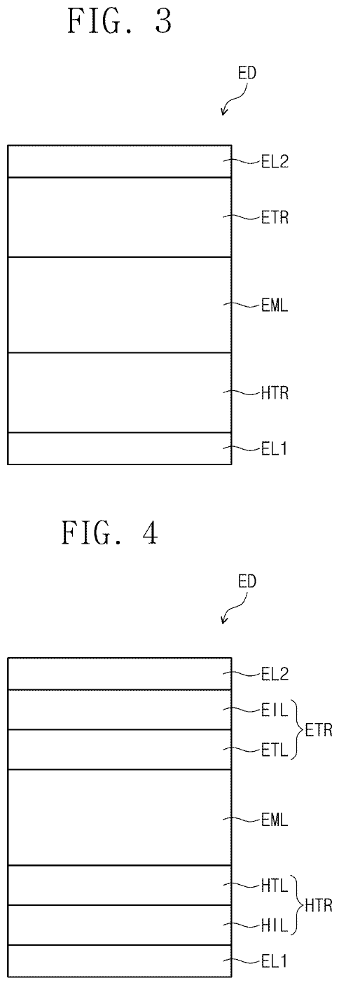

Each of the luminescence devices ED-1, ED-2 and ED-3 may have the structures of luminescence devices ED according to FIG. 3 to FIG. 6, which will be explained below. Each of the luminescence devices ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of luminescence devices ED-1, ED-2 and ED-3, which are in opening portions OH defined in a pixel definition layer PDL, are disposed, and a hole transport region HTR, an electron transport region ETR and a second electrode EL2 are provided as common layers in all luminescence devices ED-1, ED-2 and ED-3. However, the embodiments are not limited thereto. Unlike FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the luminescence devices ED-1, ED-2 and ED-3 may be patterned by an ink jet printing method.

An encapsulation layer TFE may cover the luminescence devices ED-1, ED-2 and ED-3. The encapsulation layer TFE may encapsulate the display device layer DP-ED. The encapsulation layer TFE may be a thin film encapsulating layer. The encapsulation layer TFE may be one layer or a stacked layer of multiple layers. The encapsulation layer TFE includes at least one insulating layer. The encapsulation layer TFE may include at least one inorganic layer (hereinafter, encapsulating inorganic layer). In addition, the encapsulation layer TFE may include at least one organic layer (hereinafter, encapsulating organic layer) and at least one encapsulating inorganic layer.

The encapsulating inorganic layer protects the display device layer DP-ED from at least one of moisture and oxygen, and the encapsulating organic layer protects the display device layer DP-ED from foreign materials such as dust particles. The encapsulating inorganic layer may include a silicon nitride, a silicon oxy nitride, a silicon oxide, a titanium oxide, or an aluminum oxide, without specific limitation. The encapsulating organic layer may include an acrylic compound, an epoxy-based compound, etc. The encapsulating organic layer may include a photopolymerizable organic material, without specific limitation. The encapsulation layer TFE may be disposed on the second electrode EL2 and may be disposed while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas emitting light produced from the luminescence devices ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. As described herein, each of the luminous areas PXA-R, PXA-G and PXA-B may overlap each pixel. The pixel definition layer PDL may divide the luminescence devices ED-1, ED-2 and ED-3. The emission layers EML-R, EML-G and EML-B of the luminescence devices ED-1, ED-2 and ED-3 may be disposed and divided in the opening portions OH defined in the pixel definition layer PDL.

The luminous areas PXA-R, PXA-G and PXA-B may be divided into multiple groups according to the color of light produced from the luminescence devices ED-1, ED-2 and ED-3. In the display apparatus DD shown in FIG. 1 and FIG. 2, three luminous areas PXA-R, PXA-G and PXA-B emitting red light, green light and blue light are illustrated as an embodiment. For example, the display apparatus DD may include a red luminous area PXA-R, a green luminous area PXA-G and a blue luminous area PXA-B, which are separated from each other.

In the display apparatus DD, multiple luminescence devices ED-1, ED-2 and ED-3 may emit light having different wavelength regions. For example, in an embodiment, the display apparatus DD may include a first luminescence device ED-1 emitting red light, a second luminescence device ED-2 emitting green light, and a third luminescence device ED-3 emitting blue light. That is, each of the red luminous area PXA-R, the green luminous area PXA-G, and the blue luminous area PXA-B of the display apparatus DD may overlap the first luminescence device ED-1, the second luminescence device ED-2, and the third luminescence device ED-3.

However, the embodiments are not limited thereto, and the first to third luminescence devices ED-1, ED-2 and ED-3 may emit light in the same wavelength region, or at least one thereof may emit light in a different wavelength region. For example, all the first to third luminescence devices ED-1, ED-2 and ED-3 may emit blue light.

The luminous areas PXA-R, PXA-G and PXA-B in the display apparatus DD may be arranged in a generally elongated (stripe) shape. Referring to FIG. 1, multiple red luminous areas PXA-R, multiple green luminous areas PXA-G and multiple blue luminous areas PXA-B may be arranged along a second direction axis DR2. In addition, the red luminous area PXA-R, the green luminous area PXA-G and the blue luminous area PXA-B may be alternatively arranged along a first direction axis DR1.

In FIG. 1 and FIG. 2, the areas of the luminous areas PXA-R, PXA-G and PXA-B are similarly depicted, but the embodiments are not limited thereto. The areas of the luminous areas PXA-R, PXA-G and PXA-B may be different from each other according to the wavelength region of light emitted. The areas of the luminous areas PXA-R, PXA-G and PXA-B may mean planar areas defined by the first direction axis DR1 and the second direction axis DR2.

The configuration of the luminous areas PXA-R, PXA-G and PXA-B is not limited to the features shown in FIG. 1, and the arrangement order of the red luminous areas PXA-R, the green luminous areas PXA-G and the blue luminous areas PXA-B may be provided in various combinations according to the properties of display quality required for the display apparatus DD. For example, the configuration of the luminous areas PXA-R, PXA-G and PXA-B may be a configuration sold under the trade designation PenTile matrix by Samsung Display Co., Ltd. of Gyeonggi-do, Republic of Korea, or a diamond configuration.

In addition, the areas of the luminous areas PXA-R, PXA-G and PXA-B may be different from each other. For example, in an embodiment, the area of the green luminous area PXA-G may be smaller than the area of the blue luminous area PXA-B, but the embodiments are not limited thereto.

### FIGS. 3-6

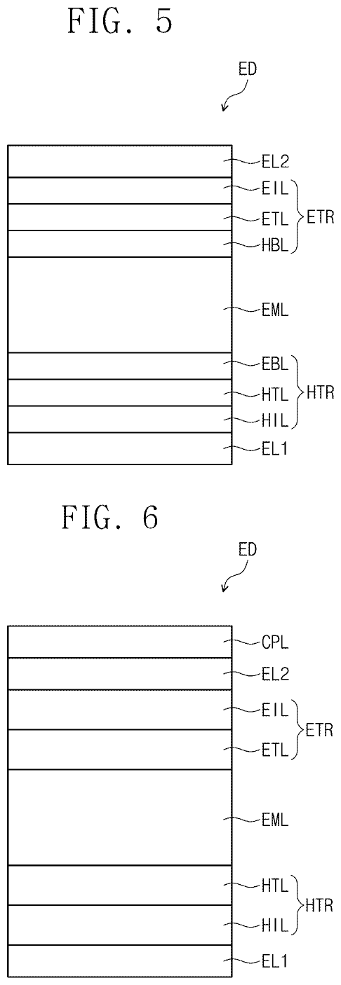

FIG. 3 is a schematic cross-sectional view of an embodiment of a luminescence device constructed according to the principles of the invention. FIG. 4 is a schematic cross-sectional view of another embodiment of a luminescence device constructed according to the principles of the invention. FIG. 5 is a schematic cross-sectional view of a further embodiment of a luminescence device constructed according to the principles of the invention. FIG. 6 is a schematic cross-sectional view of yet another embodiment of a luminescence device constructed according to the principles of the invention.

Hereinafter, FIG. 3 to FIG. 6 are cross-sectional views schematically showing various embodiments of luminescence devices The embodiments of the luminescence device ED may include a first electrode EL1, a hole transport region HTR, an emission layer EML, and a second electrode EL2 stacked in order.

When compared with FIG. 3, FIG. 4 shows the cross-sectional view of a luminescence device ED, wherein a hole transport region HTR includes a hole injection layer HIL and a hole transport layer HTL, and an electron transport region ETR includes an electron injection layer EIL and an electron transport layer ETL. When compared with FIG. 3, FIG. 5 shows the cross-sectional view of a luminescence device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a luminescence device ED including a capping layer CPL disposed on the second electrode EL2.

The first electrode EL1 has conductivity. The first electrode EL1 may be formed using a metal alloy or a conductive compound. The first electrode EL1 may be an anode or a cathode. However, the embodiments are not limited thereto. In addition, the first electrode EL1 may be a pixel electrode. The first electrode EL1 may be a transmissive electrode, a transflective electrode, or a reflective electrode. If the first electrode EL1 is the transmissive electrode, the first electrode EL1 may include a transparent metal oxide such as an indium tin oxide (ITO), an indium zinc oxide (IZO), a zinc oxide (ZnO), and an indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, compounds thereof, or mixtures thereof (for example, a mixture of Ag and Mg). Also, the first electrode EL1 may have a structure of multiple layers including a reflective layer or a transflective layer formed using the above materials, and a transmissive conductive layer formed using the ITO, IZO, ZnO, or ITZO. For example, the first electrode EL1 may include a three-layer structure of an ITO/Ag/ITO. However, the embodiments are not limited thereto. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a hole buffer layer, and an electron blocking layer EBL. The thickness of the hole transport region HTR may be, for example, from about 50 Å to about 15,000 Å. The hole transport region HTR may have a single layer formed using a single material, a single layer formed using multiple different materials, or a multilayer structure including multiple layers formed using multiple different materials.

For example, the hole transport region HTR may have the structure of a single layer of a hole injection layer HIL or a hole transport layer HTL, and may have a structure of a single layer formed using a hole injection material and a hole transport material. Otherwise, the hole transport region HTR may have a structure of a single layer formed using multiple different materials, or a structure stacked from the first electrode EL1 of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL, without limitation.

The hole transport region HTR of the luminescence device ED includes an amine compound made according to an embodiment of the invention. Embodiments of the nitrogen-containing compound are represented by Formula 1 below.

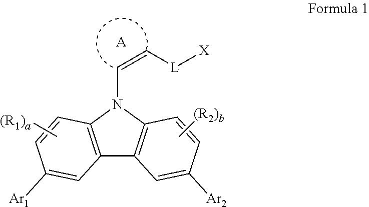

In Formula 1, X is a heteroaryl group of 2 to 30 ring-forming carbon atoms, including any one of O, S, N and Si. In Formula 1, L is a direct linkage, a substituted or unsubstituted arylene group of 6 to 30 ring-forming carbon atoms, or a substituted or unsubstituted heteroarylene group of 2 to 30 ring-forming carbon atoms. In Formula 1, ring A is a substituted or unsubstituted aryl ring of 6 to 30 ring-forming carbon atoms, or a substituted or unsubstituted heteroaryl ring of 2 to 30 ring-forming carbon atoms. In Formula 1, “a” and “b” are each independently an integer of 0 to 3, if “a” is 2 or more, multiple R₁ groups are the same or different, and if “b” is 2 or more, multiple R₂ groups are the same or different.

In Formula 1, R₁ and R₂ are each independently a hydrogen atom, a deuterium atom, a halogen atom, a nitro group, a cyano group, a hydroxyl group, a substituted or unsubstituted amine group, a substituted or unsubstituted thiol group, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or combined with an adjacent group to form a ring.

In Formula 1, Ar₁ and Ar₂ are each independently represented by Formula 2 below.

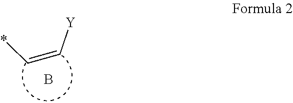

In Formula 2, Y is a substituted or unsubstituted aryl group of 6 to 30 ring-forming carbon atoms, or a substituted or unsubstituted heteroaryl group of 2 to 30 ring-forming carbon atoms, including any one of a furan ring, a thiophene ring, a pyrrole ring, and a silole ring. In Formula 2, ring B is a substituted or unsubstituted aryl ring of 6 to 30 ring-forming carbon atoms, or a substituted or unsubstituted heteroaryl ring of 2 to 30 ring-forming carbon atoms. The symbol * is a binding site to an adjacent atom.

In an embodiment, X of Formula 1 may be a substituted or unsubstituted heteroaryl group of 2 to 30 ring-forming carbon atoms, including any one of a furan ring, a thiophene ring, a pyrrole ring, and a silole ring.

In an embodiment, L of Formula 1 may be a direct linkage or a substituted or unsubstituted arylene group of 6 to 18 ring-forming carbon atoms. For example, L of Formula 1 may be a substituted or unsubstituted phenylene group, a substituted or unsubstituted biphenylene group, or a substituted or unsubstituted terphenylene group. In an embodiment, X of Formula 1 may be represented by Formula 3-1 or Formula 3-2 below.

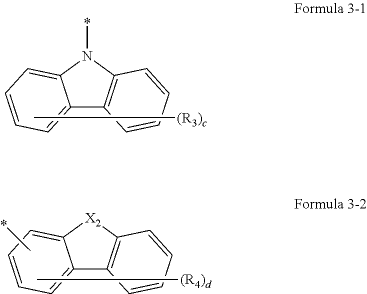

In Formula 3-1 or Formula 3-2, R₃ and R₄ may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or combined with an adjacent group to form a ring.

In Formula 3-1, “c” is an integer of 0 to 8, if “c” is 2 or more, multiple R₃ groups are the same or different. In Formula 3-2, “d” is an integer of 0 to 7, if “d” is 2 or more, multiple R₄ groups are the same or different.

In Formula 3-2, X₂ may be O, S, NAr₃, or SiAr₄Ar₅, and Ar₃ to Ar₅ may be each independently a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. The symbol * is a binding site to an adjacent atom

In an embodiment, Formula 1 may be represented by Formula 5 below:

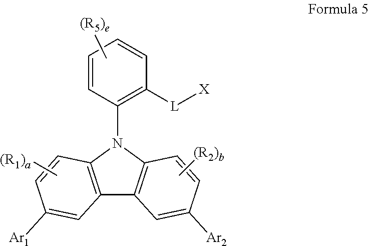

In Formula 5, R₅ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula 5, “e” is an integer of 0 to 4, if “e” is 2 or more, multiple R₅ groups are the same or different. In Formula 5, R₁, R₂, L, X, Ar₁, Ar₂, “a”, and “b” are the same as defined in Formula 1.

In an embodiment, Ar₁ and Ar₂ of Formula 1 may be the same. In an embodiment,

Formula 5 may be represented by Formula 6-1 below.

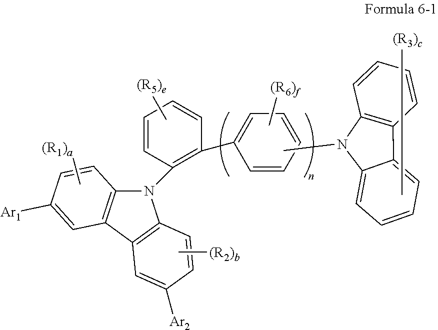

In Formula 6-1, R₃ and R₄ may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula 6-1, “c” is an integer of 0 to 8, if “c” is 2 or more, multiple R₃ groups are the same or different. In Formula 6-1, “f” is an integer of 0 to 4, if “f” is 2 or more, multiple R₆ groups are the same or different. In Formula 6-1, “n” may be an integer of 0 to 3. In Formula 6-1, R₁, R₂, R₅, Ar₁, Ar₂, “a”, “b”, and “e” are the same as defined in Formula 5.

In an embodiment, Formula 5 may be represented by Formula 6-2 below. Formula 6-2

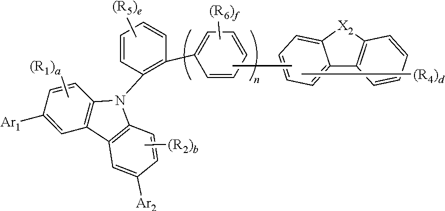

In Formula 6-2, R₄ and R₆ may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula 6-2, “d” is an integer of 0 to 7, if “d” is 2 or more, multiple R₄ groups are the same or different. In Formula 6-2, “f” is an integer of 0 to 4, if “f” is 2 or more, multiple R₆ groups are the same or different. In Formula 6-2, “n” may be an integer of 0 to 3.

In Formula 6-2, X₂ may be O, S, NAr₃, or SiAr₄Ar₅, and Ar₃ to Ar₅ may be each independently a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In Formula 6-2, R₁, R₂, R₅, Ar₁, Ar₂, “a”, “b”, and “e” are the same as defined in Formula 5. In an embodiment, Formula 2 may be represented by Formula 4 below.

### Formula 4

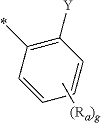

In Formula 4, Rₐ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms, or combined with an adjacent group to form a ring. In Formula 4, “g” is an integer of 0 to 4, if “g” is 2 or more, multiple Rₐ groups are the same or different. In Formula 4, Y is the same as defined in Formula 2. The symbol * is a binding site to an adjacent atom

Embodiments of the nitrogen-containing compound represented by Formula 1 may be any one selected of the compounds represented in Compound Group 1 below. However, the embodiments of the invention are not limited thereto.

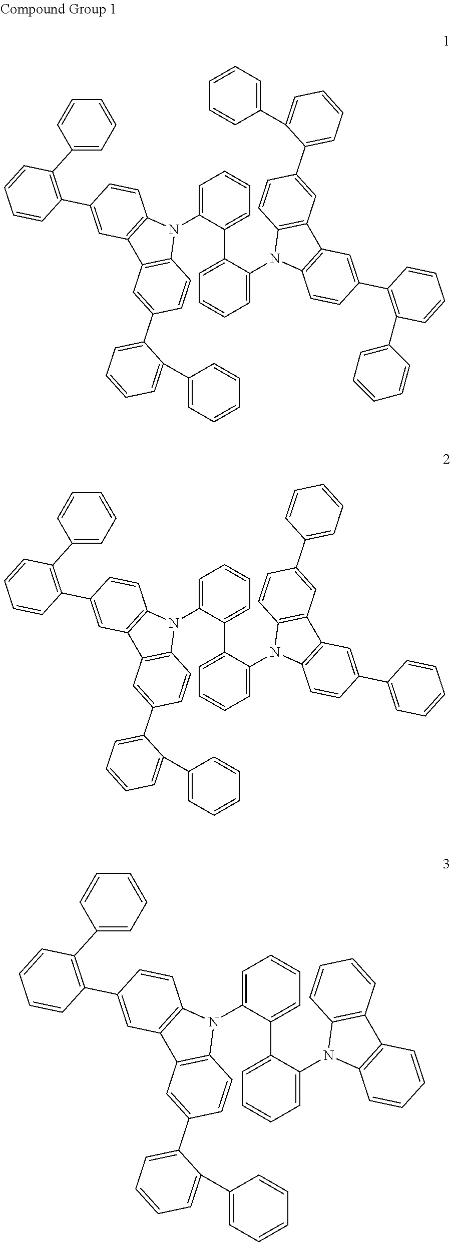 [SMILES: c1ccc(-c2ccccc2-c2ccc3c(c2)c2cc(-c4ccccc4-c4ccccc4)ccc2n3-c2ccccc2-c2ccccc2-n2c3ccc(-c4ccccc4-c4ccccc4)cc3c3cc(-c4ccccc4-c4ccccc4)ccc32)cc1 ; c1ccc(-c2ccc3c(c2)c2cc(-c4ccccc4)ccc2n3-c2ccccc2-c2ccccc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccccc2-c2ccccc2-n2c3ccccc3c3ccccc32)cc1]

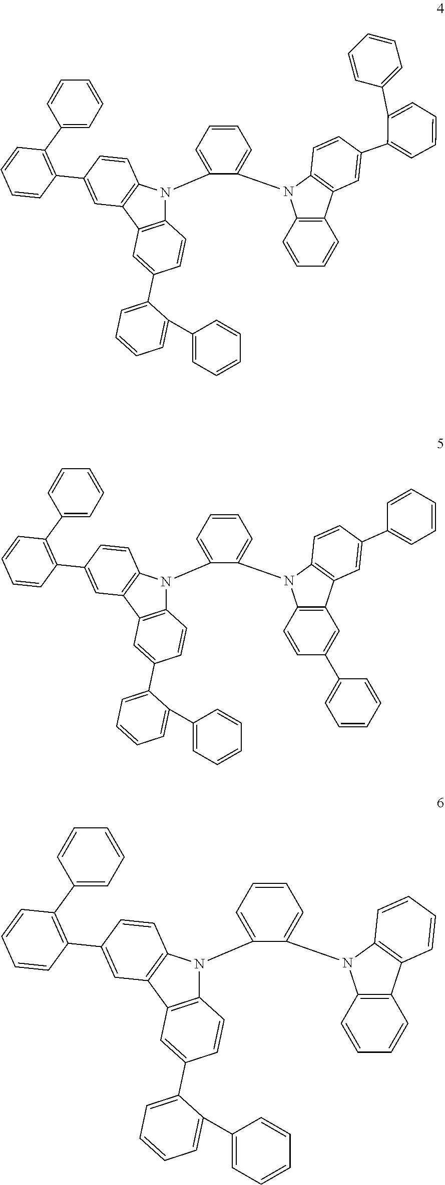 [SMILES: c1ccc(-c2ccccc2-c2ccc3c(c2)c2ccccc2n3-c2ccccc2-n2c3ccc(-c4ccccc4-c4ccccc4)cc3c3cc(-c4ccccc4-c4ccccc4)ccc32)cc1 ; c1ccc(-c2ccc3c(c2)c2cc(-c4ccccc4)ccc2n3-c2ccccc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccccc2-n2c3ccccc3c3ccccc32)cc1]

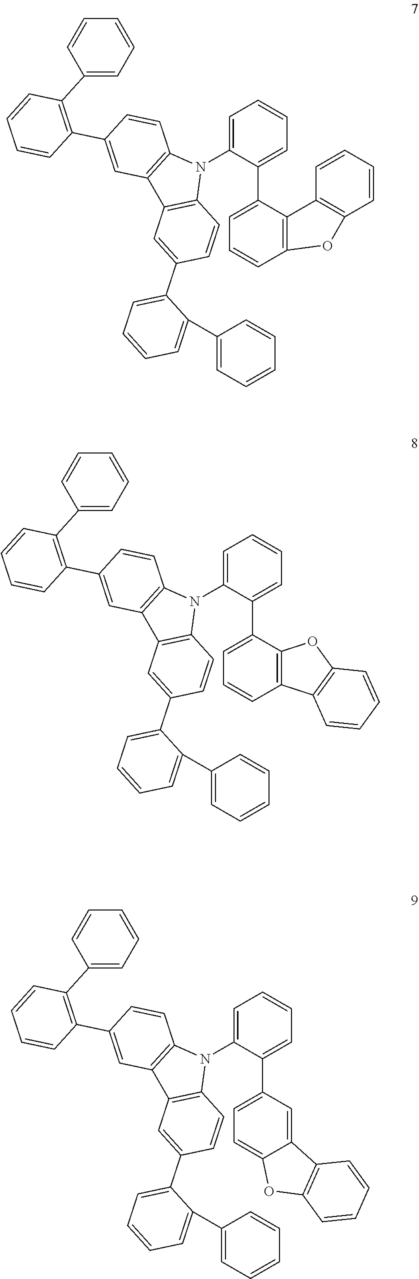 [SMILES: c1ccc(-c2ccccc2-c2ccc3c(c2)c2cc(-c4ccccc4-c4ccccc4)ccc2n3-c2ccccc2-c2cccc3oc4ccccc4c23)cc1 ; c1ccc(-c2ccccc2-c2ccc3c(c2)c2cc(-c4ccccc4-c4ccccc4)ccc2n3-c2ccccc2-c2cccc3c2oc2ccccc23)cc1 ; c1ccc(-c2ccccc2-c2ccc3c(c2)c2cc(-c4ccccc4-c4ccccc4)ccc2n3-c2ccccc2-c2ccc3oc4ccccc4c3c2)cc1]

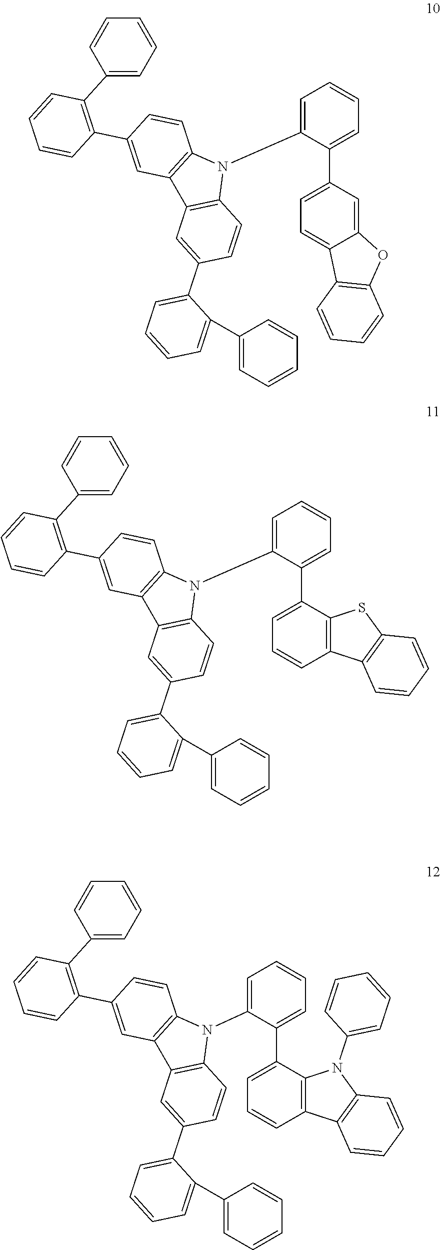 [SMILES: c1ccc(-c2ccccc2-c2ccc3c(c2)c2cc(-c4ccccc4-c4ccccc4)ccc2n3-c2ccccc2-c2ccc3c(c2)oc2ccccc23)cc1 ; c1ccc(-c2ccccc2-c2ccc3c(c2)c2cc(-c4ccccc4-c4ccccc4)ccc2n3-c2ccccc2-c2cccc3c2sc2ccccc23)cc1 ; c1ccc(-c2ccccc2-c2ccc3c(c2)c2cc(-c4ccccc4-c4ccccc4)ccc2n3-c2ccccc2-c2cccc3c4ccccc4n(-c4ccccc4)c23)cc1]

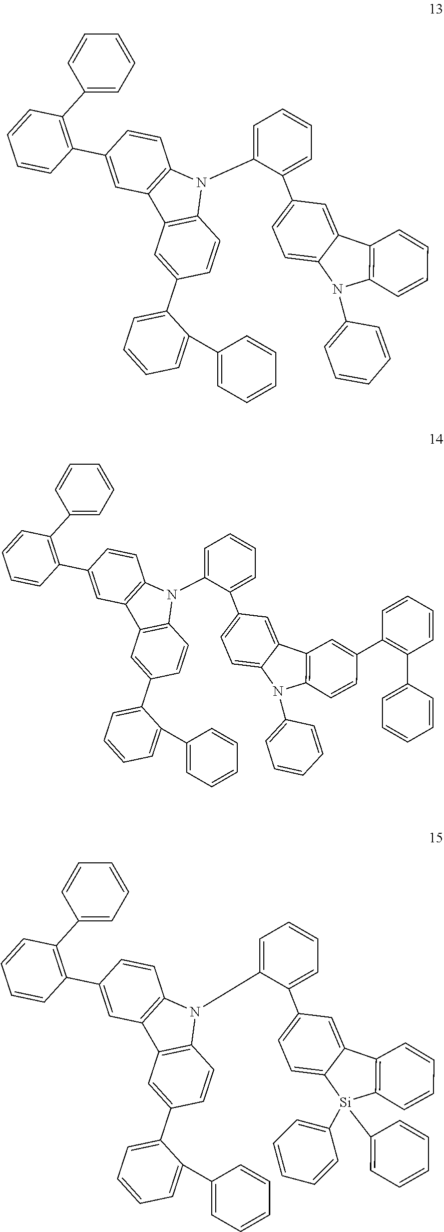 [SMILES: c1ccc(-c2ccccc2-c2ccc3c(c2)c2cc(-c4ccccc4-c4ccccc4)ccc2n3-c2ccccc2-c2ccc3c(c2)c2ccccc2n3-c2ccccc2)cc1 ; c1ccc(-c2ccccc2-c2ccc3c(c2)c2cc(-c4ccccc4-n4c5ccc(-c6ccccc6-c6ccccc6)cc5c5cc(-c6ccccc6-c6ccccc6)ccc54)ccc2n3-c2ccccc2)cc1 ; c1ccc(-c2ccccc2-c2ccc3c(c2)c2cc(-c4ccccc4-c4ccccc4)ccc2n3-c2ccccc2-c2ccc3c(c2)-c2ccccc2[Si]3(c2ccccc2)c2ccccc2)cc1]

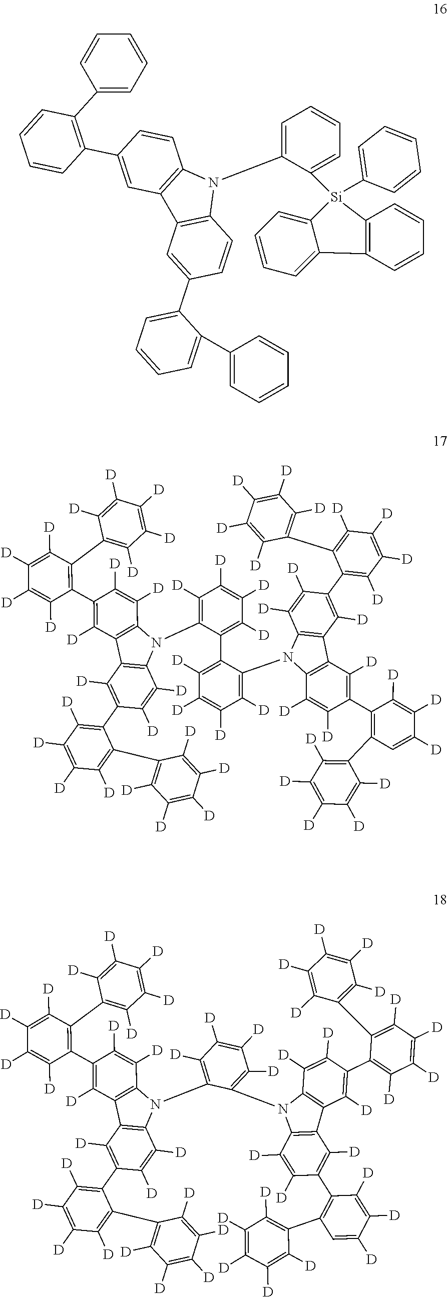

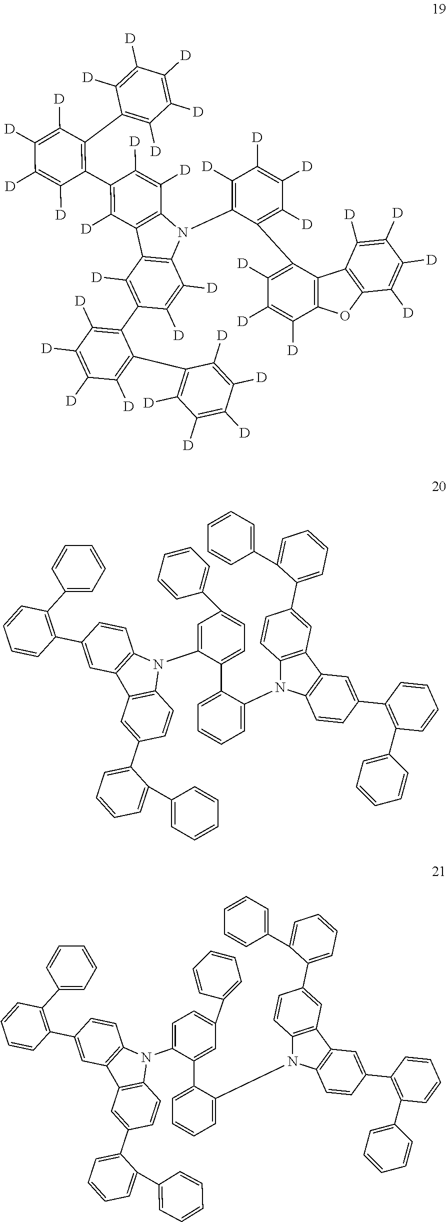

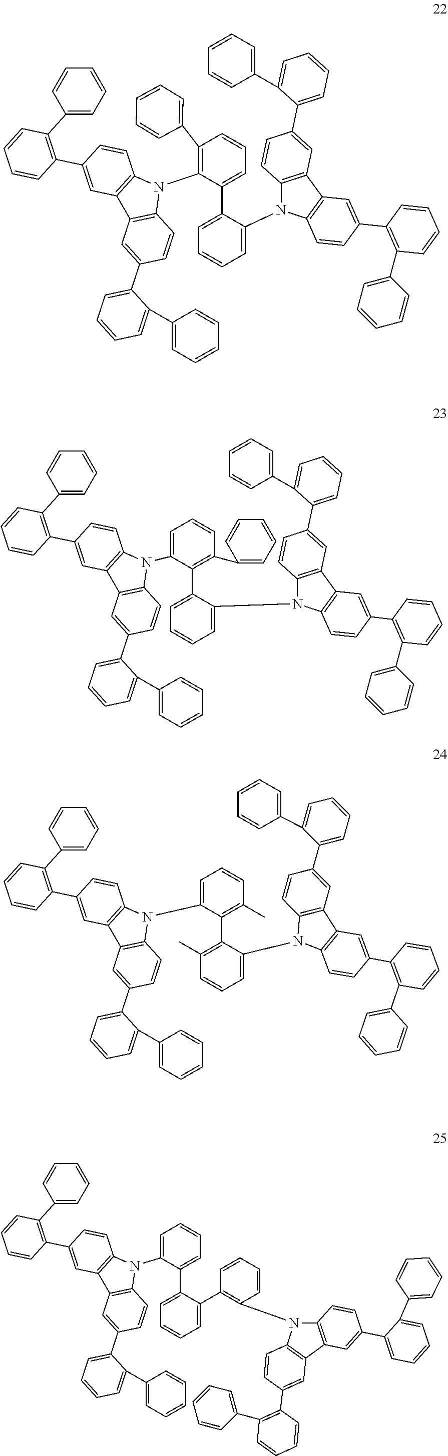

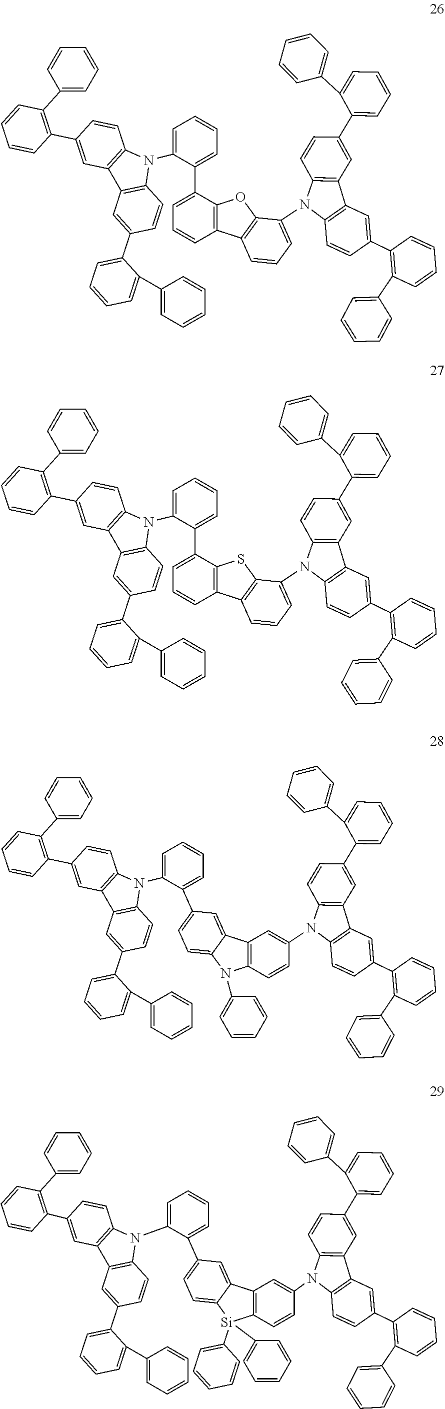

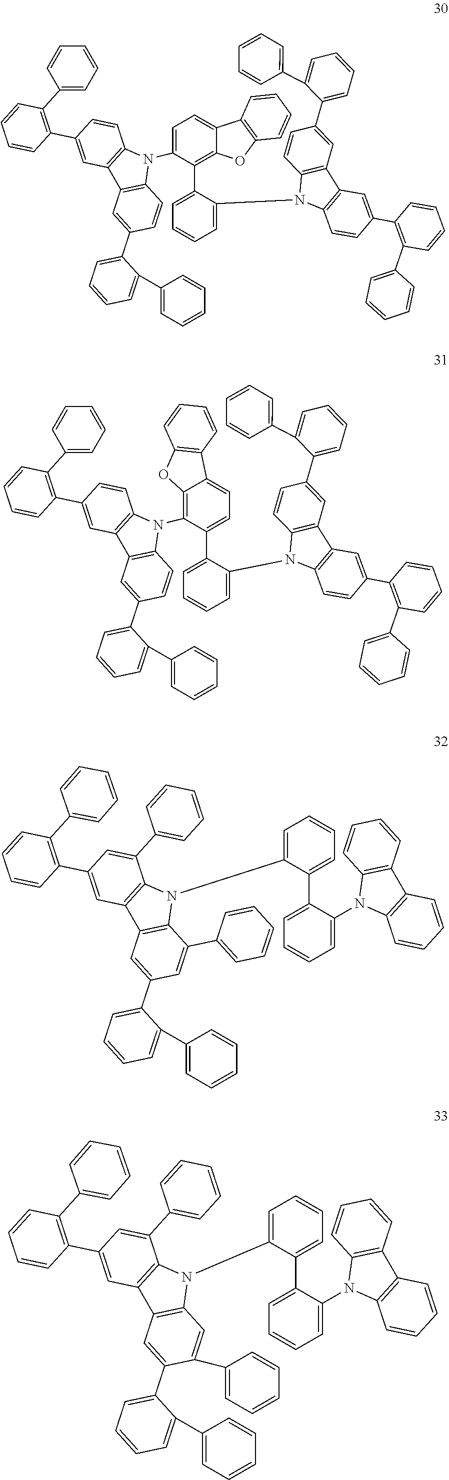

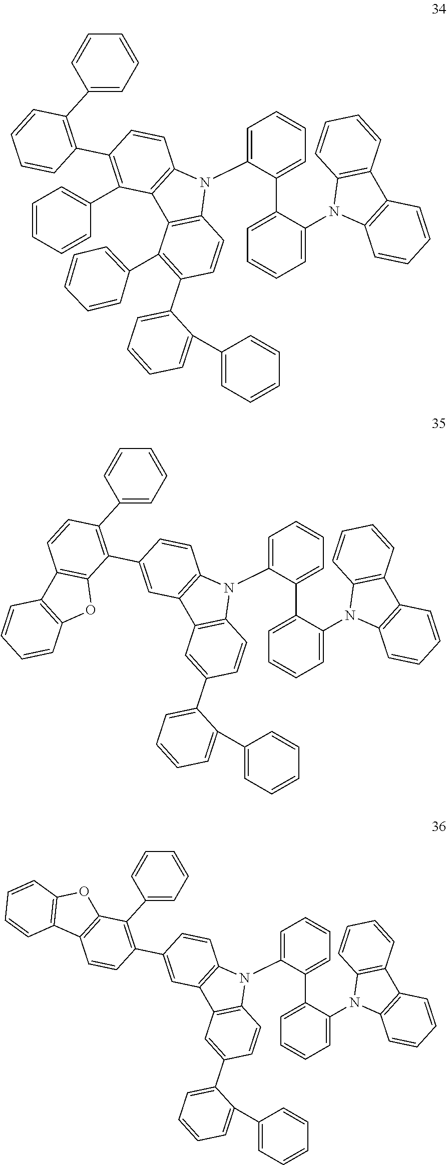 [SMILES: c1ccc(-c2ccccc2-c2ccc3c(c2-c2ccccc2)c2c(-c4ccccc4)c(-c4ccccc4-c4ccccc4)ccc2n3-c2ccccc2-c2ccccc2-n2c3ccccc3c3ccccc32)cc1 ; c1ccc(-c2ccccc2-c2ccc3c(c2)c2cc(-c4c(-c5ccccc5)ccc5c4oc4ccccc45)ccc2n3-c2ccccc2-c2ccccc2-n2c3ccccc3c3ccccc32)cc1 ; c1ccc(-c2ccccc2-c2ccc3c(c2)c2cc(-c4ccc5c(oc6ccccc65)c4-c4ccccc4)ccc2n3-c2ccccc2-c2ccccc2-n2c3ccccc3c3ccccc32)cc1]

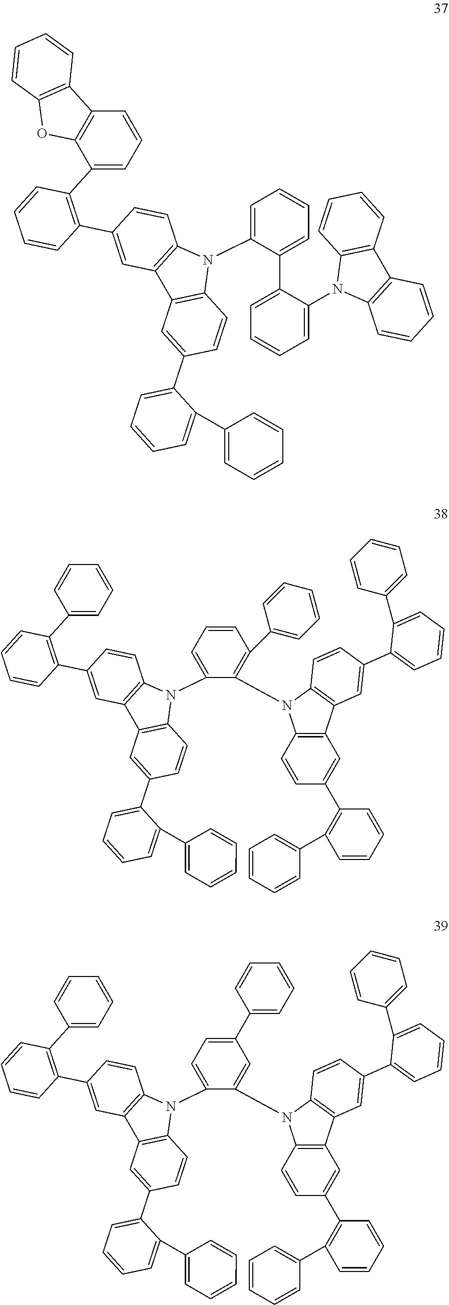 [SMILES: c1ccc(-c2ccccc2-c2ccc3c(c2)c2cc(-c4ccccc4-c4cccc5c4oc4ccccc45)ccc2n3-c2ccccc2-c2ccccc2-n2c3ccccc3c3ccccc32)cc1 ; c1ccc(-c2ccccc2-c2ccc3c(c2)c2cc(-c4ccccc4-c4ccccc4)ccc2n3-c2cccc(-c3ccccc3)c2-n2c3ccc(-c4ccccc4-c4ccccc4)cc3c3cc(-c4ccccc4-c4ccccc4)ccc32)cc1 ; c1ccc(-c2ccc(-n3c4ccc(-c5ccccc5-c5ccccc5)cc4c4cc(-c5ccccc5-c5ccccc5)ccc43)c(-n3c4ccc(-c5ccccc5-c5ccccc5)cc4c4cc(-c5ccccc5-c5ccccc5)ccc43)c2)cc1]

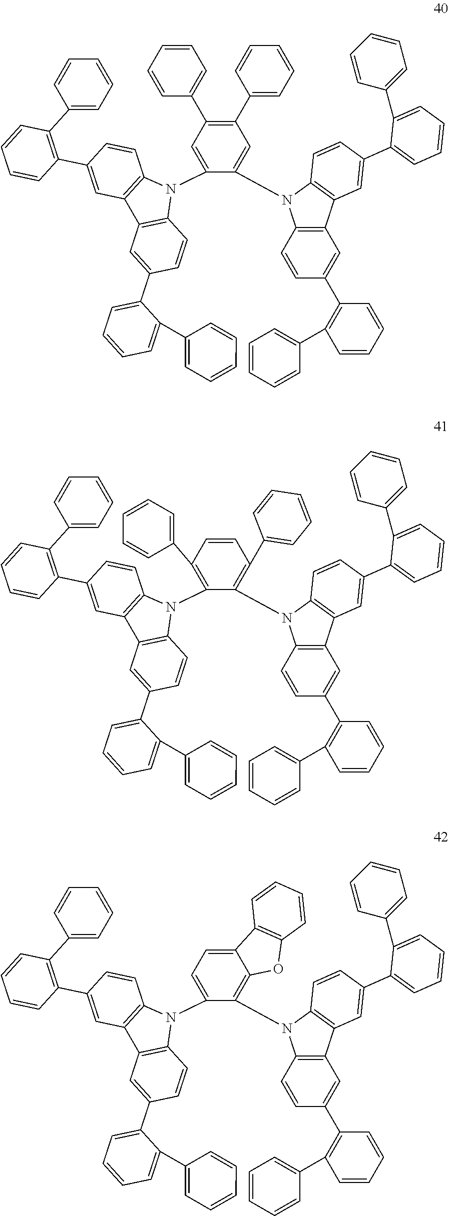 [SMILES: c1ccc(-c2ccccc2-c2ccc3c(c2)c2cc(-c4ccccc4-c4ccccc4)ccc2n3-c2cc(-c3ccccc3)c(-c3ccccc3)cc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2c(-c3ccccc3)ccc(-c3ccccc3)c2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccc3c(oc4ccccc43)c2-n2c3ccc(-c4ccccc4-c4ccccc4)cc3c3cc(-c4ccccc4-c4ccccc4)ccc32)cc1]

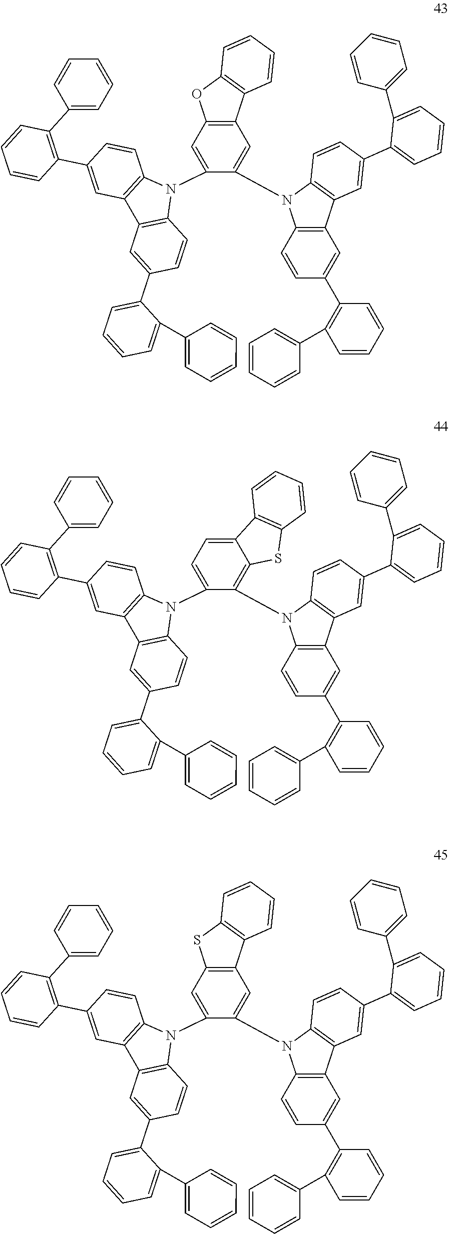 [SMILES: c1ccc(-c2ccccc2-c2ccc3c(c2)c2cc(-c4ccccc4-c4ccccc4)ccc2n3-c2cc3oc4ccccc4c3cc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccc3c(sc4ccccc43)c2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2cc3sc4ccccc4c3cc2-n2c3ccc(-c4ccccc4-c4ccccc4)cc3c3cc(-c4ccccc4-c4ccccc4)ccc32)cc1]

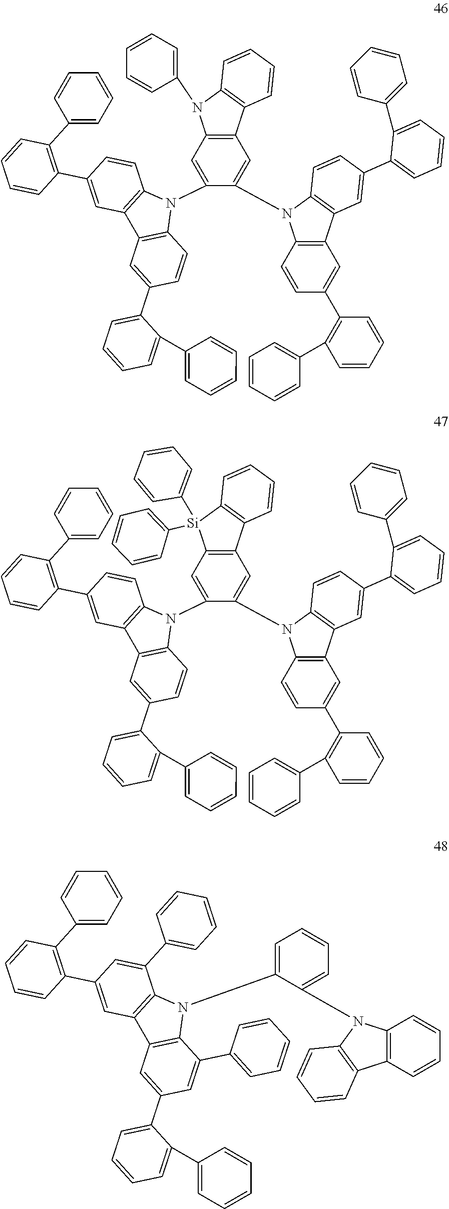 [SMILES: c1ccc(-c2ccccc2-c2ccc3c(c2)c2cc(-c4ccccc4-c4ccccc4)ccc2n3-c2cc3c4ccccc4n(-c4ccccc4)c3cc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2cc3c(cc2-n2c4ccc(-c5ccccc5-c5ccccc5)cc4c4cc(-c5ccccc5-c5ccccc5)ccc42)[Si](c2ccccc2)(c2ccccc2)c2ccccc2-3)cc1 ; c1ccc(-c2ccccc2-c2cc(-c3ccccc3)c3c(c2)c2cc(-c4ccccc4-c4ccccc4)cc(-c4ccccc4)c2n3-c2ccccc2-n2c3ccccc3c3ccccc32)cc1]

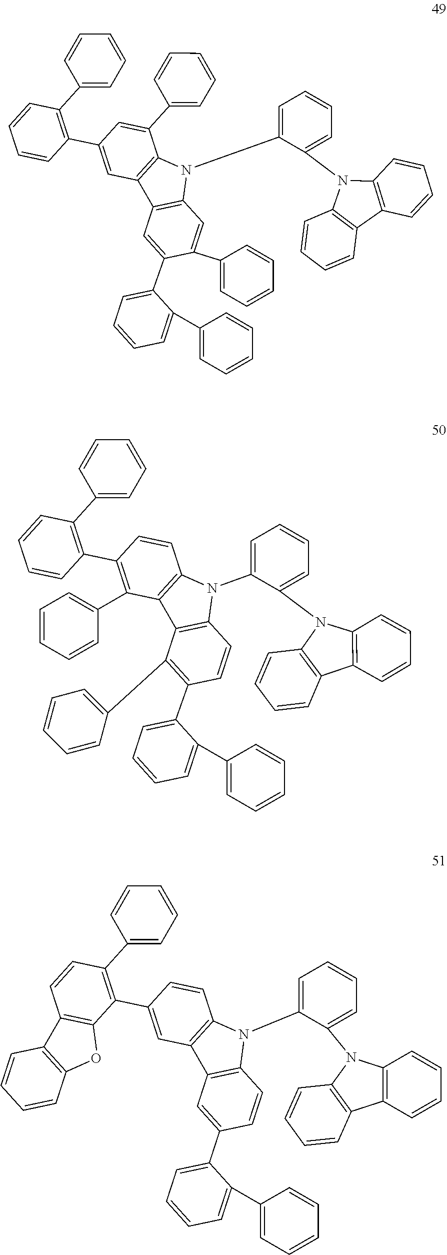 [SMILES: c1ccc(-c2ccccc2-c2cc(-c3ccccc3)c3c(c2)c2cc(-c4ccccc4-c4ccccc4)c(-c4ccccc4)cc2n3-c2ccccc2-n2c3ccccc3c3ccccc32)cc1 ; c1ccc(-c2ccccc2-c2ccc3c(c2-c2ccccc2)c2c(-c4ccccc4)c(-c4ccccc4-c4ccccc4)ccc2n3-c2ccccc2-n2c3ccccc3c3ccccc32)cc1 ; c1ccc(-c2ccccc2-c2ccc3c(c2)c2cc(-c4c(-c5ccccc5)ccc5c4oc4ccccc45)ccc2n3-c2ccccc2-n2c3ccccc3c3ccccc32)cc1]

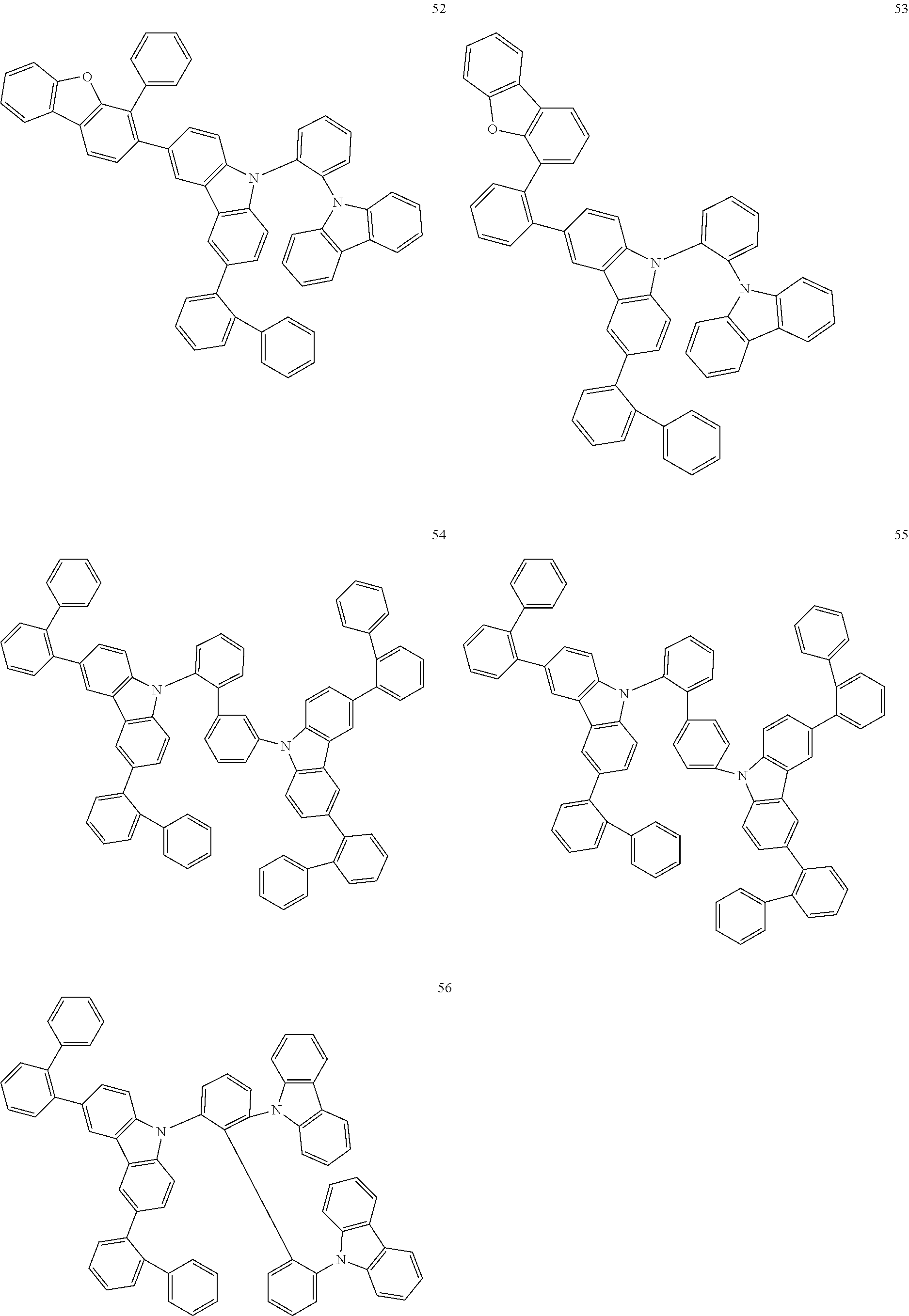

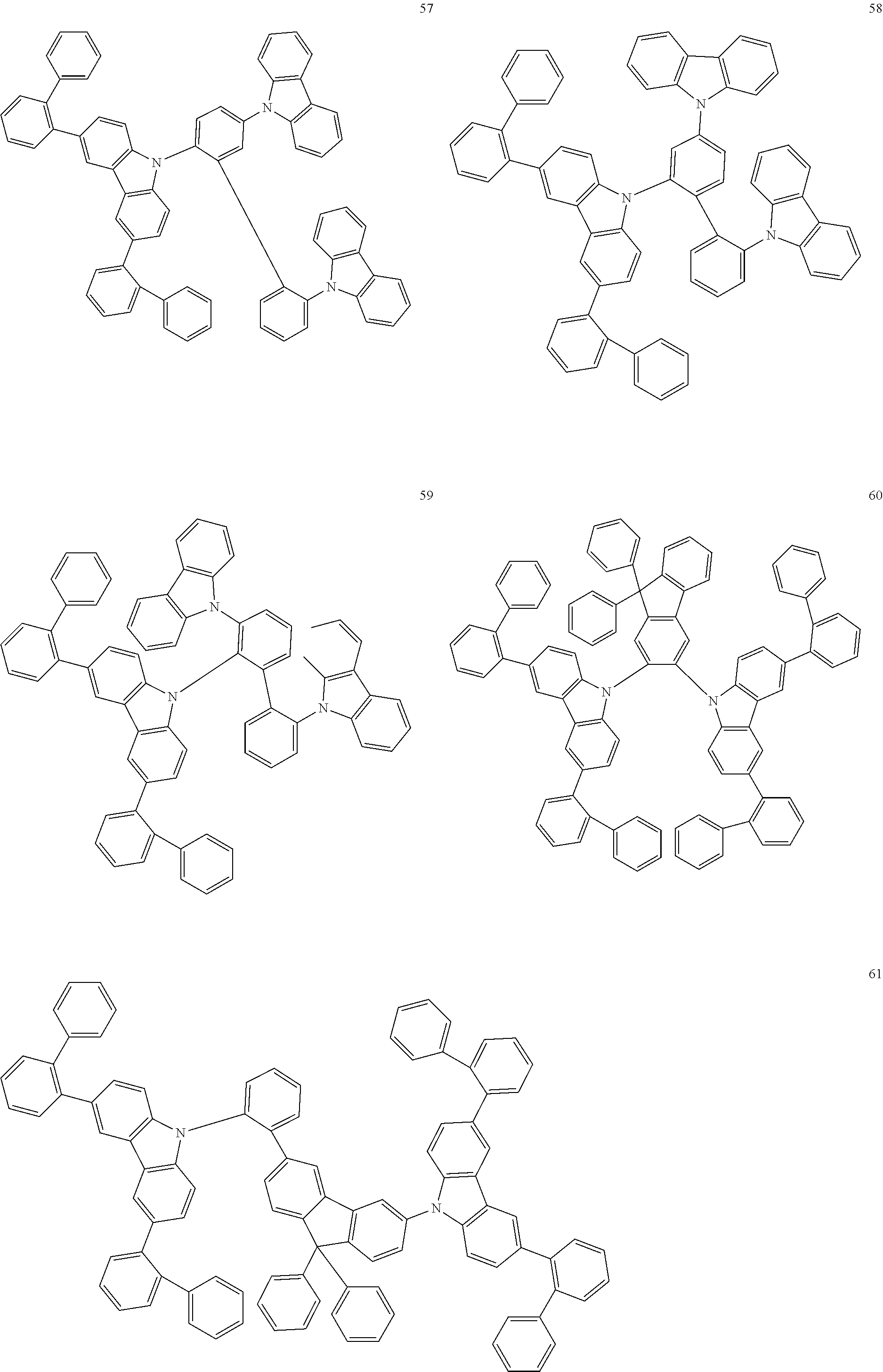

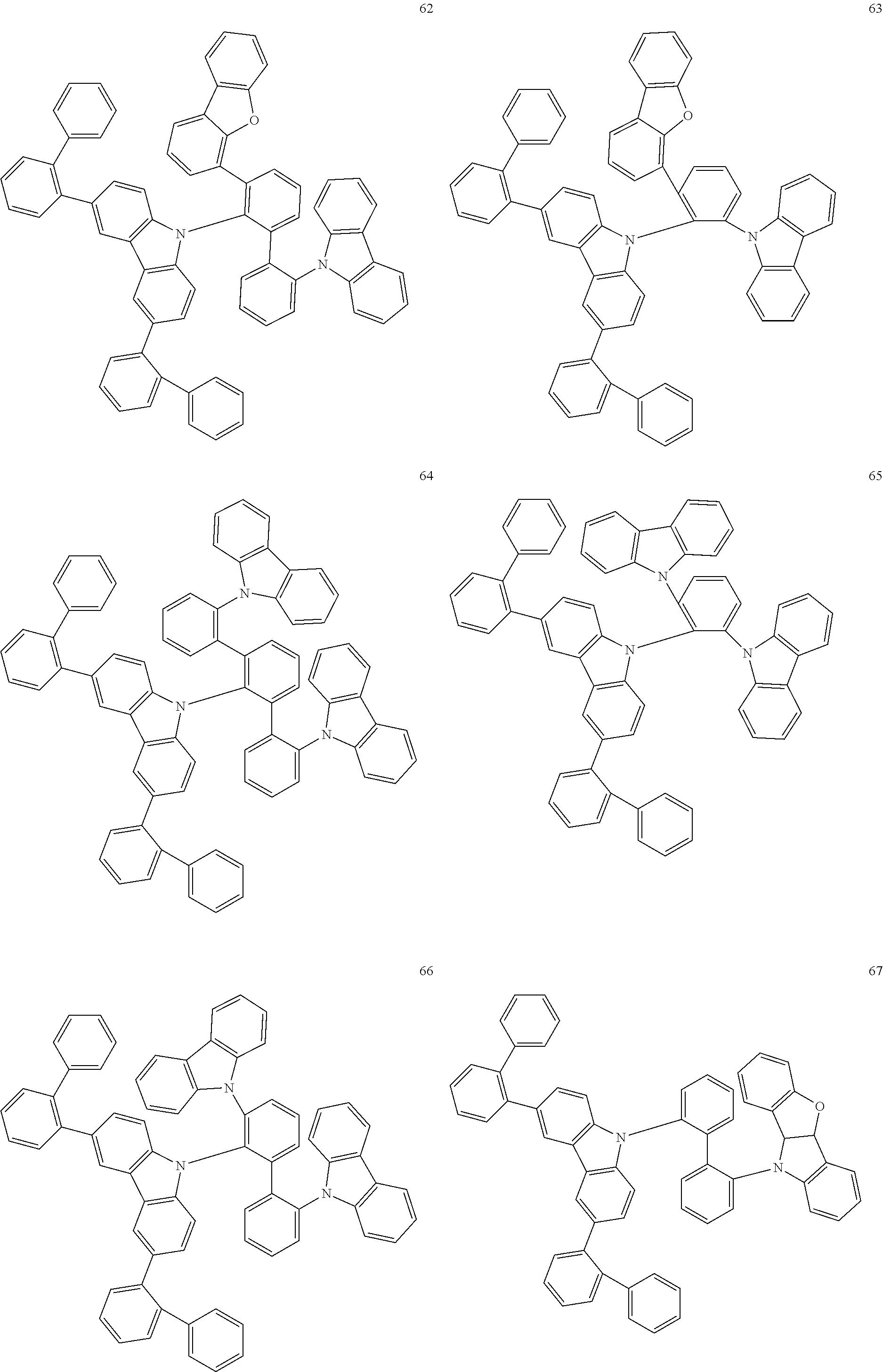

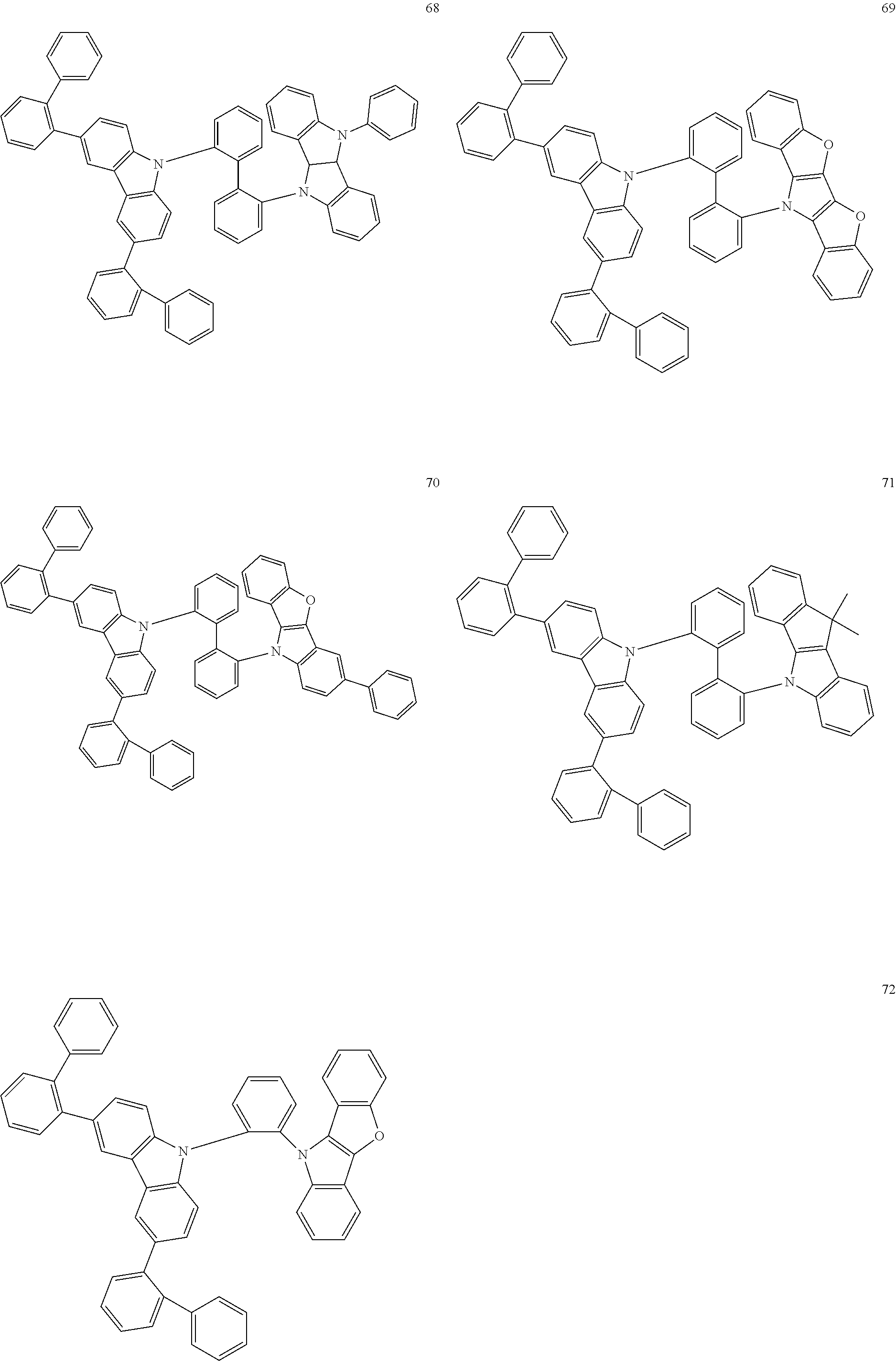

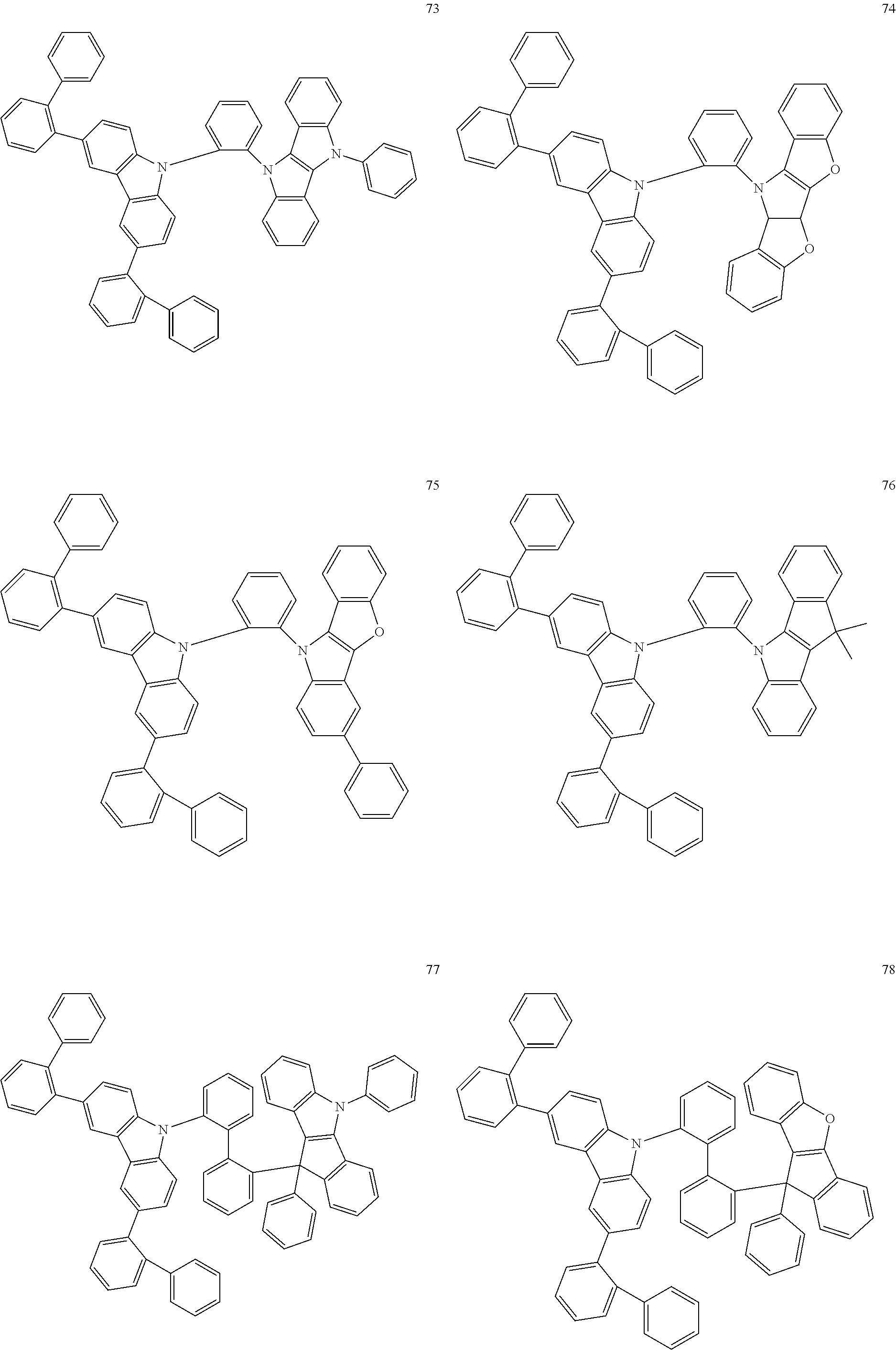

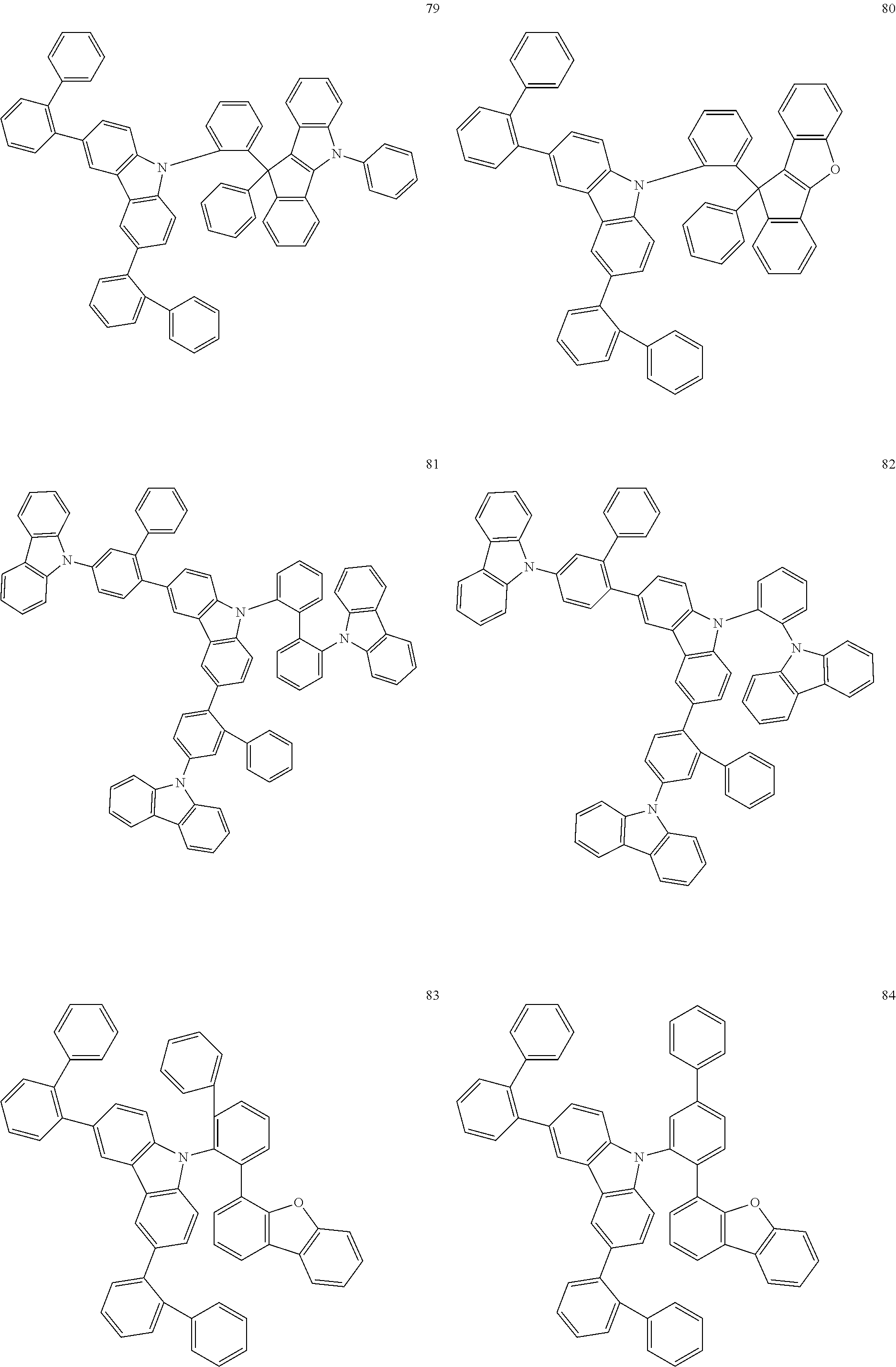

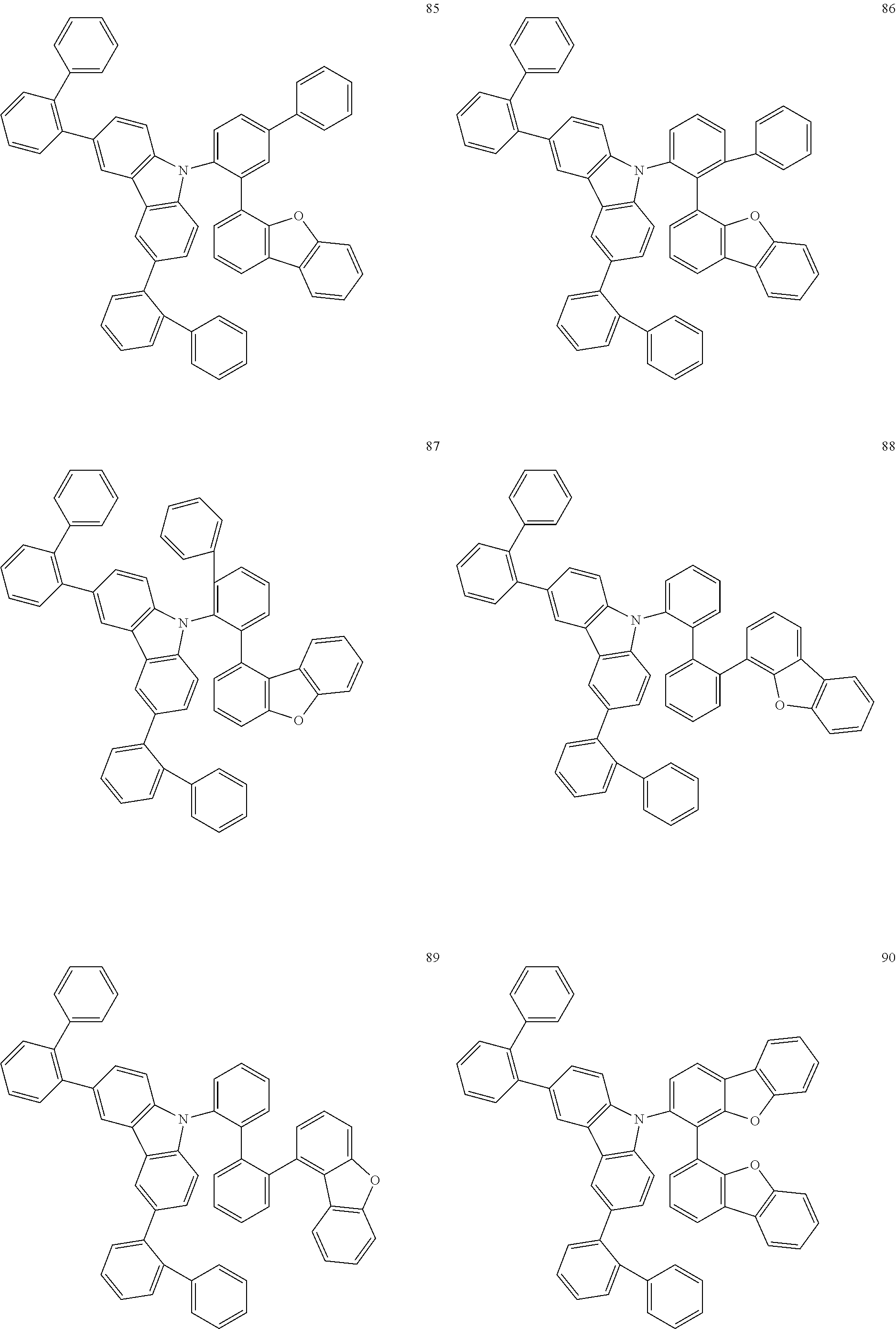

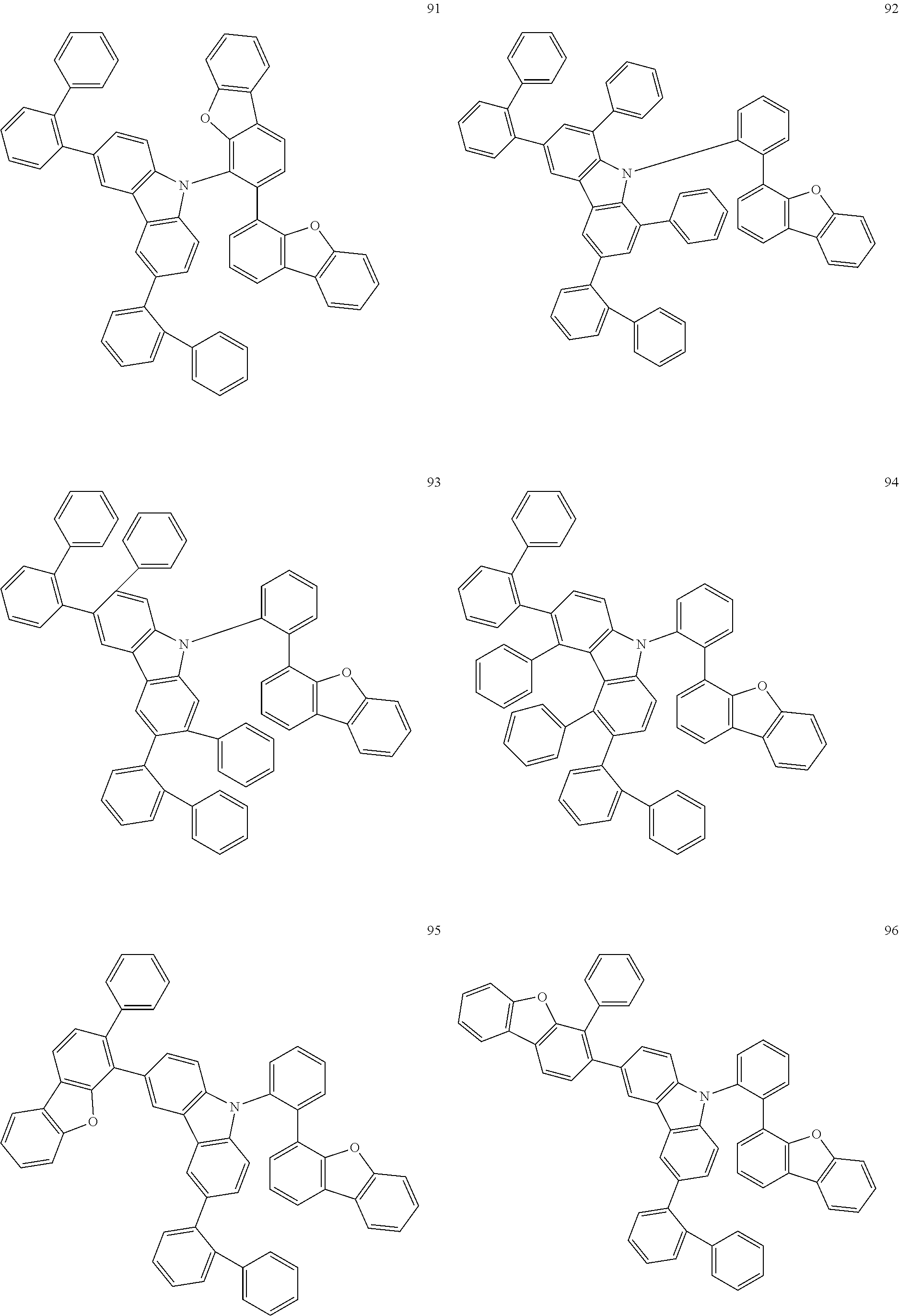

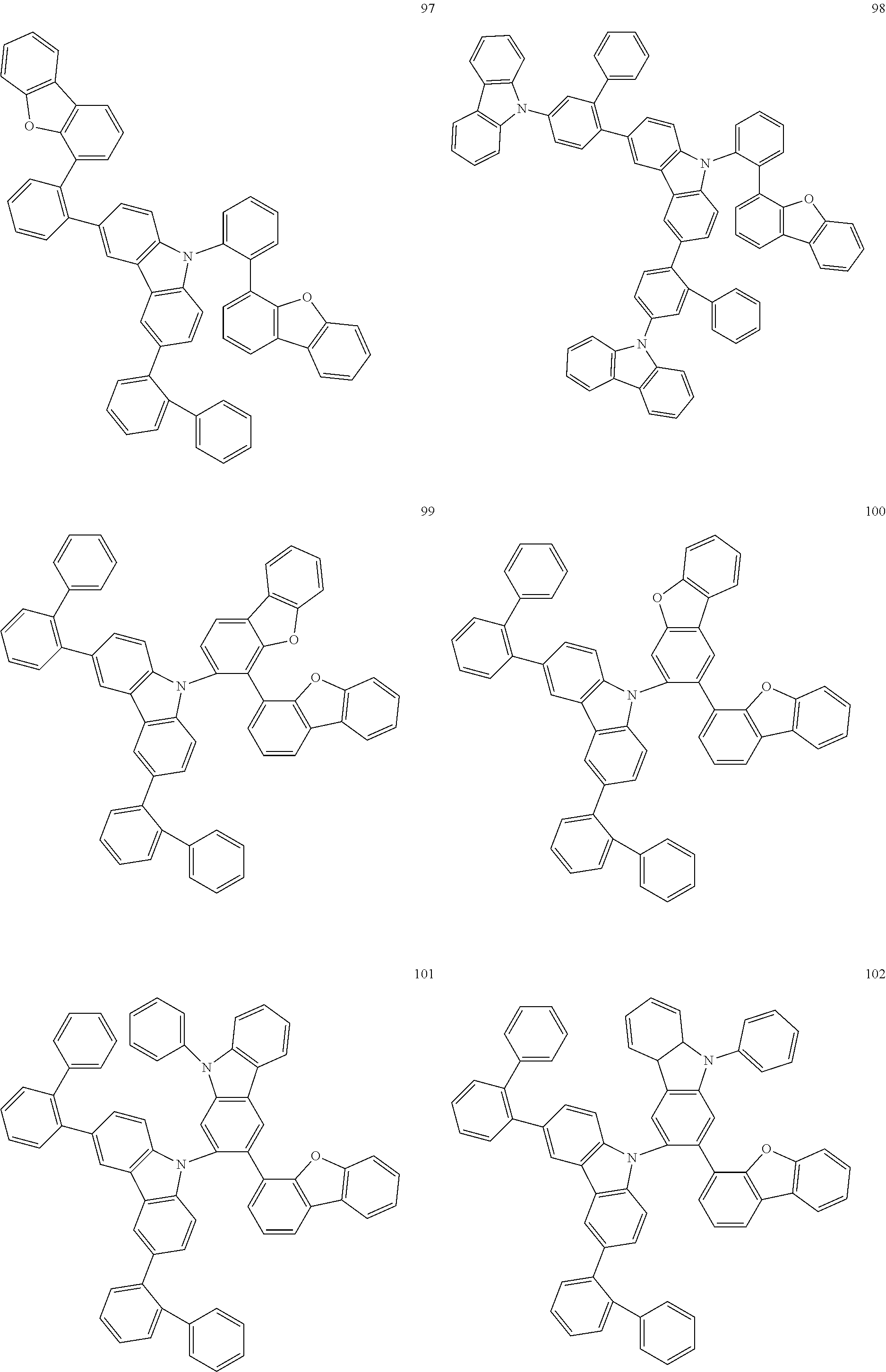

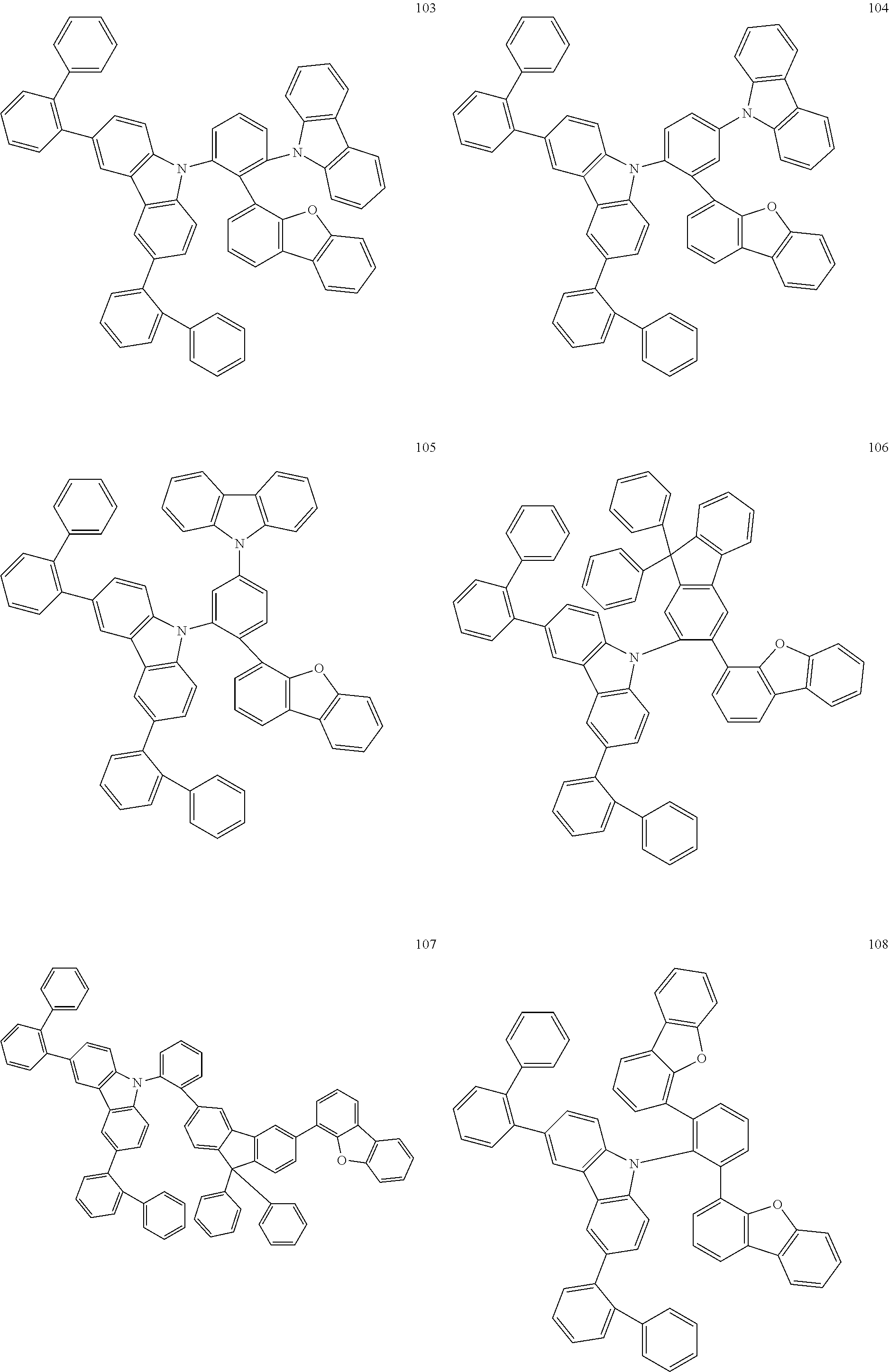

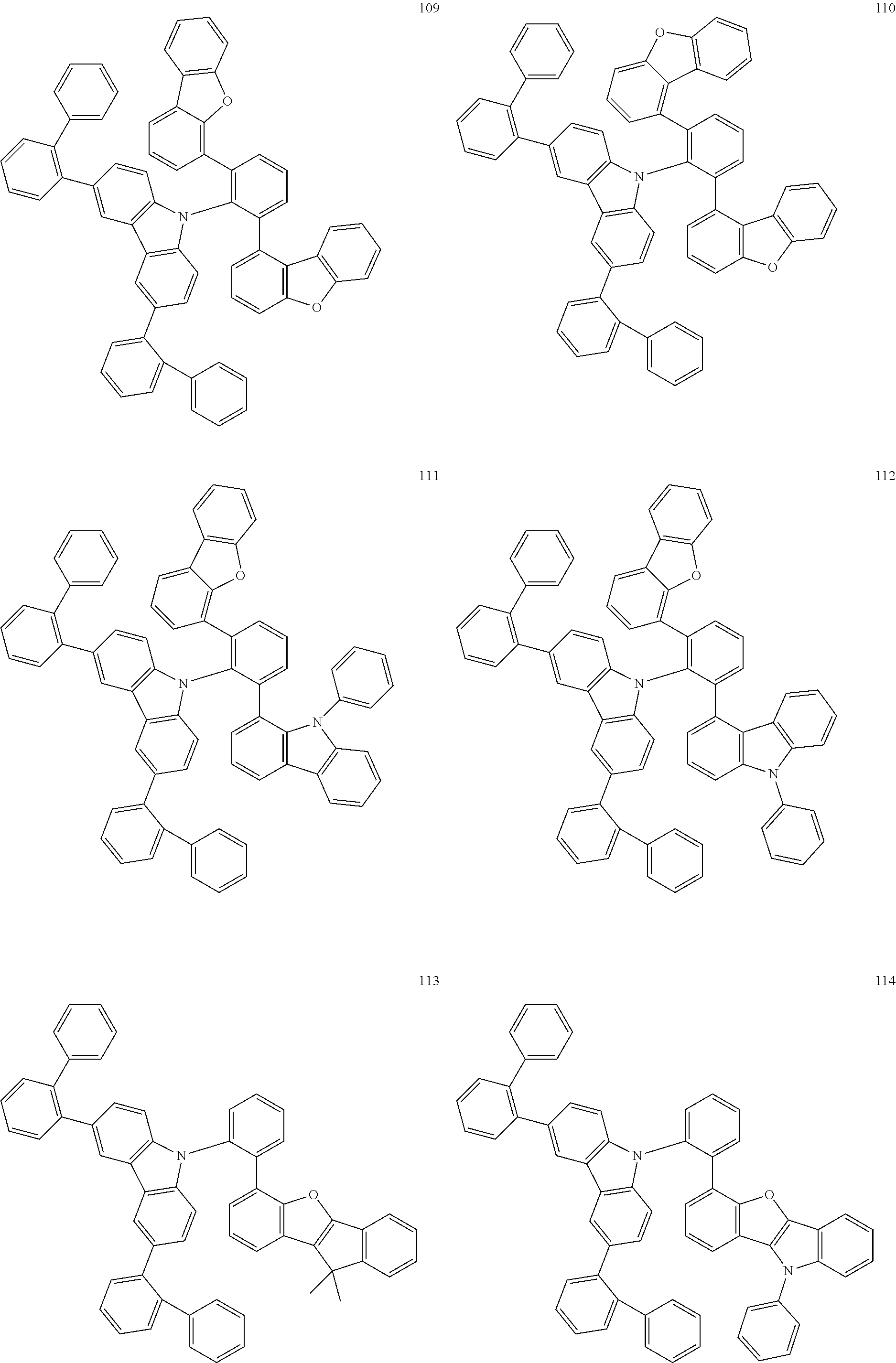

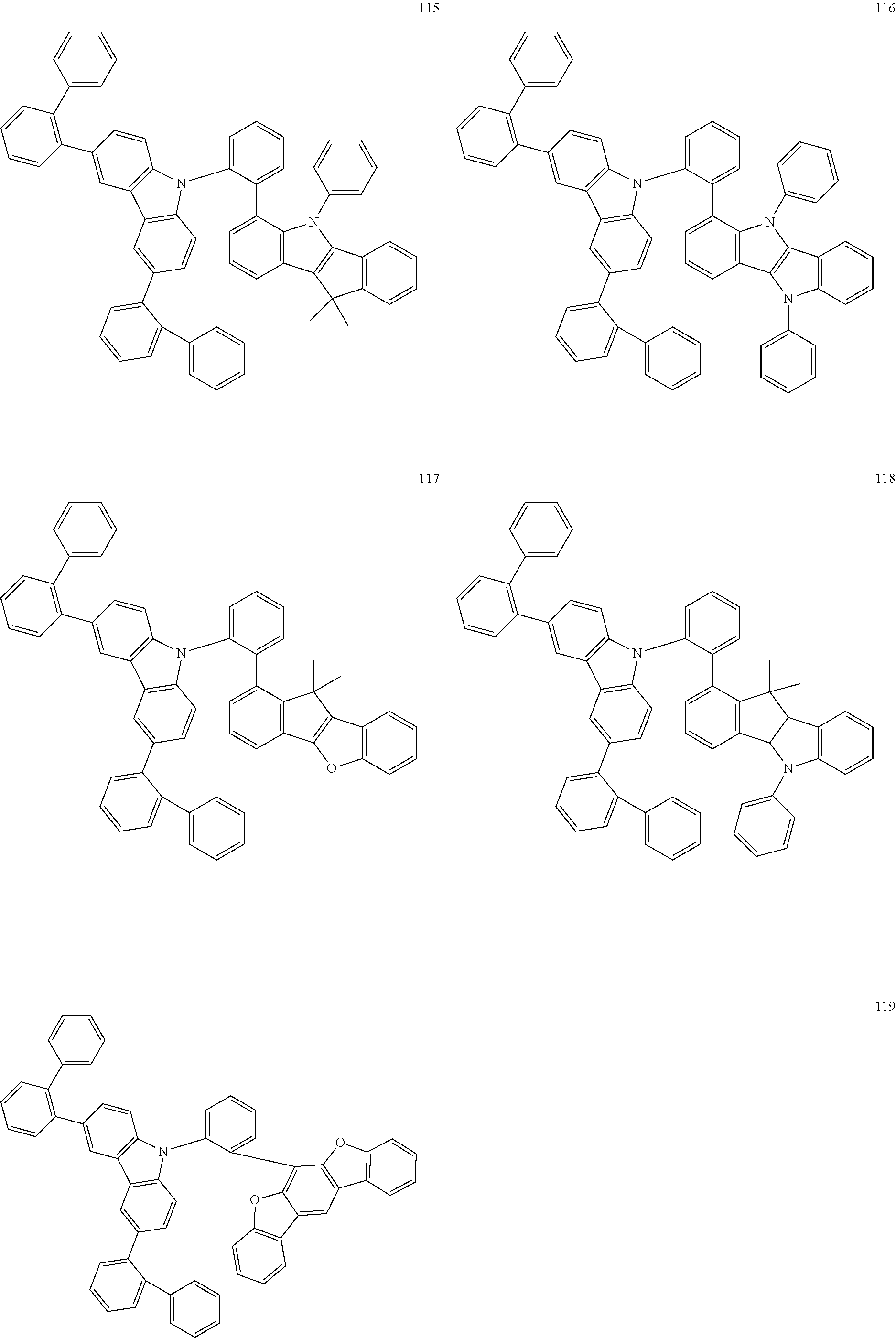

Referring to FIG. 3 to FIG. 6 again, embodiments of the luminescence device ED will be explained. As described above, a hole transport region HTR includes an embodiment of the aforementioned nitrogen-containing compound. For example, the hole transport region HTR includes the nitrogen-containing compound represented by Formula 1.

If the hole transport region HTR has a multilayer structure having multiple layers, any one layer of the multiple layers may include the nitrogen-containing compound represented by Formula 1. For example, a hole transport region HTR may include a hole injection layer HIL disposed on a first electrode EL1 and a hole transport layer HTL disposed on the hole injection layer HIL, and the hole transport layer HTL may include the nitrogen-containing compound represented by Formula 1. However, the embodiments are not limited thereto, and for example, the hole injection layer HIL may include the nitrogen-containing compound represented by Formula 1.

The hole transport region HTR may include one or two or more types of the nitrogen-containing compound represented by Formula 1. For example, the hole transport region HTR may include at least one selected from the compounds represented in Compound Groups 1 to 3. The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may further include a compound represented by Formula H-1 below:

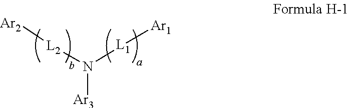

In Formula H-1 above, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. When “a” and “b” may be each independently an integer of 0 to 10. If “a” or “b” is an integer of 2 or more, multiple L₁ and L₂ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. In addition, in Formula H-1, Ar₃ may be a substituted or unsubstituted aryl group of 6 to 30 ring-forming carbon atoms.

The compound represented by Formula H-1 may be a monoamine compound. Otherwise, the compound represented by Formula H-1 may be a diamine compound in which at least one of Ar₁ to Ar₃ includes an amine group as a substituent. In addition, the compound represented by Formula H-1 may be a carbazole-based compound in which at least one of Ar₁ to Ar₃ includes a substituted or unsubstituted carbazole group, or a fluorene-based compound in which at least one of Ar₁ to Ar₃ includes a substituted or unsubstituted fluorene group.

The compound represented by Formula H-1 may be represented by any one of the compounds in Compound Group H below. However, the compounds shown in Compound Group H are only illustrations, and the compound represented by Formula H-1 is not limited to the compounds represented in Compound Group H below.

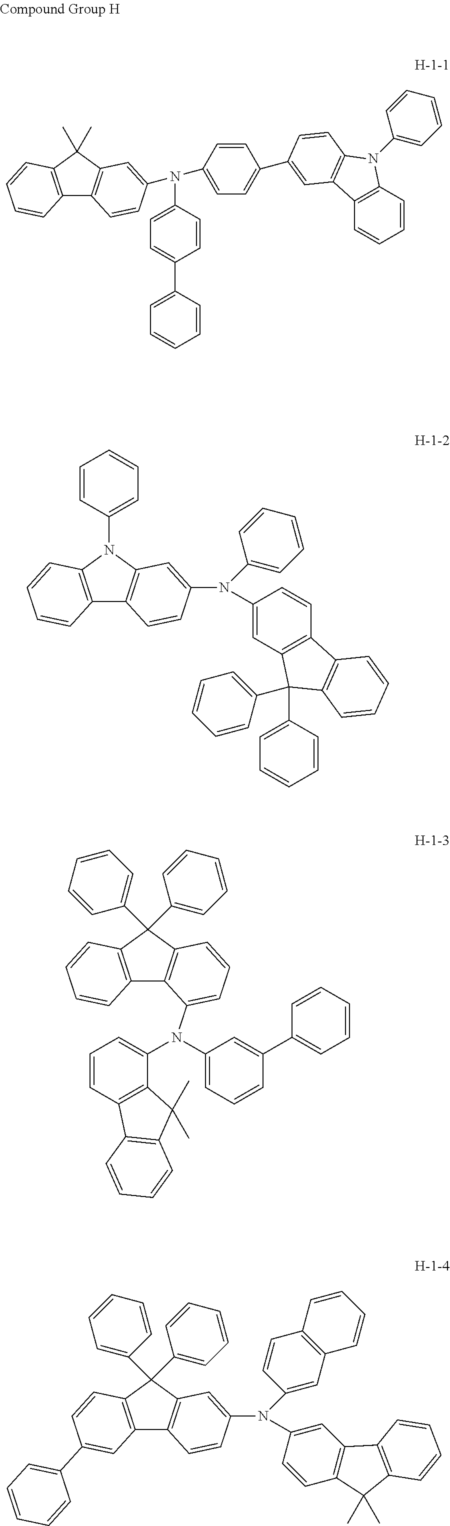 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

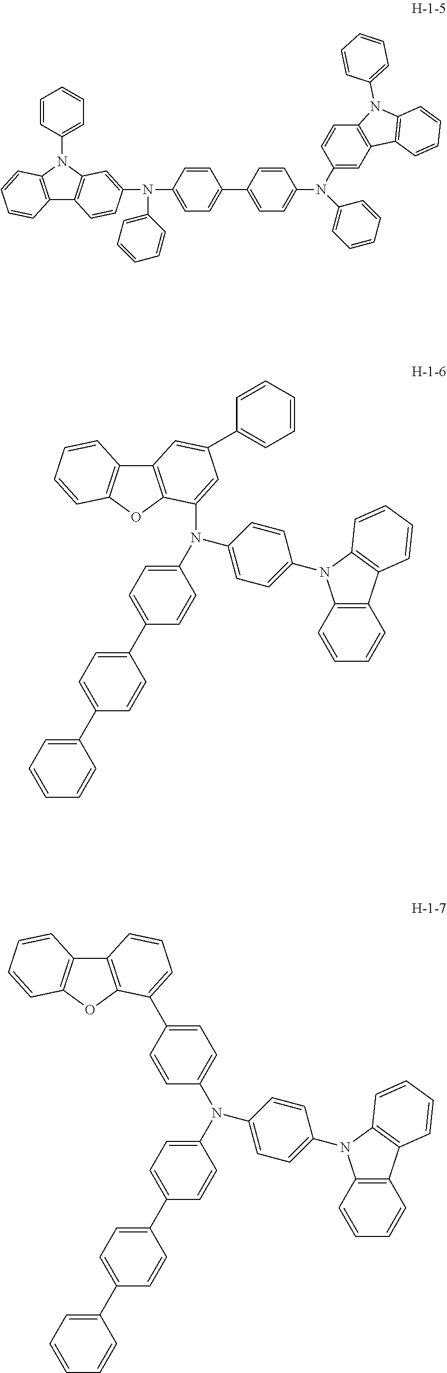 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

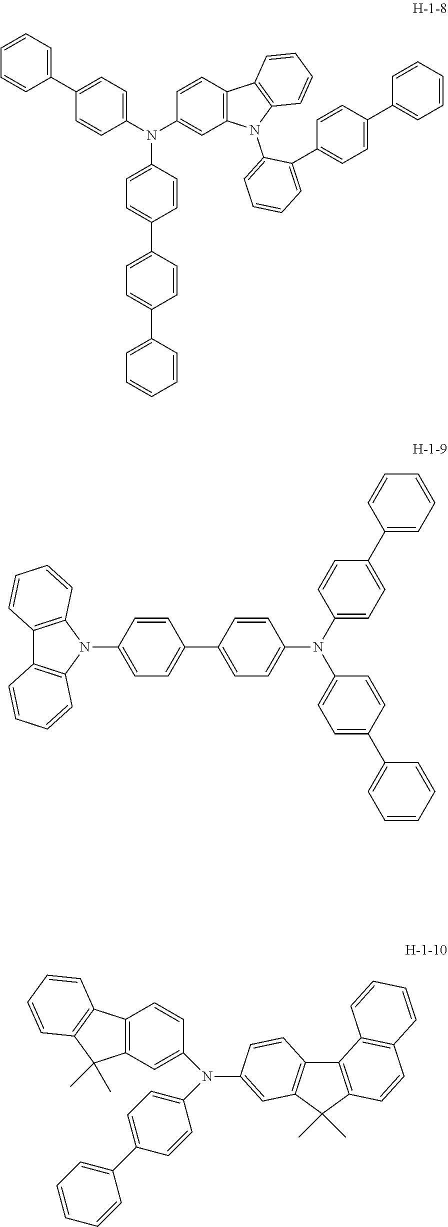 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

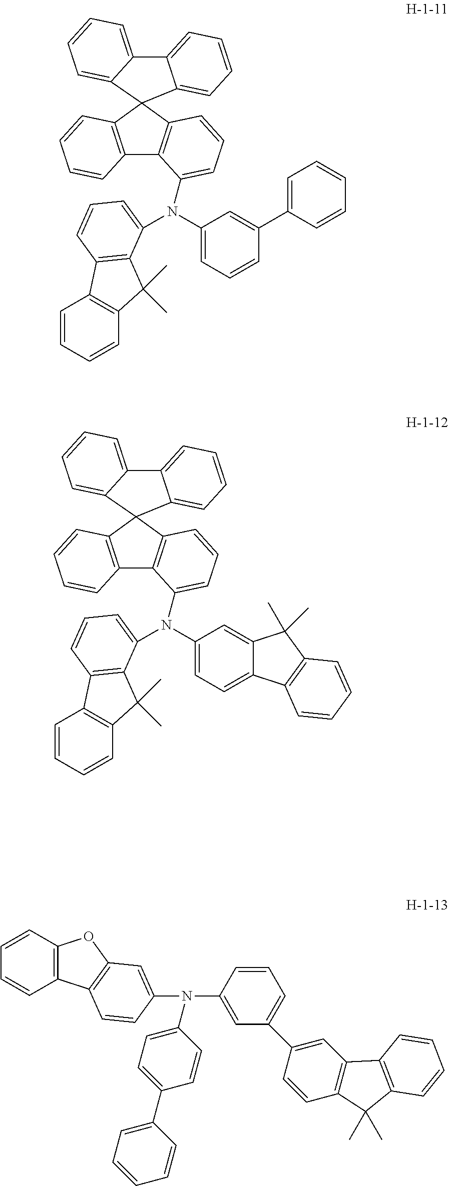 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

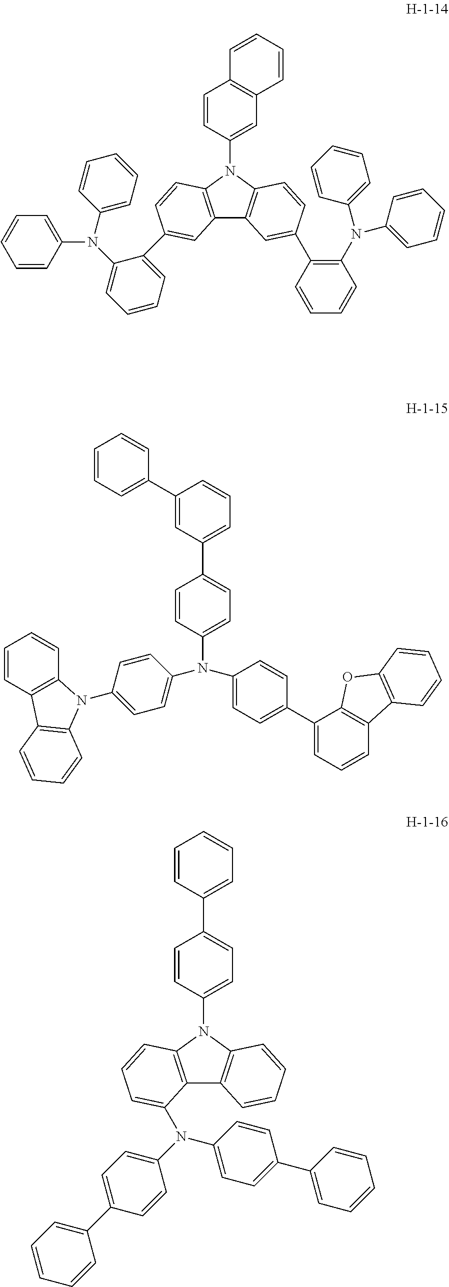 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

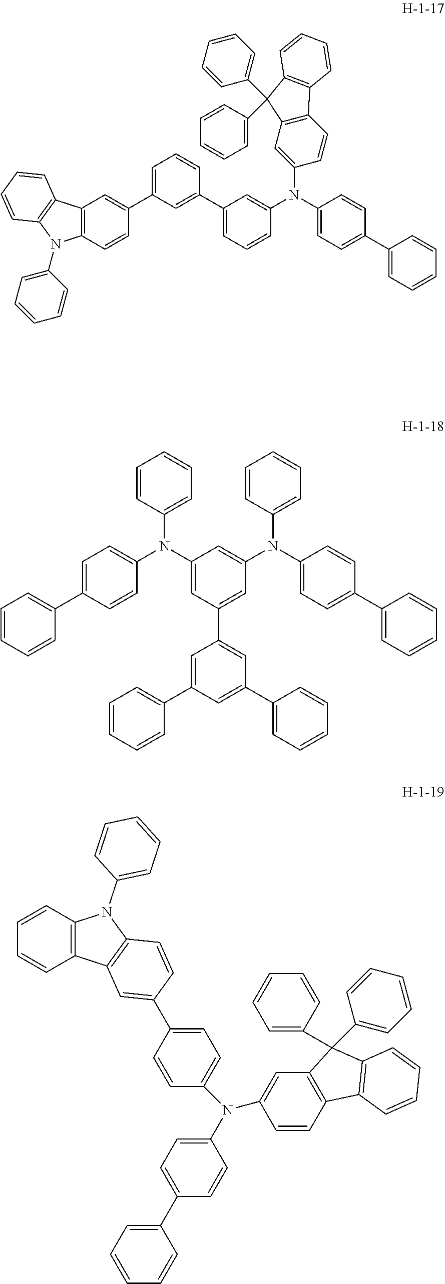 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N-diphenyl-benzidine (NPB), triphenylamine-including polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include, for example, carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 1,3-di(carbazol-9-yl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc. The hole transport region HTR may include the compounds of the hole transport region in at least one of the hole injection layer HIL, hole transport layer HTL, and electron blocking layer EBL.

The thickness of the hole transport region HTR may be from about 100 Å to about 10,000 Å, for example, from about 100 Å to about 5,000 Å. The thickness of the hole injection region HIL may be, for example, from about 30 Å to about 1,000 Å. The thickness of the hole transport layer HTL may be from about 30 Å to about 1,000 Å. For example, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include any one of quinone derivatives, metal oxides, and cyano group-containing compounds, without limitation. For example, non-limiting examples of the p-dopant may include quinone derivatives such as tetracyanoquinodimethane (TCNQ) and 2,3,5,6-tetrafluoro-7,7′,8,8-tetracyanoquinodimethane (F4-TCNQ), metal oxides such as a tungsten oxide and a molybdenum oxide, etc., without limitation.

As described above, the hole transport region HTR may further include at least one of a hole buffer layer or an electron blocking layer EBL in addition to the hole injection layer HIL and the hole transport layer HTL. The hole buffer layer may compensate for a resonance distance according to the wavelength of light emitted from an emission layer EML and, although not wanting to be bound by theory, may increase light emitting efficiency. As materials included in the hole buffer layer, materials which may be included in the hole transport region HTR may be used. The electron blocking layer EBL is a layer playing the role of blocking the injection of electrons from an electron transport region ETR to a hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

In an embodiment the luminescence device ED, the emission layer EML may include anthracene derivatives, pyrene derivatives, fluoranthene derivatives, chrysene derivatives, dihydrobenzanthracene derivatives, or triphenylene derivatives. Particularly, the emission layer EML may further include anthracene derivatives or pyrene derivatives.

In the luminescence devices ED shown in FIG. 3 to FIG. 6, the emission layer EML may include a host and a dopant, and the emission layer EML may include a compound represented by Formula E-1 below. The compound represented by Formula E-1 below may be used as a fluorescence host material.

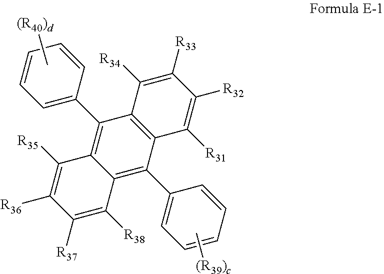

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring. The variables R₃₁ to R₄₀ may be combined with an adjacent group to form a saturated hydrocarbon ring, an unsaturated hydrocarbon ring, a saturated heterocycle or an unsaturated heterocycle.

In Formula E-1, “c” and “d” may be each independently an integer of 0 to 5.

Formula E-1 may be represented by any one of Compound E1 to Compound E19 below.

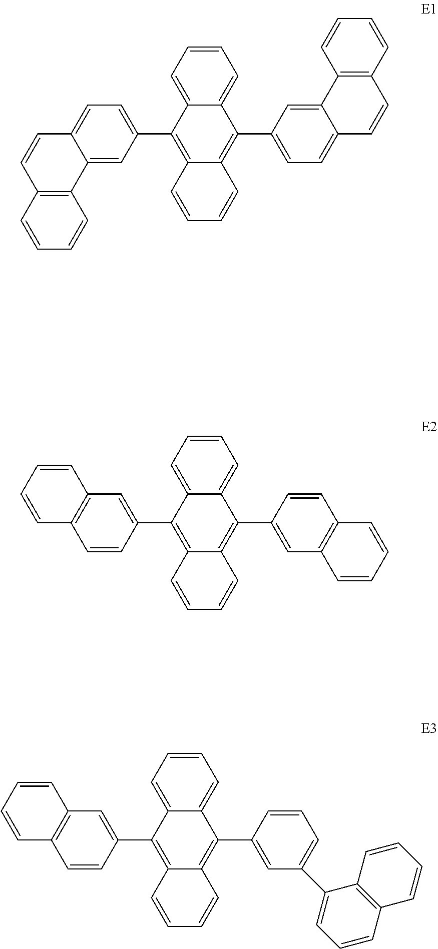

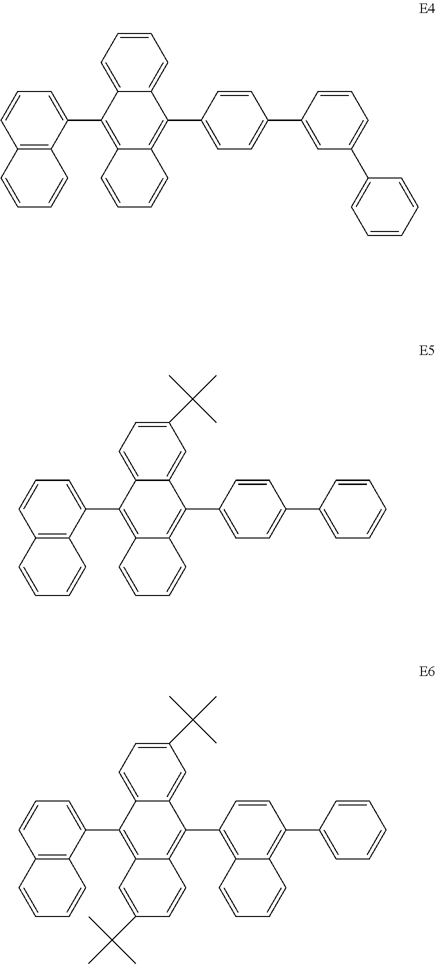

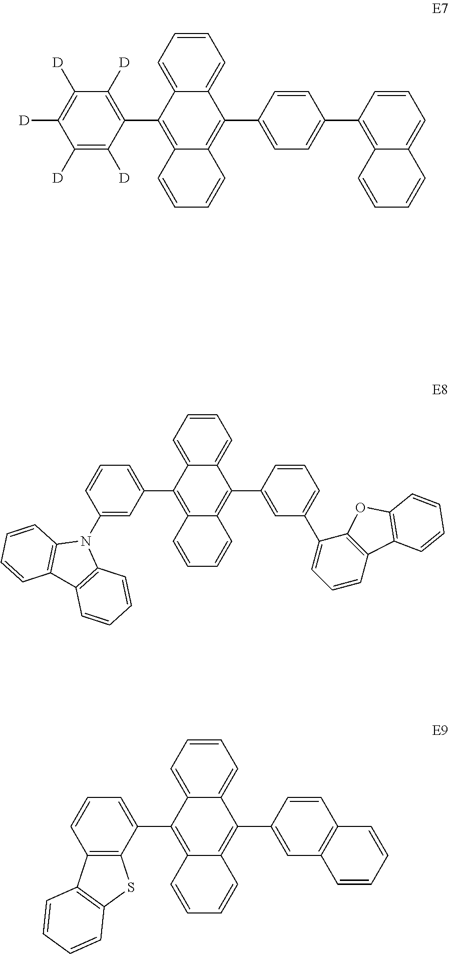 [SMILES: [2H]c1c([2H])c([2H])c(-c2c3ccccc3c(-c3ccc(-c4cccc5ccccc45)cc3)c3ccccc23)c([2H])c1[2H] ; c1cc(-c2c3ccccc3c(-c3cccc(-n4c5ccccc5c5ccccc54)c3)c3ccccc23)cc(-c2cccc3c2oc2ccccc23)c1 ; c1ccc2cc(-c3c4ccccc4c(-c4cccc5c4sc4ccccc45)c4ccccc34)ccc2c1]

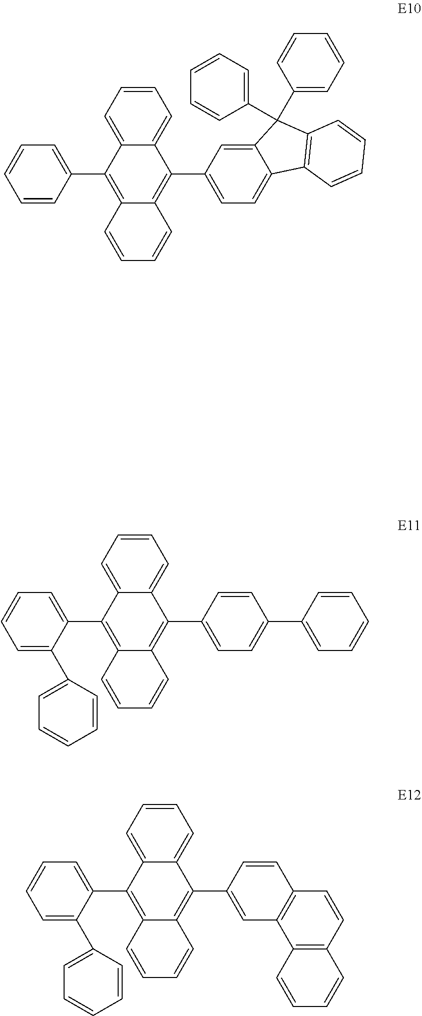

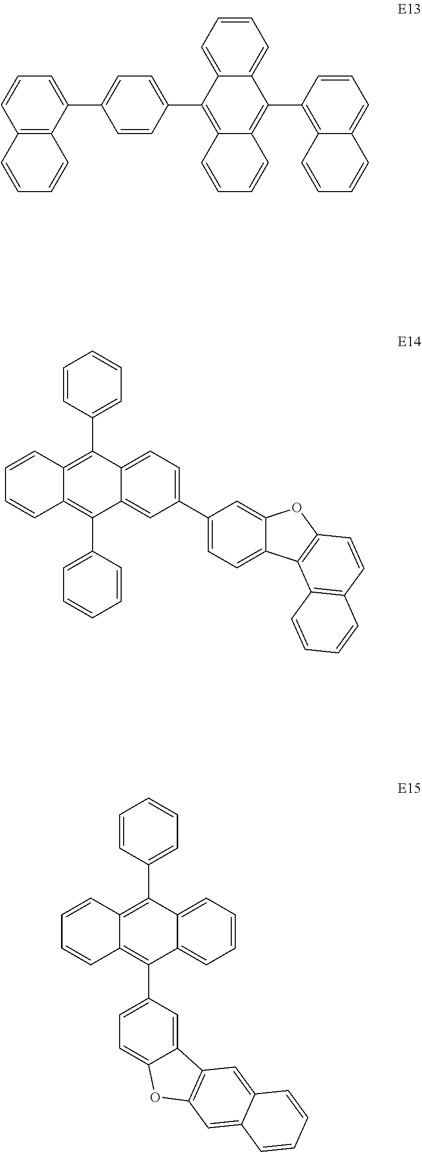

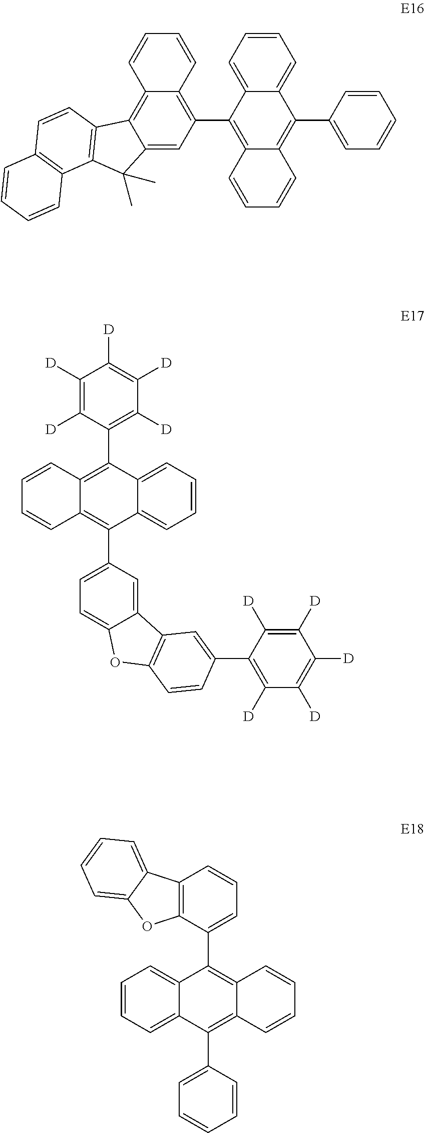

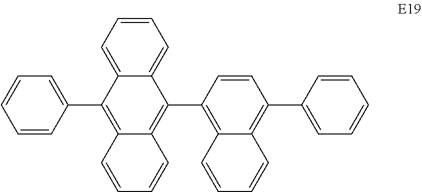 [SMILES: c1ccc(-c2ccc(-c3c4ccccc4c(-c4ccccc4)c4ccccc34)c3ccccc23)cc1]

The emission layer EML may include a compound represented by Formula E-2a or Formula E-2b below. The compound represented by Formula E-2a or Formula E-2b below may be used as a phosphorescence host material.

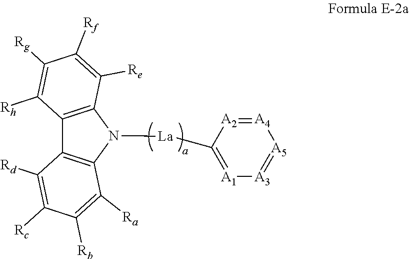

In Formula E-2b, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. If “a” is an integer of 2 or more, multiple Lₐ's may be each independently a substituted or unsubstituted arylene group of 6 to 30 ring-forming carbon atoms, or a substituted or unsubstituted heteroarylene group of 2 to 30 ring-forming carbon atoms.

In addition, in Formula E-2a, A₁ to A₅ may be each independently N or CRi. The variables Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. The variables Rₐ to Rᵢ may be combined with an adjacent group to form a hydrocarbon ring or a heterocycle including N, O, S, etc. as a ring-forming atom. In Formula E-2a, two or three selected from A₁ to A₅ may be N, and the remainder may be CRᵢ.

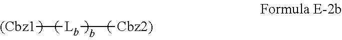

In Formula E-2b, Cbz1 and Cbz2 may be each independently an unsubstituted carbazole group, or a carbazole group substituted with an aryl group of 6 to 30 ring-forming carbon atoms. The variable L_{b} may be a direct linkage, a substituted or unsubstituted arylene group of 6 to 30 ring-forming carbon atoms, or a substituted or unsubstituted heteroarylene group of 2 to 30 ring-forming carbon atoms. The variable “b” is an integer of 0 to 10, and if “b” is an integer of 2 or more, multiple L_{b}'s may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one of the compounds in Compound Group E-2 below. However, the compounds shown in Compound Group E-2 below are only illustrations, and the compound represented by Formula E-2a or Formula E-2b is not limited to the compounds represented in Compound Group E-2 below.

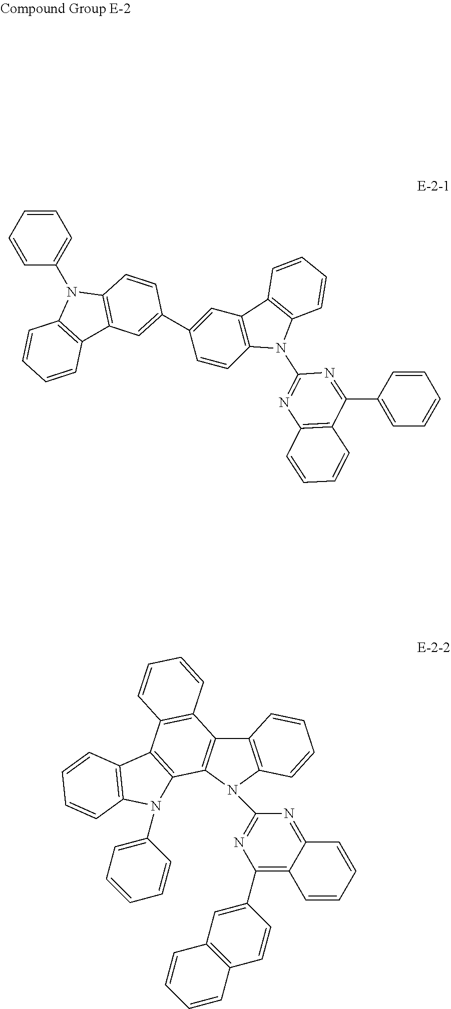 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1]

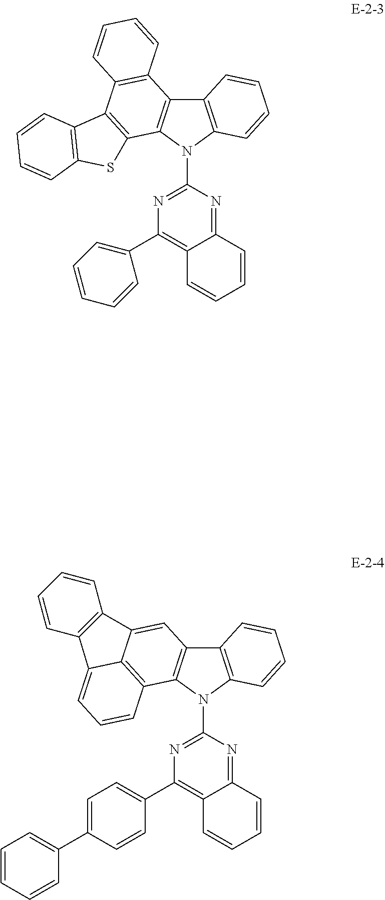 [SMILES: c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

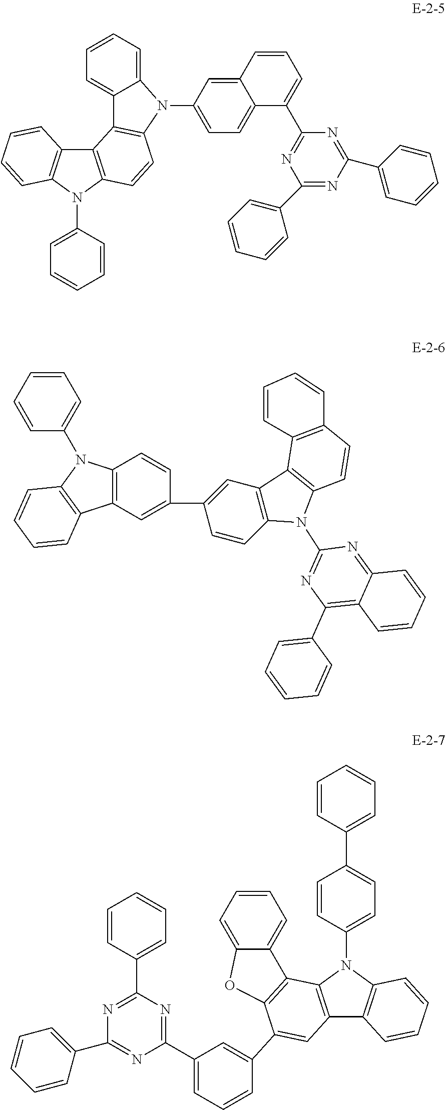 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

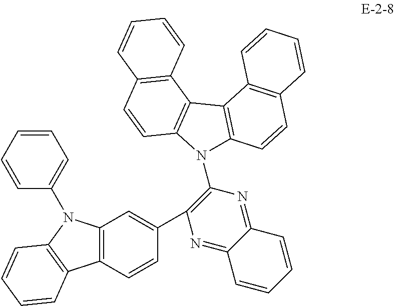 [SMILES: c1ccc(-n2c3ccccc3c3ccc(-c4nc5ccccc5nc4-n4c5ccc6ccccc6c5c5c6ccccc6ccc54)cc32)cc1]

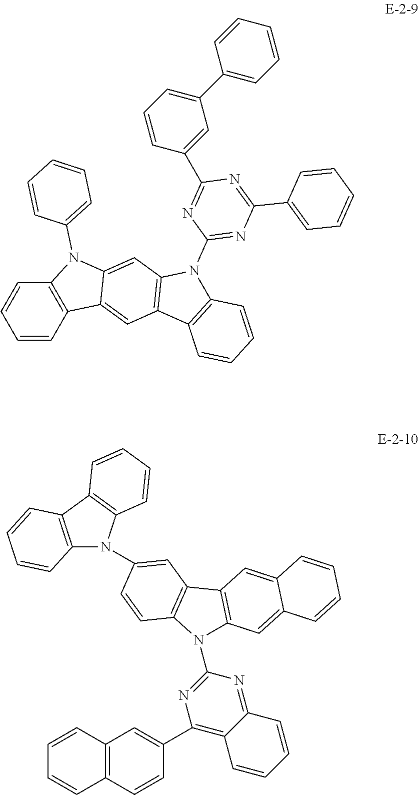 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

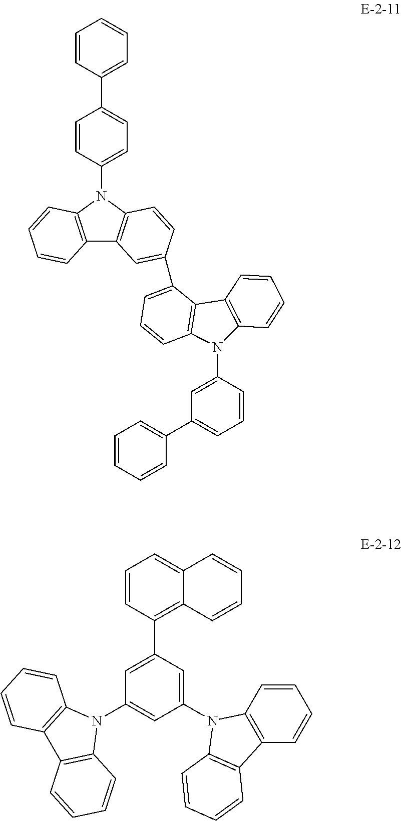 [SMILES: c1ccc(-c2ccc(-n3c4ccccc4c4cc(-c5cccc6c5c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

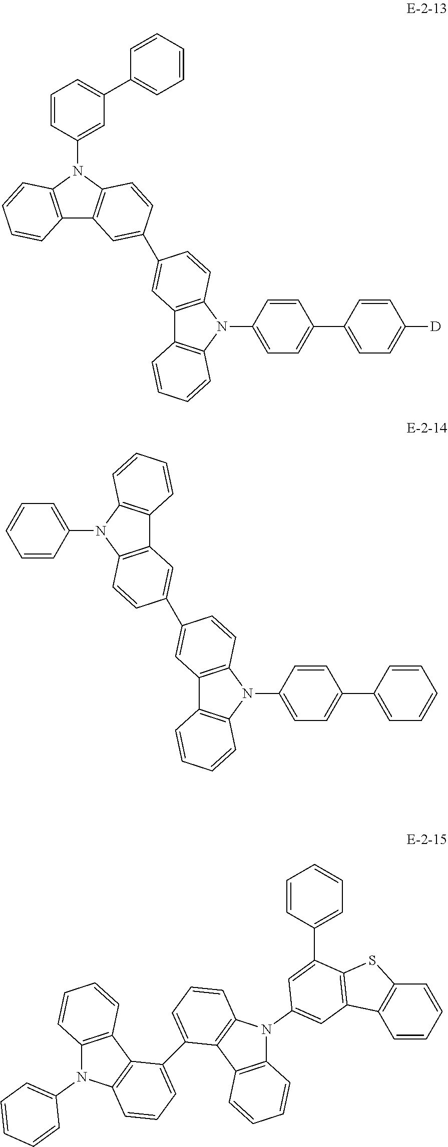 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

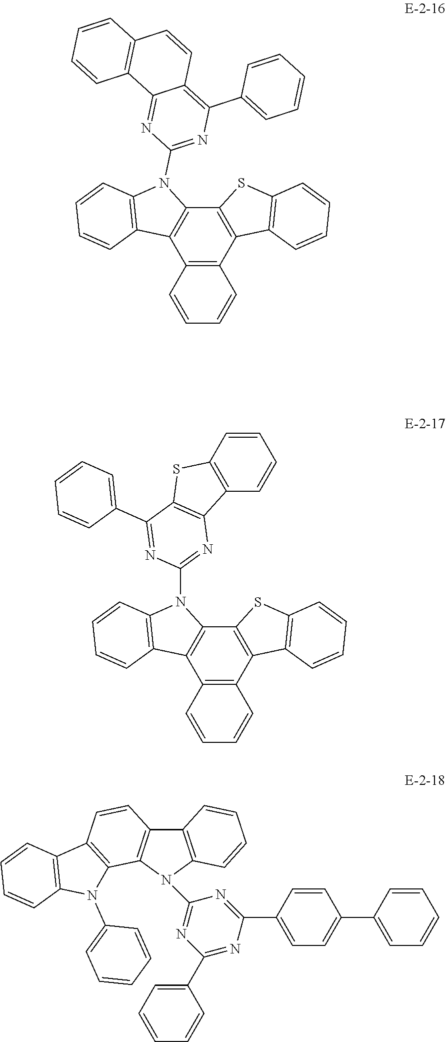 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

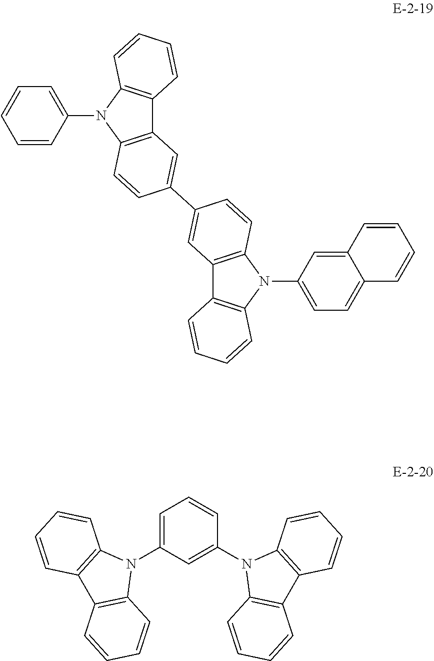 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1]

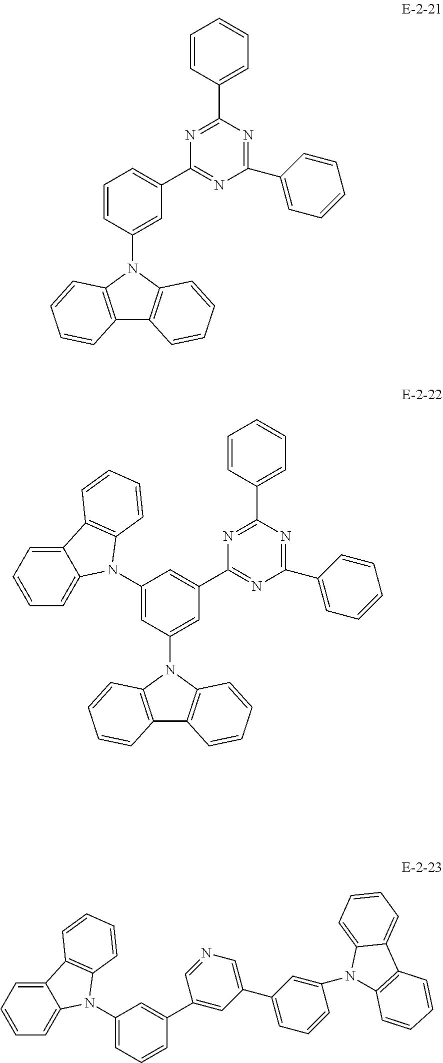 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

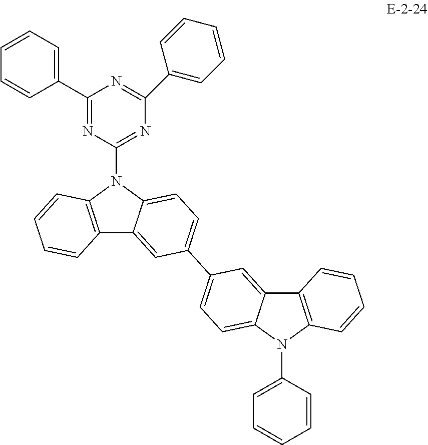 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-known in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiments are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material.

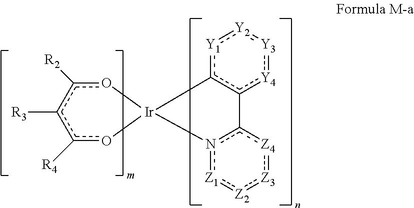

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant. The compound represented by Formula M-a may be represented by any one of Compounds M-a1 to M-a25 below. However, Compounds M-a1 to M-a25 below are illustrations, and the compound represented by Formula M-a is not limited to the compounds represented by Compounds M-a1 to M-a25 below.

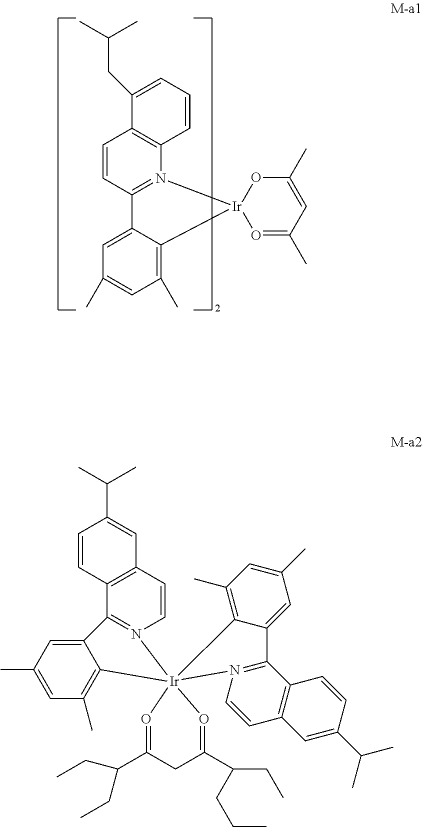

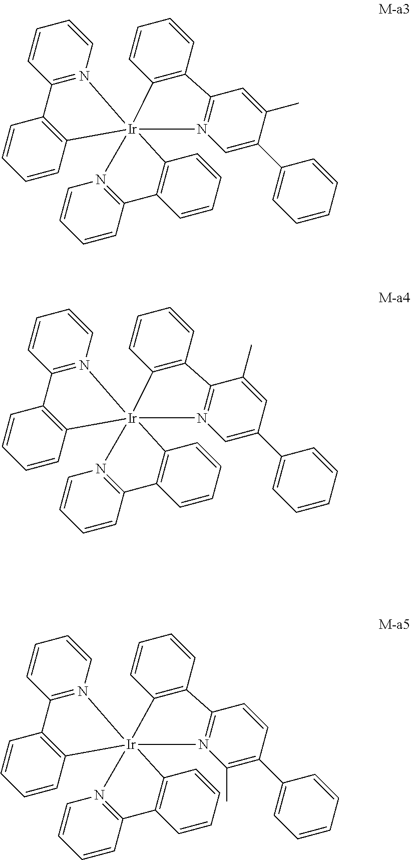

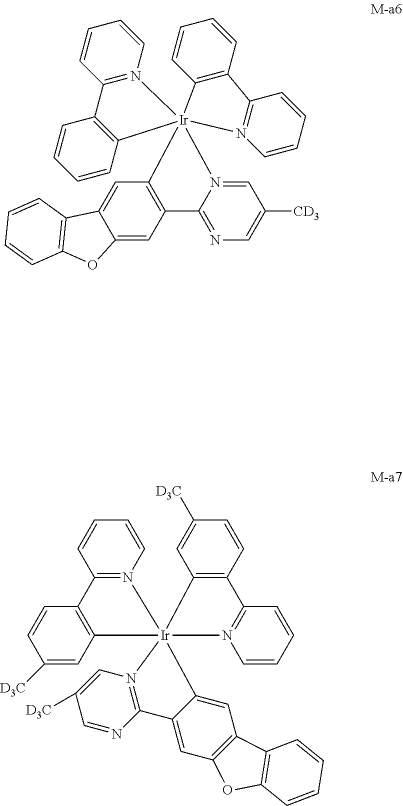

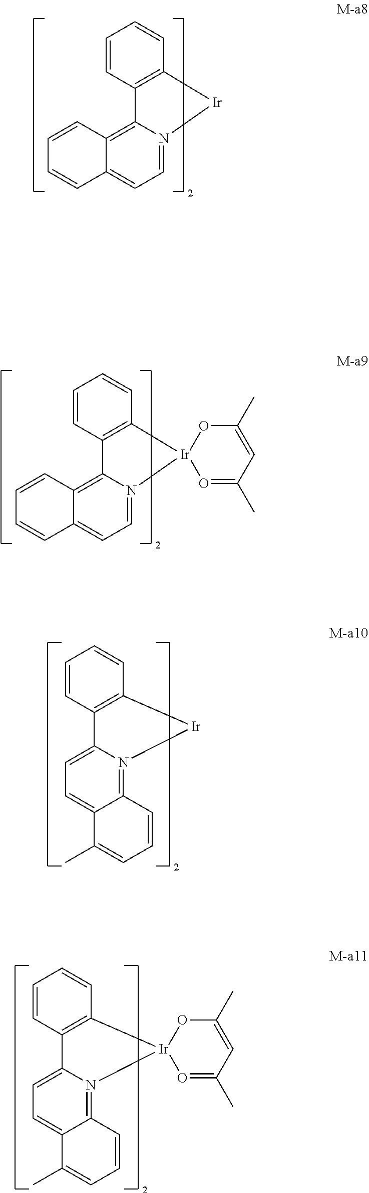

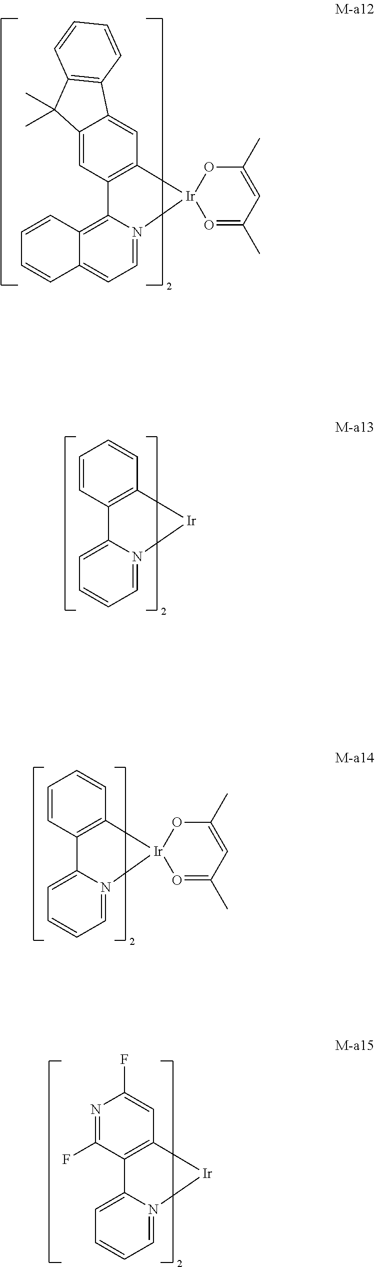

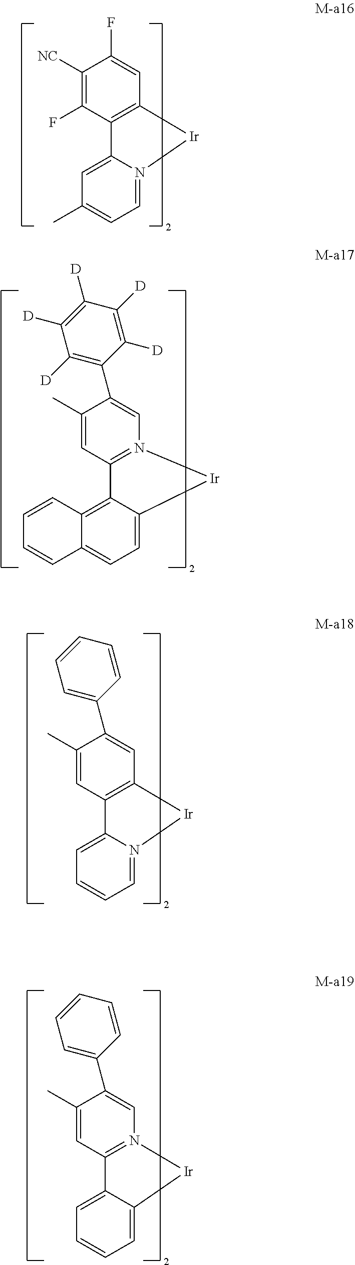

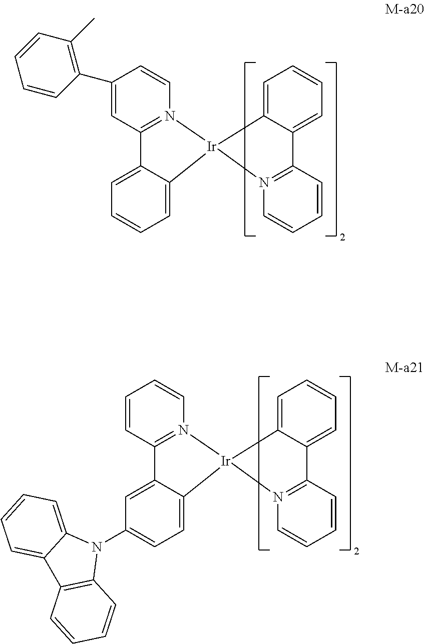

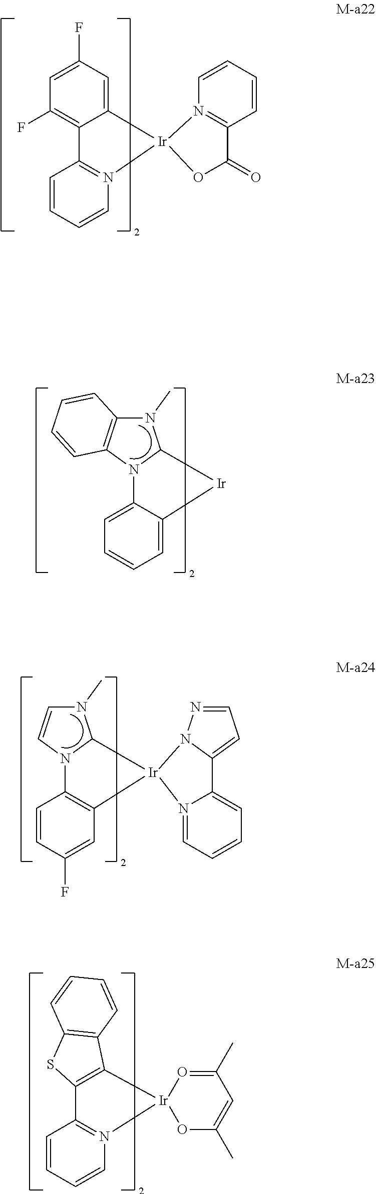

Compound M-a1 and Compound M-a2 may be used as red dopant materials, and Compound M-a3 and Compound M-a4 may be used as green dopant materials.

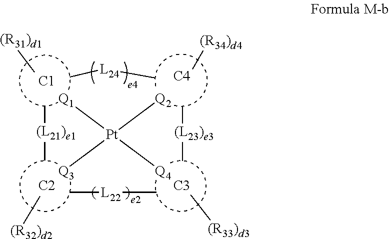

In Formula M-b, Q₁ to Q₄ are each independently C or N, C1 to C4 are each independently a substituted or unsubstituted hydrocarbon ring of 5 to 30 ring-forming carbon atoms, or a substituted or unsubstituted heterocycle of 2 to 30 ring-forming carbon atoms. The variables L₂₁ to L₂₄ are each independently a direct linkage,

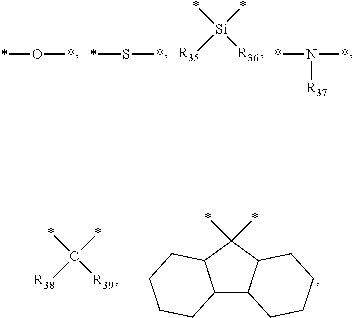

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 are each independently 0 or 1. The variables R₃₁ to R₃₉ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 are each independently an integer of 0 to 4.

The compound represented by Formula M-b may be used as a blue phosphorescence dopant or a green phosphorescence dopant. The compound represented by Formula M-b may be represented by any one of the compounds below. However, the compounds below are illustrations, and the compound represented by Formula M-b is not limited to the compounds represented below.

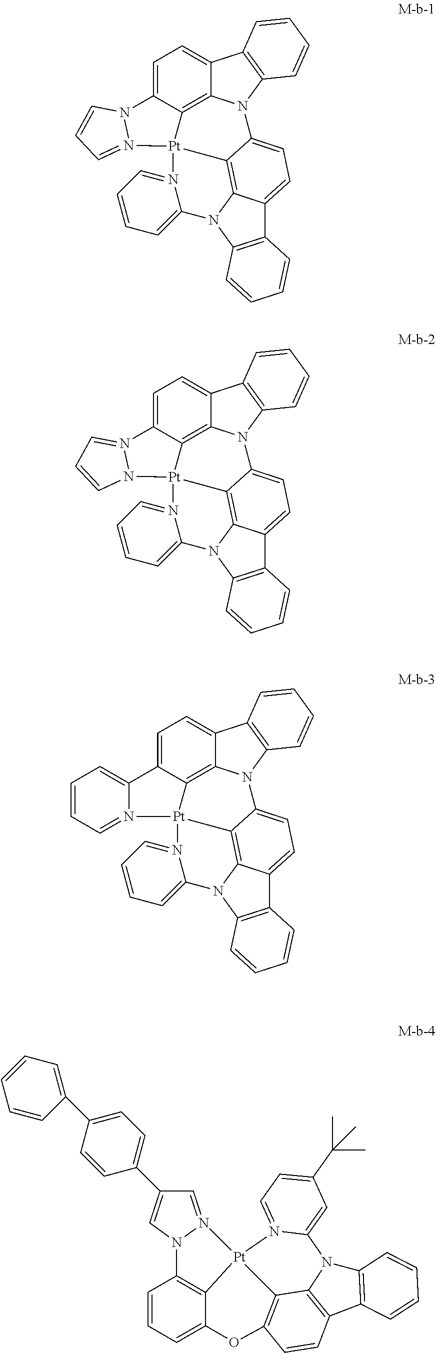

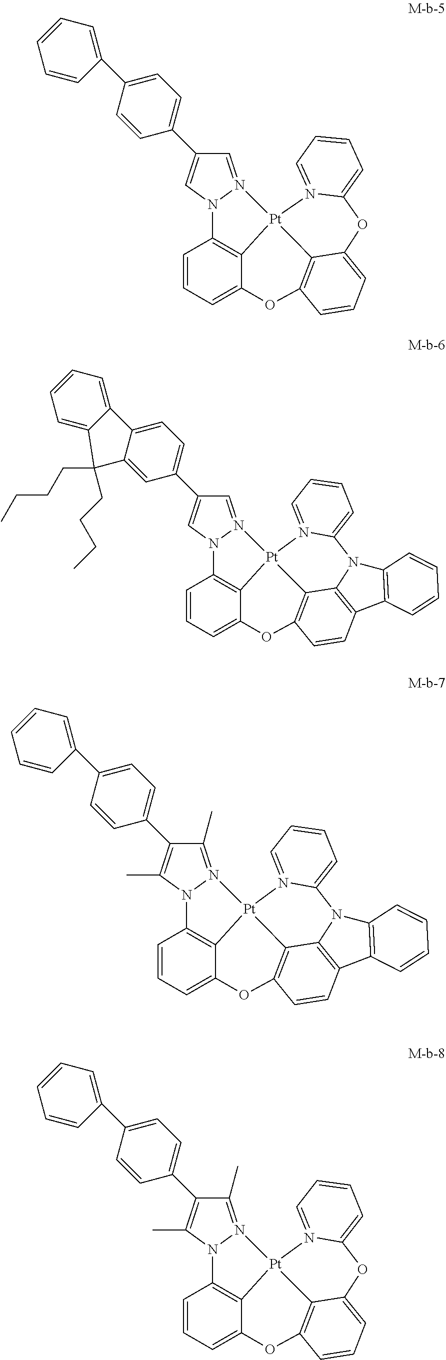

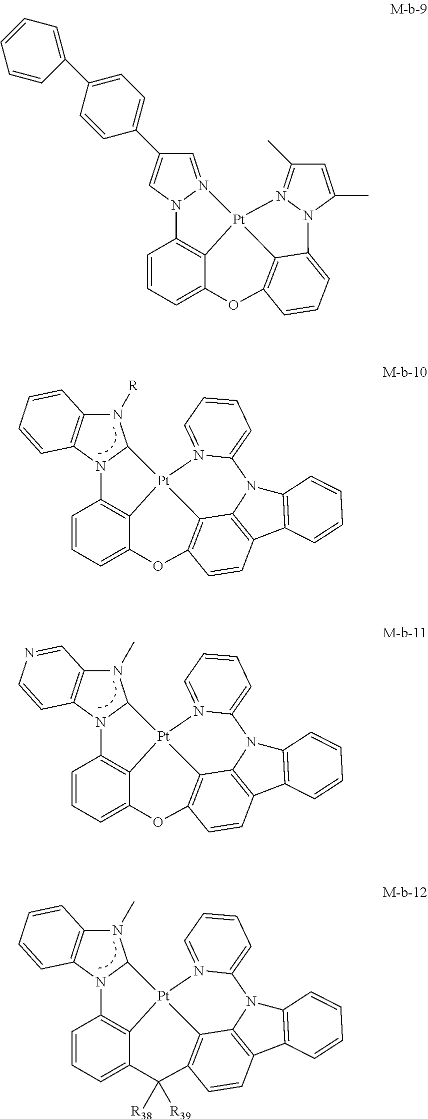

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one of Formula F-a to Formula F-c below. The compounds represented by Formula F-a to Formula F-c below may be used as fluorescence dopant materials.

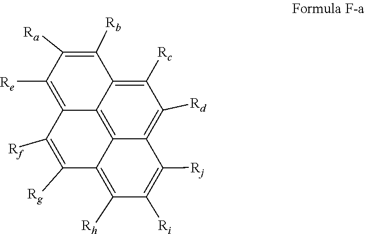

In Formula F-a, two selected from Rₐ to Rⱼ may be each independently substituted with *—NAr₁Ar₂. The remainder not substituted with *—NAr₁Ar₂ of Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In *—NAr₁Ar₂, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one of Ar₁ and Ar₂ may be a heteroaryl group including O or S as a ring-forming atom.

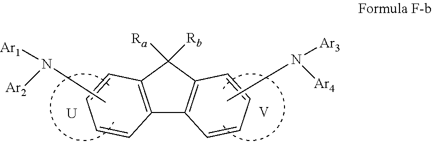

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms. In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may be each independently 0 or 1. For example, in Formula F-b, if the number of U or V is 1, one ring forms a fused ring at the designated part by U or V, and if the number of U or V is 0, a ring is not present at the designated part by U or V. Particularly, if the number of U is 0, and the number of V is 1, or if the number of U is 1, and the number of V is 0, a fused ring having the fluorene core of Formula F-b may be a ring compound with four rings. In addition, if the number of both U and V is 0, the fused ring of Formula F-b may be a ring compound with three rings. In addition, if the number of both U and V is 1, a fused ring having the fluorene core of Formula F-b may be a ring compound with five rings.

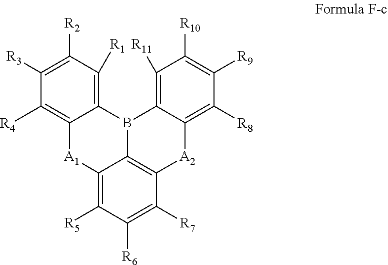

In Formula F-c, A₁ and A₂ may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. The variables R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may be each independently combined with the substituents of an adjacent ring to form a fused ring. For example, if A₁ and A₂ may be each independently NRₘ, A₁ may be combined with R₄ or R₅ to form a ring. In addition, A₂ may be combined with R₇ or R₈ to form a ring.

In an embodiment, the emission layer EML may include as a known dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may include a known phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). Particularly, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, the embodiments are not limited thereto.

The emission layer EML may include a quantum dot material. The core of the quantum dot may be selected from a compound of Groups II-VI, a compound of Groups III-V, a compound of Groups I, III, and VI, a compound of Groups III-V, a compound of Groups III, II, and V, a compound of Groups IV-VI, an element of Group IV, a compound of Group IV group, and combinations thereof.

The compound of Groups II-VI may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The compound of Groups III-VI may include a binary compound such as In₂S₃, and In₂Se₃, a ternary compound such as InGaS₃, and InGaSe₃, or optional combinations thereof. The compound of Groups I, III, and VI may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The compound of Groups III-V may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. The compound of Groups III-V may further include a metal of Group II. For example, InZnP, etc. may be selected as a compound of Groups III, II, and V.

The compound of Groups IV-VI may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The element of Group IV may be selected from the group consisting of Si, Ge, and a mixture thereof. The compound of Group IV may be a binary compound selected from the group consisting of SiC, SiGe, and a mixture thereof.

In this case, the binary compound, the ternary compound or the quaternary compound may be present at uniform concentration in a particle or may be present at a partially different concentration distribution state in the same particle. In addition, a core/shell structure in which one quantum dot wraps another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing the chemical deformation of the core to maintain semiconductor properties and/or a charging layer for imparting the quantum dot with electrophoretic properties. The shell may have a single layer or a multilayer. The interface of the core and shell may have concentration gradient of decreasing the concentration of elements present in the shell toward the center. Examples of the shell of the quantum dot may include a metal or a non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but the embodiments are not limited thereto. Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiments are not limited thereto.

The quantum dot may have a full width of half maximum (FWHM) of emission wavelength spectrum of about 45 nm or less, preferably, about 40 nm or less, more preferably, about 30 nm or less. Within this range, color purity or color reproducibility may be improved. In addition, light emitted via such quantum dot is emitted in all directions, and light view angle properties may be improved.

In addition, the shape of the quantum dot may be generally used shapes in the art, without specific limitation. More particularly, the shape of a generally spherical, a generally pyramidal, a generally multi-armed, or a generally cubic nanoparticle, or a generally nanotube-shaped, a generally nanowire-shaped, a generally nanofiber-shaped, or a generally nanoplate-shaped particle, etc. may be used. The quantum dot may control the color of light emitted according to the particle size, and accordingly, the quantum dot may have various emission colors such as blue, red and green.

In the luminescence devices ED shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, the embodiments are not limited thereto. The electron transport region ETR may have a single layer formed using a single material, a single layer formed using multiple different materials, or a multilayer structure having multiple layers formed using multiple different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1 below.

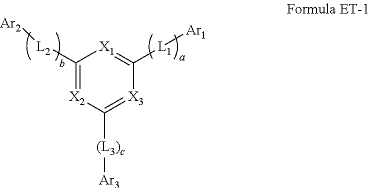

In Formula ET-1, at least one of X₁ to X₃ is N, and the remainder are CRₐ. The variable Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. The variables Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may be each independently an integer of 0 to 10. In Formula ET-1, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. If “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, the embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one of Compounds ET1 to ET36 below.

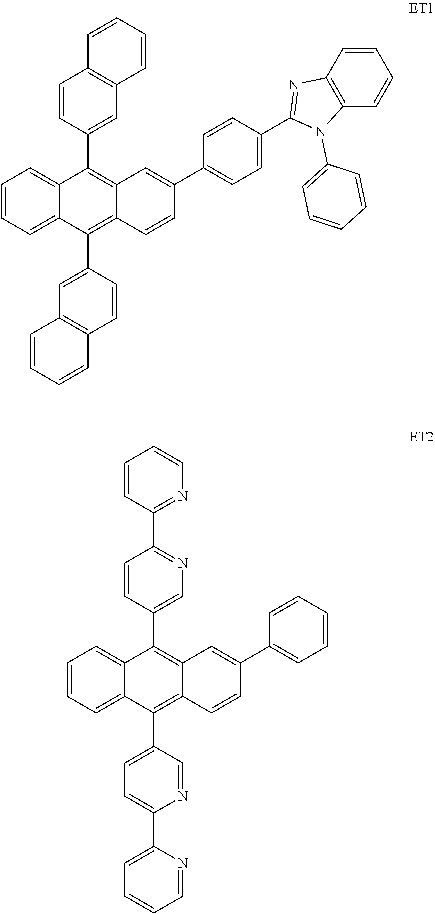 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1]

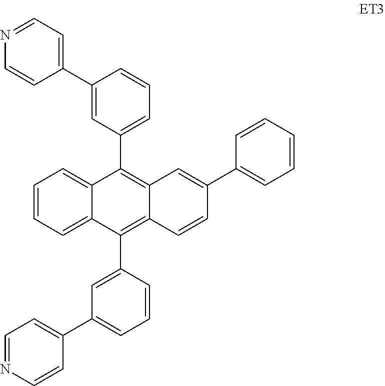 [SMILES: c1ccc(-c2ccc3c(-c4cccc(-c5ccncc5)c4)c4ccccc4c(-c4cccc(-c5ccncc5)c4)c3c2)cc1]

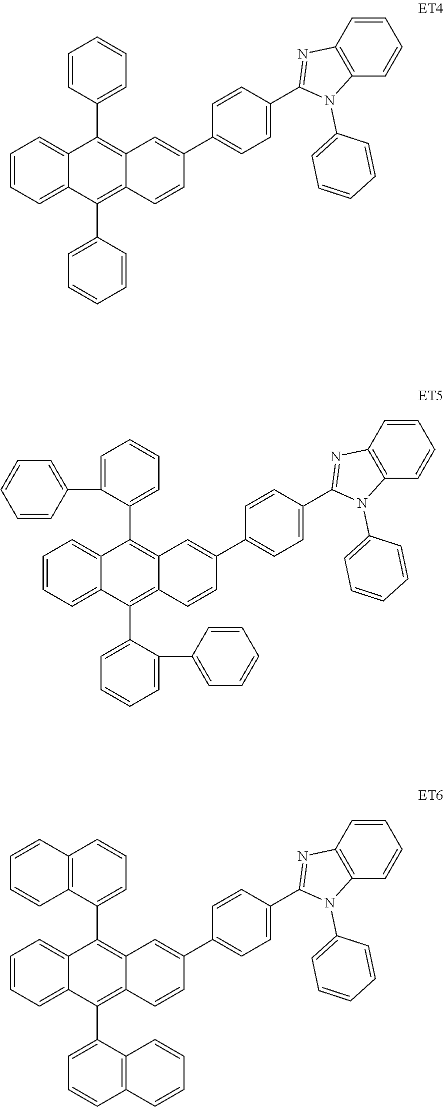 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

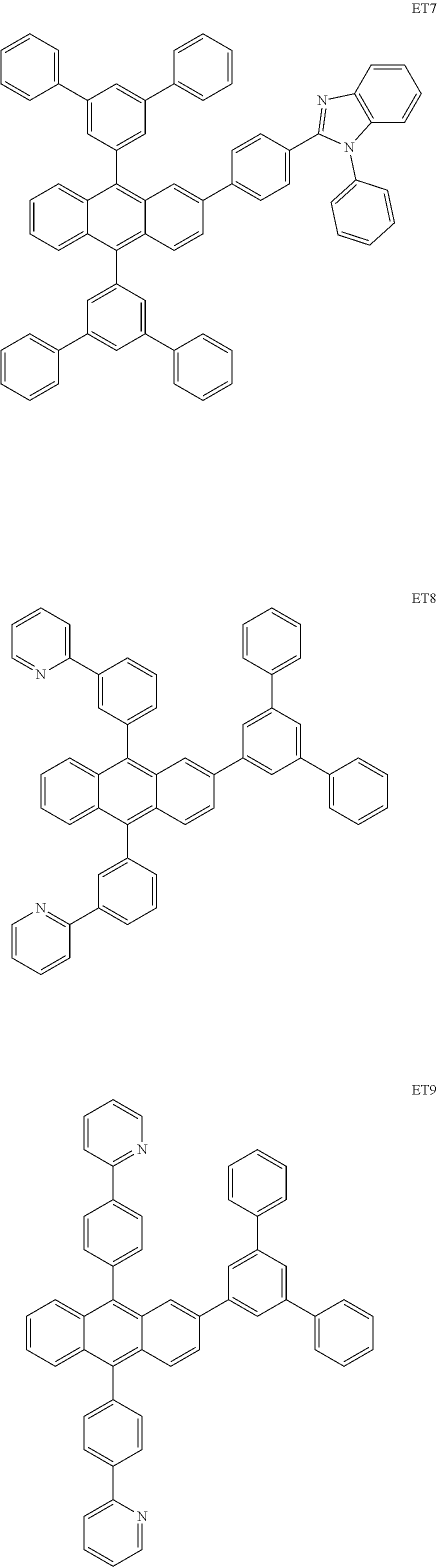 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

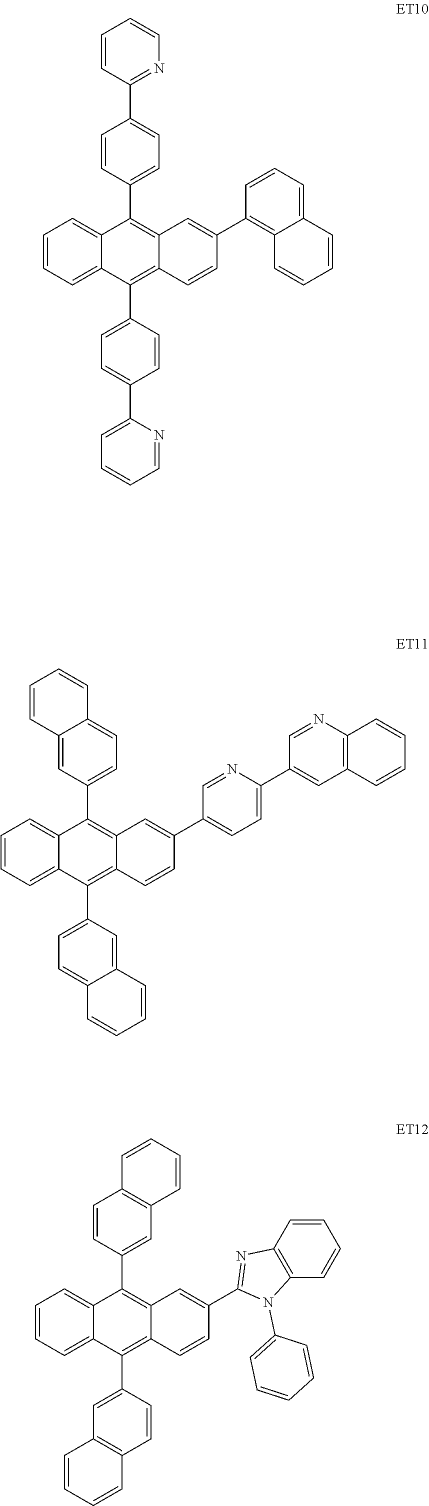 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

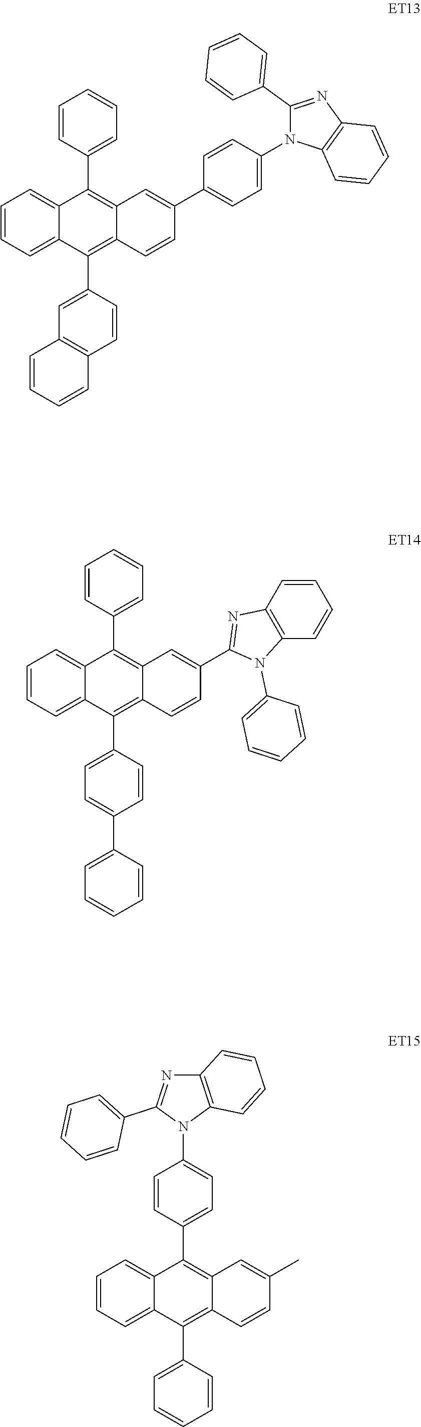 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

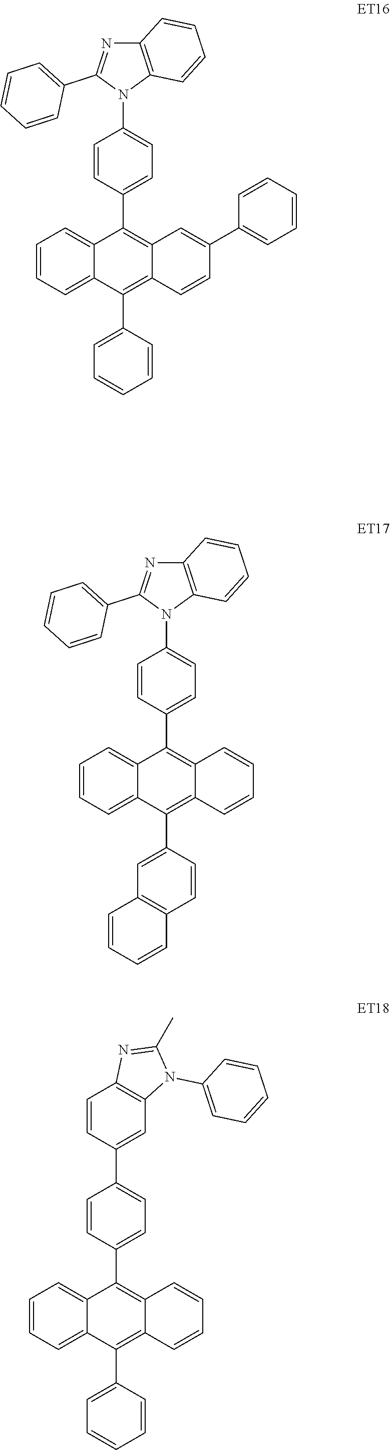 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

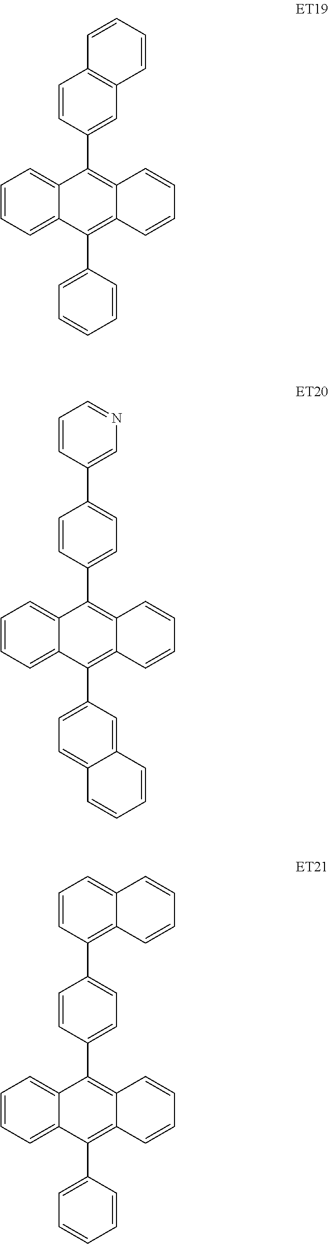

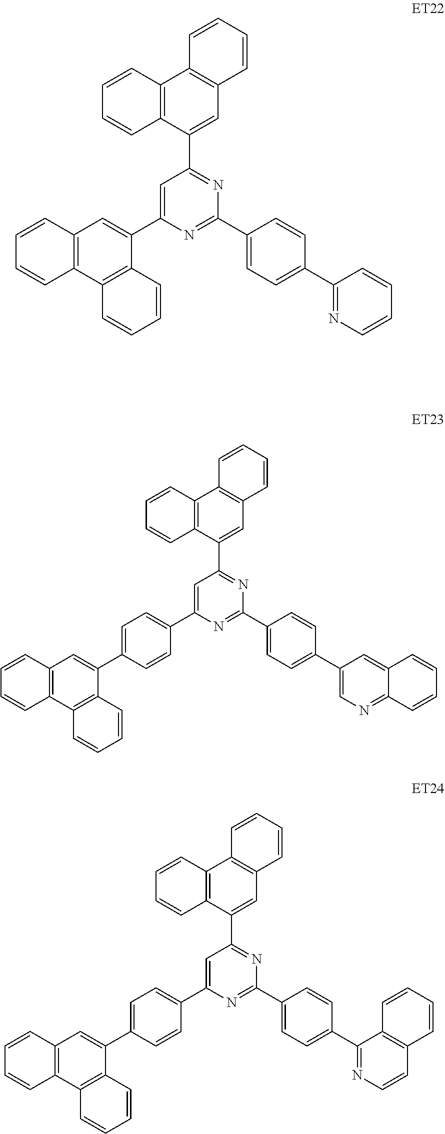 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5ccc(-c6cc7ccccc7c7ccccc67)cc5)cc(-c5cc6ccccc6c6ccccc56)n4)cc3)nccc2c1]

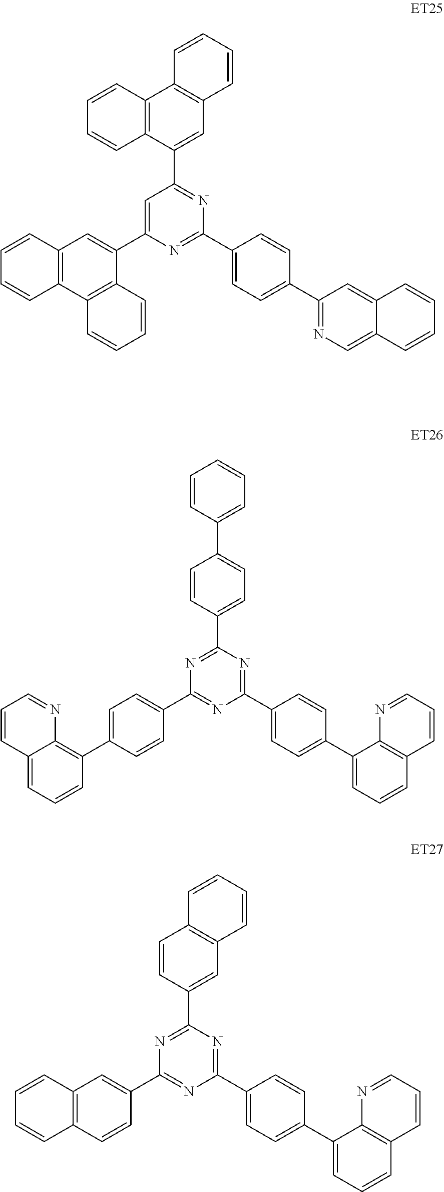 [SMILES: c1ccc2cc(-c3ccc(-c4nc(-c5cc6ccccc6c6ccccc56)cc(-c5cc6ccccc6c6ccccc56)n4)cc3)ncc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

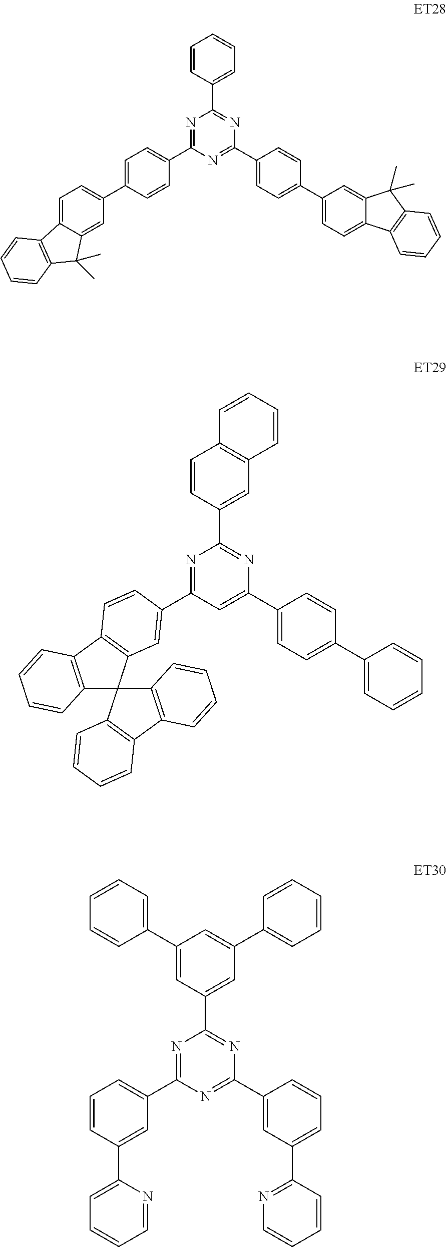 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

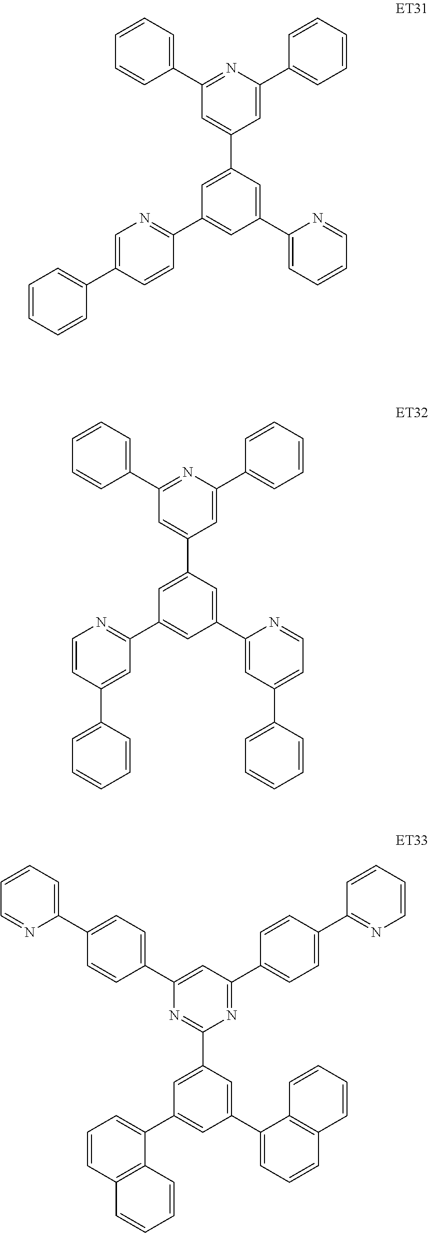 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

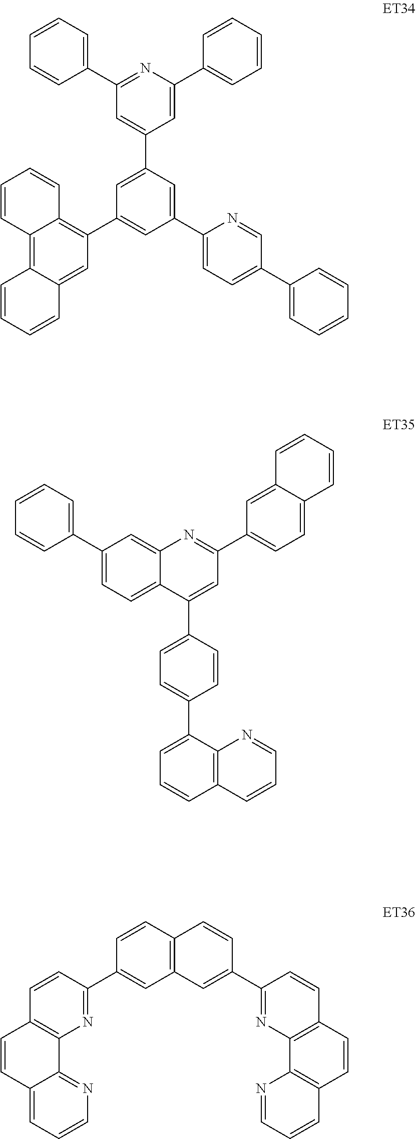 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and KI, a metal in lanthanoides such as Yb, or a co-depositing material of the metal halide and the metal in lanthanoides. For example, the electron transport region ETR may include KI:Yb, RbI:Yb, etc., as the co-depositing material. The electron transport region ETR may use a metal oxide such as Li₂O and BaO, or 8-hydroxy-lithium quinolate (Liq). However, the embodiments are not limited thereto. The electron transport region ETR also may be formed using a mixture material of an electron transport material and an insulating organo metal salt. The organo metal salt may be a material having an energy band gap of about 4 electron volt (eV) or more. Particularly, the organo metal salt may include, for example, metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP) or 4,7-diphenyl-1,10-phenanthroline (Bphen) in addition to the above-described materials. However, the embodiments are not limited thereto.

The electron transport region ETR may include the compounds of the electron transport region in at least one of an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the above-described range, satisfactory electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but the embodiments are not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode or a reflective electrode. If the second electrode EL2 is the transmissive electrode, the second electrode EL2 may include a transparent metal oxide, for example, an ITO, an IZO, a ZnO, an ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds including thereof, or mixtures thereof (for example, AgMg, AgYb, or MgAg). Otherwise, the second electrode EL2 may have a multilayered structure including a reflective layer or a transflective layer formed using the above-described materials and a transparent conductive layer formed using an ITO, an IZO, a ZnO, an ITZO, etc. For example, the second electrode EL2 may include the aforementioned metal materials, combinations of two or more metal materials selected from the aforementioned metal materials, or oxides of the aforementioned metal materials.

The second electrode EL2 may be connected with an auxiliary electrode. If the second electrode EL2 is connected with the auxiliary electrode, the resistance of the second electrode EL2 may decrease. On the second electrode EL2 in the luminescence device ED, a capping layer CPL may be further disposed. The capping layer CPL may include a multilayer or a single layer.

In an embodiment, the capping layer CPL may be an organic layer or an inorganic layer. For example, if the capping layer CPL includes an inorganic material, the inorganic material may include an alkali metal compound such as LiF, an alkaline earth metal compound such as MgF₂, SiON, SiNₓ, SiO_{y}, etc.

For example, if the capping layer CPL includes an organic material, the organic material may include 2,2′-Dimethyl-N,N′-di-[(1-naphthyl)-N,N-diphenyl]-1,1′-biphenyl-4,4′-diamine (α-NPD), NPB, TPD, m-MTDATA, Alq₃, Copper(II) phthalocyanine (CuPc), N4,N4,N4′,N4′-tetra(biphenyl-4-yl) biphenyl-4,4′-diamine (TPD15), 4,4′,4″-tris(carbazol sol-9-yl) triphenylamine (TCTA), etc., or includes an epoxy resin, or acrylate such as methacrylate. In addition, a capping layer CPL may include at least one of Compounds P1 to P5 below, but the embodiments are not limited thereto.

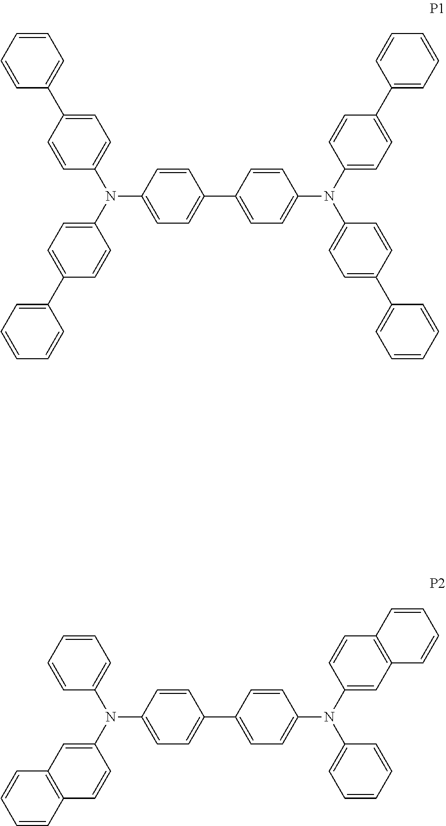 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

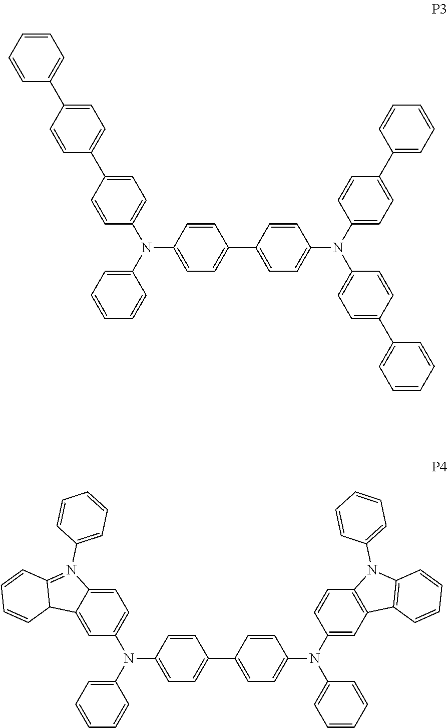

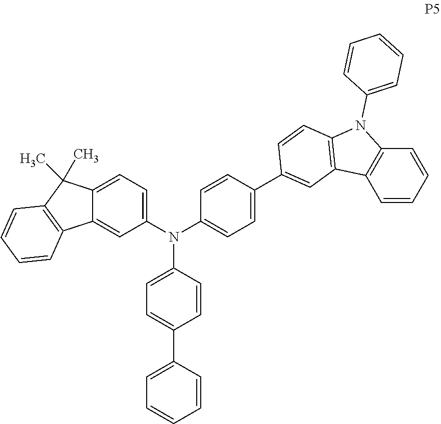 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

The refractive index of the capping layer CPL may be about 1.6 or more. Particularly, the refractive index of the capping layer CPL with respect to light in a wavelength range of about 550 nm to about 660 nm may be about 1.6 or more.

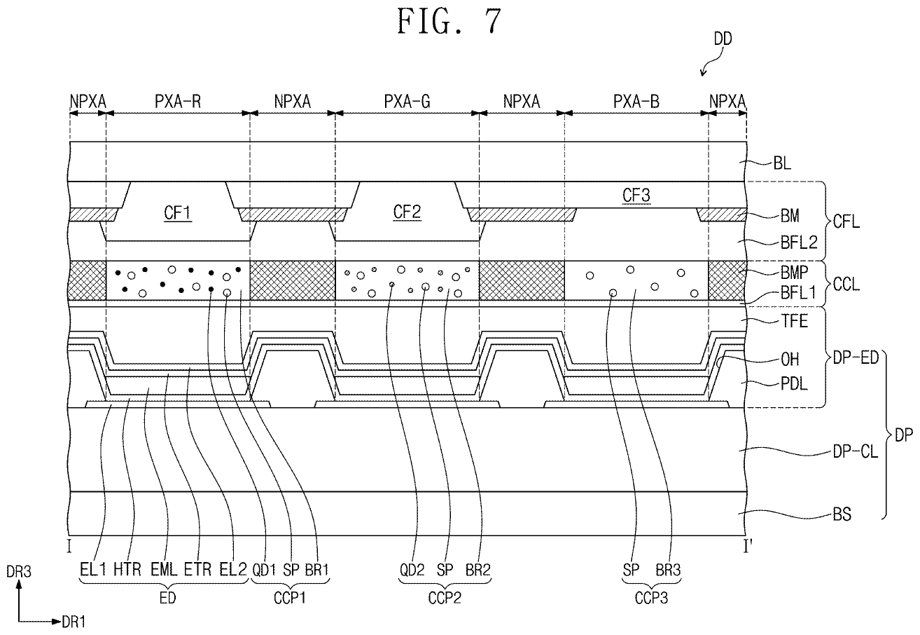

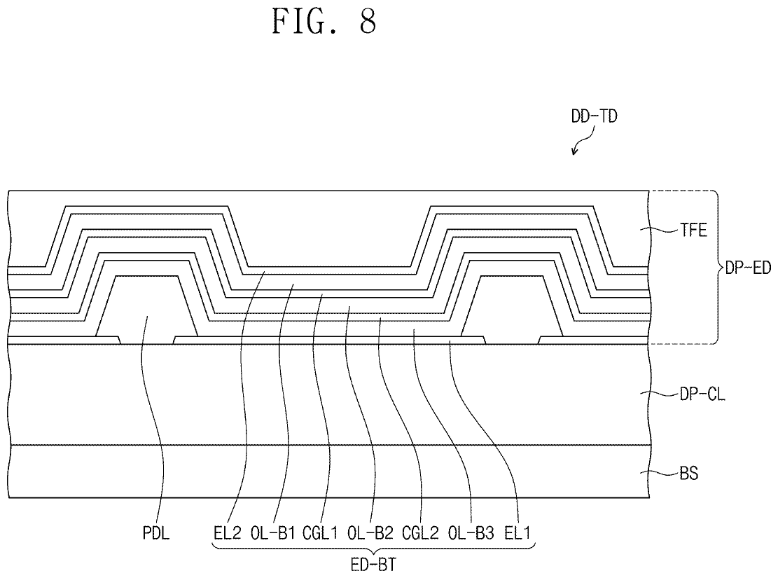

FIG. 7 is a cross-sectional view of another embodiment of a display apparatus including a luminescence device constructed according to the principles of the invention. FIG. 8 is a cross-sectional view of a further embodiment of a display apparatus including a luminescence device constructed according to the principles of the invention.

In the explanation of the embodiments of the display apparatuses shown in FIG. 7 and FIG. 8, repetitive descriptions of features overlapping with the explanation on FIG. 1 to FIG. 6 will not be explained again, but the different features mainly will be explained to avoid redundancy. Referring to FIG. 7, the display apparatus DD may include a display panel DP including a display device layer DP-ED, a light controlling layer CCL disposed on the display panel DP and a color filter layer CFL.

In an embodiment shown in FIG. 7, the display panel DP includes a base layer BS, a circuit layer DP-CL provided on the base layer BS and a display device layer DP-ED, and the display device layer DP-ED may include a luminescence device ED. The luminescence device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The same structures of the luminescence devices of FIG. 3 to FIG. 6 may be applied to the structure of the luminescence device ED shown in FIG. 7.

Referring to FIG. 7, the emission layer EML may be disposed in an opening part OH defined in a pixel definition layer PDL. For example, the emission layer EML divided by the pixel definition layer PDL and overlapping each of the luminous areas PXA-R, PXA-G and PXA-B may emit light in the same wavelength region. In the display apparatus DD, the emission layer EML may emit blue light. Unlike what is shown in FIG. 7, in another embodiment, the emission layer EML may be provided as a common layer for all luminous areas PXA-R, PXA-G and PXA-B.

The light controlling layer CCL may be disposed on the display panel DP. The light controlling layer CCL may include a light converter. The light converter may be a quantum dot or a phosphor. The light converter may transform the wavelength of light and then emit. That is, the light controlling layer CCL may be a layer including a quantum dot or a layer including a phosphor. The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be disposed between the separated light controlling parts CCP1, CCP2 and CCP3, but the embodiments are not limited thereto. In FIG. 7, the partition pattern BMP is shown as not overlapping the light controlling parts CCP1, CCP2 and CCP3, but at least a portion of the edge of the light controlling parts CCP1, CCP2 and CCP3 may overlap the partition pattern BMP.

The light controlling layer CCL may include a first light controlling part CCP1 including a first quantum dot QD1 converting first color light provided from the luminescence device ED into second color light, a second light controlling part CCP2 including a second quantum dot QD2 converting first color light into third color light, and a third light controlling part CCP3 transmitting first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the luminescence device ED. For example, the first quantum dot QD1 may be a red quantum dot, and the second quantum dot QD2 may be a green quantum dot. The same explanation as described above may be applied to the quantum dots QD1 and QD2.

In addition, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one of a TiO₂, ZnO, Al₂O₃, SiO₂, and hollow silica. The scatterer SP may include at least one of the TiO₂, ZnO, Al₂O₃, SiO₂, and hollow silica, or may be a mixture of two or more materials selected of the TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2, and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of various resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may be disposed on the light controlling parts CCP1, CCP2 and CCP3 to block the exposure of the light controlling parts

CCP1, CCP2 and CCP3 to humidity/oxygen. The barrier layer BFL1 may cover the light controlling parts CCP1, CCP2 and CCP3. In addition, the barrier layer BFL2 may be provided between the light controlling parts CCP1, CCP2 and CCP3 and a color filter layer CFL.

The barrier layers BFL1 and BFL2 may include at least one inorganic layer. That is, the barrier layers BFL1 and BFL2 may be formed by including an inorganic material. For example, the barrier layers BFL1 and BFL2 may be formed by including a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide and a silicon oxynitride or a metal thin film securing light transmittance. The barrier layers BFL1 and BFL2 may further include an organic layer. The barrier layers BFL1 and BFL2 may be composed of a single layer of multiple layers.

In the display apparatus DD, the color filter layer CFL may be disposed on the light controlling layer CCL. For example, the color filter layer CFL may be disposed directly on the light controlling layer CCL. In this case, the barrier layer BFL2 may be omitted.

The color filter layer CFL may include a light blocking part BM and filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or a dye, the second filter CF2 may include a green pigment or a dye, and the third filter CF3 may include a blue pigment or a dye. But, the embodiments are not limited thereto, and the third filter CF3 may not include the pigment or dye. The third filter CF3 may include a polymer photosensitive resin and not include a pigment or a dye. The third filter CF3 may be transparent. The third filter CF3 may be formed using a transparent photosensitive resin.

In addition, in an embodiment, the first filter CF1 and the second filter CF2 may be yellow filters. The first filter CF1 and the second filter CF2 may be provided in one unitary body. The light blocking part BM may be a black matrix. The light blocking part BM may be formed by including an organic light blocking material or an inorganic light blocking material including a black pigment or a black dye. The light blocking part BM may prevent or reduce light leakage phenomenon and define the boundaries between adjacent filters CF1, CF2 and CF3. In addition, in an embodiment, the light blocking part BM may be formed as a blue filter.

Each of the first to third filters CF1, CF2 and CF3 may be disposed corresponding to each of a red luminous area PXA-R, green luminous area PXA-G, and blue luminous area PXA-B. On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, the embodiments are not limited thereto, and the base substrate BL may be an inorganic layer, an organic layer or a composite material layer. In addition, in an embodiment unlike the drawing, the base substrate BL may be omitted.

Particularly, in FIG. 8, the cross-sectional view of a portion corresponding to the display panel DP in FIG. 7 is shown. In a display apparatus DD-TD, the luminescence device ED-BT may include multiple light emitting structures OL-B1, OL-B2 and OL-B3. The luminescence device ED-BT may include oppositely disposed first electrode EL1 and second electrode EL2, and the multiple light emitting structures OL-B1, OL-B2 and OL-B3 stacked in order in the thickness direction and provided between the first electrode EL1 and the second electrode EL2. Each of the light emitting structures OL-B1, OL-B2 and OL-B3 may include an emission layer EML (FIG. 7), and a hole transport region HTR and an electron transport region ETR disposed with the emission layer EML (FIG. 7) therebetween.

That is, the luminescence device ED-BT included in the display apparatus DD-TD may be a luminescence device of a tandem structure including multiple emission layers. In the embodiment shown in FIG. 8, light emitted from the light emitting structures OL-B1, OL-B2 and OL-B3 may be all blue light. However, the embodiments are not limited thereto, and the wavelength regions of light emitted from the light emitting structures OL-B1, OL-B2 and OL-B3 may be different from each other. For example, the luminescence device ED-BT including the multiple light emitting structures OL-B1, OL-B2 and OL-B3 emitting light in different wavelength regions may emit white light.

Between neighboring light emitting structures OL-B1, OL-B2 and OL-B3, charge generating layers CGL1 and CGL2 may be disposed. The charge generating layers CGL1 and CGL2 may include a p-type charge generating layer and/or an n-type charge generating layer.

Hereinafter, the principles of the invention will be particularly explained referring to illustrative embodiments and comparative embodiments. The embodiments are only illustrations to assist the understanding of the invention, and the scope of the invention is not limited thereto.

### Synthetic Examples

Embodiments of the nitrogen-containing compound may be synthesized, for example, as follows. However, the synthetic methods are not limited to the examples below.

1. Synthesis of Intermediate Compound A

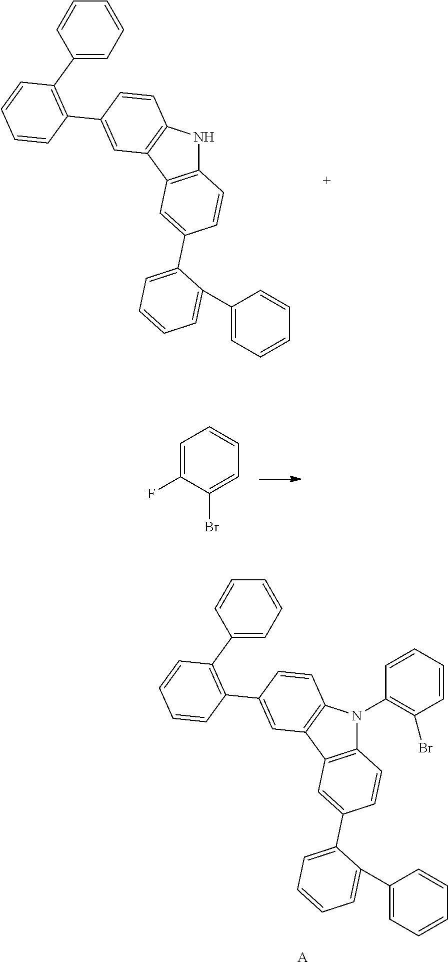

Under an argon (Ar) atmosphere, to a 500 milliliter (mL), three-neck flask, 26.5 gram (g) or 56.1 millimole (mmol) of 3,6-di([1,1′-biphenyl]-2-yl)-9H-carbazole, 7.3 mL (67.3 mmol) of 2-bromofluorobenzene, 36.6 g (112 mmol) of cesium carbonate (Cs₂CO₃), and 112 mL of dimethylformamide (DMF) were added, and stirred at about 150° C. for about 8 hours. After cooling in the air, water was added to the reaction solution, and a white precipitate was obtained by filtering. The crude product thus obtained was separated by column chromatography (silica gel), and 32.5 g (yield 92 percent (%)) of Compound A of a white solid was obtained. The molecular weight of Compound A measured by fast atom bombardment/mass spectroscopy (FAB-MS) was 626.

2. Synthesis of Intermediate Compound B

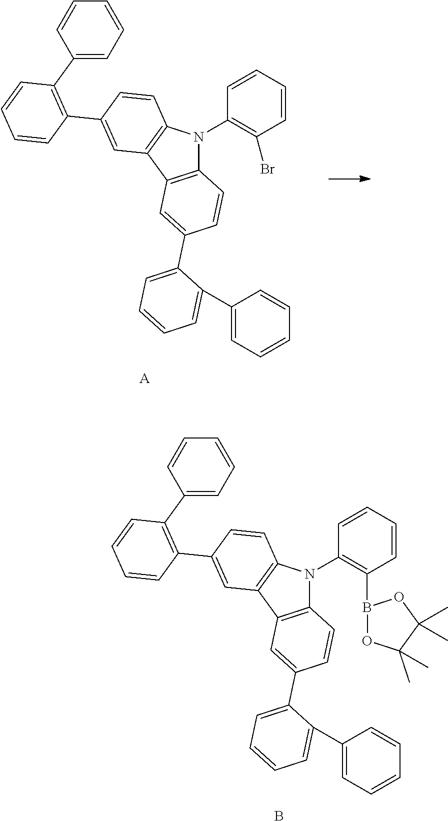

Under an Ar atmosphere, to a 100 mL, three-neck flask, 16.3 g (26.0 mmol) of Intermediate Compound A, 9.90 g (39.0 mmol) of bis(pinacolato)diboron, 1.06 g (1.3 mmol) of cyclopentyl(diphenyl)phosphane; dichloropalladium; iron (PdCl₂(dppf)), 8.24 g (84.0 mmol) of potassium acetate (KOAc), and 130 mL of dioxane were added, and stirred at about 120° C. for about 24 hours. After cooling in the air, water was added to the reaction solution, an organic layer was separated, and a solvent was distilled off. The crude product thus obtained was separated by column chromatography (silica gel), and 11.7 g (yield 67%) of Compound B of a white solid was obtained. The molecular weight of Compound B measured by FAB-MS was 673.

3. Synthesis of Compound 1

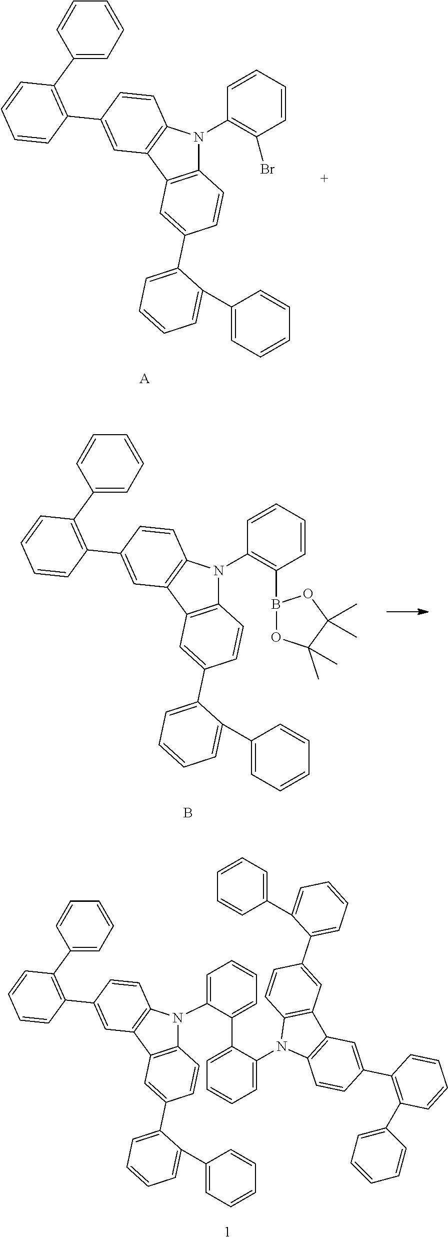

Under an Ar atmosphere, to a 100 mL, three-neck flask, 3.01 g (4.8 mmol) of Intermediate Compound A, 3.57 g (5.3 mmol) of Intermediate Compound B, 0.054 g (0.24 mmol) of Pd(OAc)₂, 0.27 g (1.0 mmol) of triphenylphosphine (PPh₃), 1.99 g (14.4 mmol) of potassium carbonate (K₂CO₃), 24 mL of toluene, 7 mL of ethanol, and 2 mL of water were added, and stirred at about 100° C. for about 4 hours. After cooling in the air, water was added to the reaction solution, an organic layer was separated, and a solvent was distilled off. The crude product thus obtained was separated by column chromatography (silica gel), and 3.62 g (yield 69%) of Compound 1 of a white solid was obtained. The molecular weight of Compound 1 measured by FAB-MS was 1093. In addition, the chemical shift values δ of Compound 1 measured by proton nuclear magnetic resonance (¹H-NMR (trichloro(deuterio)methane (CDCl₃))) were 8.20 (2H), 8.03 (2H), 7.91-7.77 (18H), 7.72-7.65 (12H), 7.52-7.48 (8H), 7.38-7.29 (14H).

4. Synthesis of Compound 3

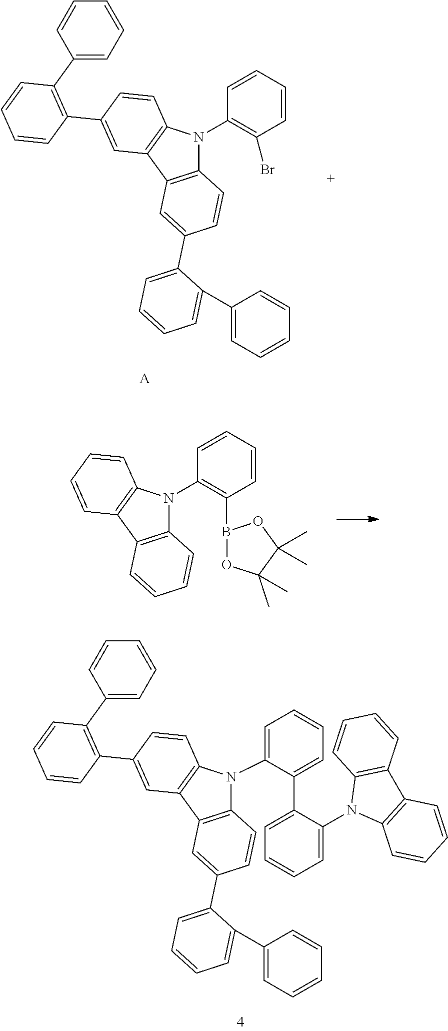

Under an Ar atmosphere, to a 100 mL, three-neck flask, 3.76 g (6.0 mmol) of Intermediate Compound A, 2.44 g (6.6 mmol) of 9-(2-(4,4,5,5-tetramethyl-1,3,2-dioxaborolan-2-yl)phenyl)-9H-carbazole, 0.067 g (0.30 mmol) of palladium(2+); diacetate (Pd(OAc)₂), 0.31 g (1.2 mmol) of PPh₃, 2.49 g (18.0 mmol) of K₂CO₃, 30 mL of toluene, 9 mL of ethanol, and 3 mL of water were added, and stirred at about 100° C. for about 4 hours. After cooling in the air, water was added to the reaction solution, an organic layer was separated, and a solvent was distilled off. The crude product thus obtained was separated by column chromatography (silica gel), and 3.41 g (yield 72%) of Compound 3 of a white solid was obtained. The molecular weight of Compound 3 measured by FAB-MS was 788. In addition, the chemical shift values δ of Compound 3 measured by ¹H-NMR (CDCl₃) were 8.45 (1H), 8.20 (1H), 8.11-8.01 (2H), 7.91-7.67 (19H), 7.52-7.23 (15H), 7.12-7.04 (2H).

5. Synthesis of Compound 4

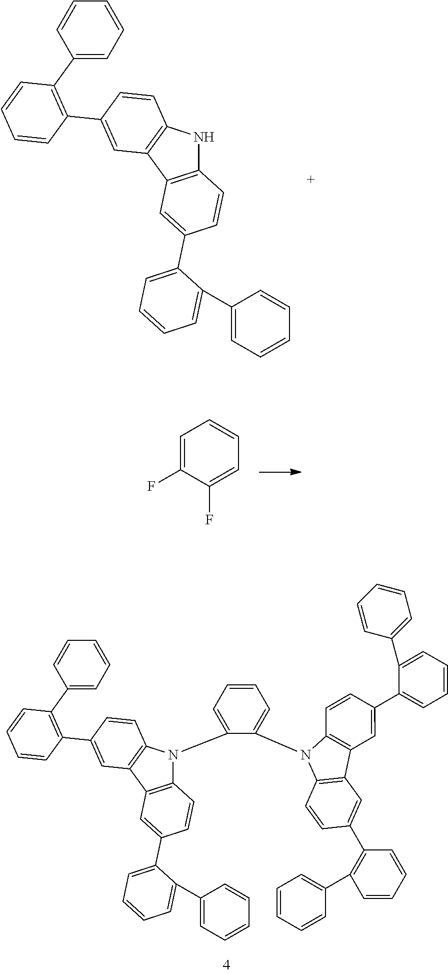 [SMILES: c1ccc(-c2ccccc2-c2ccc3[nH]c4ccc(-c5ccccc5-c5ccccc5)cc4c3c2)cc1 ; Fc1ccccc1F ; c1ccc(-c2ccccc2-c2ccc3c(c2)c2cc(-c4ccccc4-c4ccccc4)ccc2n3-c2ccccc2-n2c3ccc(-c4ccccc4-c4ccccc4)cc3c3cc(-c4ccccc4-c4ccccc4)ccc32)cc1]

Under an Ar atmosphere, to a 300 mL, three-neck flask, 26.5 g (56.1 mmol) of 3,6-di([1,1′-biphenyl]-2-yl)-9H-carbazole, 2.7 mL (27.5 mmol) of o-difluorobenzene, 36.0 g (110 mmol) of Cs₂CO₃, and 110 mL of DMF were added, and stirred at about 100° C. for about 8 hours, and then, stirred at about 150° C. for about 24 hours. After cooling in the air, water was added to the reaction solution, and a white precipitate produced was obtained by filtering. The crude product thus obtained was separated by column chromatography (silica gel), and 8.11 g (yield 29%) of Compound 4 of a white solid was obtained. The molecular weight of Compound 4 measured by FAB-MS was 1017. In addition, the chemical shift values δ of Compound 4 measured by ¹H-NMR (CDCl₃) were 8.20 (2H), 8.03 (2H), 7.91-7.69 (26H), 7.58-7.48 (10H), 7.38-7.29 (12H).

6. Synthesis of Compound 6

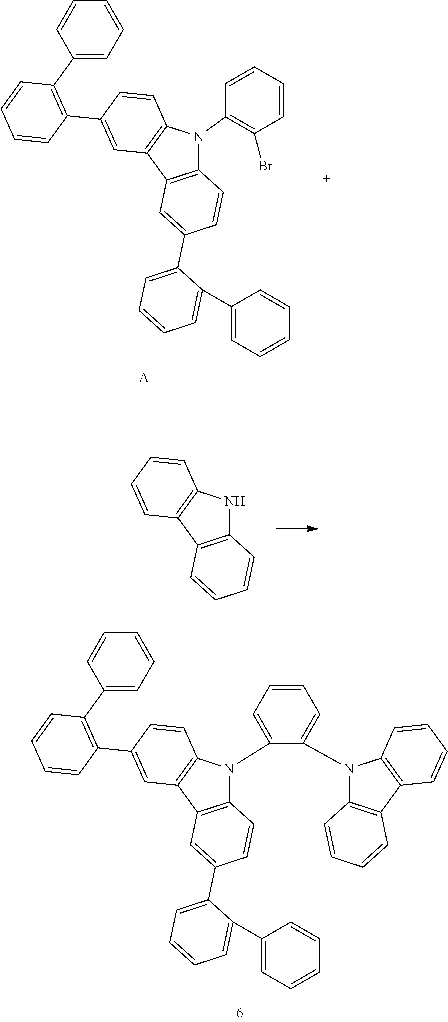

Under an Ar atmosphere, to a 100 mL, three-neck flask, 3.76 g (6.0 mmol) of Intermediate Compound A, 2.01 g (12.0 mmol) of carbazole, 0.11 g (0.60 mmol) of copper(I) iodide (CuI), 0.22 g (1.2 mmol) of 1,10-phenanthroline, 0.67 g (12.0 mmol) of potassium hydroxide (KOH), and 12 mL of o-dichlorobenzene were added, and stirred at about 180° C. for about 24 hours. After cooling in the air, water was added to the reaction solution, an organic layer was separated, and a solvent was distilled off. The crude product thus obtained was separated by column chromatography (silica gel), and 1.67 g (yield 39%) of Compound 6 of a white solid was obtained. The molecular weight of Compound 6 measured by FAB-MS was 712. In addition, the chemical shift values δ of Compound 6 measured by ¹H-NMR (CDCl₃) were 8.45 (1H), 8.20 (1H), 8.11-8.01 (2H), 7.91-7.65 (15H), 7.52-7.23 (15H), 7.12-7.04 (2H).

7. Synthesis of Compound 8

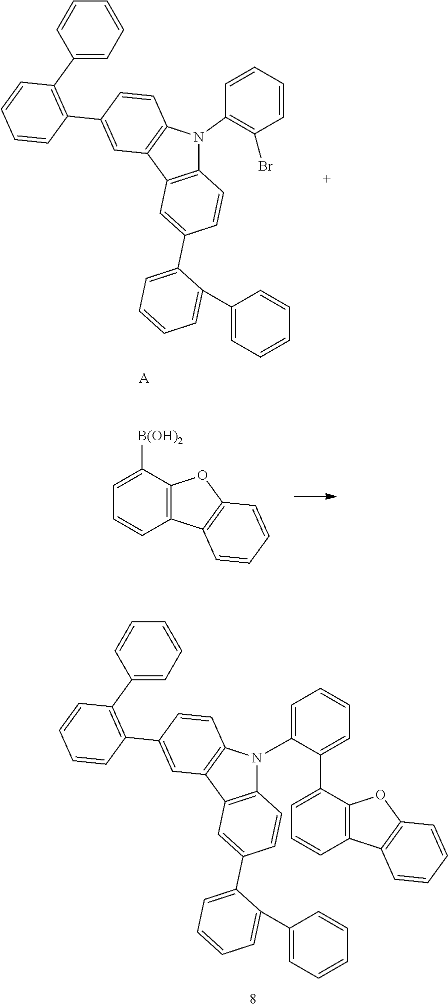

Under an Ar atmosphere, to a 100 mL, three-neck flask, 3.76 g (6.0 mmol) of Intermediate Compound A, 1.40 g (6.6 mmol) of dibenzo[b,d]furan-4-ylboronic acid, 0.067 g (0.30 mmol) of Pd(OAc)₂, 0.31 g (1.2 mmol) of PPh₃, 2.49 g (18.0 mmol) of K₂CO₃, 30 mL of toluene, 9 mL of ethanol, and 3 mL of water were added, and stirred at about 100° C. for about 4 hours. After cooling in the air, water was added to the reaction solution, an organic layer was separated, and a solvent was distilled off. The crude product thus obtained was separated by column chromatography (silica gel), and 3.38 g (yield 79%) of Compound 8 of a white solid was obtained. The molecular weight of Compound 8 measured by FAB-MS was 713. In addition, the chemical shift values δ of Compound 8 measured by ¹H-NMR (CDCl₃) were 8.20 (1H), 8.05-7.79 (13H), 7.72-7.65 (6H), 7.52-7.19 (15H).

8. Synthesis of Compound 7

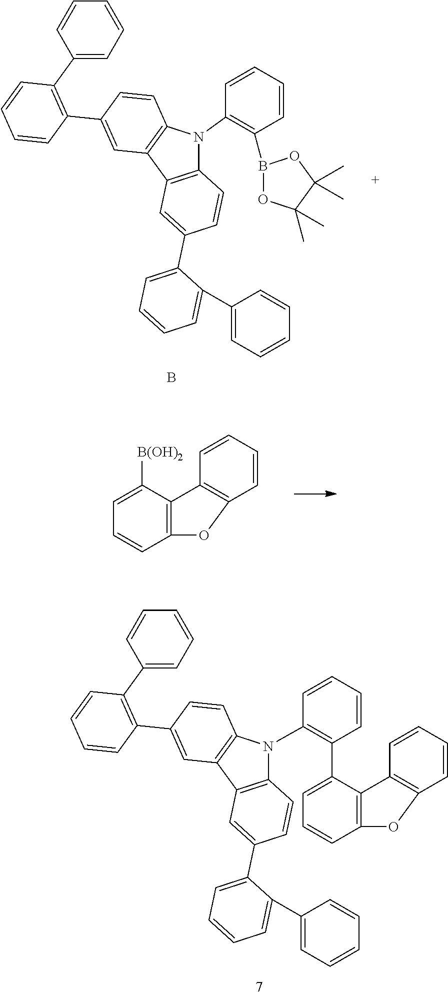

Under an Ar atmosphere, to a 100 mL, three-neck flask, 4.04 g (6.0 mmol) of Intermediate Compound B, 1.63 g (6.6 mmol) of 1-bromodibenzo[b,d]furan, 0.067 g (0.30 mmol) of Pd(OAc)₂, 0.31 g (1.2 mmol) of PPh₃, 2.49 g (18.0 mmol) of K₂CO₃, 30 mL of toluene, 9 mL of ethanol, and 3 mL of water were added, and stirred at about 100° C. for about 4 hours. After cooling in the air, water was added to the reaction solution, an organic layer was separated, and a solvent was distilled off. The crude product thus obtained was separated by column chromatography (silica gel), and 3.51 g (yield 82%) of Compound 7 of a white solid was obtained. The molecular weight of Compound 7 measured by FAB-MS was 713. In addition, the chemical shift values δ of Compound 7 measured by ¹H-NMR (CDCl₃) were 8.20 (1H), 8.03 (1H), 7.91-7.65 (17H), 7.59 (1H), 7.52-7.19 (15H).

9. Synthesis of Compound 14

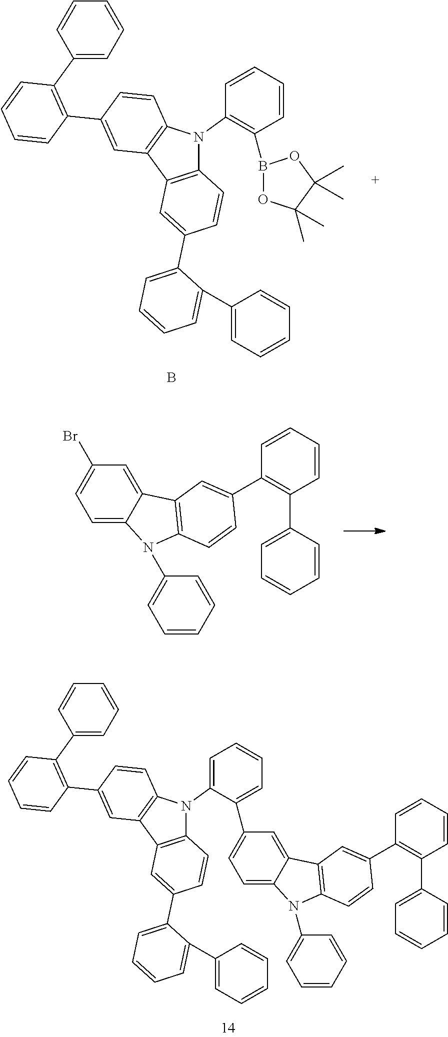

Under an Ar atmosphere, to a 100 mL, three-neck flask, 4.04 g (6.0 mmol) of Intermediate Compound B, 3.13 g (6.6 mmol) of 3-([1,1′-biphenyl]-2-yl)-6-bromo-9-phenyl-9H-carbazole, 0.067 g (0.30 mmol) of Pd(OAc)₂, 0.31 g (1.2 mmol) of PPh₃, 2.49 g (18.0 mmol) of K₂CO₃, 30 mL of toluene, 9 mL of ethanol, and 3 mL of water were added, and stirred at about 100° C. for about 4 hours. After cooling in the air, water was added to the reaction solution, an organic layer was separated, and a solvent was distilled off. The crude product thus obtained was separated by column chromatography (silica gel), and 4.80 g (yield 85%) of Compound 14 of a white solid was obtained. The molecular weight of Compound 14 measured by FAB-MS was 941. In addition, the chemical shift values δ of Compound 14 measured by ¹H-NMR (CDCl₃) were 8.22-8.18 (2H), 8.05-8.01 (2H), 7.91-7.77 (14H), 7.72-7.65 (9H), 7.52-7.29 (21H).

### Examples of Manufacturing Devices

Luminescence devices were manufactured using Example Compounds and Comparative Compounds below as materials for a hole transport region.

### Example Compounds

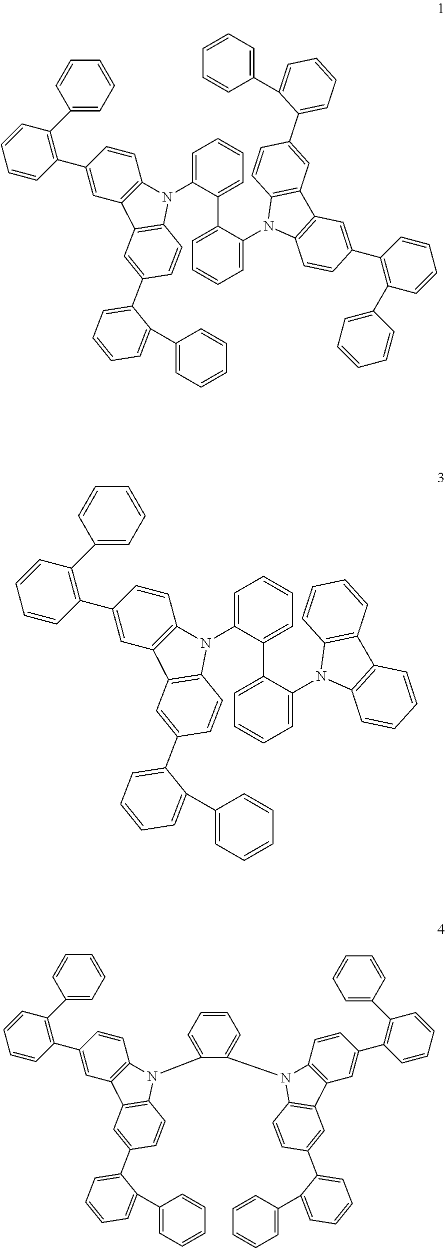 [SMILES: c1ccc(-c2ccccc2-c2ccc3c(c2)c2cc(-c4ccccc4-c4ccccc4)ccc2n3-c2ccccc2-c2ccccc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccccc2-c2ccccc2-n2c3ccccc3c3ccccc32)cc1 ; c1ccc(-c2ccccc2-c2ccc3c(c2)c2cc(-c4ccccc4-c4ccccc4)ccc2n3-c2ccccc2-n2c3ccc(-c4ccccc4-c4ccccc4)cc3c3cc(-c4ccccc4-c4ccccc4)ccc32)cc1]

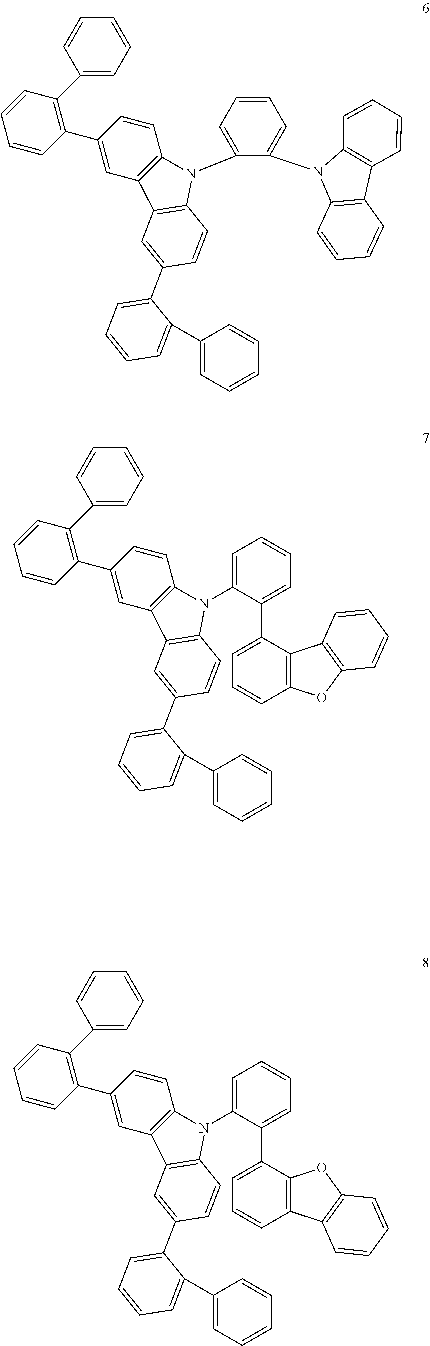 [SMILES: c1ccc(-c2ccccc2-c2ccc3c(c2)c2cc(-c4ccccc4-c4ccccc4)ccc2n3-c2ccccc2-n2c3ccccc3c3ccccc32)cc1 ; c1ccc(-c2ccccc2-c2ccc3c(c2)c2cc(-c4ccccc4-c4ccccc4)ccc2n3-c2ccccc2-c2cccc3oc4ccccc4c23)cc1 ; c1ccc(-c2ccccc2-c2ccc3c(c2)c2cc(-c4ccccc4-c4ccccc4)ccc2n3-c2ccccc2-c2cccc3c2oc2ccccc23)cc1]

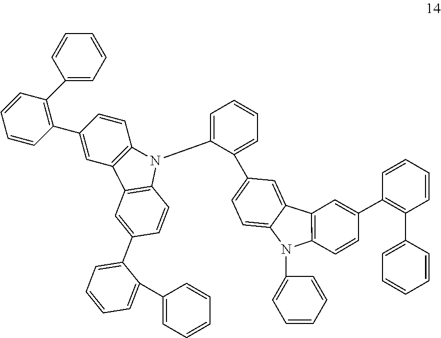 [SMILES: c1ccc(-c2ccccc2-c2ccc3c(c2)c2cc(-c4ccccc4-n4c5ccc(-c6ccccc6-c6ccccc6)cc5c5cc(-c6ccccc6-c6ccccc6)ccc54)ccc2n3-c2ccccc2)cc1]

### Comparative Compounds

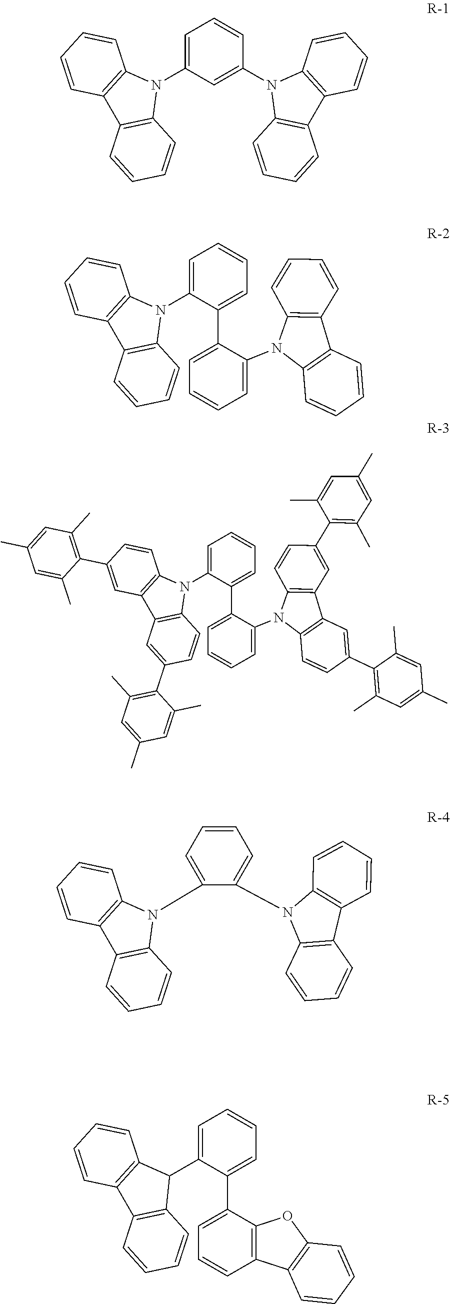

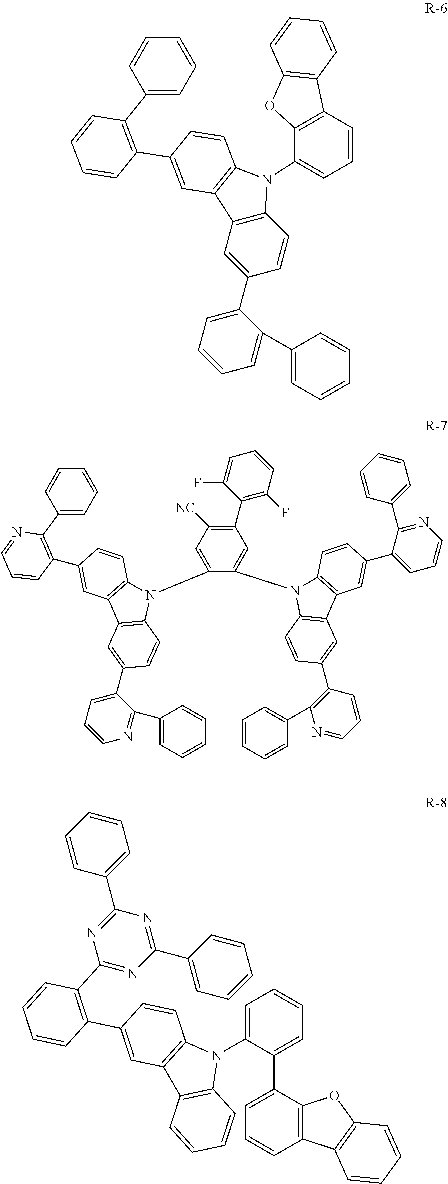 [SMILES: c1ccc(-c2ccccc2-c2ccc3c(c2)c2cc(-c4ccccc4-c4ccccc4)ccc2n3-c2cccc3c2oc2ccccc23)cc1 ; N#Cc1cc(-n2c3ccc(-c4cccnc4-c4ccccc4)cc3c3cc(-c4cccnc4-c4ccccc4)ccc32)c(-n2c3ccc(-c4cccnc4-c4ccccc4)cc3c3cc(-c4cccnc4-c4ccccc4)ccc32)cc1-c1c(F)cccc1F ; c1ccc(-c2nc(-c3ccccc3)nc(-c3ccccc3-c3ccc4c(c3)c3ccccc3n4-c3ccccc3-c3cccc4c3oc3ccccc34)n2)cc1]

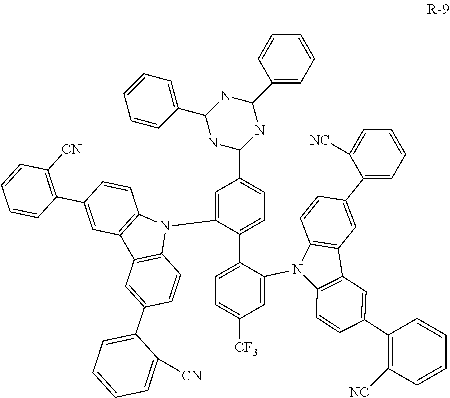 [SMILES: N#Cc1ccccc1-c1ccc2c(c1)c1cc(-c3ccccc3C#N)ccc1n2-c1cc(C2NC(c3ccccc3)NC(c3ccccc3)N2)ccc1-c1ccc(C(F)(F)F)cc1-n1c2ccc(-c3ccccc3C#N)cc2c2cc(-c3ccccc3C#N)ccc21]

The luminescence devices of the Examples and Comparative Examples were manufactured by the method below. On a glass substrate, the compound ITO with a thickness of about 150 nm was patterned, washed with ultrapure water, and treated with ultraviolet (UV) ozone for about 10 minutes to form a first electrode. Next, the compound HATCN was deposited to a thickness of about 10 nm, the compound NPB was deposited to a thickness of about 40 nm, and the Example Compound or Comparative Compound was deposited to a thickness of about 5 nm to form a hole transport region. After that, an emission layer was formed using 9-[3-(3-carbazol-9-ylphenyl)phenyl]carbazole (mCBP) doped with 8% iridium(III) bis(4,6-difluorophenylpyridinato-N,C2′)picolinate (Flrpic) to a thickness of about 20 nm. On the emission layer, a layer with a thickness of about 10 nm was formed using 9-naphthalen-1-yl-10-naphthalen-2-ylanthracene (BmPyPhB), a layer with a thickness of about 30 nm was formed using 1,3,5-tris(1-phenyl-1H-benzo[d]imidazole-2-yl)benzene (TPBi), and a layer with a thickness of about 2 nanometer (nm) was formed using Liq to form an electron transport region. Then, a second electrode with a thickness of about 100 nm was formed using aluminum (Al). All layers were formed by a vacuum deposition method.

The values measured according to Examples 1 to 7 and Comparative Examples 1 to 6 are shown in Table 1 below. Current efficiency was measured at about 10 mA/cm², and half-life was measured by measuring luminance half hour at an initial luminance of about 1000 cd/m² to achieve. All are shown by % based on Comparative Example 1.

TABLE 1

| | | Current | |
|---|---|---|---|
| | | efficiency | Lifespan |
| | Hole transport region | (%) | (%) |
| Example 1 | Example Compound 1 | 110% | 180% |
| Example 2 | Example Compound 3 | 115% | 130% |
| Example 3 | Example Compound 4 | 120% | 160% |
| Example 4 | Example Compound 6 | 110% | 120% |
| Example 5 | Example Compound 7 | 125% | 190% |
| Example 6 | Example Compound 8 | 120% | 210% |
| Example 7 | Example Compound 14 | 130% | 170% |
| Comparative | Comparative Compound R-1 | 100% | 100% |
| Example 1 | | | |
| Comparative | Comparative Compound R-2 | 105% | 90% |
| Example 2 | | | |
| Comparative | Comparative Compound R-3 | 95% | 20% |
| Example 3 | | | |
| Comparative | Comparative Compound R-4 | 105% | 80% |
| Example 4 | | | |
| Comparative | Comparative Compound R-5 | 100% | 60% |
| Example 5 | | | |
| Comparative | Comparative Compound R-6 | 105% | 110% |
| Example 6 | | | |
| Comparative | Comparative Compound R-7 | 80% | 10% |
| Example 7 | | | |
| Comparative | Comparative Compound R-8 | 75% | 10% |
| Example 8 | | | |
| Comparative | Comparative Compound R-9 | 30% | 15% |
| Example 9 | | | |

Table 1 shows that Examples 1 to 7 all exhibited significantly and unexpectedly superior characteristics in terms of long lifespan and high efficiency simultaneously when compared to Comparative Examples 1 to 9. The nitrogen-containing compound made according to the principles and embodiments of the invention are used in a hole transport region to contribute to the increase of the efficiency and lifespan of a luminescence device. Embodiments of the nitrogen-containing compound made according to the principles of the invention had high hole transport properties and stability, and according to the increase of a triplet energy (Ti) level, if used in a hole transport layer which is adjacent to the emission layer of a blue phosphorescence emitting device, high efficiency and achieved long lifespan. Although not wanting to be bound by theory, an aryl group having a substituent at an ortho position was introduced to highly reactive position 3 of a carbazole, to secure stability and to further twist to increase a T1 level. In addition, a fused unit having a heteroaryl group at an ortho position was included as a substituent on N of carbazole, and tolerance to electrons could be improved and the increase of lifespan could be achieved. In Comparative Example 2, no substituents were present at positions 3/6 of carbazole, and it is considered that the reactivity at these parts was high, and lifespan was reduced.

In Comparative Example 3, mesityl groups were present at 3/6 positions of carbazole, and a substituent was present at an ortho position, but because the compound is unstable with the mesityl group, it is considered that lifespan was reduced. In Comparative

Example 4, no substituents were present at positions 3/6 of carbazole, and it is considered that the reactivity at these parts was high, and lifespan was reduced. In Comparative Example 5, no substituents were present at positions 3/6 of carbazole, and it is considered that the reactivity at these parts was high, and lifespan was reduced. In Comparative Example 6, since a heteroaryl group was directly linked to carbazole, the bond between the heteroaryl group and the carbazole was easily cleaved, and it is considered that lifespan was reduced.

In Comparative Examples 7 to 9, an electron acceptable unit such as a pyridine ring, a triazine ring, and a cyano group is included in a molecule, lowest unoccupied molecular orbital (LUMO) was deep, the function as an electron blocking layer was not carried out, and the carrier balance of a device was largely collapsed, thereby markedly reducing efficiency and lifespan.

Luminescence device constructed according to the principles and embodiments of the invention have excellent efficiency. Embodiments of the nitrogen-containing compound made according to the principles of the invention may be used as the material of a hole transport region of a luminescence device, and thereby improve the efficiency of the luminescence device.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A luminescence device, comprising:
a first electrode;
a hole transport region disposed on the first electrode;
an emission layer disposed on the hole transport region;
an electron transport region disposed on the emission layer; and
a second electrode disposed on the electron transport region,
wherein the hole transport region comprises a compound of Formula 1: 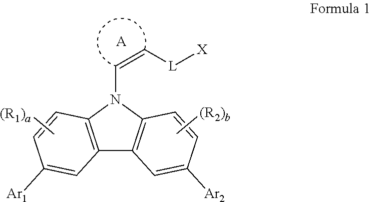
wherein in the Formula 1,
X is a heteroaryl group of 2 to 30 ring-forming carbon atoms, comprising any one of O, S, N or Si,
L is a direct linkage, a substituted or an unsubstituted arylene group of 6 to 30 ring-forming carbon atoms, or a substituted or an unsubstituted heteroarylene group of 2 to 30 ring-forming carbon atoms,
ring A is a substituted or an unsubstituted aryl ring of 6 to 30 ring-forming carbon atoms, or a substituted or an unsubstituted heteroaryl ring of 2 to 30 ring-forming carbon atoms,
a and b are each, independently from one another, an integer of 0 to 3,
R₁ and R₂ are each, independently from one another, a hydrogen atom, a deuterium atom, a halogen atom, a nitro group, a cyano group, a hydroxyl group, a substituted or an unsubstituted amine group, a substituted or an unsubstituted thiol group, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring, and
Ar₁ and Ar₂ are each, independently from one another, of Formula 2: 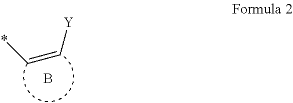
wherein in the Formula 2,
Y is a substituted or an unsubstituted aryl group of 6 to 30 ring-forming carbon atoms, or a substituted or an unsubstituted heteroaryl group of 2 to 30 ring-forming carbon atoms, comprising any one of a furan ring, a thiophene ring, a pyrrole ring, or a silole ring,
* is a binding site to an adjacent atom, and
ring B is a substituted or an unsubstituted aryl ring of 6 to 30 ring-forming carbon atoms, or a substituted or an unsubstituted heteroaryl ring of 2 to 30 ring-forming carbon atoms.

2. The luminescence device of claim 1, wherein the hole transport region comprises:
a hole injection layer disposed on the first electrode;
a hole transport layer disposed on the hole injection layer, and
the compound of the Formula 1 is included in at least one of the hole injection layer and the hole transport layer.

3. The luminescence device of claim 1, wherein the hole transport region comprises:
a hole transport layer disposed on the first electrode;
an electron blocking layer disposed on the hole transport layer, and
the electron blocking layer comprises the compound of the Formula 1.

4. The luminescence device of claim 1, wherein the X is a substituted or an unsubstituted heteroaryl group of 2 to 30 ring-forming carbon atoms, comprising any one of a furan ring, a thiophene ring, a pyrrole ring, or a silole ring.

5. The luminescence device of claim 4, wherein X of the Formula 1 is of Formula 3-1 or Formula 3-2: 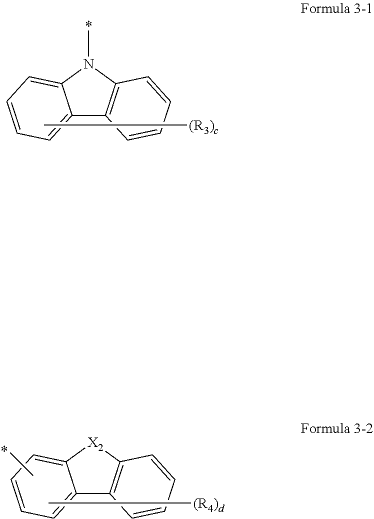
wherein in Formula 3-1 or Formula 3-2,
R₃ and R₄ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring,
c is an integer of 0 to 8,
d is an integer of 0 to 7,
* is a binding site to an adjacent atom,
X₂ is O, S, NAr₃, or SiAr₄Ar₅, and
Ar₃ to Ar₅ are each, independently from one another, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms.

6. The luminescence device of claim 1, wherein the ring B of the Formula 2 is a substituted or an unsubstituted aryl ring of 6 to 18 ring-forming carbon atoms, or a substituted or an unsubstituted heteroaryl ring of 2 to 30 ring-forming carbon atoms, comprising any one of a furan ring, a thiophene ring, a pyrrole ring, or a silole ring.

7. The luminescence device of claim 1, wherein the Formula 2 is of Formula 4: 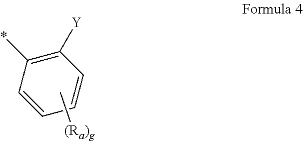
wherein in the Formula 4,
Rₐ is a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring,
g is an integer of 0 to 4,
* is a binding site to an adjacent atom, and
Y has the same meaning as in the Formula 2 of claim 1.

8. The luminescence device of claim 1, wherein L of the Formula 1 is a direct linkage or a substituted or an unsubstituted arylene group of 6 to 18 carbon atoms.

9. The luminescence device of claim 1, wherein The Formula 1 is of Formula 5: 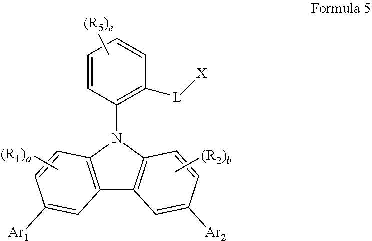
wherein in the Formula 5,
R₅ is a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
e is an integer of 0 to 4, and
R₁, R₂, L, X, Ar₁, Ar₂, a, and b have, independently from one another, the same meaning as in the Formula 1 of claim 1.

10. The luminescence device of claim 1, wherein Ar₁ and Ar₂ of the Formula 1 are the same.

11. The luminescence device of claim 9, wherein the Formula 5 is of Formula 6-1: 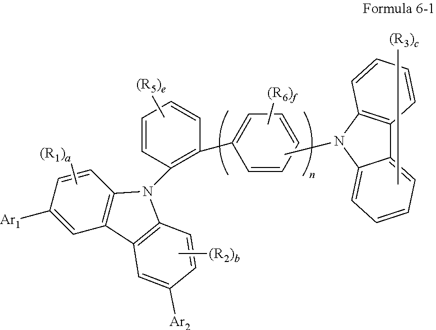
wherein in Formula 6-1,
R₃ and R₆ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
c is an integer of 0 to 8,
f is an integer of 0 to 4,
n is an integer of 0 to 3, and
R₁, R₂, R₅, Ar₁, Ar₂, a, b, and e have, independently from one another, the same meaning as in the Formula 5 of claim 9.

12. The luminescence device of claim 9, wherein the Formula 5 is of Formula 6-2: 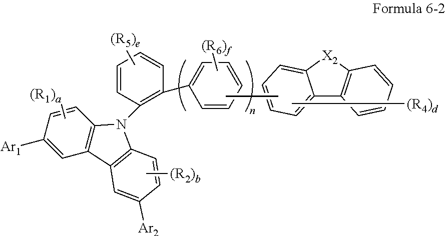
wherein in Formula 6-2,
R₄ and R₆ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
d is an integer of 0 to 7,
f is an integer of 0 to 4,
n is an integer of 0 to 3,
X₂ is O, S, NAr₃, or SiAr₄Ar₅,
Ar₃ to Ar₅ are each, independently from one another, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, and
R₁, R₂, R₅, Ar₁, Ar₂, a, b, and e have, independently from one another, the same meaning as in the Formula 5 of claim 9.

13. The luminescence device of claim 1, wherein the compound of the Formula 1 is at least one compound of Compound Group 1:

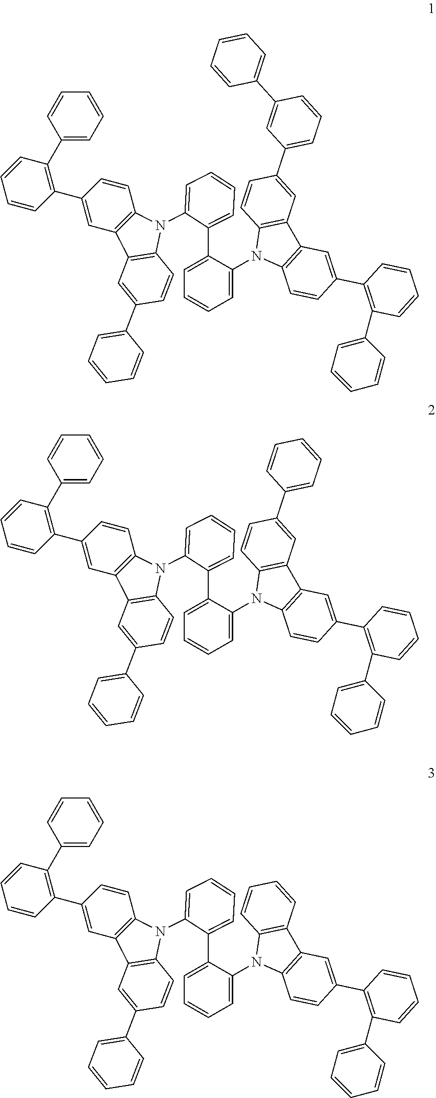 [SMILES: c1ccc(-c2cccc(-c3ccc4c(c3)c3cc(-c5ccccc5-c5ccccc5)ccc3n4-c3ccccc3-c3ccccc3-n3c4ccc(-c5ccccc5)cc4c4cc(-c5ccccc5-c5ccccc5)ccc43)c2)cc1 ; c1ccc(-c2ccc3c(c2)c2cc(-c4ccccc4-c4ccccc4)ccc2n3-c2ccccc2-c2ccccc2-n2c3ccc(-c4ccccc4)cc3c3cc(-c4ccccc4-c4ccccc4)ccc32)cc1 ; c1ccc(-c2ccc3c(c2)c2cc(-c4ccccc4-c4ccccc4)ccc2n3-c2ccccc2-c2ccccc2-n2c3ccccc3c3cc(-c4ccccc4-c4ccccc4)ccc32)cc1]

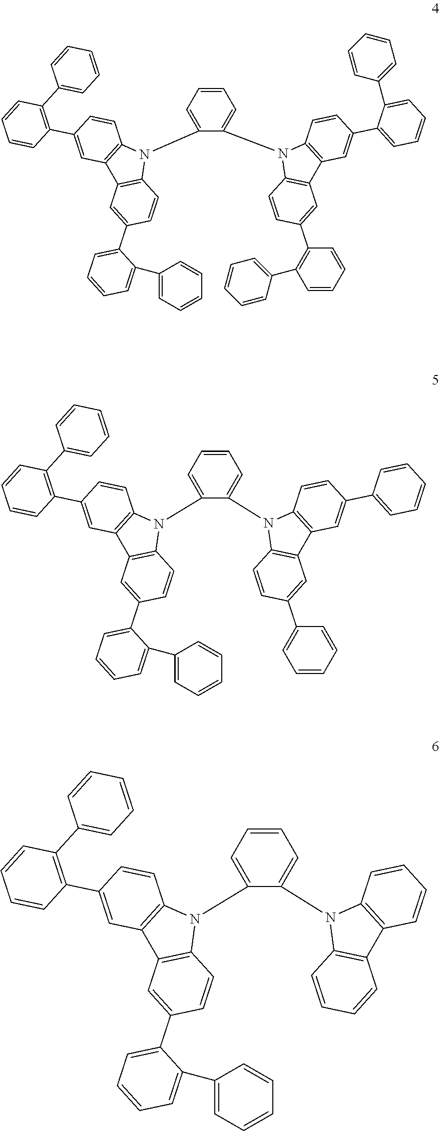 [SMILES: c1ccc(-c2ccccc2-c2ccc3c(c2)c2cc(-c4ccccc4-c4ccccc4)ccc2n3-c2ccccc2-n2c3ccc(-c4ccccc4-c4ccccc4)cc3c3cc(-c4ccccc4-c4ccccc4)ccc32)cc1 ; c1ccc(-c2ccc3c(c2)c2cc(-c4ccccc4)ccc2n3-c2ccccc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccccc2-n2c3ccccc3c3ccccc32)cc1]

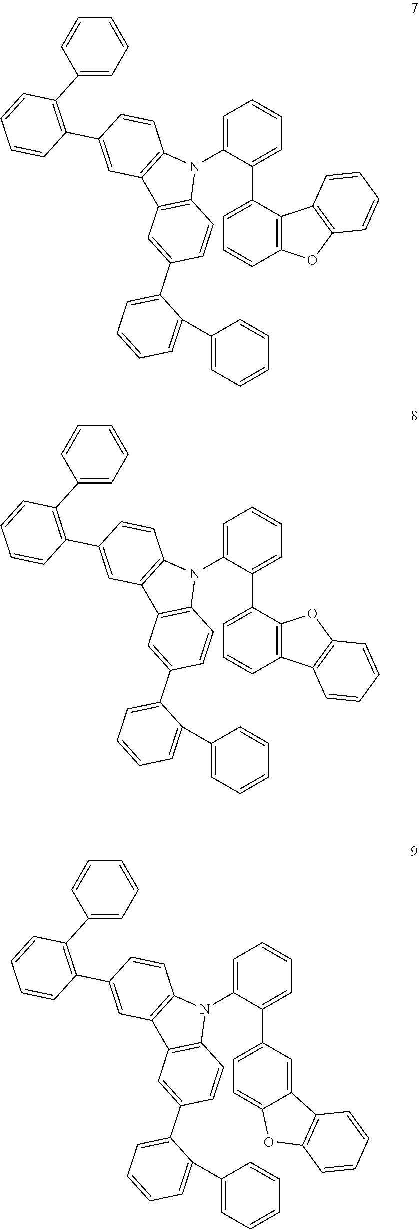 [SMILES: c1ccc(-c2ccccc2-c2ccc3c(c2)c2cc(-c4ccccc4-c4ccccc4)ccc2n3-c2ccccc2-c2cccc3oc4ccccc4c23)cc1 ; c1ccc(-c2ccccc2-c2ccc3c(c2)c2cc(-c4ccccc4-c4ccccc4)ccc2n3-c2ccccc2-c2cccc3c2oc2ccccc23)cc1 ; c1ccc(-c2ccccc2-c2ccc3c(c2)c2cc(-c4ccccc4-c4ccccc4)ccc2n3-c2ccccc2-c2ccc3oc4ccccc4c3c2)cc1]

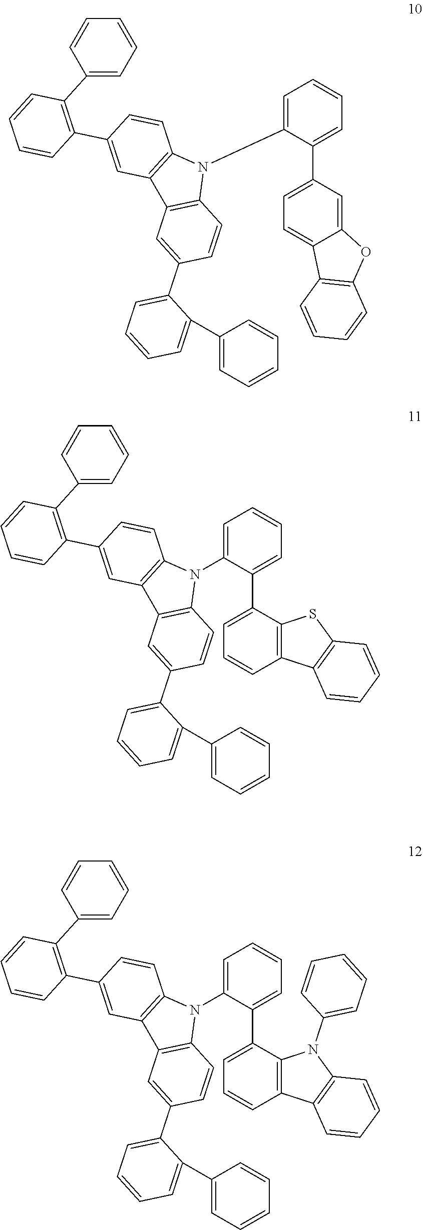 [SMILES: c1ccc(-c2ccccc2-c2ccc3c(c2)c2cc(-c4ccccc4-c4ccccc4)ccc2n3-c2ccccc2-c2ccc3c(c2)oc2ccccc23)cc1 ; c1ccc(-c2ccccc2-c2ccc3c(c2)c2cc(-c4ccccc4-c4ccccc4)ccc2n3-c2ccccc2-c2cccc3c2sc2ccccc23)cc1 ; c1ccc(-c2ccccc2-c2ccc3c(c2)c2cc(-c4ccccc4-c4ccccc4)ccc2n3-c2ccccc2-c2cccc3c4ccccc4n(-c4ccccc4)c23)cc1]

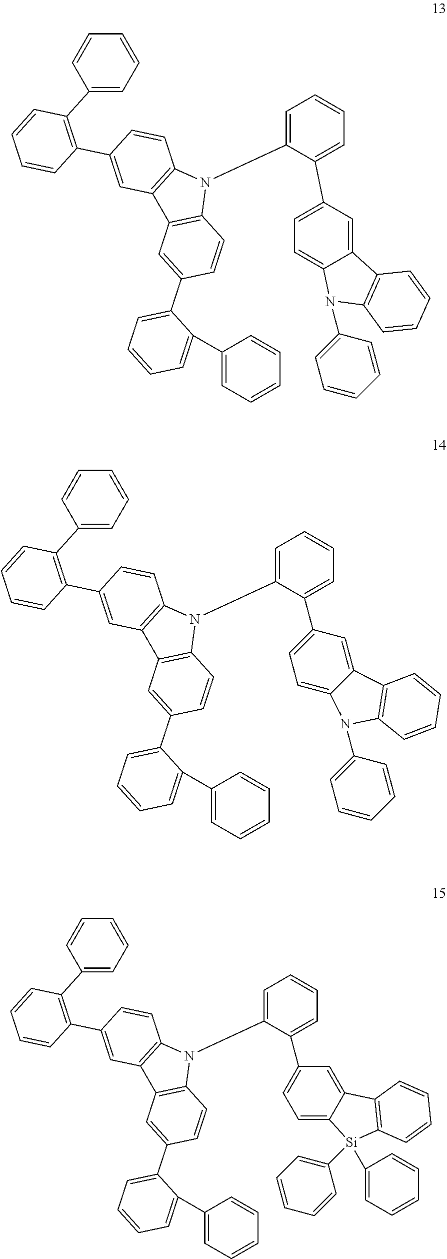 [SMILES: c1ccc(-c2ccccc2-c2ccc3c(c2)c2cc(-c4ccccc4-c4ccccc4)ccc2n3-c2ccccc2-c2ccc3c(c2)c2ccccc2n3-c2ccccc2)cc1 ; c1ccc(-c2ccccc2-c2ccc3c(c2)c2cc(-c4ccccc4-c4ccccc4)ccc2n3-c2ccccc2-c2ccc3c(c2)c2ccccc2n3-c2ccccc2)cc1 ; c1ccc(-c2ccccc2-c2ccc3c(c2)c2cc(-c4ccccc4-c4ccccc4)ccc2n3-c2ccccc2-c2ccc3c(c2)-c2ccccc2[Si]3(c2ccccc2)c2ccccc2)cc1]

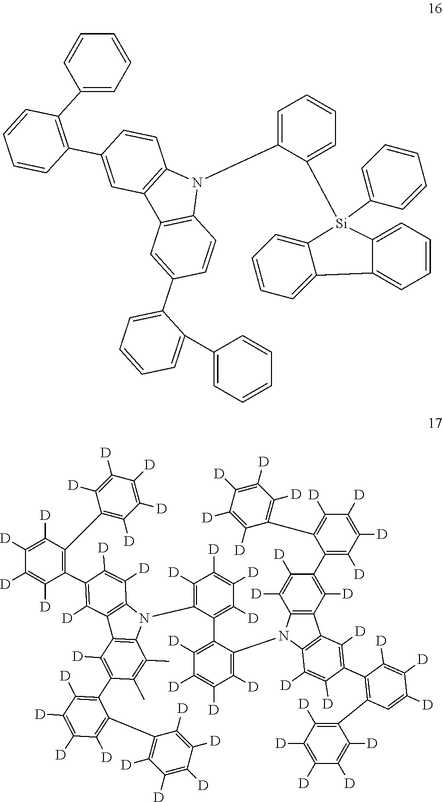

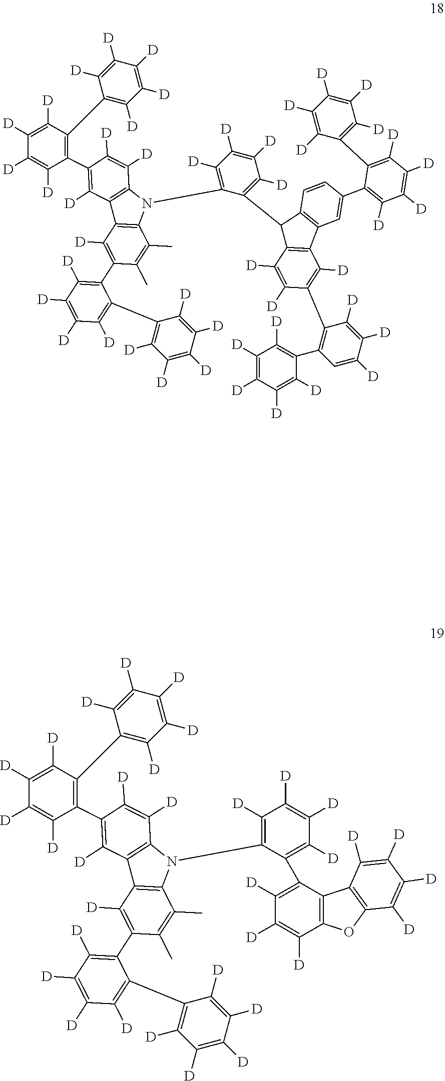

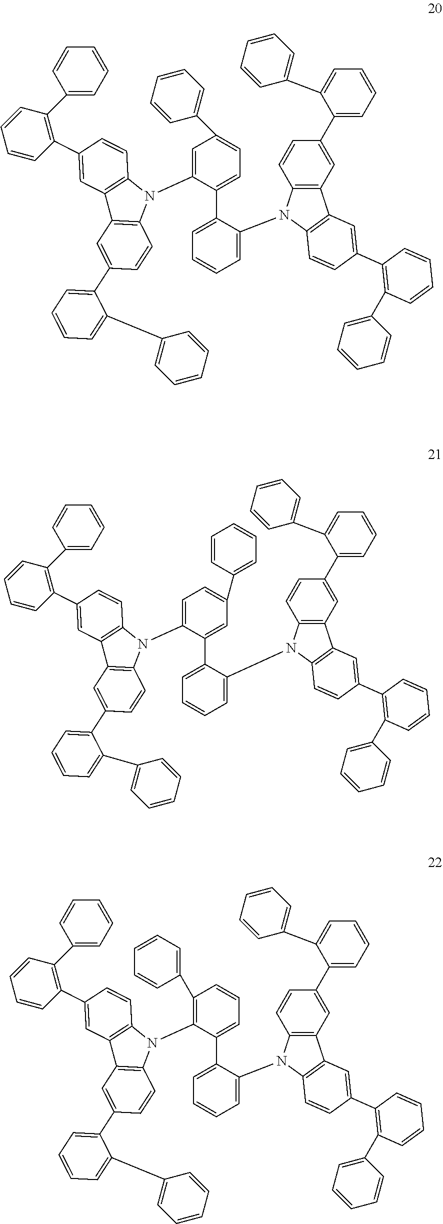 [SMILES: c1ccc(-c2ccc(-c3ccccc3-n3c4ccc(-c5ccccc5-c5ccccc5)cc4c4cc(-c5ccccc5-c5ccccc5)ccc43)c(-n3c4ccc(-c5ccccc5-c5ccccc5)cc4c4cc(-c5ccccc5-c5ccccc5)ccc43)c2)cc1 ; c1ccc(-c2ccc(-n3c4ccc(-c5ccccc5-c5ccccc5)cc4c4cc(-c5ccccc5-c5ccccc5)ccc43)c(-c3ccccc3-n3c4ccc(-c5ccccc5-c5ccccc5)cc4c4cc(-c5ccccc5-c5ccccc5)ccc43)c2)cc1 ; c1ccc(-c2ccccc2-c2ccc3c(c2)c2cc(-c4ccccc4-c4ccccc4)ccc2n3-c2ccccc2-c2cccc(-c3ccccc3)c2-n2c3ccc(-c4ccccc4-c4ccccc4)cc3c3cc(-c4ccccc4-c4ccccc4)ccc32)cc1]

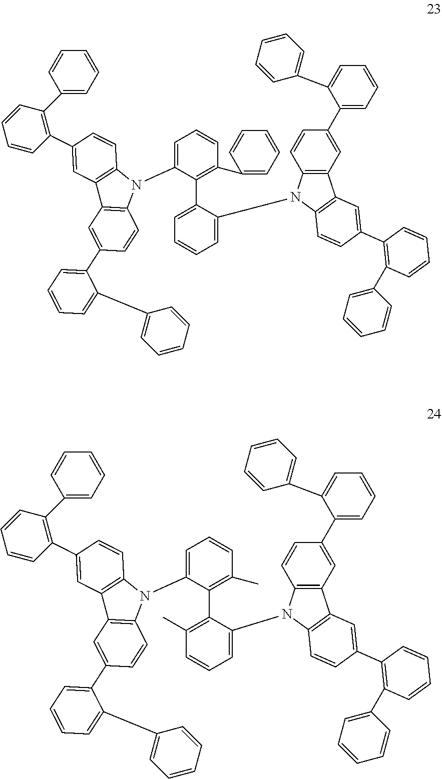 [SMILES: c1ccc(-c2ccccc2-c2ccc3c(c2)c2cc(-c4ccccc4-c4ccccc4)ccc2n3-c2ccccc2-c2c(-c3ccccc3)cccc2-n2c3ccc(-c4ccccc4-c4ccccc4)cc3c3cc(-c4ccccc4-c4ccccc4)ccc32)cc1 ; Cc1cccc(-n2c3ccc(-c4ccccc4-c4ccccc4)cc3c3cc(-c4ccccc4-c4ccccc4)ccc32)c1-c1c(C)cccc1-n1c2ccc(-c3ccccc3-c3ccccc3)cc2c2cc(-c3ccccc3-c3ccccc3)ccc21]

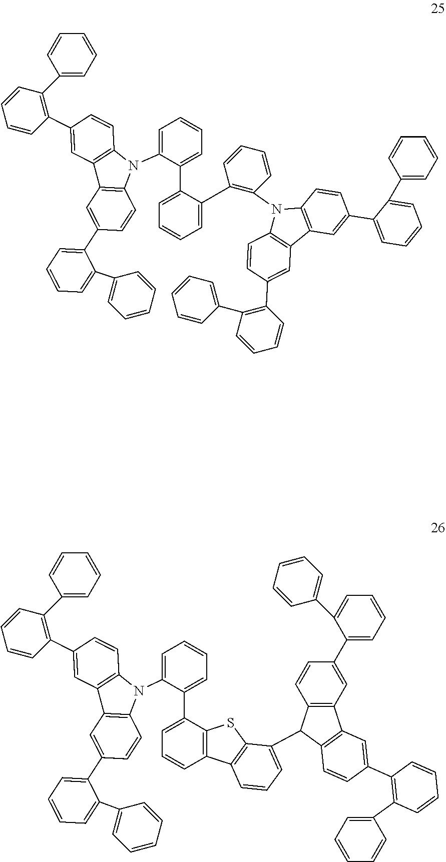 [SMILES: c1ccc(-c2ccccc2-c2ccc3c(c2)c2cc(-c4ccccc4-c4ccccc4)ccc2n3-c2ccccc2-c2ccccc2-c2ccccc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)-c2cc(-c4ccccc4-c4ccccc4)ccc2C3c2cccc3c2sc2c(-c4ccccc4-n4c5ccc(-c6ccccc6-c6ccccc6)cc5c5cc(-c6ccccc6-c6ccccc6)ccc54)cccc23)cc1]

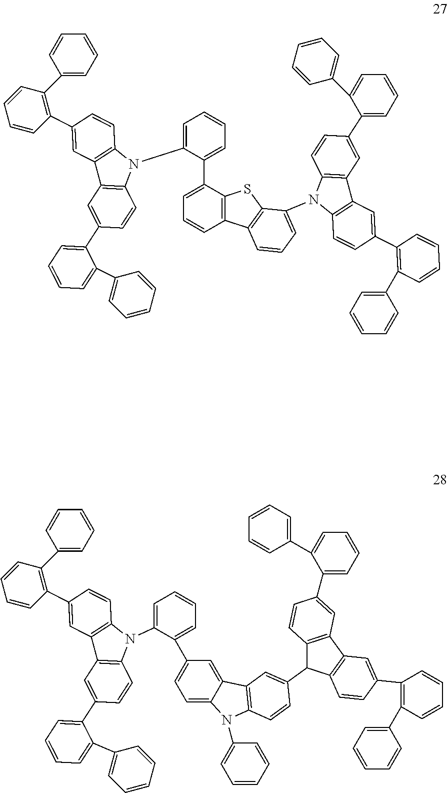 [SMILES: c1ccc(-c2ccccc2-c2ccc3c(c2)c2cc(-c4ccccc4-c4ccccc4)ccc2n3-c2ccccc2-c2cccc3c2sc2c(-n4c5ccc(-c6ccccc6-c6ccccc6)cc5c5cc(-c6ccccc6-c6ccccc6)ccc54)cccc23)cc1 ; c1ccc(-c2ccccc2-c2ccc3c(c2)-c2cc(-c4ccccc4-c4ccccc4)ccc2C3c2ccc3c(c2)c2cc(-c4ccccc4-n4c5ccc(-c6ccccc6-c6ccccc6)cc5c5cc(-c6ccccc6-c6ccccc6)ccc54)ccc2n3-c2ccccc2)cc1]

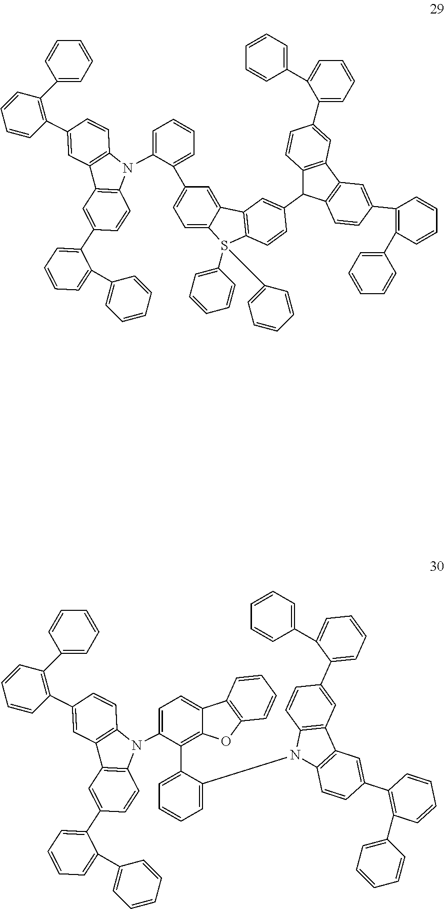 [SMILES: c1ccc(-c2ccccc2-c2ccc3c(c2)-c2cc(-c4ccccc4-c4ccccc4)ccc2C3c2ccc3c(c2)-c2cc(-c4ccccc4-n4c5ccc(-c6ccccc6-c6ccccc6)cc5c5cc(-c6ccccc6-c6ccccc6)ccc54)ccc2S3(c2ccccc2)c2ccccc2)cc1 ; c1ccc(-c2ccccc2-c2ccc3c(c2)c2cc(-c4ccccc4-c4ccccc4)ccc2n3-c2ccccc2-c2c(-n3c4ccc(-c5ccccc5-c5ccccc5)cc4c4cc(-c5ccccc5-c5ccccc5)ccc43)ccc3c2oc2ccccc23)cc1]

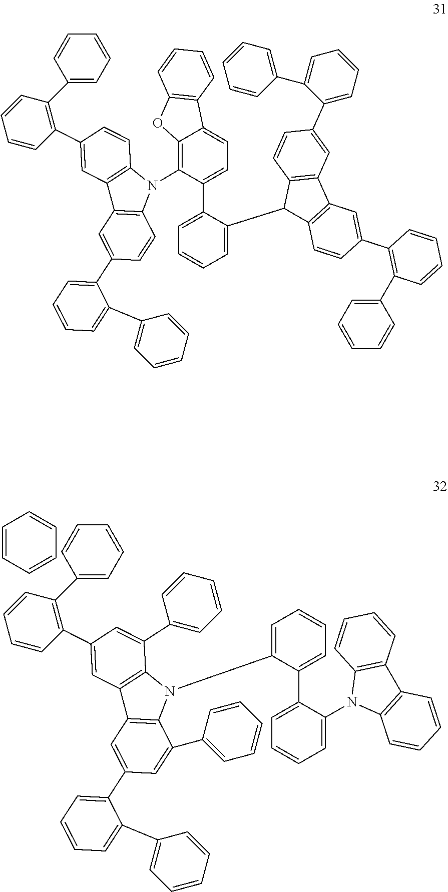

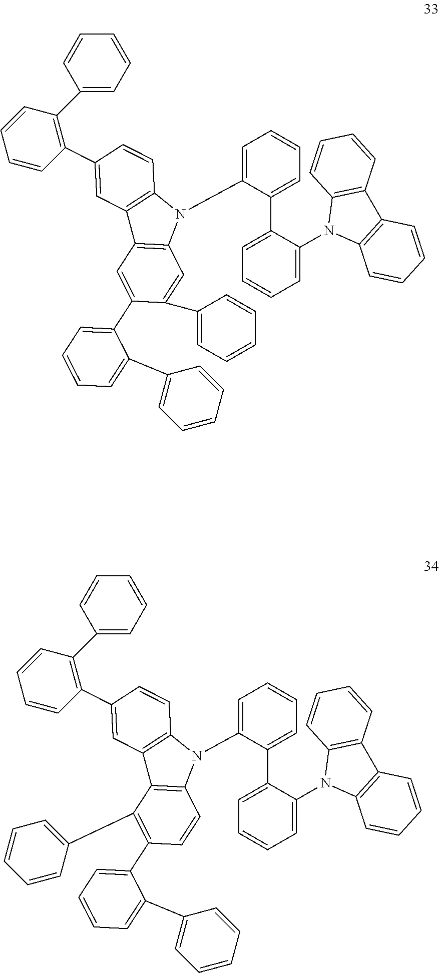 [SMILES: c1ccc(-c2ccccc2-c2ccc3c(c2)c2cc(-c4ccccc4-c4ccccc4)c(-c4ccccc4)cc2n3-c2ccccc2-c2ccccc2-n2c3ccccc3c3ccccc32)cc1 ; c1ccc(-c2ccccc2-c2ccc3c(c2)c2c(-c4ccccc4)c(-c4ccccc4-c4ccccc4)ccc2n3-c2ccccc2-c2ccccc2-n2c3ccccc3c3ccccc32)cc1]

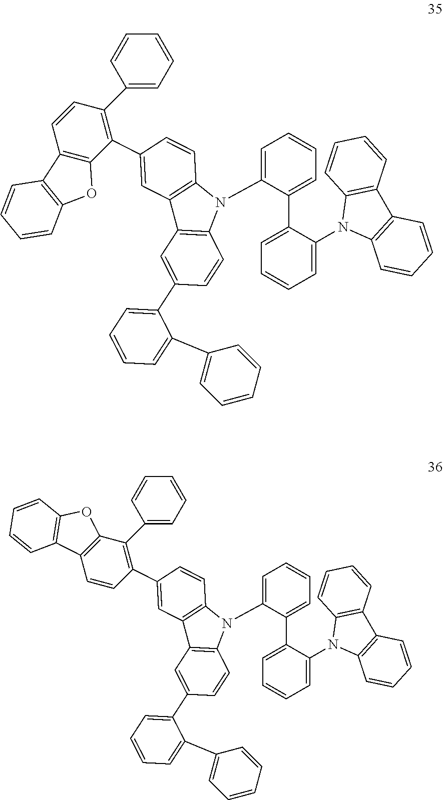 [SMILES: c1ccc(-c2ccccc2-c2ccc3c(c2)c2cc(-c4c(-c5ccccc5)ccc5c4oc4ccccc45)ccc2n3-c2ccccc2-c2ccccc2-n2c3ccccc3c3ccccc32)cc1 ; c1ccc(-c2ccccc2-c2ccc3c(c2)c2cc(-c4ccc5c(oc6ccccc65)c4-c4ccccc4)ccc2n3-c2ccccc2-c2ccccc2-n2c3ccccc3c3ccccc32)cc1]

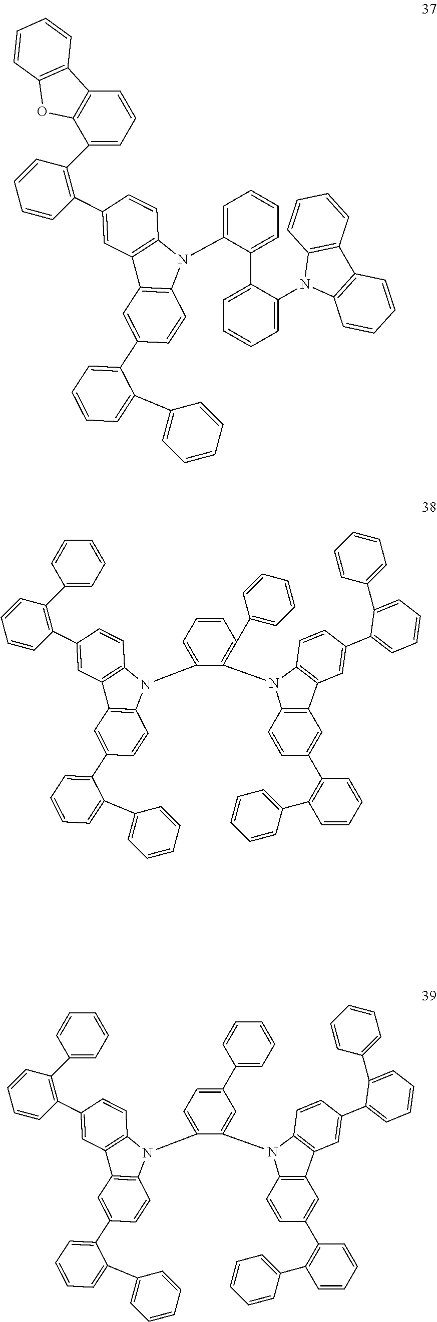 [SMILES: c1ccc(-c2ccccc2-c2ccc3c(c2)c2cc(-c4ccccc4-c4cccc5c4oc4ccccc45)ccc2n3-c2ccccc2-c2ccccc2-n2c3ccccc3c3ccccc32)cc1 ; c1ccc(-c2ccccc2-c2ccc3c(c2)c2cc(-c4ccccc4-c4ccccc4)ccc2n3-c2cccc(-c3ccccc3)c2-n2c3ccc(-c4ccccc4-c4ccccc4)cc3c3cc(-c4ccccc4-c4ccccc4)ccc32)cc1 ; c1ccc(-c2ccc(-n3c4ccc(-c5ccccc5-c5ccccc5)cc4c4cc(-c5ccccc5-c5ccccc5)ccc43)c(-n3c4ccc(-c5ccccc5-c5ccccc5)cc4c4cc(-c5ccccc5-c5ccccc5)ccc43)c2)cc1]

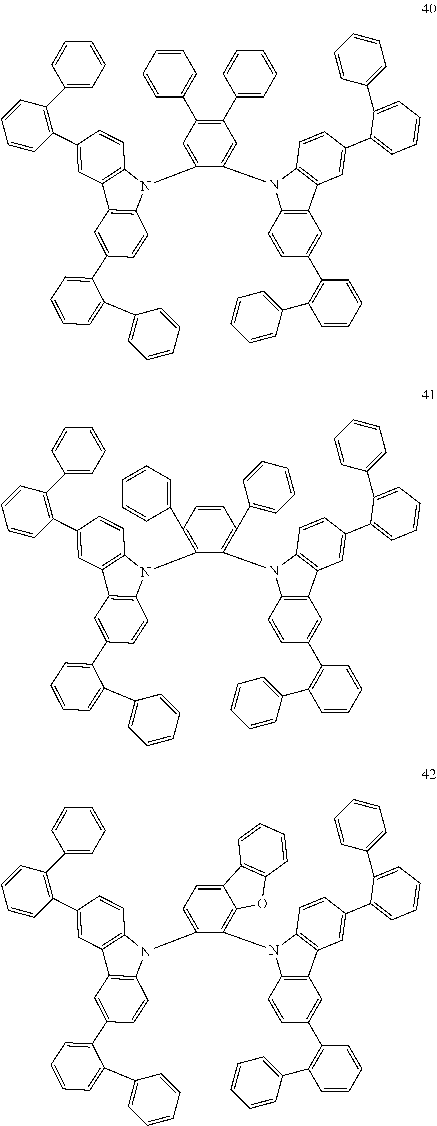 [SMILES: c1ccc(-c2ccccc2-c2ccc3c(c2)c2cc(-c4ccccc4-c4ccccc4)ccc2n3-c2cc(-c3ccccc3)c(-c3ccccc3)cc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2c(-c3ccccc3)ccc(-c3ccccc3)c2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccc3c(oc4ccccc43)c2-n2c3ccc(-c4ccccc4-c4ccccc4)cc3c3cc(-c4ccccc4-c4ccccc4)ccc32)cc1]

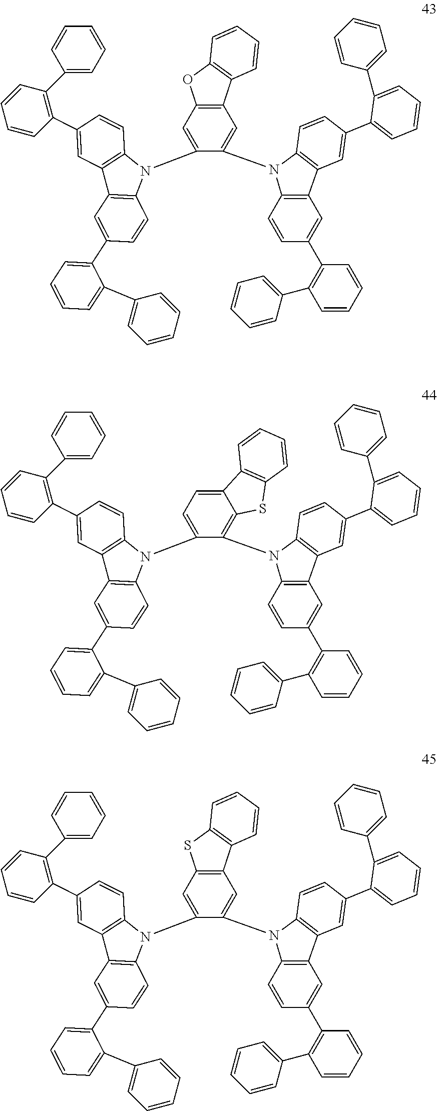 [SMILES: c1ccc(-c2ccccc2-c2ccc3c(c2)c2cc(-c4ccccc4-c4ccccc4)ccc2n3-c2cc3oc4ccccc4c3cc2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2ccc3c(sc4ccccc43)c2-n2c3ccc(-c4ccccc4-c4ccccc4)cc3c3cc(-c4ccccc4-c4ccccc4)ccc32)cc1 ; c1ccc(-c2ccccc2-c2ccc3c(c2)c2cc(-c4ccccc4-c4ccccc4)ccc2n3-c2cc3sc4ccccc4c3cc2-n2c3ccc(-c4ccccc4-c4ccccc4)cc3c3cc(-c4ccccc4-c4ccccc4)ccc32)cc1]

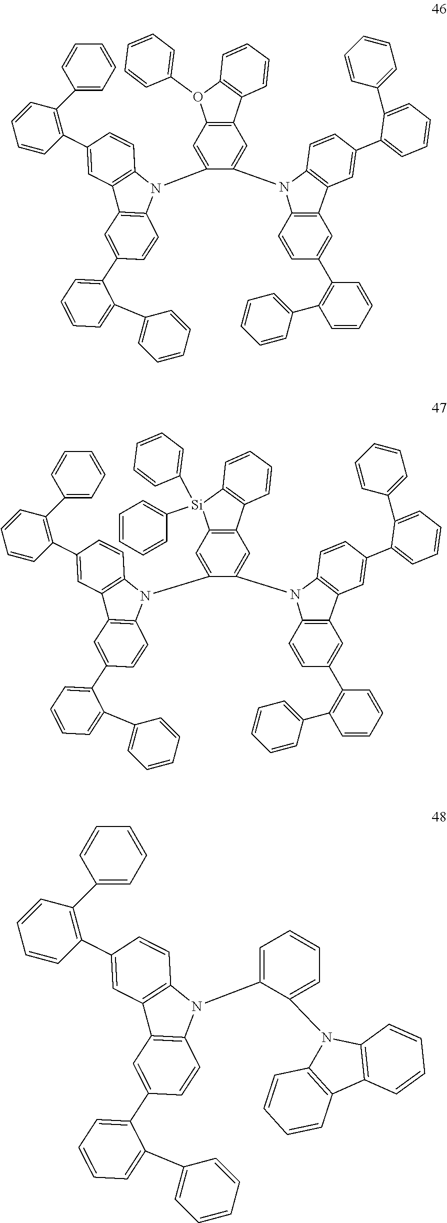

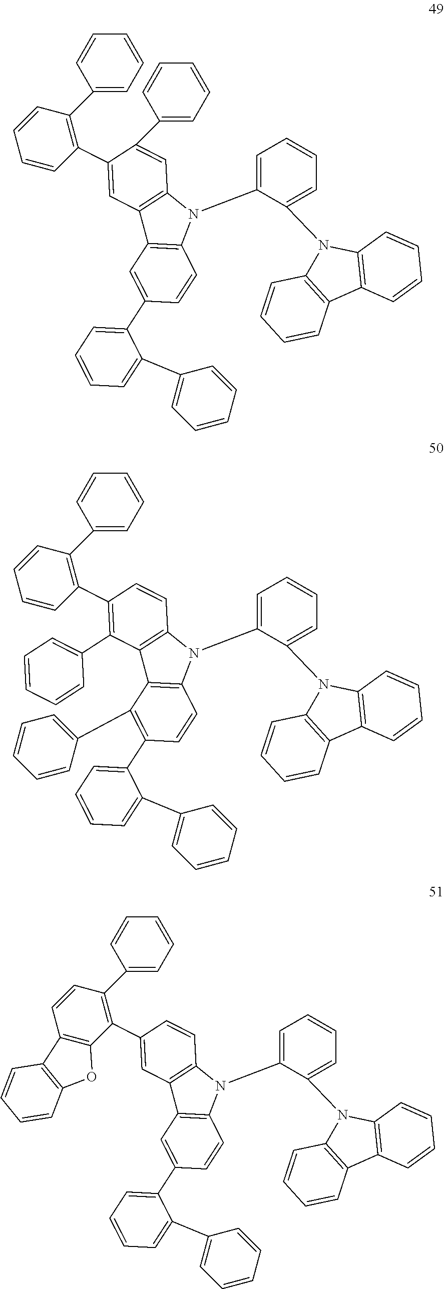 [SMILES: c1ccc(-c2ccccc2-c2ccc3c(c2)c2cc(-c4ccccc4-c4ccccc4)c(-c4ccccc4)cc2n3-c2ccccc2-n2c3ccccc3c3ccccc32)cc1 ; c1ccc(-c2ccccc2-c2ccc3c(c2-c2ccccc2)c2c(-c4ccccc4)c(-c4ccccc4-c4ccccc4)ccc2n3-c2ccccc2-n2c3ccccc3c3ccccc32)cc1 ; c1ccc(-c2ccccc2-c2ccc3c(c2)c2cc(-c4c(-c5ccccc5)ccc5c4oc4ccccc45)ccc2n3-c2ccccc2-n2c3ccccc3c3ccccc32)cc1]

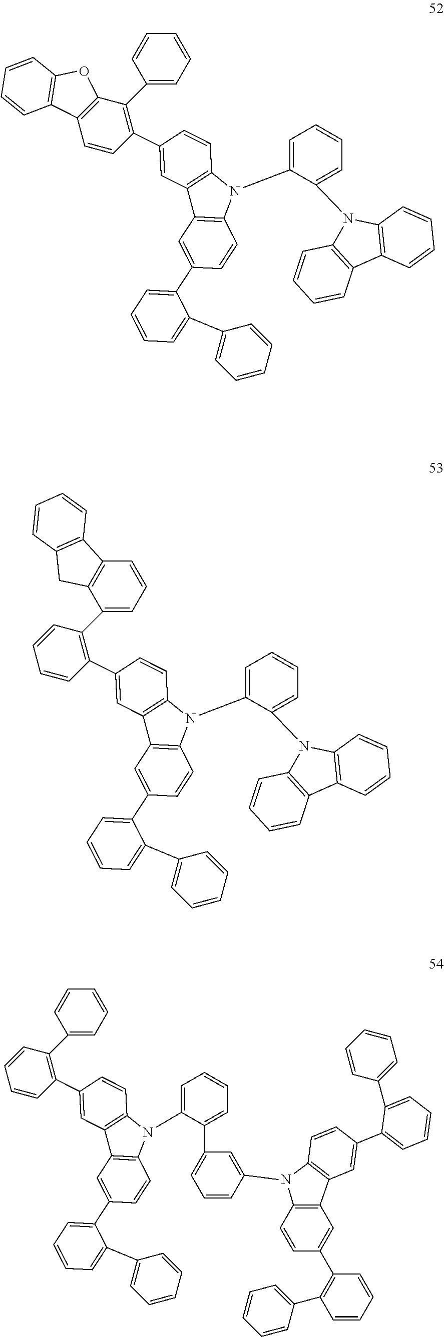 [SMILES: c1ccc(-c2ccccc2-c2ccc3c(c2)c2cc(-c4ccc5c(oc6ccccc65)c4-c4ccccc4)ccc2n3-c2ccccc2-n2c3ccccc3c3ccccc32)cc1 ; c1ccc(-c2ccccc2-c2ccc3c(c2)c2cc(-c4ccccc4-c4cccc5c4Cc4ccccc4-5)ccc2n3-c2ccccc2-n2c3ccccc3c3ccccc32)cc1 ; c1ccc(-c2ccccc2-c2ccc3c(c2)c2cc(-c4ccccc4-c4ccccc4)ccc2n3-c2cccc(-c3ccccc3-n3c4ccc(-c5ccccc5-c5ccccc5)cc4c4cc(-c5ccccc5-c5ccccc5)ccc43)c2)cc1]

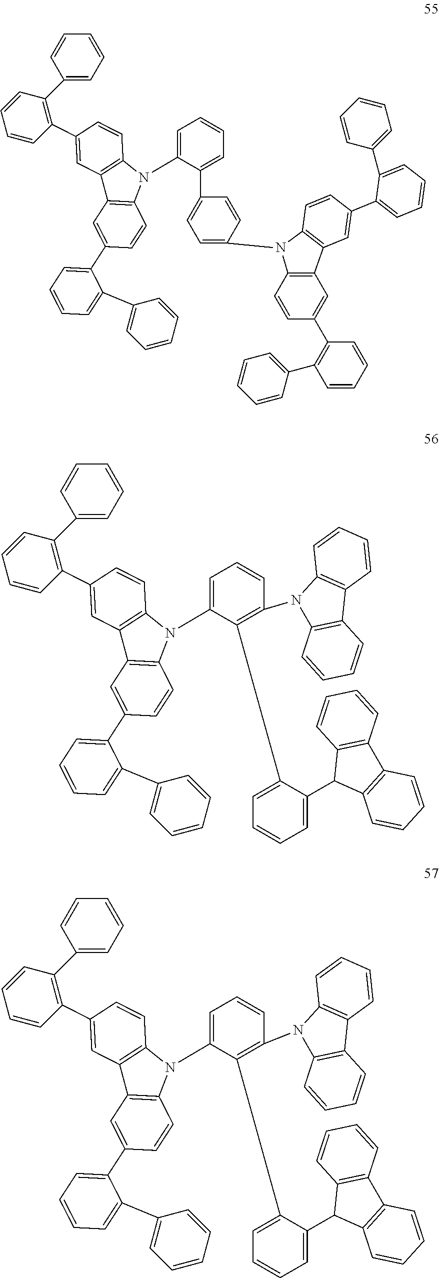 [SMILES: c1ccc(-c2ccccc2-c2ccc3c(c2)c2cc(-c4ccccc4-c4ccccc4)ccc2n3-c2ccc(-c3ccccc3-n3c4ccc(-c5ccccc5-c5ccccc5)cc4c4cc(-c5ccccc5-c5ccccc5)ccc43)cc2)cc1 ; c1ccc(-c2ccccc2-c2ccc3c(c2)c2cc(-c4ccccc4-c4ccccc4)ccc2n3-c2cccc(-n3c4ccccc4c4ccccc43)c2-c2ccccc2C2c3ccccc3-c3ccccc32)cc1 ; c1ccc(-c2ccccc2-c2ccc3c(c2)c2cc(-c4ccccc4-c4ccccc4)ccc2n3-c2cccc(-n3c4ccccc4c4ccccc43)c2-c2ccccc2C2c3ccccc3-c3ccccc32)cc1]

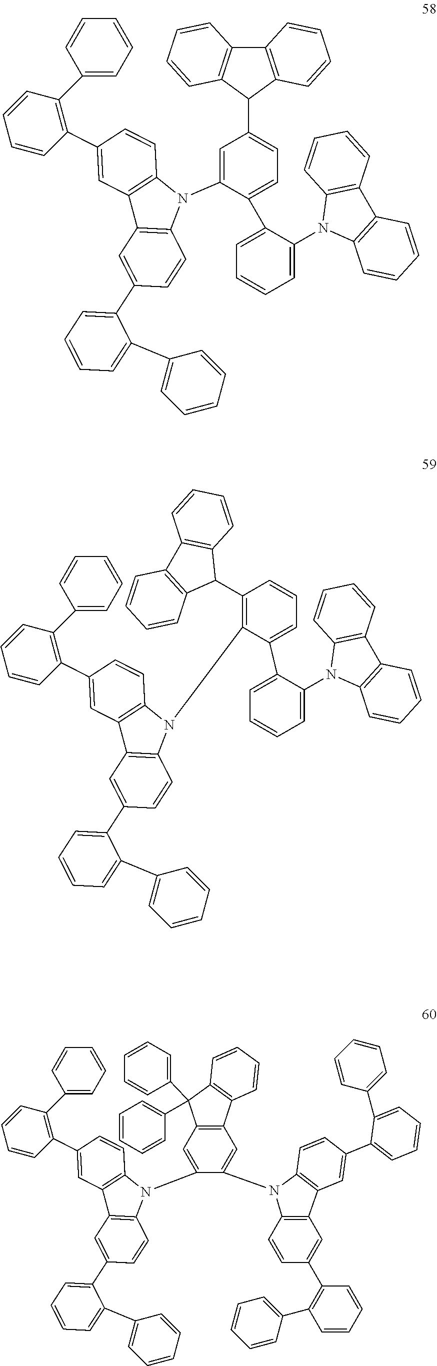 [SMILES: c1ccc(-c2ccccc2-c2ccc3c(c2)c2cc(-c4ccccc4-c4ccccc4)ccc2n3-c2cc(C3c4ccccc4-c4ccccc43)ccc2-c2ccccc2-n2c3ccccc3c3ccccc32)cc1 ; c1ccc(-c2ccccc2-c2ccc3c(c2)c2cc(-c4ccccc4-c4ccccc4)ccc2n3-c2c(-c3ccccc3-n3c4ccccc4c4ccccc43)cccc2C2c3ccccc3-c3ccccc32)cc1 ; c1ccc(-c2ccccc2-c2ccc3c(c2)c2cc(-c4ccccc4-c4ccccc4)ccc2n3-c2cc3c(cc2-n2c4ccc(-c5ccccc5-c5ccccc5)cc4c4cc(-c5ccccc5-c5ccccc5)ccc42)C(c2ccccc2)(c2ccccc2)c2ccccc2-3)cc1]

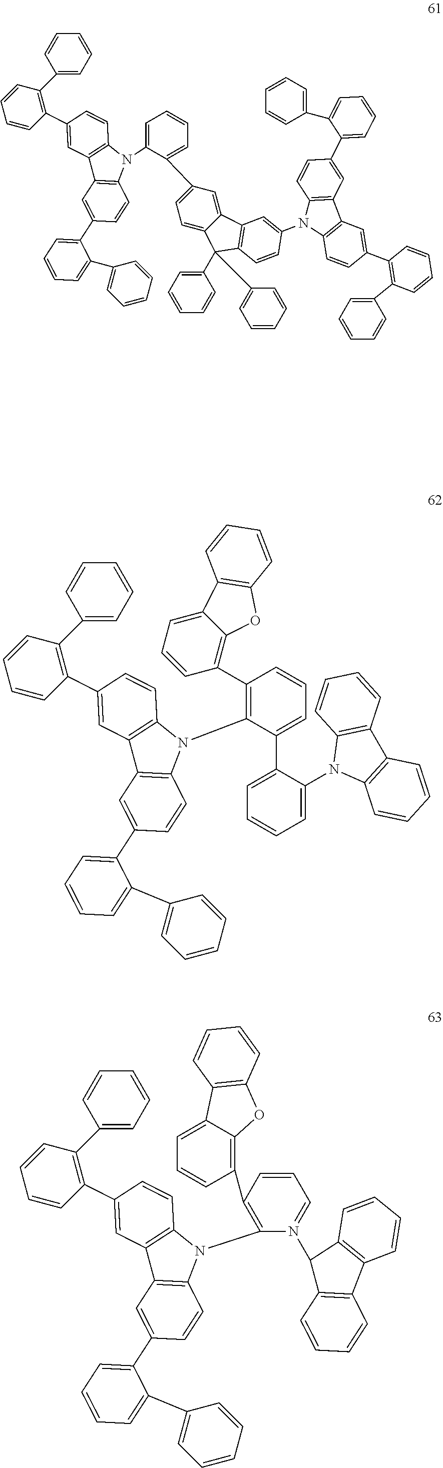

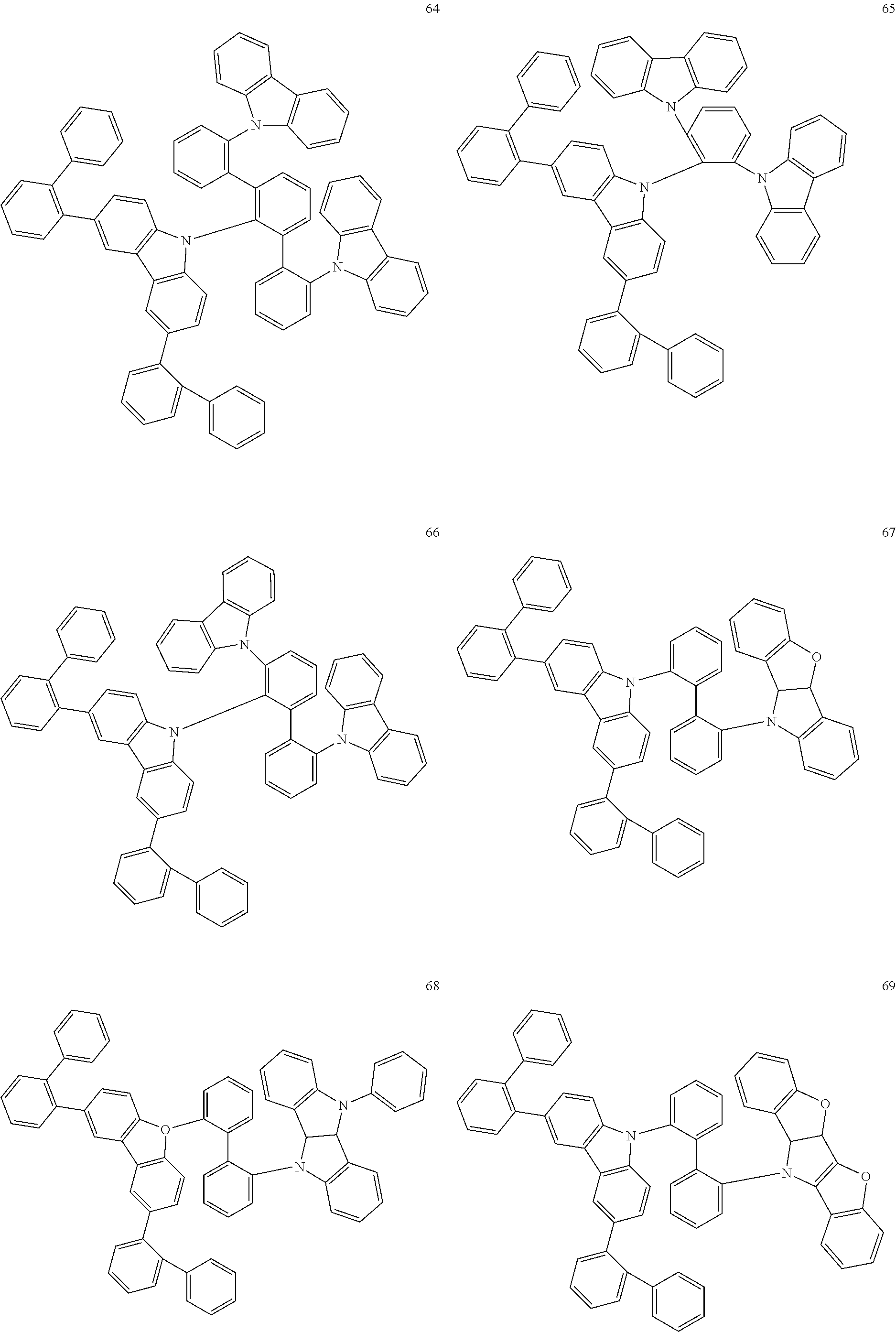

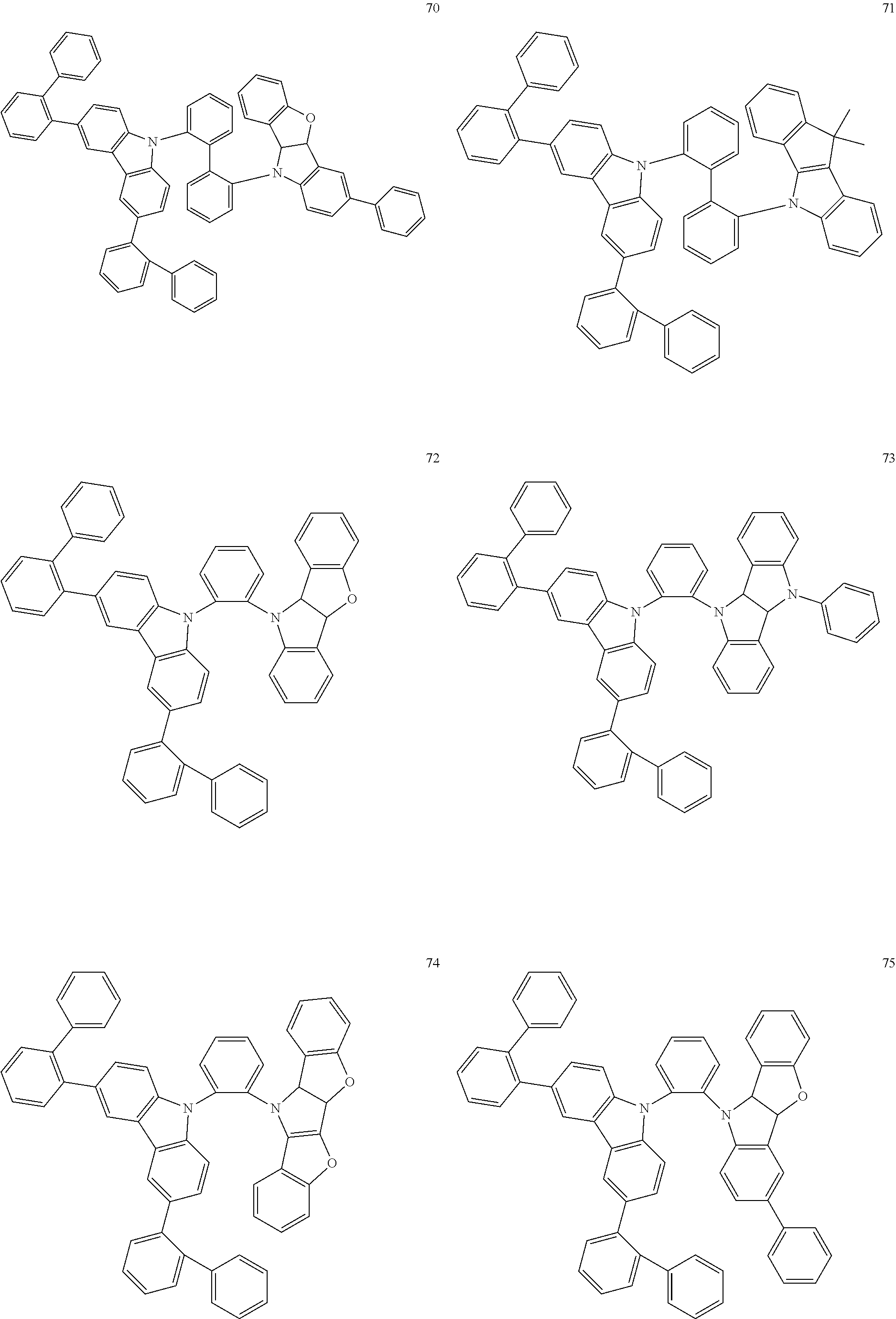

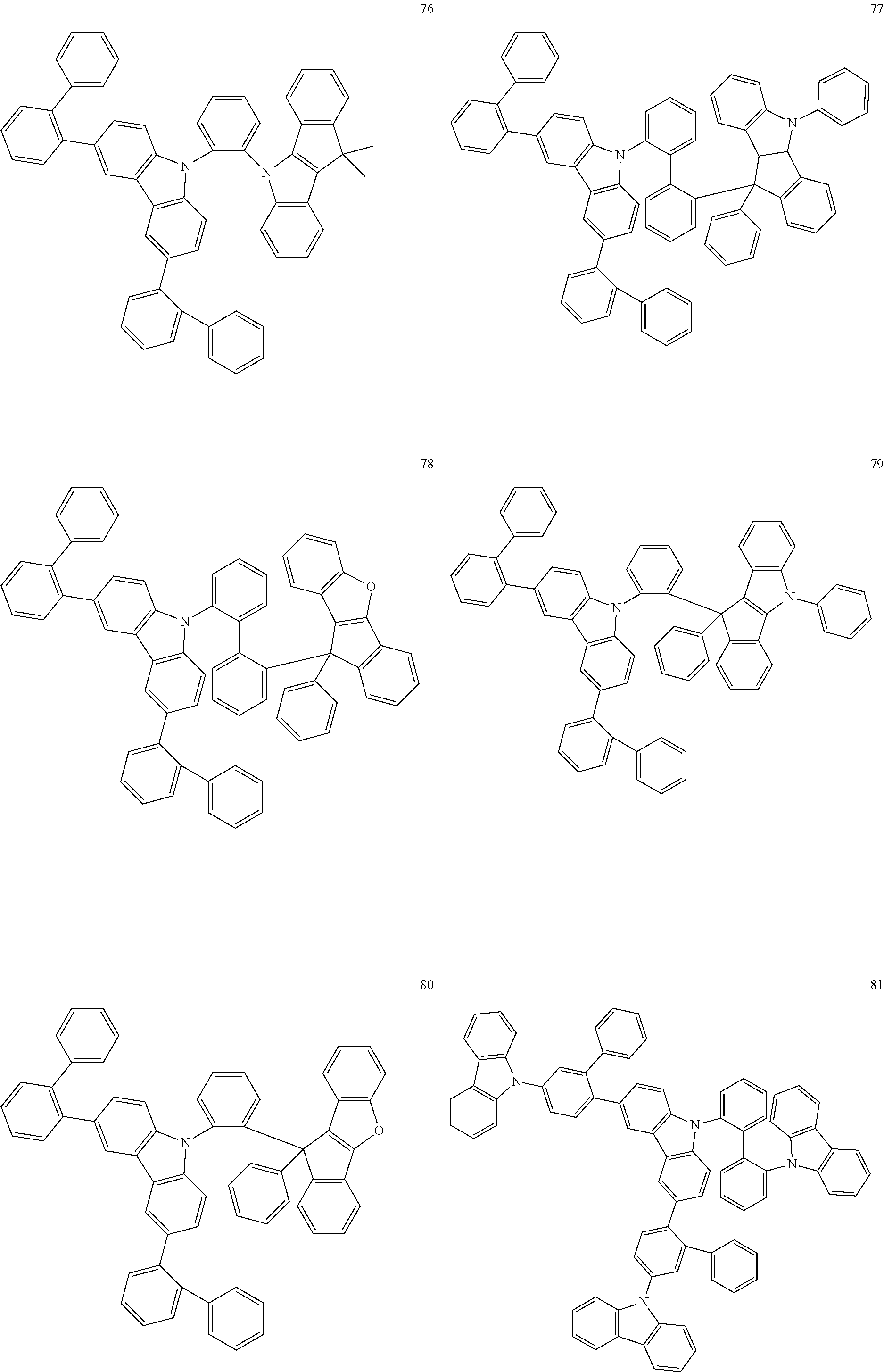

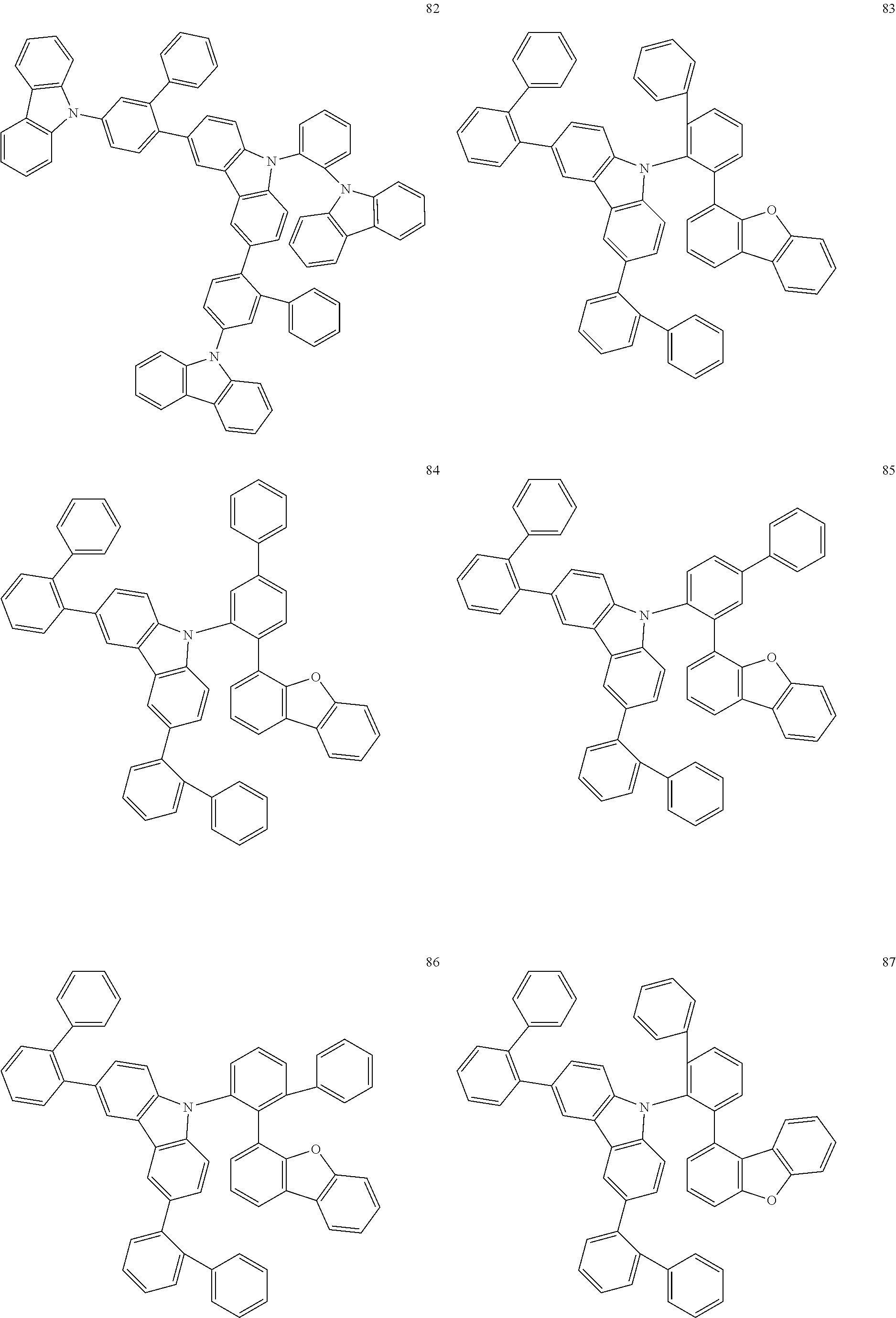

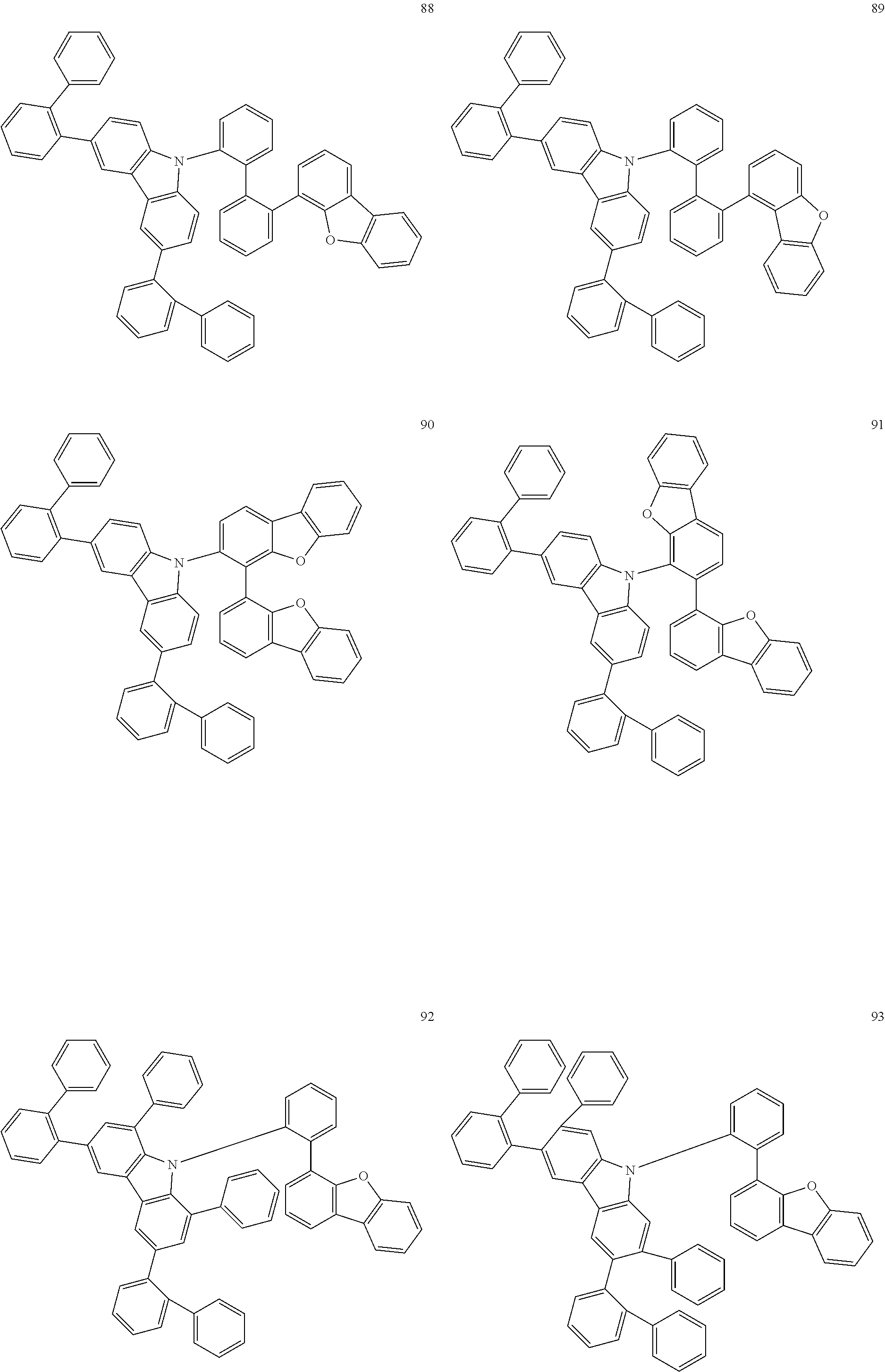

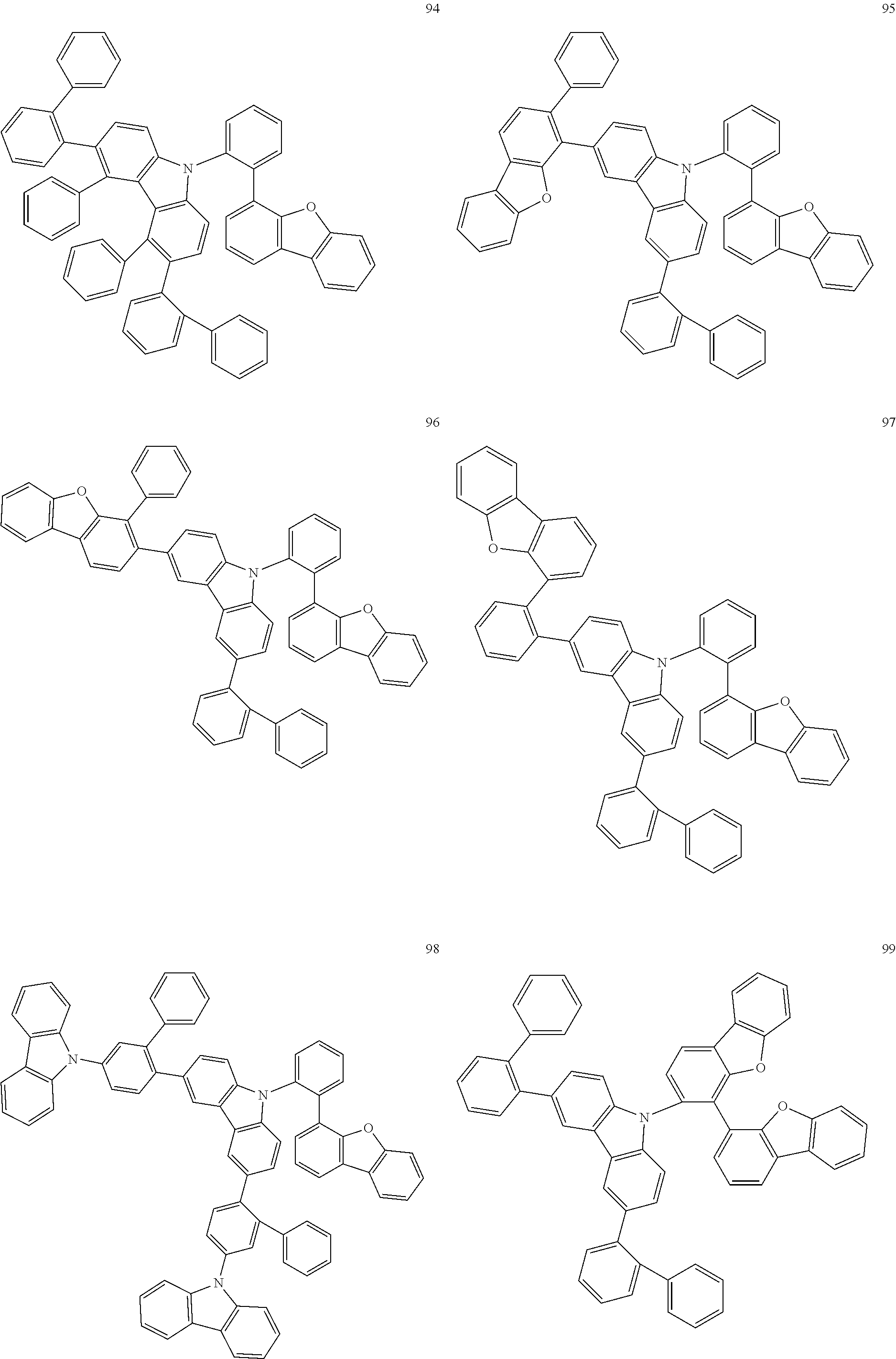

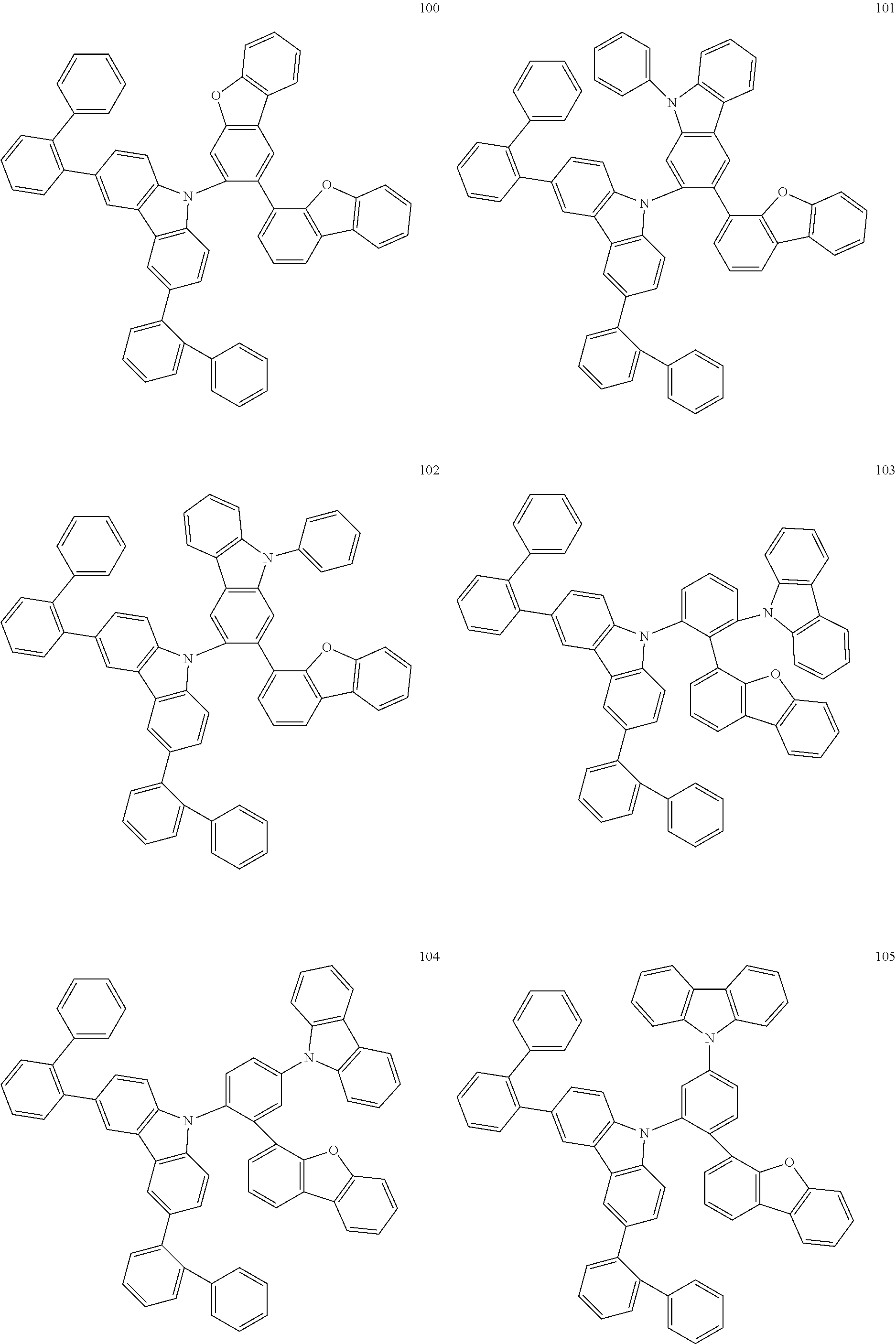

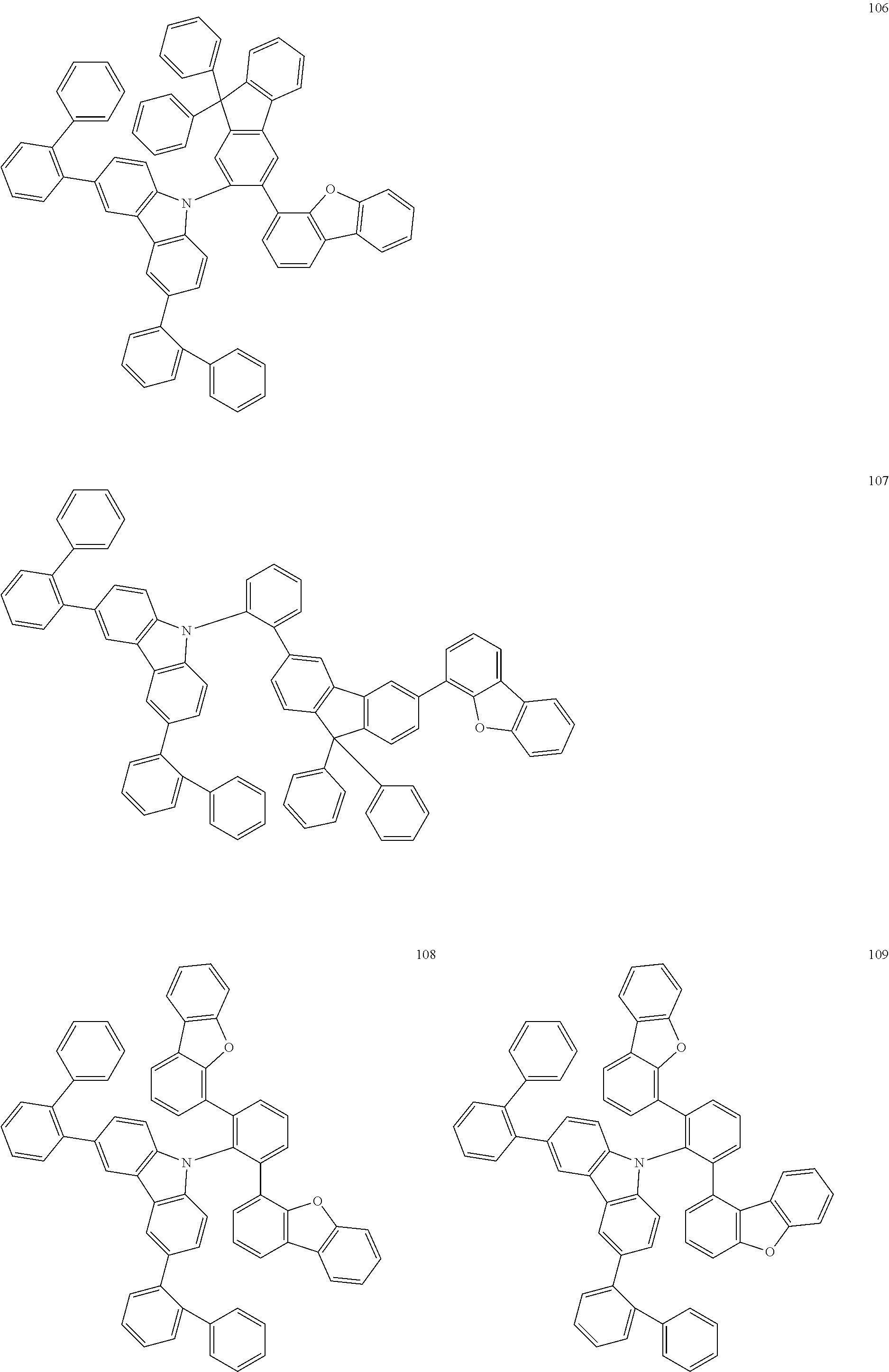 [SMILES: c1ccc(-c2ccccc2-c2ccc3c(c2)c2cc(-c4ccccc4-c4ccccc4)ccc2n3-c2cc3c(cc2-c2cccc4c2oc2ccccc24)-c2ccccc2C3(c2ccccc2)c2ccccc2)cc1 ; c1ccc(-c2ccccc2-c2ccc3c(c2)c2cc(-c4ccccc4-c4ccccc4)ccc2n3-c2ccccc2-c2ccc3c(c2)-c2cc(-c4cccc5c4oc4ccccc45)ccc2C3(c2ccccc2)c2ccccc2)cc1 ; c1ccc(-c2ccccc2-c2ccc3c(c2)c2cc(-c4ccccc4-c4ccccc4)ccc2n3-c2c(-c3cccc4c3oc3ccccc34)cccc2-c2cccc3c2oc2ccccc23)cc1 ; c1ccc(-c2ccccc2-c2ccc3c(c2)c2cc(-c4ccccc4-c4ccccc4)ccc2n3-c2c(-c3cccc4c3oc3ccccc34)cccc2-c2cccc3oc4ccccc4c23)cc1]

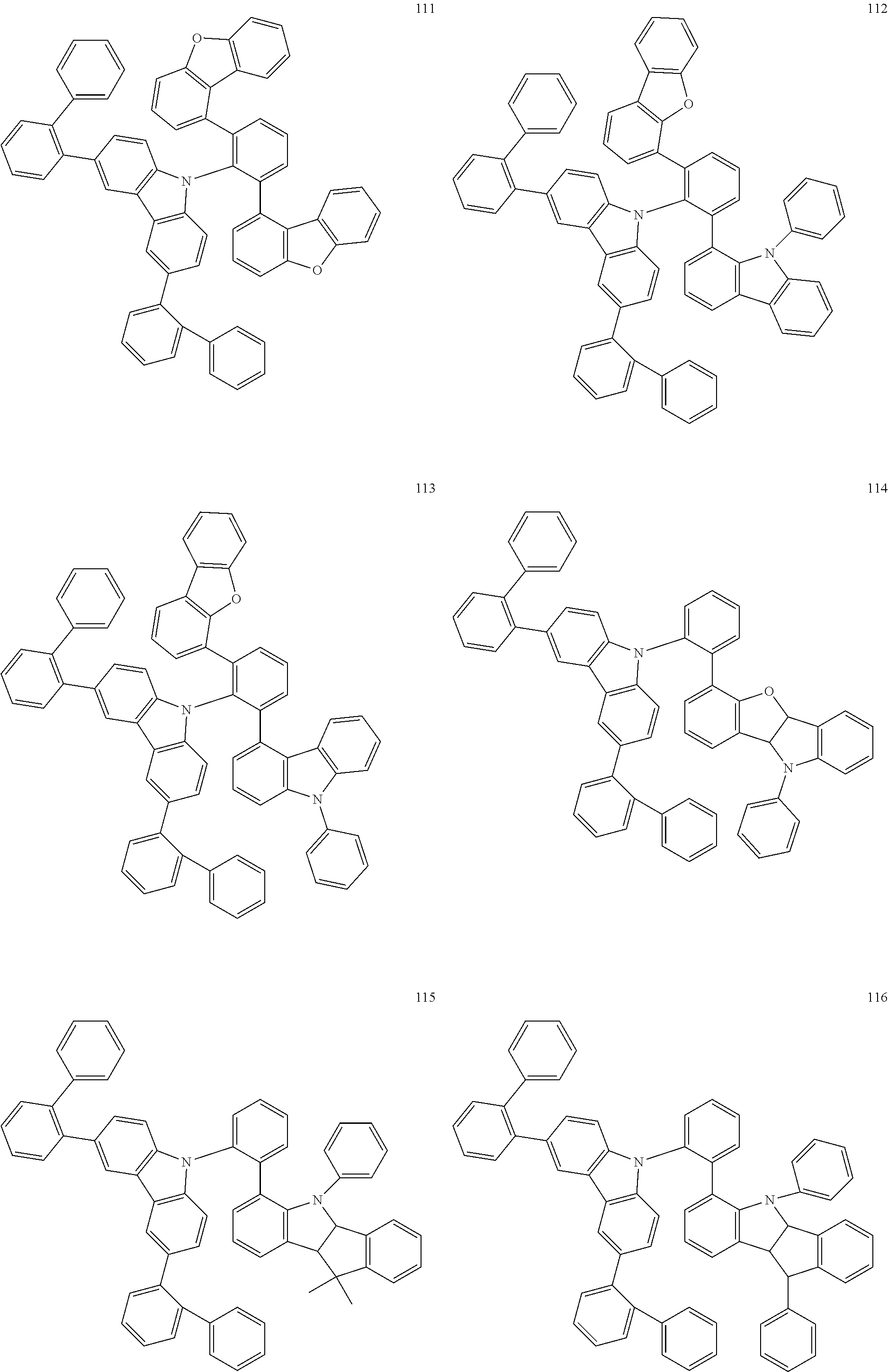

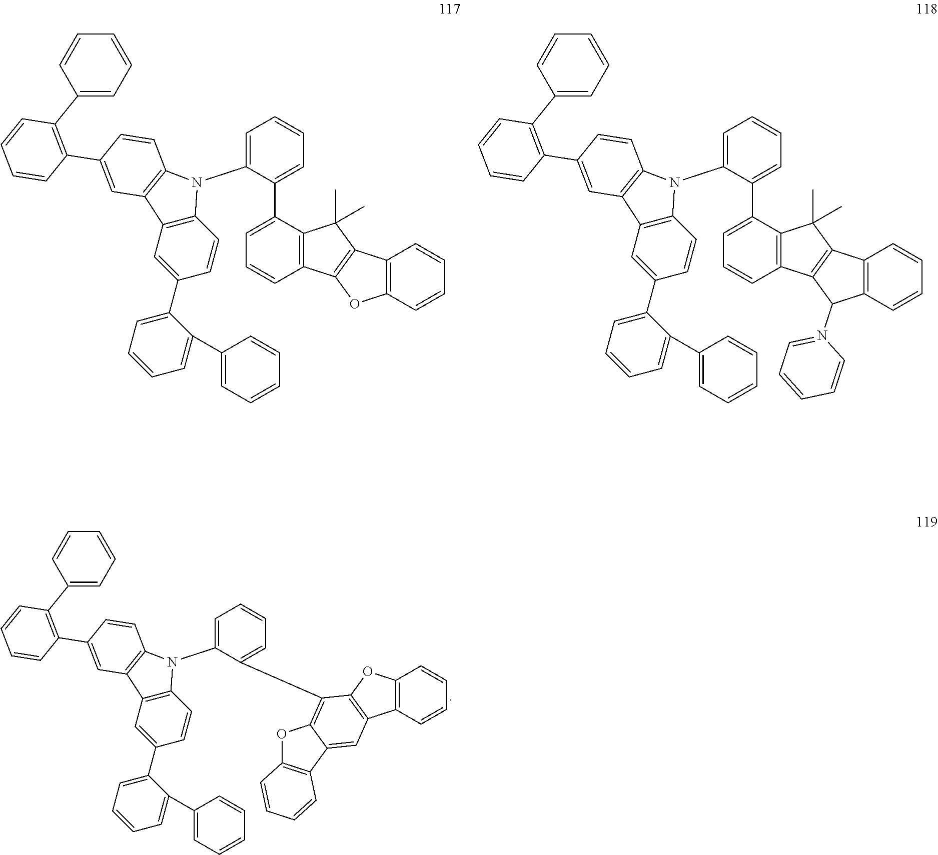

14. A compound of Formula 1: 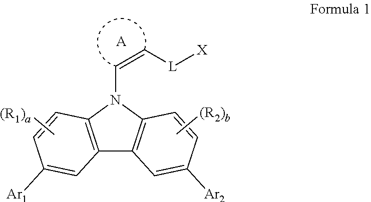
wherein in The Formula 1,
X is a heteroaryl group of 2 to 30 ring-forming carbon atoms, comprising any one of O, S, N or Si,
L is a direct linkage, a substituted or an unsubstituted arylene group of 6 to 30 ring-forming carbon atoms, or a substituted or an unsubstituted heteroarylene group of 2 to 30 ring-forming carbon atoms,
ring A is a substituted or an unsubstituted aryl ring of 6 to 30 ring-forming carbon atoms, or a substituted or an unsubstituted heteroaryl ring of 2 to 30 ring-forming carbon atoms,
a and b are each, independently from one another, an integer of 0 to 3,
R₁ and R₂ are each, independently from one another, a hydrogen atom, a deuterium atom, a halogen atom, a nitro group, a cyano group, a hydroxyl group, a substituted or an unsubstituted amine group, a substituted or an unsubstituted thiol group, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring, and
Ar₁ and Ar₂ are each, independently from one another, of Formula 2: 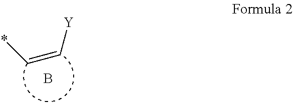
wherein in the Formula 2,
Y is a substituted or an unsubstituted aryl group of 6 to 30 ring-forming carbon atoms, or a substituted or an unsubstituted heteroaryl group of 2 to 30 ring-forming carbon atoms, comprising any one of a furan ring, a thiophene ring, a pyrrole ring, or a silole ring,
* is a binding site to an adjacent atom, and
ring B is a substituted or an unsubstituted aryl ring of 6 to 30 ring-forming carbon atoms, or a substituted or an unsubstituted heteroaryl ring of 2 to 30 ring-forming carbon atoms.

15. The compound of claim 14, wherein X of the Formula 1 is of Formula 3-1 or Formula 3-2: 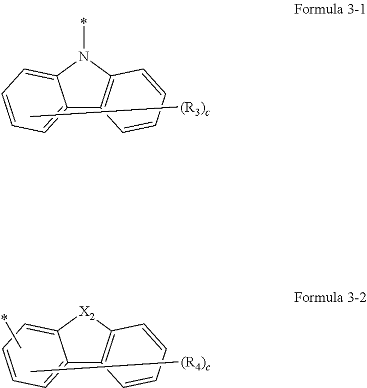
wherein in Formula 3-1 or Formula 3-2,
R₃ and R₄ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring,
c is an integer of 0 to 8,
d is an integer of 0 to 7,
X₂ is O, S, NAr₃, or SiAr₄Ar₅, and
Ar₃ to Ar₅ are each, independently from one another, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms.

16. The compound of claim 14, wherein the Formula 2 is of Formula 4: 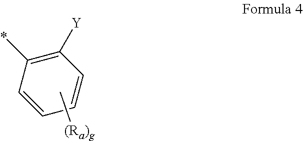
wherein in the Formula 4,
Rₐ is a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, a substituted or an unsubstituted heteroaryl group of 2 to 30 ring-forming carbon atoms, or combined with an adjacent group to form a ring,
g is an integer of 0 to 4,
* is a binding site to an adjacent atom, and
Y has the same meaning as in the Formula 2 of claim 14.

17. The compound of claim 14, wherein the Formula 1 is of Formula 5: 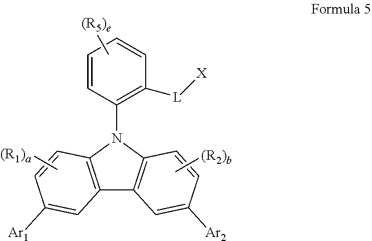
wherein in the Formula 5,
R₅ is a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
e is an integer of 0 to 4, and
R₁, R₂, L, X, Ar₁, Ar₂, a, and b have, independently from one another, the same meaning as in The Formula 1 of claim 14.

18. The compound of claim 17, wherein the Formula 5 is of Formula 6-1: 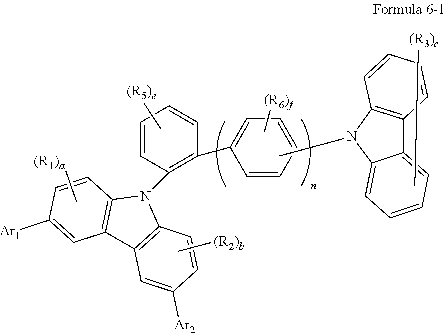
wherein in Formula 6-1,
R₃ and R₆ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
c is an integer of 0 to 8,
f is an integer of 0 to 4,
n is an integer of 0 to 3, and
R₁, R₂, R₅, Ar₁, Ar₂, a, b, and e have, independently from one another, the same meaning as in the Formula 5 of claim 17.

19. The compound of claim 17, wherein the Formula 5 is of Formula 6-2: 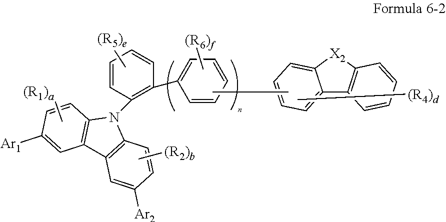
wherein in Formula 6-2,
R₄ and R₆ are each, independently from one another, a hydrogen atom, a deuterium atom, a halogen atom, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, or combined with an adjacent group to form a ring,
d is an integer of 0 to 7,
f is an integer of 0 to 4,
n is an integer of 0 to 3,
X₂ is O, S, NAr₃, or SiAr₄Ar₅,
Ar₃ to Ar₅ are each, independently from one another, a substituted or an unsubstituted alkyl group of 1 to 20 carbon atoms, a substituted or an unsubstituted aryl group of 6 to 30 ring-forming carbon atoms, or a substituted or an unsubstituted heteroaryl group of 2 to 30 ring-forming carbon atoms, and
R₁, R₂, R₅, Ar₁, Ar₂, a, b, and e have, independently from one another, the same meaning as in in the Formula 5 of claim 17.

20. The compound of claim 14, wherein the compound of the Formula 1 is at least one compound of Compound Group 1: 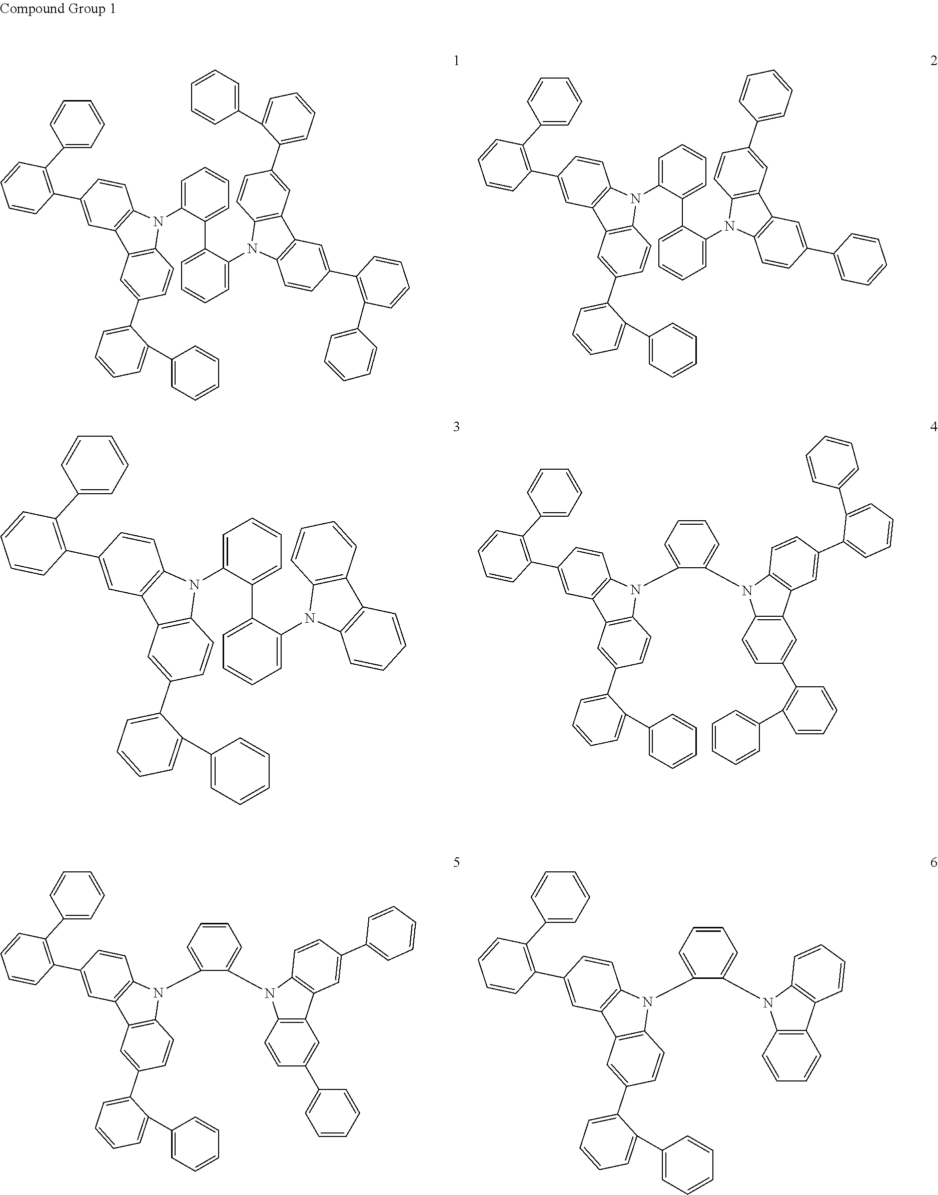 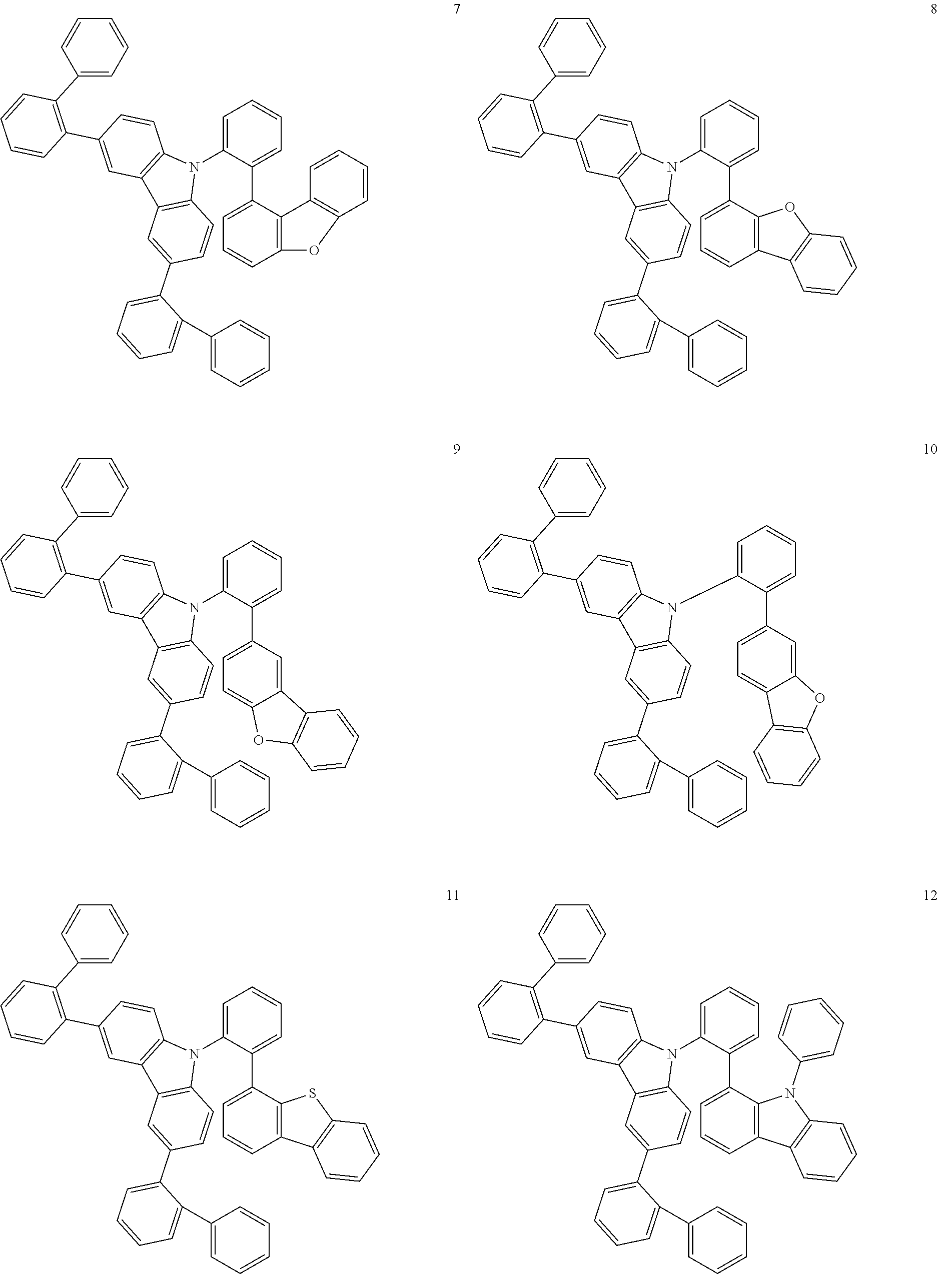 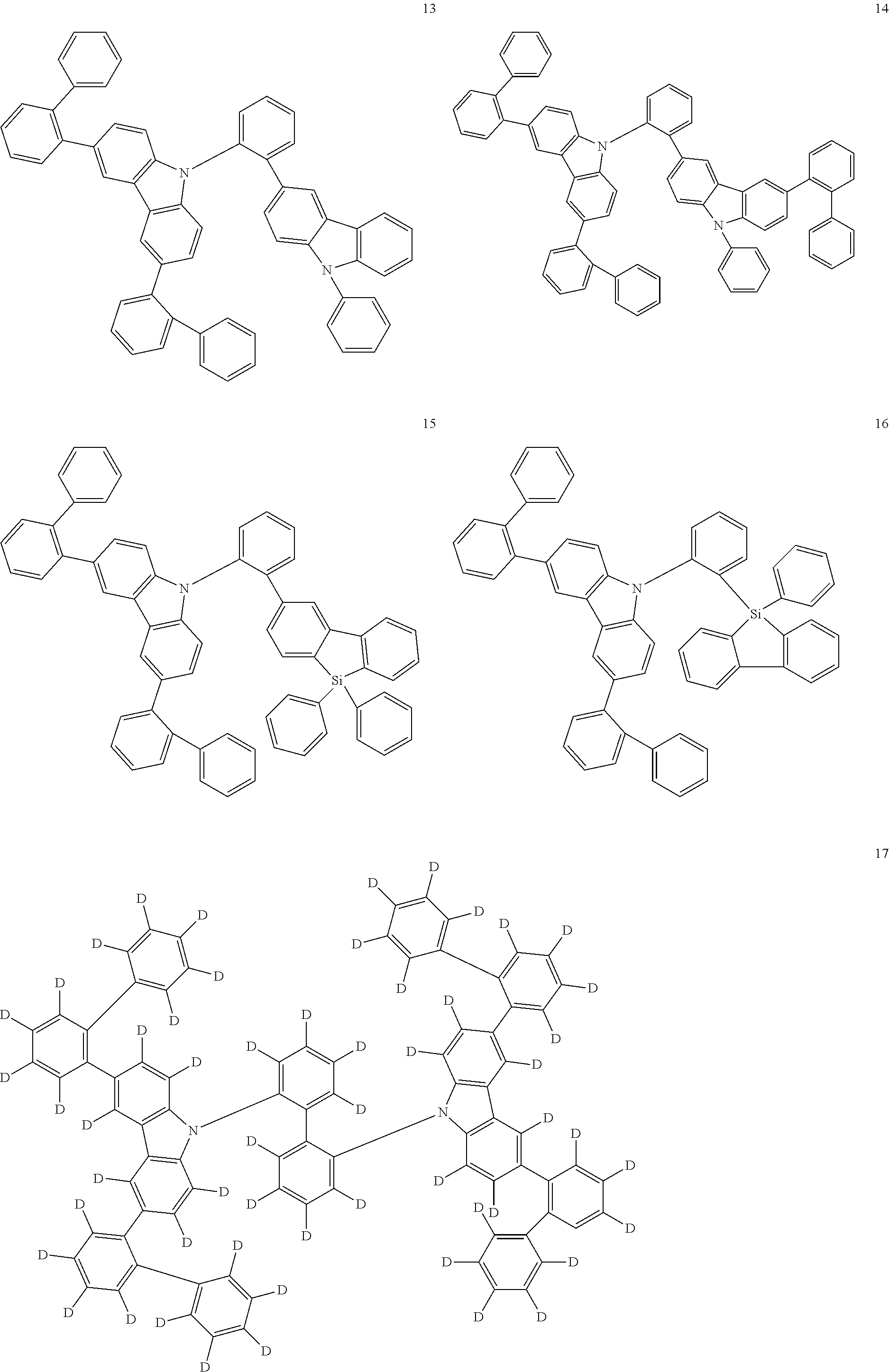 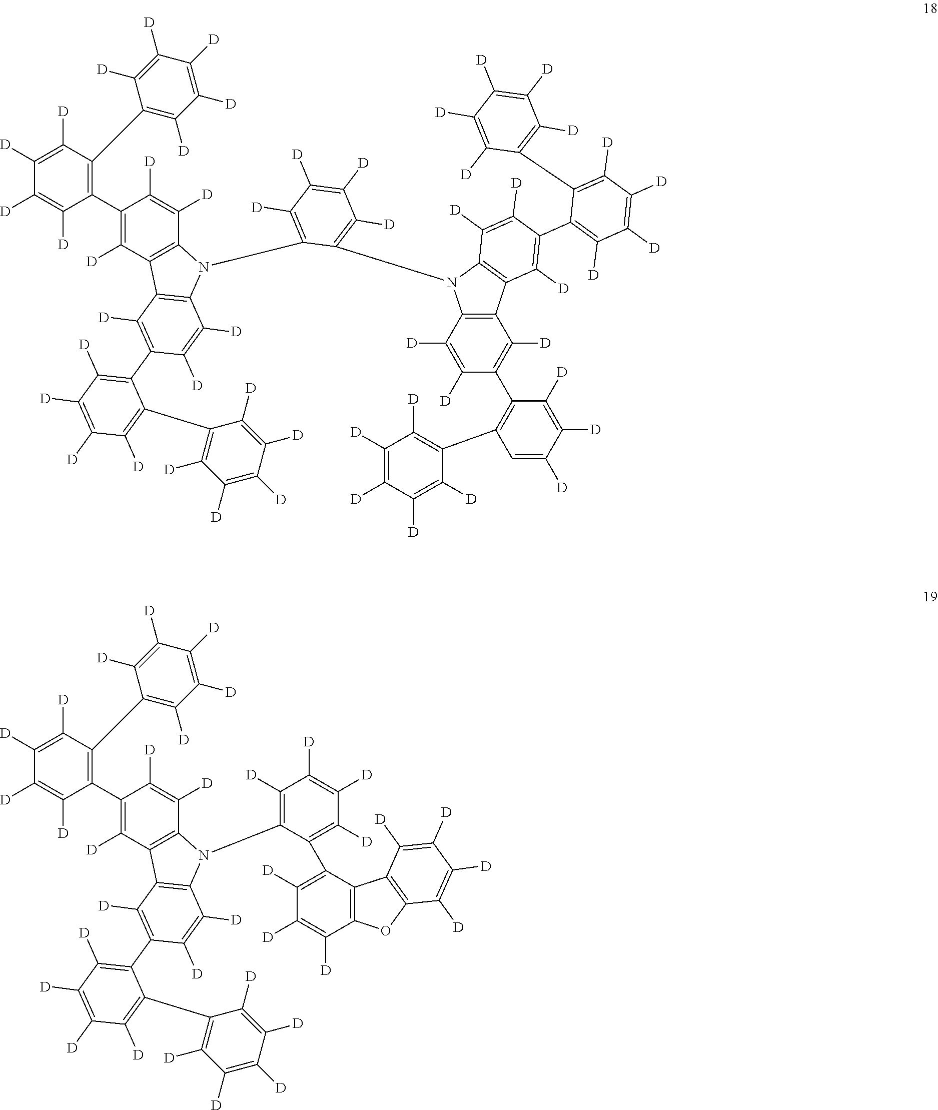 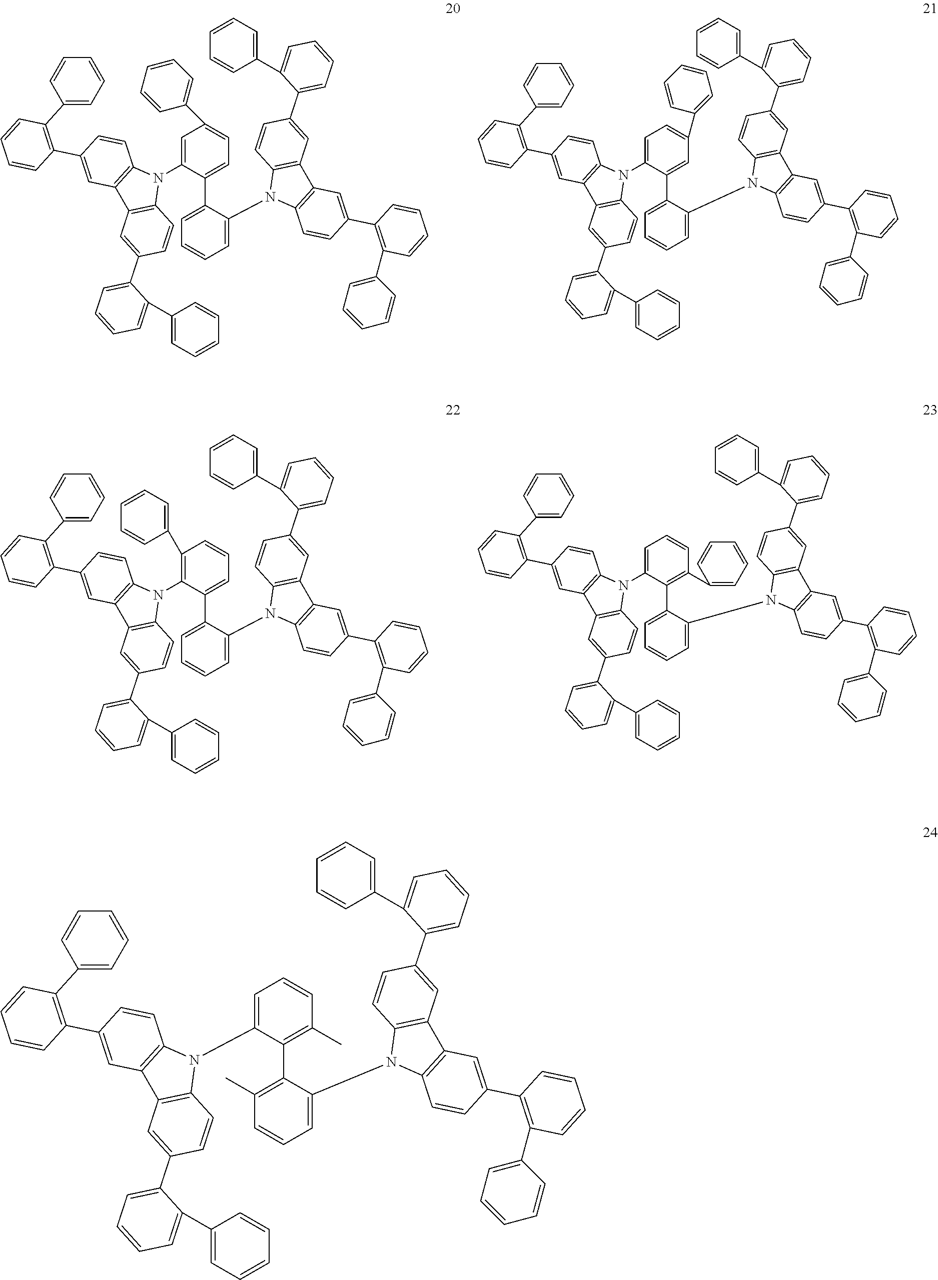 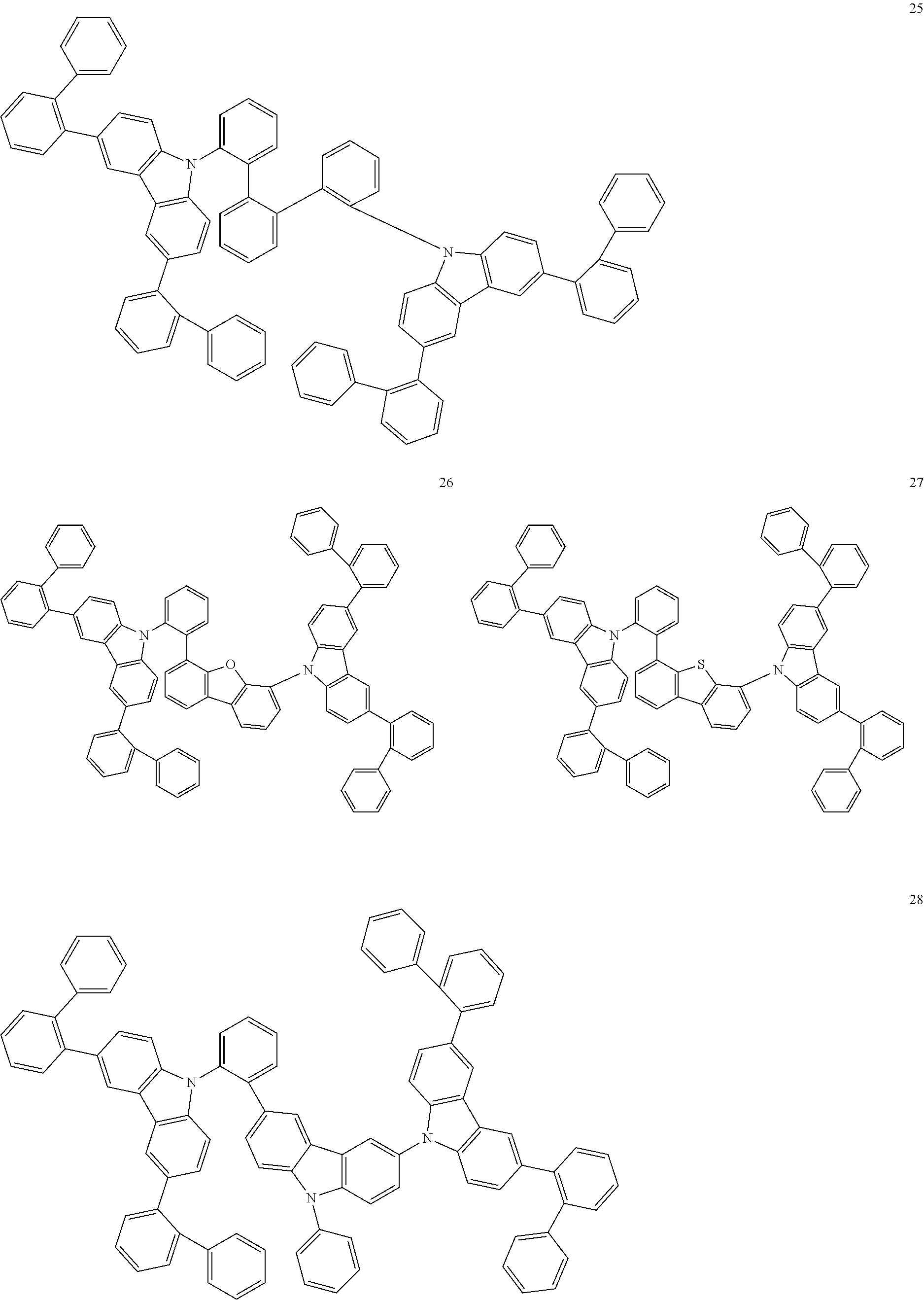 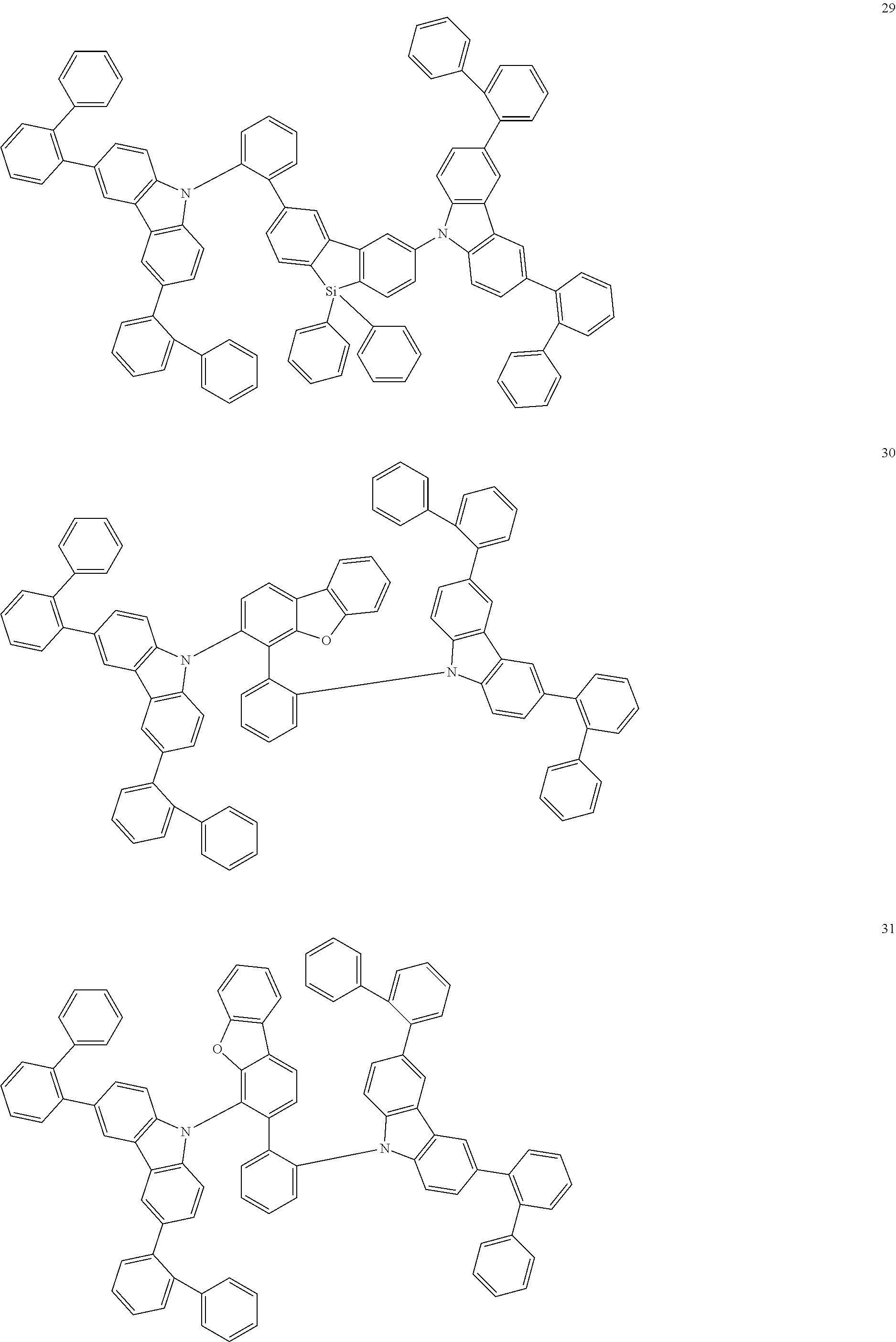 [SMILES: c1ccc(-c2ccccc2-c2ccc3c(c2)c2cc(-c4ccccc4-c4ccccc4)ccc2n3-c2ccc3c(c2)-c2cc(-c4ccccc4-n4c5ccc(-c6ccccc6-c6ccccc6)cc5c5cc(-c6ccccc6-c6ccccc6)ccc54)ccc2[Si]3(c2ccccc2)c2ccccc2)cc1 ; c1ccc(-c2ccccc2-c2ccc3c(c2)c2cc(-c4ccccc4-c4ccccc4)ccc2n3-c2ccccc2-c2c(-n3c4ccc(-c5ccccc5-c5ccccc5)cc4c4cc(-c5ccccc5-c5ccccc5)ccc43)ccc3c2oc2ccccc23)cc1 ; c1ccc(-c2ccccc2-c2ccc3c(c2)c2cc(-c4ccccc4-c4ccccc4)ccc2n3-c2ccccc2-c2ccc3c(oc4ccccc43)c2-n2c3ccc(-c4ccccc4-c4ccccc4)cc3c3cc(-c4ccccc4-c4ccccc4)ccc32)cc1] 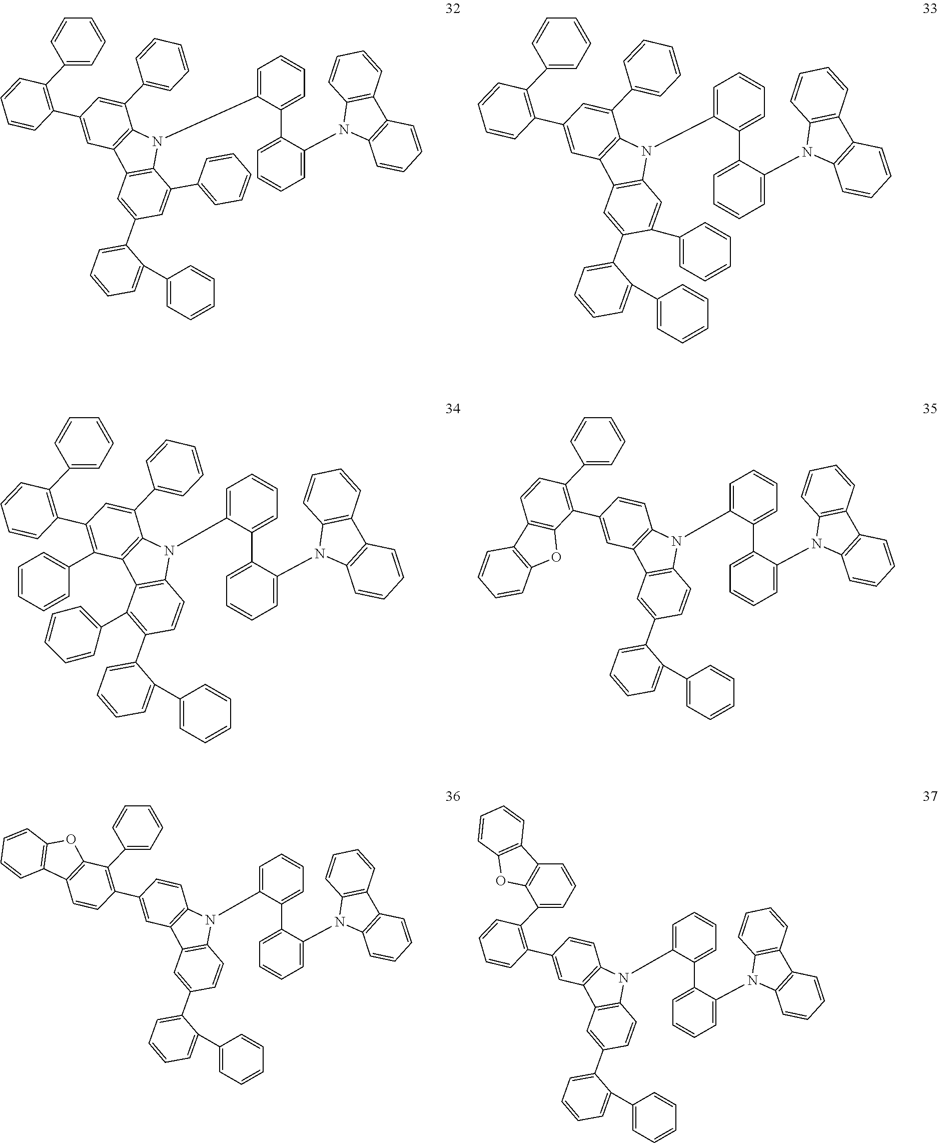 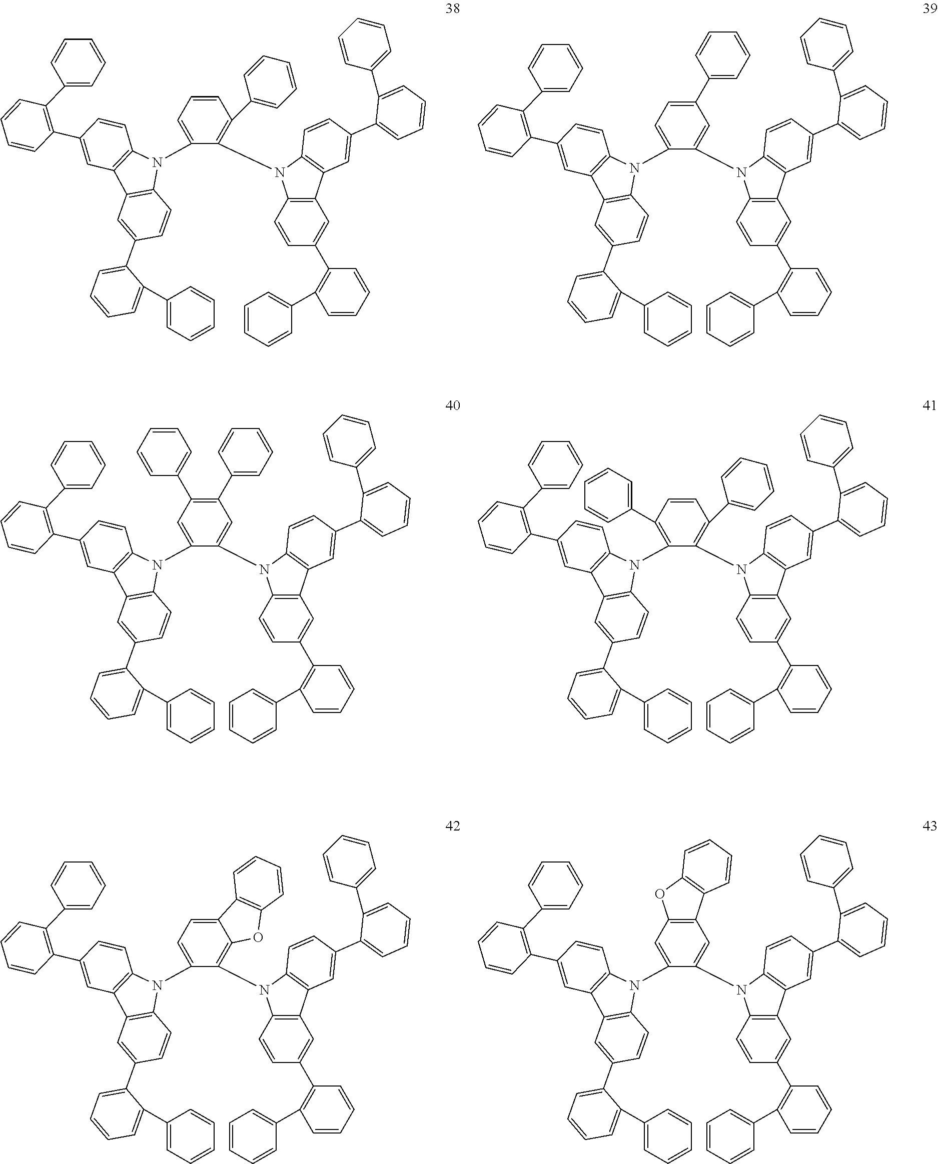 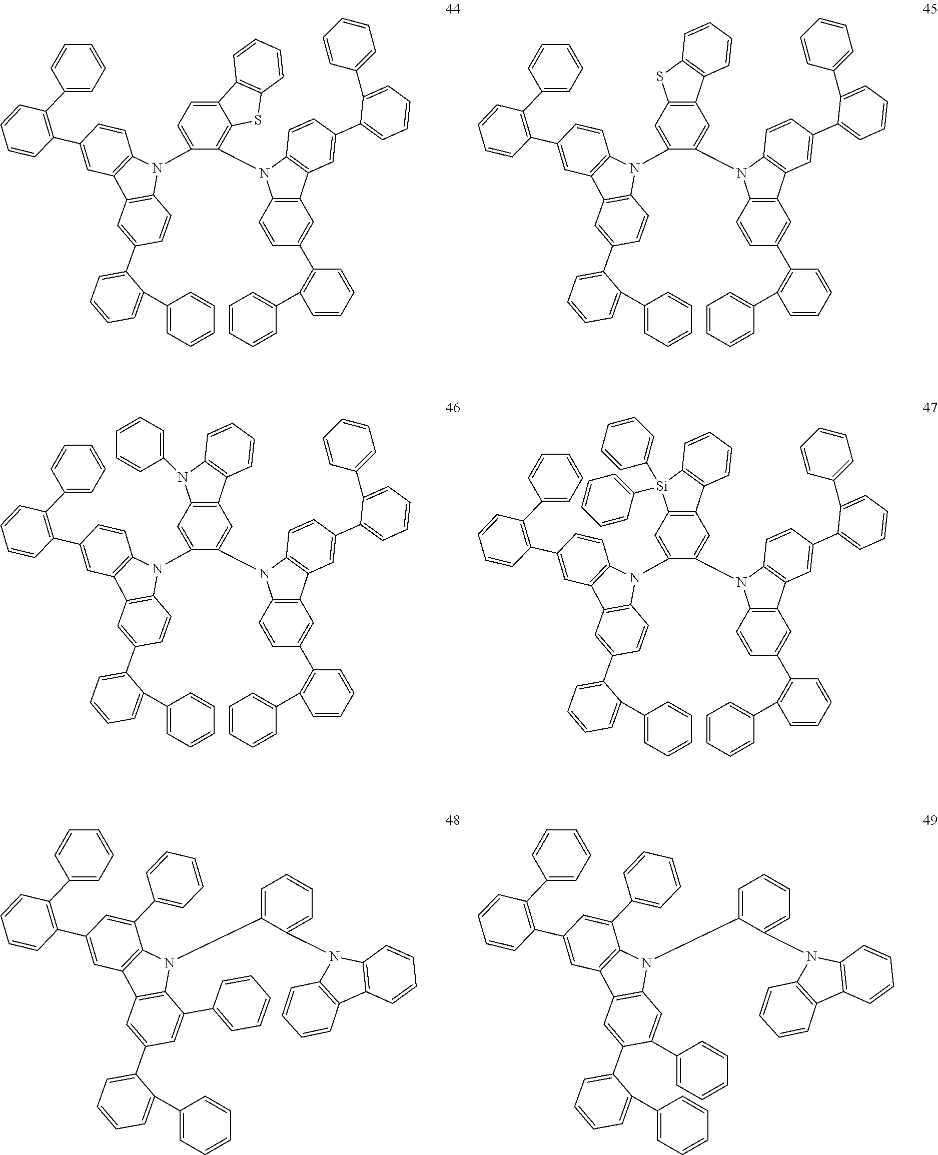 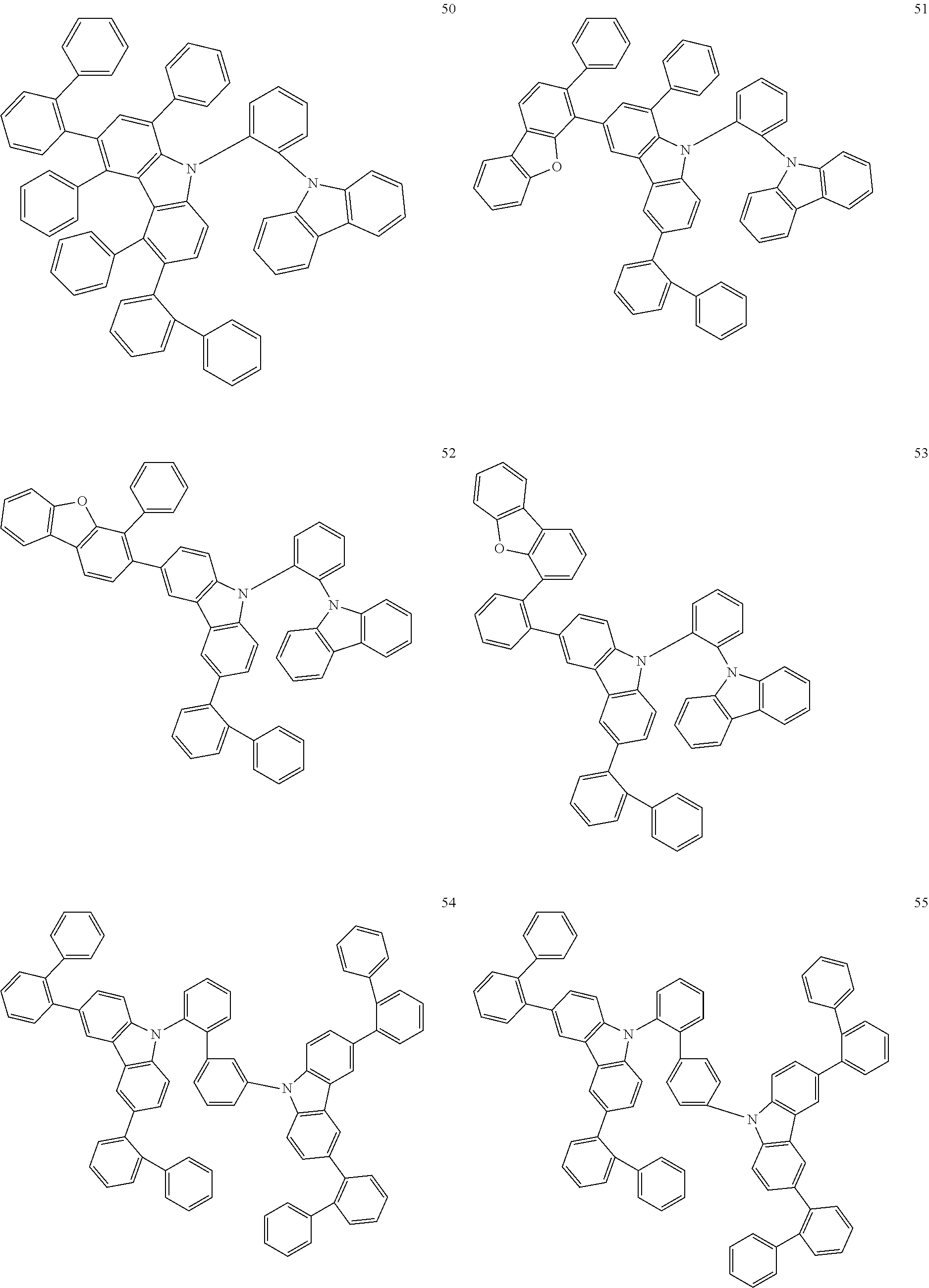 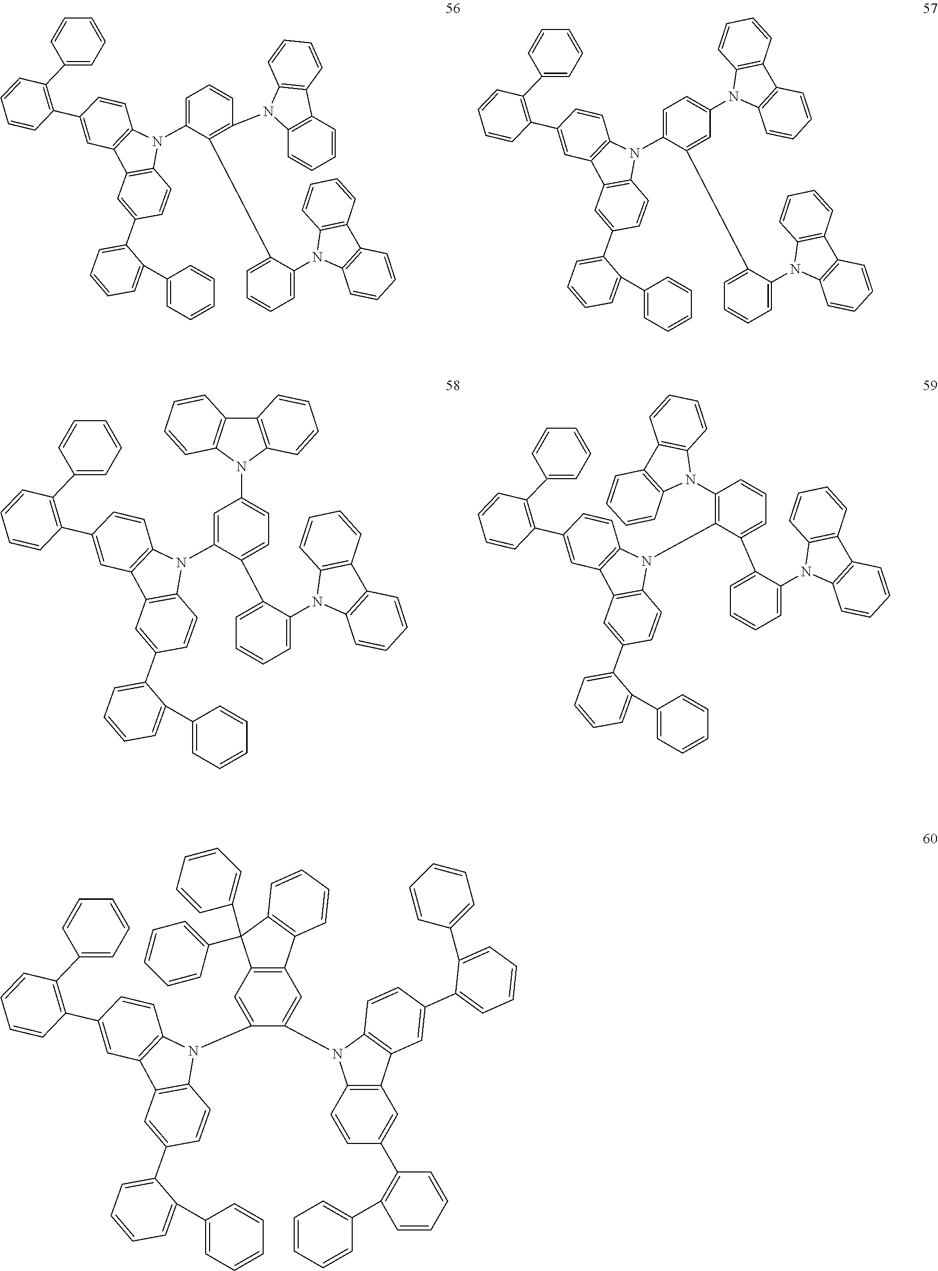 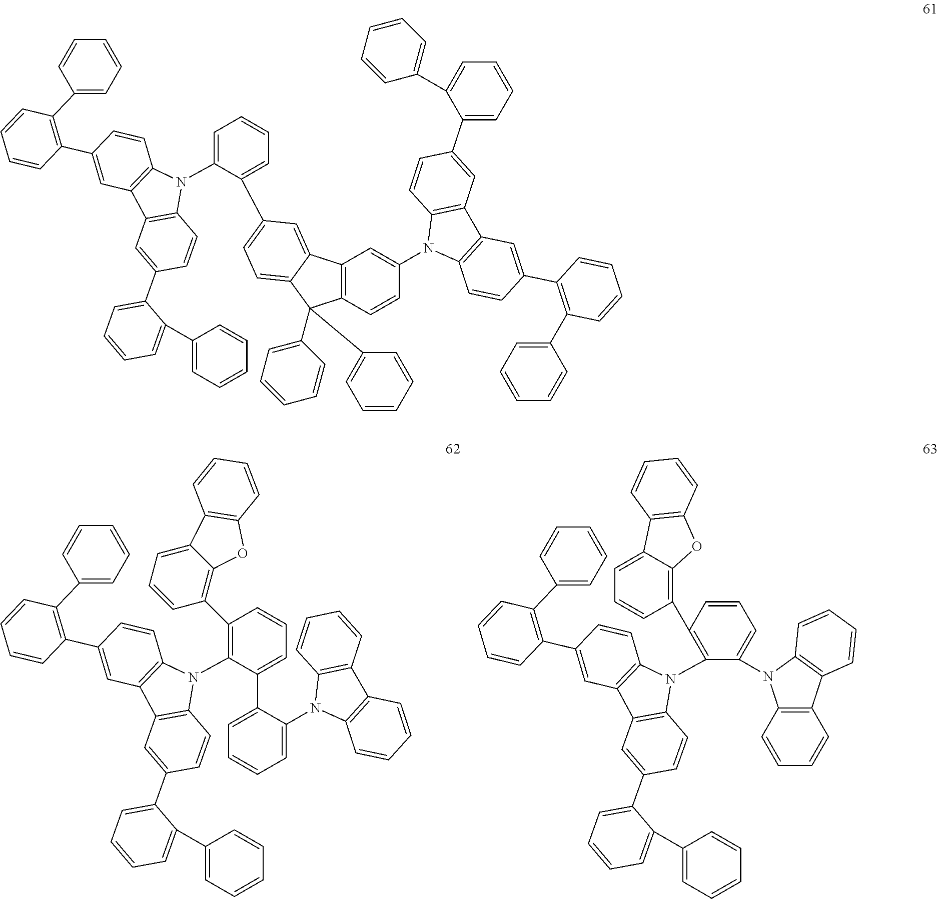 [SMILES: c1ccc(-c2ccccc2-c2ccc3c(c2)c2cc(-c4ccccc4-c4ccccc4)ccc2n3-c2ccc3c(c2)-c2cc(-c4ccccc4-n4c5ccc(-c6ccccc6-c6ccccc6)cc5c5cc(-c6ccccc6-c6ccccc6)ccc54)ccc2C3(c2ccccc2)c2ccccc2)cc1 ; c1ccc(-c2ccccc2-c2ccc3c(c2)c2cc(-c4ccccc4-c4ccccc4)ccc2n3-c2c(-c3ccccc3-n3c4ccccc4c4ccccc43)cccc2-c2cccc3c2oc2ccccc23)cc1 ; c1ccc(-c2ccccc2-c2ccc3c(c2)c2cc(-c4ccccc4-c4ccccc4)ccc2n3-c2c(-c3cccc4c3oc3ccccc34)cccc2-n2c3ccccc3c3ccccc32)cc1] 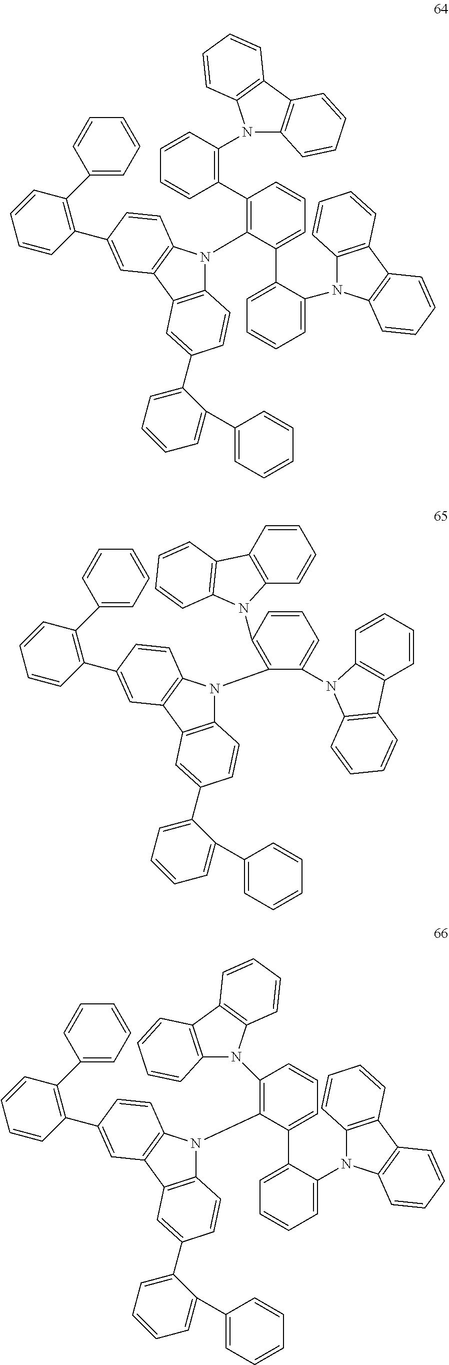 [SMILES: c1ccc(-c2ccccc2-c2ccc3c(c2)c2cc(-c4ccccc4-c4ccccc4)ccc2n3-c2c(-c3ccccc3-n3c4ccccc4c4ccccc43)cccc2-c2ccccc2-n2c3ccccc3c3ccccc32)cc1 ; c1ccc(-c2ccccc2-c2ccc3c(c2)c2cc(-c4ccccc4-c4ccccc4)ccc2n3-c2c(-n3c4ccccc4c4ccccc43)cccc2-n2c3ccccc3c3ccccc32)cc1 ; c1ccc(-c2ccccc2-c2ccc3c(c2)c2cc(-c4ccccc4-c4ccccc4)ccc2n3-c2c(-c3ccccc3-n3c4ccccc4c4ccccc43)cccc2-n2c3ccccc3c3ccccc32)cc1] 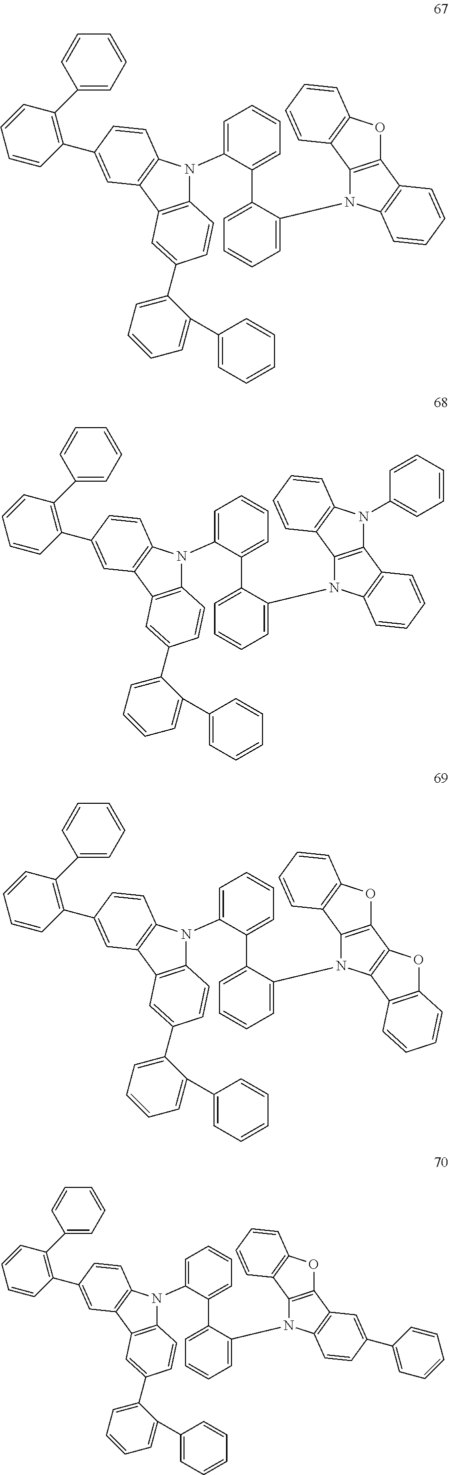 [SMILES: c1ccc(-c2ccccc2-c2ccc3c(c2)c2cc(-c4ccccc4-c4ccccc4)ccc2n3-c2ccccc2-c2ccccc2-n2c3ccccc3c3oc4ccccc4c32)cc1 ; c1ccc(-c2ccccc2-c2ccc3c(c2)c2cc(-c4ccccc4-c4ccccc4)ccc2n3-c2ccccc2-c2ccccc2-n2c3ccccc3c3c2c2ccccc2n3-c2ccccc2)cc1 ; c1ccc(-c2ccccc2-c2ccc3c(c2)c2cc(-c4ccccc4-c4ccccc4)ccc2n3-c2ccccc2-c2ccccc2-n2c3c4ccccc4oc3c3oc4ccccc4c32)cc1 ; c1ccc(-c2ccc3c(c2)c2oc4ccccc4c2n3-c2ccccc2-c2ccccc2-n2c3ccc(-c4ccccc4-c4ccccc4)cc3c3cc(-c4ccccc4-c4ccccc4)ccc32)cc1] 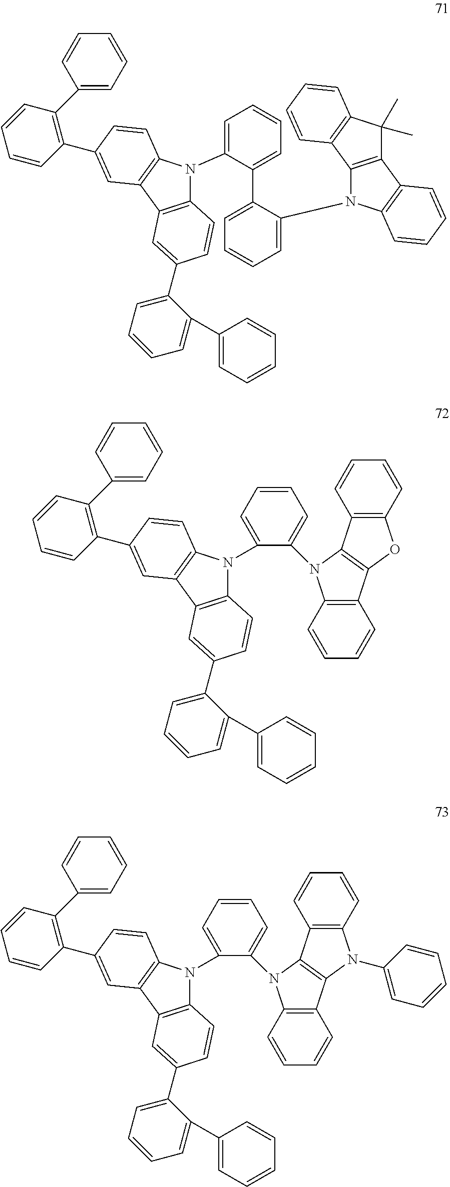 [SMILES: CC1(C)c2ccccc2-c2c1c1ccccc1n2-c1ccccc1-c1ccccc1-n1c2ccc(-c3ccccc3-c3ccccc3)cc2c2cc(-c3ccccc3-c3ccccc3)ccc21 ; c1ccc(-c2ccccc2-c2ccc3c(c2)c2cc(-c4ccccc4-c4ccccc4)ccc2n3-c2ccccc2-n2c3ccccc3c3oc4ccccc4c32)cc1 ; c1ccc(-c2ccccc2-c2ccc3c(c2)c2cc(-c4ccccc4-c4ccccc4)ccc2n3-c2ccccc2-n2c3ccccc3c3c2c2ccccc2n3-c2ccccc2)cc1] 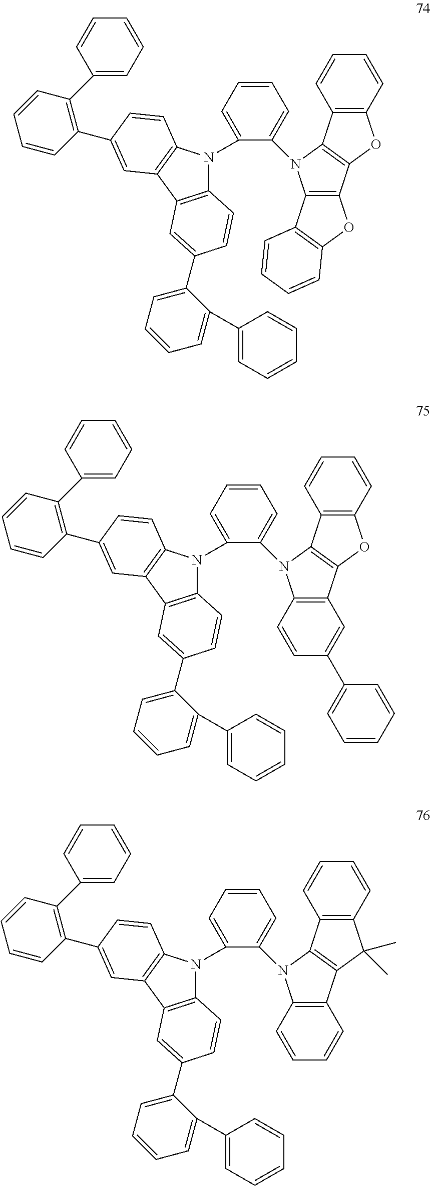 [SMILES: c1ccc(-c2ccccc2-c2ccc3c(c2)c2cc(-c4ccccc4-c4ccccc4)ccc2n3-c2ccccc2-n2c3c4ccccc4oc3c3oc4ccccc4c32)cc1 ; c1ccc(-c2ccc3c(c2)c2oc4ccccc4c2n3-c2ccccc2-n2c3ccc(-c4ccccc4-c4ccccc4)cc3c3cc(-c4ccccc4-c4ccccc4)ccc32)cc1 ; CC1(C)c2ccccc2-c2c1c1ccccc1n2-c1ccccc1-n1c2ccc(-c3ccccc3-c3ccccc3)cc2c2cc(-c3ccccc3-c3ccccc3)ccc21] 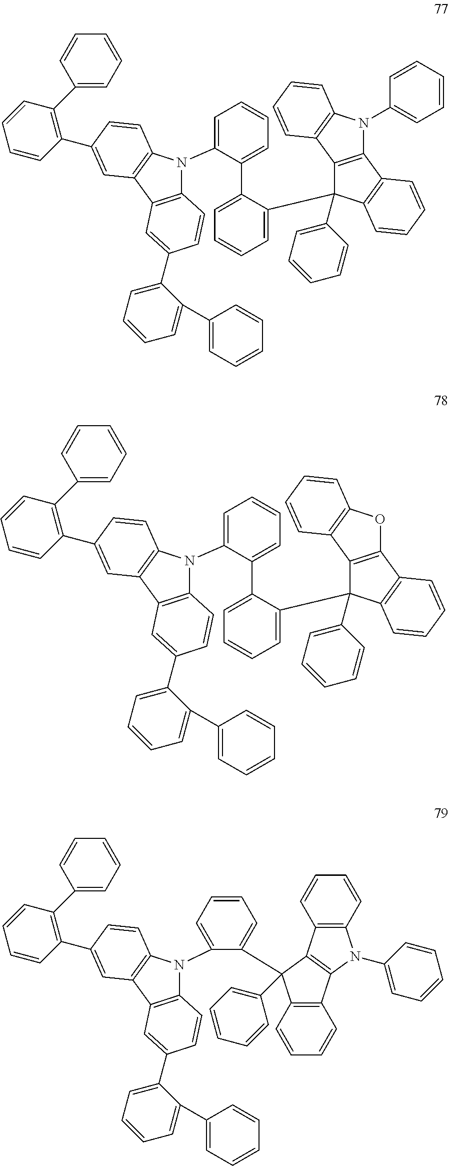 [SMILES: c1ccc(-c2ccccc2-c2ccc3c(c2)c2cc(-c4ccccc4-c4ccccc4)ccc2n3-c2ccccc2-c2ccccc2C2(c3ccccc3)c3ccccc3-c3c2c2ccccc2n3-c2ccccc2)cc1 ; c1ccc(-c2ccccc2-c2ccc3c(c2)c2cc(-c4ccccc4-c4ccccc4)ccc2n3-c2ccccc2-c2ccccc2C2(c3ccccc3)c3ccccc3-c3oc4ccccc4c32)cc1 ; c1ccc(-c2ccccc2-c2ccc3c(c2)c2cc(-c4ccccc4-c4ccccc4)ccc2n3-c2ccccc2C2(c3ccccc3)c3ccccc3-c3c2c2ccccc2n3-c2ccccc2)cc1] 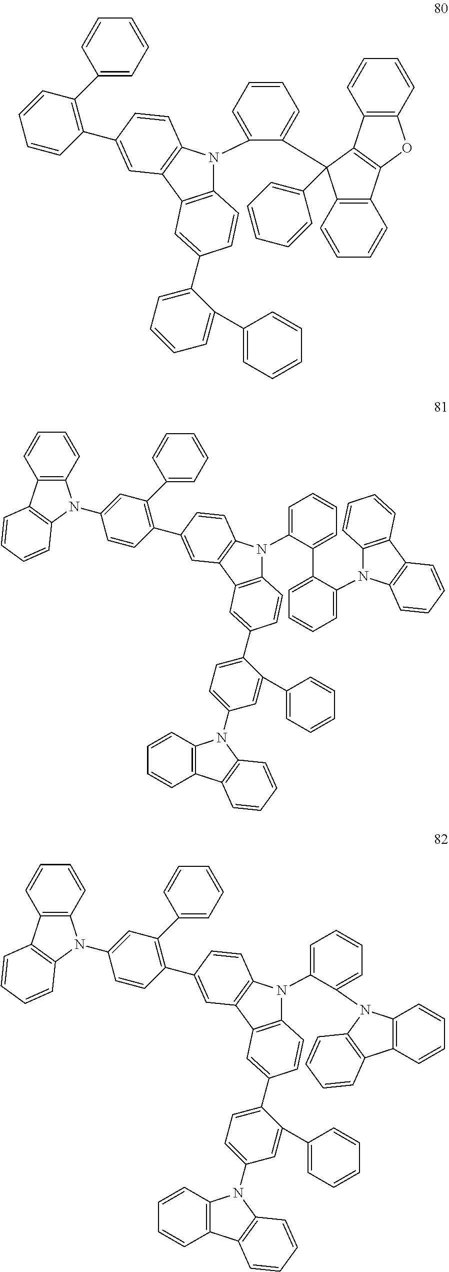 [SMILES: c1ccc(-c2ccccc2-c2ccc3c(c2)c2cc(-c4ccccc4-c4ccccc4)ccc2n3-c2ccccc2C2(c3ccccc3)c3ccccc3-c3oc4ccccc4c32)cc1 ; c1ccc(-c2cc(-n3c4ccccc4c4ccccc43)ccc2-c2ccc3c(c2)c2cc(-c4ccc(-n5c6ccccc6c6ccccc65)cc4-c4ccccc4)ccc2n3-c2ccccc2-c2ccccc2-n2c3ccccc3c3ccccc32)cc1 ; c1ccc(-c2cc(-n3c4ccccc4c4ccccc43)ccc2-c2ccc3c(c2)c2cc(-c4ccc(-n5c6ccccc6c6ccccc65)cc4-c4ccccc4)ccc2n3-c2ccccc2-n2c3ccccc3c3ccccc32)cc1] 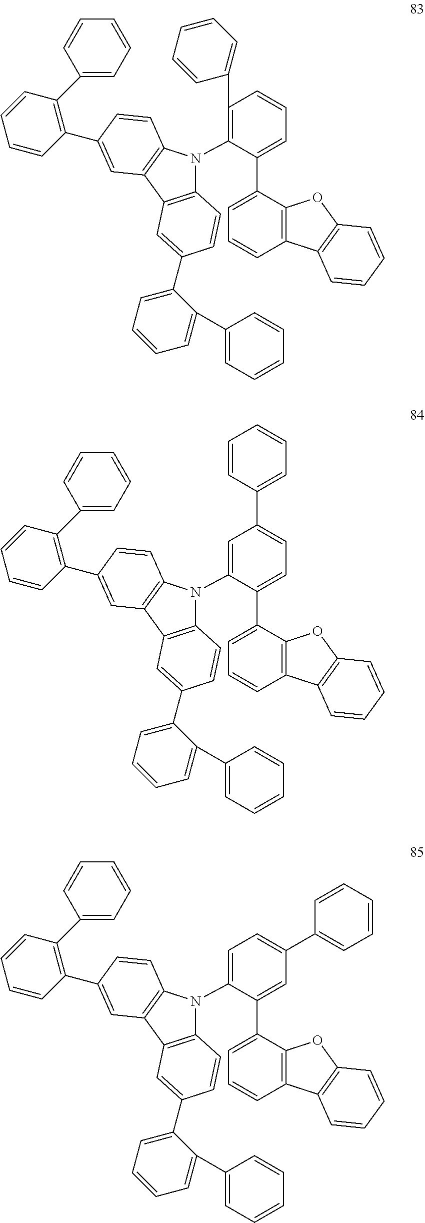 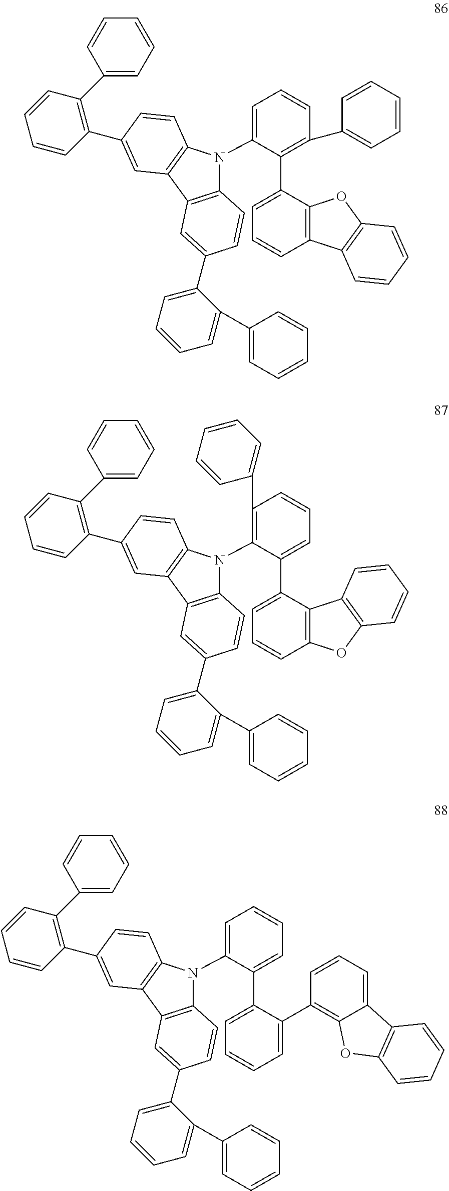 [SMILES: c1ccc(-c2ccccc2-c2ccc3c(c2)c2cc(-c4ccccc4-c4ccccc4)ccc2n3-c2cccc(-c3ccccc3)c2-c2cccc3c2oc2ccccc23)cc1 ; c1ccc(-c2ccccc2-c2ccc3c(c2)c2cc(-c4ccccc4-c4ccccc4)ccc2n3-c2c(-c3ccccc3)cccc2-c2cccc3oc4ccccc4c23)cc1 ; c1ccc(-c2ccccc2-c2ccc3c(c2)c2cc(-c4ccccc4-c4ccccc4)ccc2n3-c2ccccc2-c2ccccc2-c2cccc3c2oc2ccccc23)cc1] 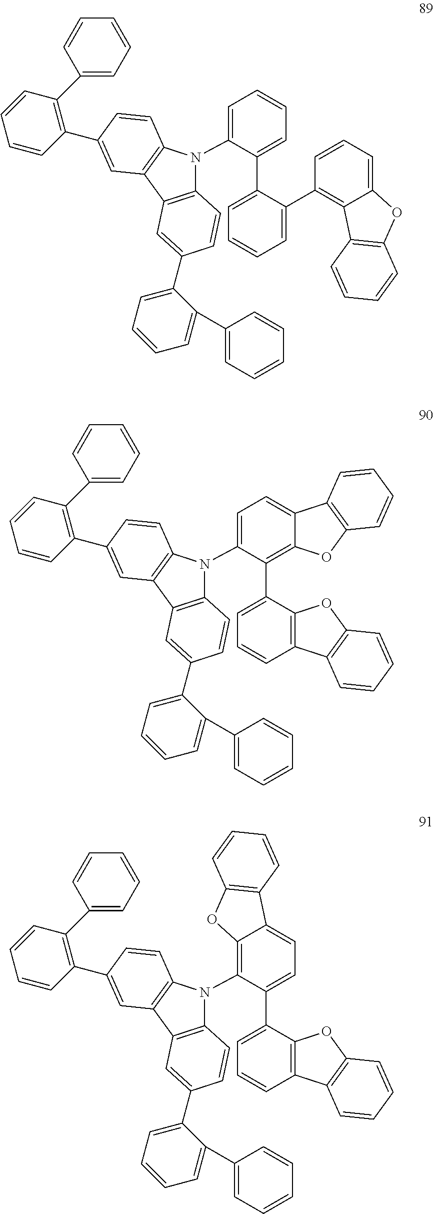 [SMILES: c1ccc(-c2ccccc2-c2ccc3c(c2)c2cc(-c4ccccc4-c4ccccc4)ccc2n3-c2ccccc2-c2ccccc2-c2cccc3oc4ccccc4c23)cc1 ; c1ccc(-c2ccccc2-c2ccc3c(c2)c2cc(-c4ccccc4-c4ccccc4)ccc2n3-c2ccc3c(oc4ccccc43)c2-c2cccc3c2oc2ccccc23)cc1 ; c1ccc(-c2ccccc2-c2ccc3c(c2)c2cc(-c4ccccc4-c4ccccc4)ccc2n3-c2c(-c3cccc4c3oc3ccccc34)ccc3c2oc2ccccc23)cc1] 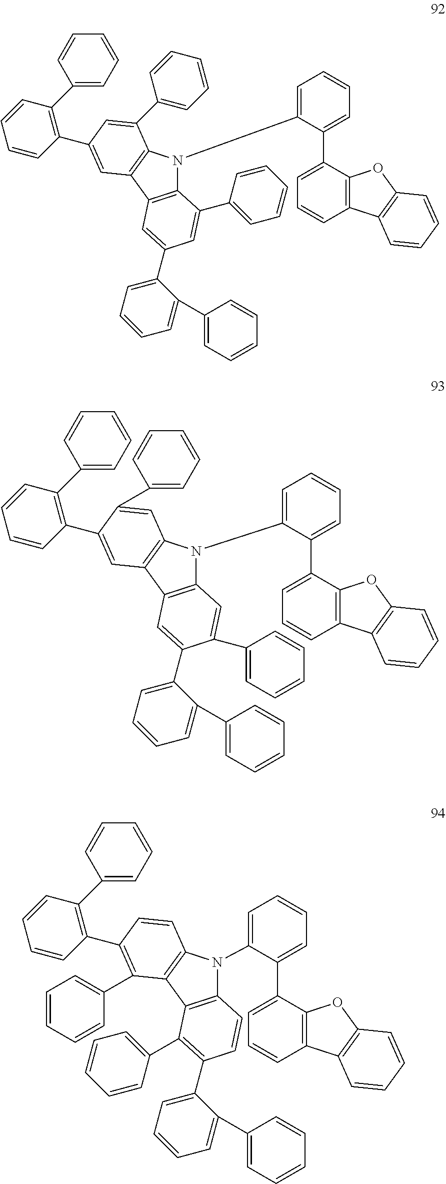 [SMILES: c1ccc(-c2ccccc2-c2cc(-c3ccccc3)c3c(c2)c2cc(-c4ccccc4-c4ccccc4)cc(-c4ccccc4)c2n3-c2ccccc2-c2cccc3c2oc2ccccc23)cc1 ; c1ccc(-c2ccccc2-c2cc3c4cc(-c5ccccc5-c5ccccc5)c(-c5ccccc5)cc4n(-c4ccccc4-c4cccc5c4oc4ccccc45)c3cc2-c2ccccc2)cc1 ; c1ccc(-c2ccccc2-c2ccc3c(c2-c2ccccc2)c2c(-c4ccccc4)c(-c4ccccc4-c4ccccc4)ccc2n3-c2ccccc2-c2cccc3c2oc2ccccc23)cc1] 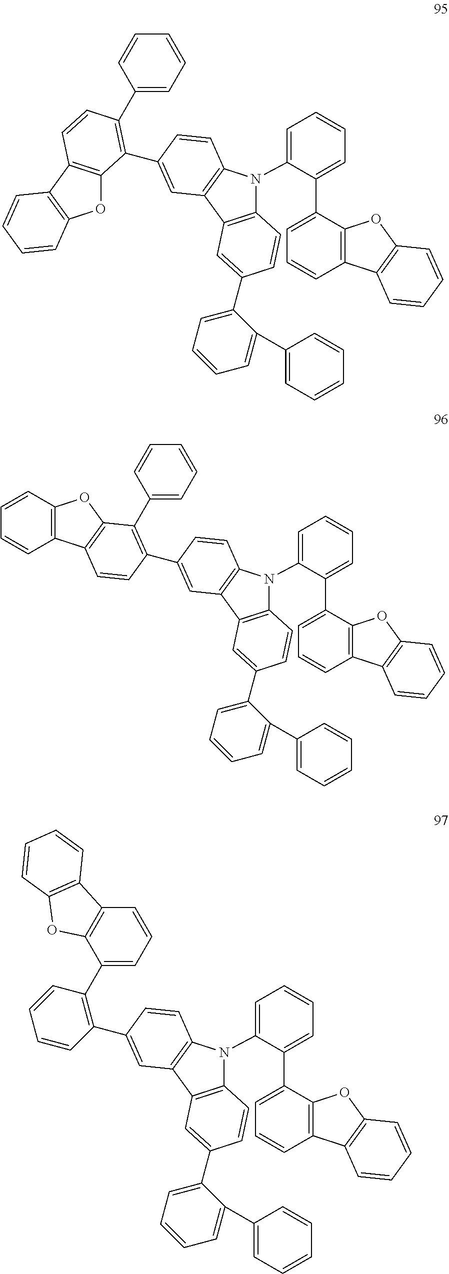 [SMILES: c1ccc(-c2ccccc2-c2ccc3c(c2)c2cc(-c4c(-c5ccccc5)ccc5c4oc4ccccc45)ccc2n3-c2ccccc2-c2cccc3c2oc2ccccc23)cc1 ; c1ccc(-c2ccccc2-c2ccc3c(c2)c2cc(-c4ccc5c(oc6ccccc65)c4-c4ccccc4)ccc2n3-c2ccccc2-c2cccc3c2oc2ccccc23)cc1 ; c1ccc(-c2ccccc2-c2ccc3c(c2)c2cc(-c4ccccc4-c4cccc5c4oc4ccccc45)ccc2n3-c2ccccc2-c2cccc3c2oc2ccccc23)cc1] 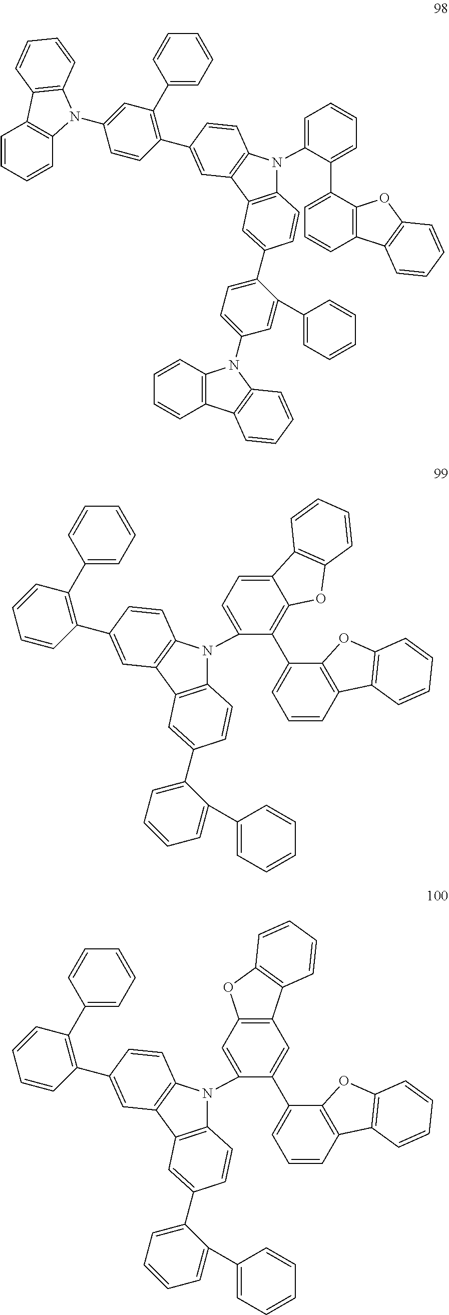 [SMILES: c1ccc(-c2cc(-n3c4ccccc4c4ccccc43)ccc2-c2ccc3c(c2)c2cc(-c4ccc(-n5c6ccccc6c6ccccc65)cc4-c4ccccc4)ccc2n3-c2ccccc2-c2cccc3c2oc2ccccc23)cc1 ; c1ccc(-c2ccccc2-c2ccc3c(c2)c2cc(-c4ccccc4-c4ccccc4)ccc2n3-c2ccc3c(oc4ccccc43)c2-c2cccc3c2oc2ccccc23)cc1 ; c1ccc(-c2ccccc2-c2ccc3c(c2)c2cc(-c4ccccc4-c4ccccc4)ccc2n3-c2cc3oc4ccccc4c3cc2-c2cccc3c2oc2ccccc23)cc1] 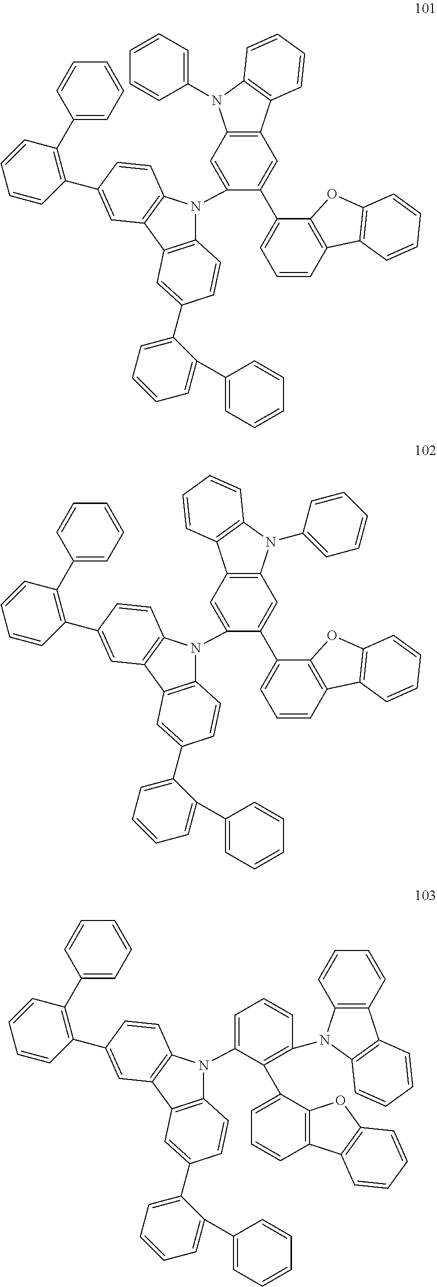 [SMILES: c1ccc(-c2ccccc2-c2ccc3c(c2)c2cc(-c4ccccc4-c4ccccc4)ccc2n3-c2cc3c(cc2-c2cccc4c2oc2ccccc24)c2ccccc2n3-c2ccccc2)cc1 ; c1ccc(-c2ccccc2-c2ccc3c(c2)c2cc(-c4ccccc4-c4ccccc4)ccc2n3-c2cc3c4ccccc4n(-c4ccccc4)c3cc2-c2cccc3c2oc2ccccc23)cc1 ; c1ccc(-c2ccccc2-c2ccc3c(c2)c2cc(-c4ccccc4-c4ccccc4)ccc2n3-c2cccc(-n3c4ccccc4c4ccccc43)c2-c2cccc3c2oc2ccccc23)cc1] 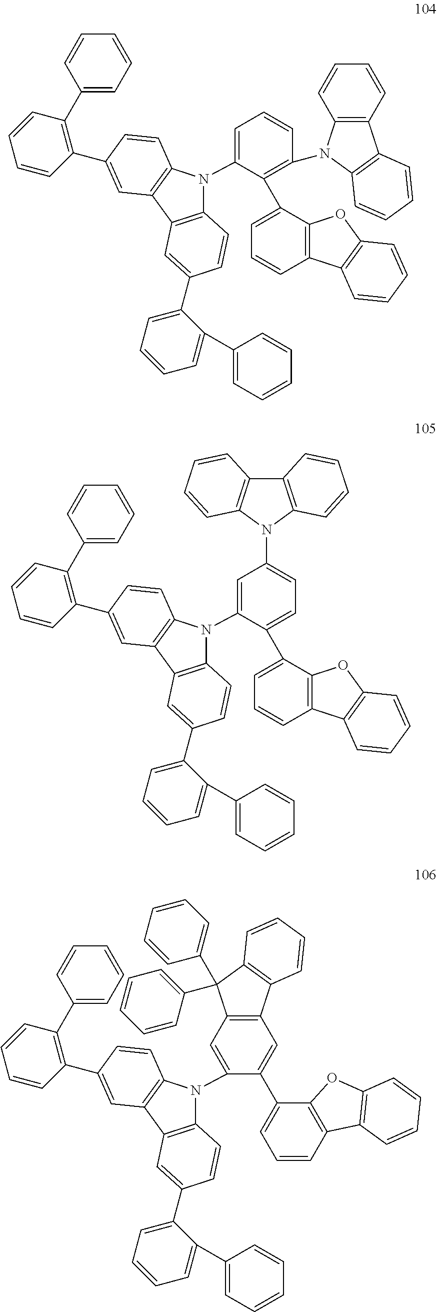 [SMILES: c1ccc(-c2ccccc2-c2ccc3c(c2)c2cc(-c4ccccc4-c4ccccc4)ccc2n3-c2cccc(-n3c4ccccc4c4ccccc43)c2-c2cccc3c2oc2ccccc23)cc1 ; c1ccc(-c2ccccc2-c2ccc3c(c2)c2cc(-c4ccccc4-c4ccccc4)ccc2n3-c2cc(-n3c4ccccc4c4ccccc43)ccc2-c2cccc3c2oc2ccccc23)cc1 ; c1ccc(-c2ccccc2-c2ccc3c(c2)c2cc(-c4ccccc4-c4ccccc4)ccc2n3-c2cc3c(cc2-c2cccc4c2oc2ccccc24)-c2ccccc2C3(c2ccccc2)c2ccccc2)cc1] 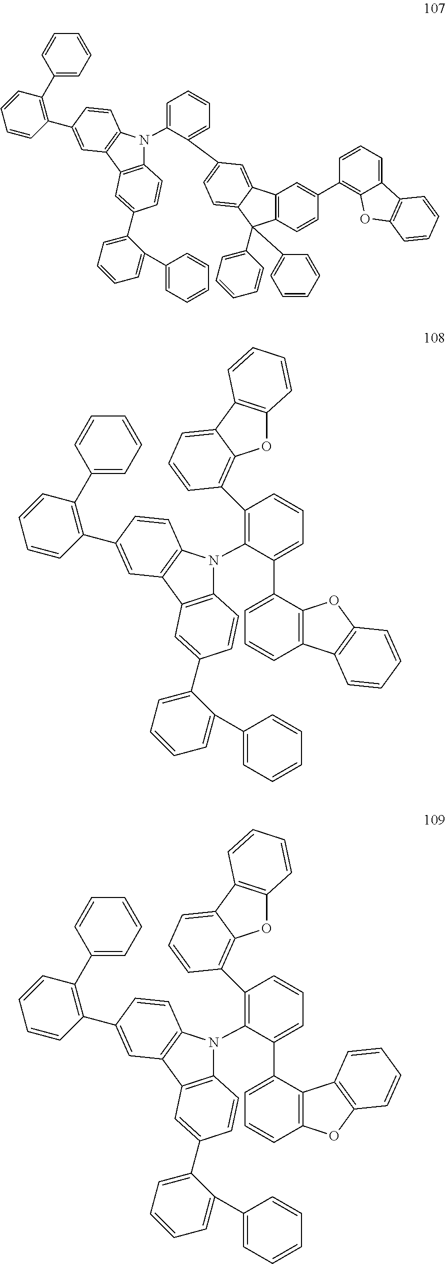 [SMILES: c1ccc(-c2ccccc2-c2ccc3c(c2)c2cc(-c4ccccc4-c4ccccc4)ccc2n3-c2ccccc2-c2ccc3c(c2)-c2cc(-c4cccc5c4oc4ccccc45)ccc2C3(c2ccccc2)c2ccccc2)cc1 ; c1ccc(-c2ccccc2-c2ccc3c(c2)c2cc(-c4ccccc4-c4ccccc4)ccc2n3-c2c(-c3cccc4c3oc3ccccc34)cccc2-c2cccc3c2oc2ccccc23)cc1 ; c1ccc(-c2ccccc2-c2ccc3c(c2)c2cc(-c4ccccc4-c4ccccc4)ccc2n3-c2c(-c3cccc4c3oc3ccccc34)cccc2-c2cccc3oc4ccccc4c23)cc1] 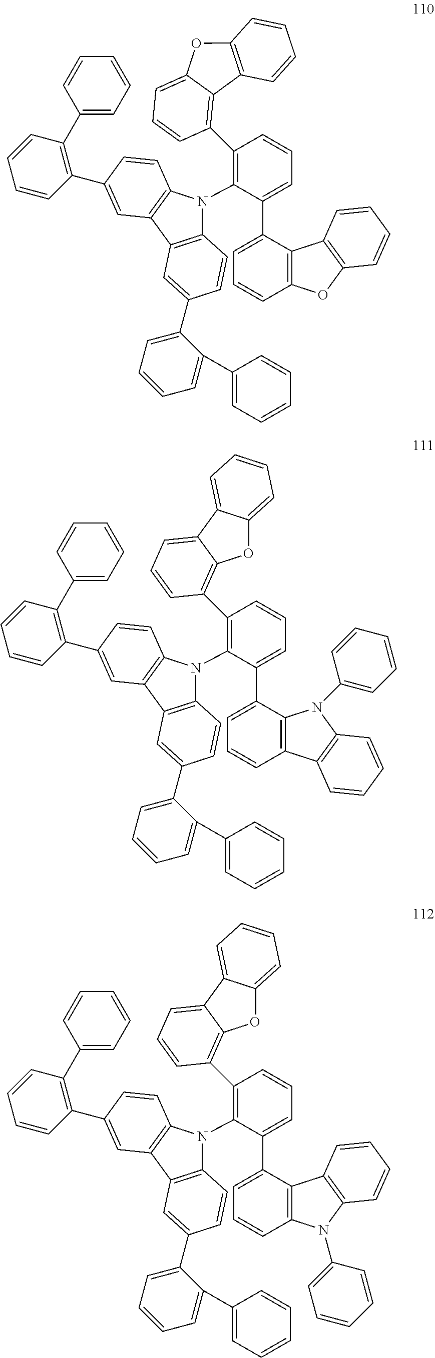 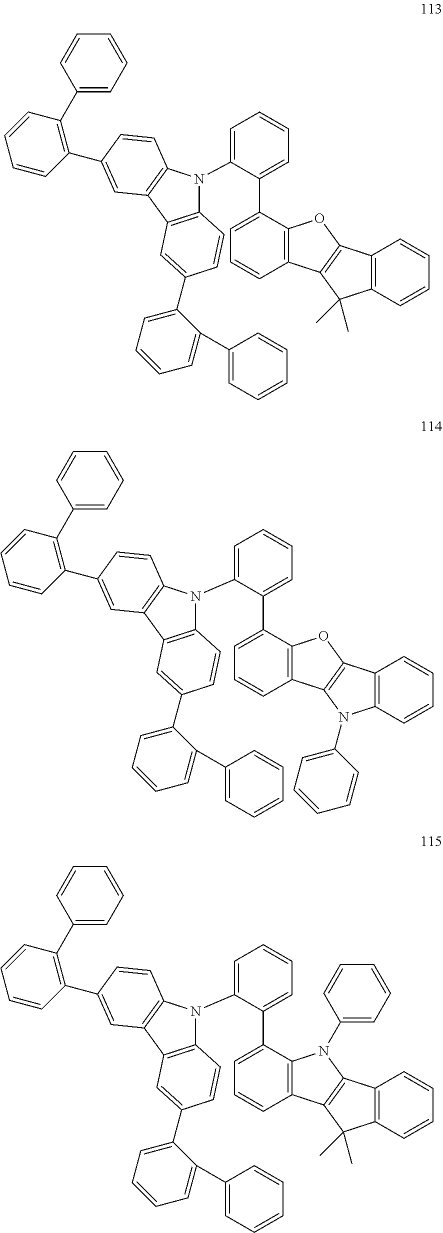 [SMILES: CC1(C)c2ccccc2-c2oc3c(-c4ccccc4-n4c5ccc(-c6ccccc6-c6ccccc6)cc5c5cc(-c6ccccc6-c6ccccc6)ccc54)cccc3c21 ; c1ccc(-c2ccccc2-c2ccc3c(c2)c2cc(-c4ccccc4-c4ccccc4)ccc2n3-c2ccccc2-c2cccc3c2oc2c4ccccc4n(-c4ccccc4)c32)cc1 ; CC1(C)c2ccccc2-c2c1c1cccc(-c3ccccc3-n3c4ccc(-c5ccccc5-c5ccccc5)cc4c4cc(-c5ccccc5-c5ccccc5)ccc43)c1n2-c1ccccc1] 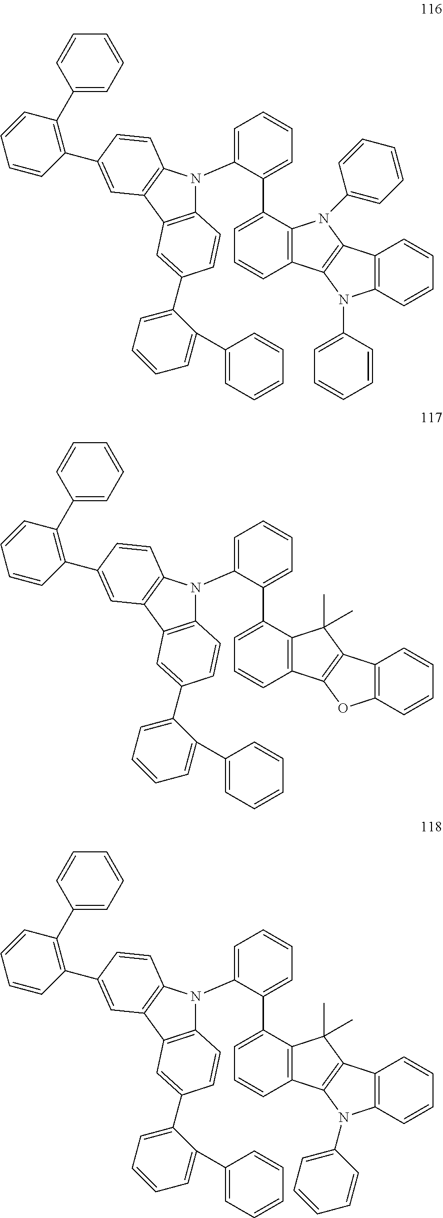 [SMILES: c1ccc(-c2ccccc2-c2ccc3c(c2)c2cc(-c4ccccc4-c4ccccc4)ccc2n3-c2ccccc2-c2cccc3c2n(-c2ccccc2)c2c4ccccc4n(-c4ccccc4)c32)cc1 ; CC1(C)c2c(-c3ccccc3-n3c4ccc(-c5ccccc5-c5ccccc5)cc4c4cc(-c5ccccc5-c5ccccc5)ccc43)cccc2-c2oc3ccccc3c21 ; CC1(C)c2c(-c3ccccc3-n3c4ccc(-c5ccccc5-c5ccccc5)cc4c4cc(-c5ccccc5-c5ccccc5)ccc43)cccc2-c2c1c1ccccc1n2-c1ccccc1] 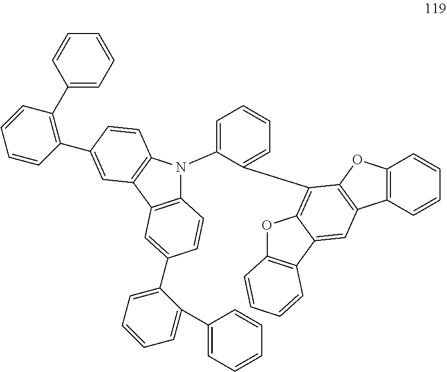 [SMILES: c1ccc(-c2ccccc2-c2ccc3c(c2)c2cc(-c4ccccc4-c4ccccc4)ccc2n3-c2ccccc2-c2c3oc4ccccc4c3cc3c2oc2ccccc23)cc1]
